(19) United States
(12) Patent Application Publication
(10) Pub. No.: US 2022/0175776 A1
(43) Pub. Date: Jun. 9, 2022

(54) COMBINATION OF CHIR99021 AND VALPROIC ACID FOR TREATING HEARING LOSS

(71) Applicant: Frequency Therapeutics, Inc., Lexington, MA (US)

(72) Inventors: Carl LEBEL, Swampscott, MA (US); Jenna HERBY, Fort Mill, SC (US); Will MCLEAN, North Haven, CT (US); Ashley HINTON, Farmington, CT (US); Moraye BEAR, Woodland Hills, CA (US); Christopher LOOSE, Winchester, MA (US)

(73) Assignee: Frequency Therapeutics, Inc., Lexington, MA (US)

(21) Appl. No.: 17/602,355

(22) PCT Filed: Apr. 8, 2020
(86) PCT No.: PCT/US2020/027316

§ 371 Date: Oct. 8, 2021

Related U.S. Application Data
(60) Provisional application No. 62/831,167, filed on Apr. 8, 2019.
(60) Provisional application No. 62/831,169, filed on Apr. 8, 2019.
(60) Provisional application No. 62/831,170, filed on Apr. 8, 2019.

(51) Int. Cl. A61K 31/506; A61K 31/19; A61K 47/10; A61K 47/20; A61K 9/00; A61P 27/16

(52) U.S. Cl. CPC A61K 31/506; A61K 31/19; A61P 27/16; A61K 47/20; A61K 9/0046; A61K 47/10

(57) ABSTRACT

Provided herein are compound(s) for use in treating sensorineural hearing loss in a human patient, for example, hair cell regeneration agent(s) for use in treating sensorineural hearing loss in a human. Also provided are methods of treating sensorineural hearing loss in a human by administering certain compound(s), for example, hair cell regeneration agent(s).

### RELATED APPLICATIONS

This application claims priority to U.S. Provisional Application No. 62/831,167 filed Apr. 8, 2019, U.S. Provisional Application No. 62/831,169 filed Apr. 8, 2019, and U.S. Provisional Application No. 62/831,170 Apr. 8, 2019 the contents of which are incorporated by reference in their entireties.

### FIELD OF THE INVENTION

The present disclosure relates to certain compound(s) for use in treating sensorineural hearing loss in a human patient.

### BACKGROUND OF THE INVENTION

Sensorineural hearing loss (SNHL) accounts for about 90% of all hearing loss (Li et al., Adv. Drug Deliv. Rev. 108, 2-12, 2017), with leading causes of SNHL being advanced age, ototoxic medications, and noise exposure (Liberman & Kujawa, Hear. Res. 349, 138-147, 2017).

SNHL typically arises from damage to and loss of sensory transducer cells (hair cells) within the sensory epithelium of the cochlea in the inner ear. Hair cells are susceptible to damage and, although some species such as birds, fish, and amphibians can regenerate hair cells throughout life, mammals lack this regenerative ability (Fujioka et al., Trends Neurosci. 38, 139-44, 2015). SNHL is defined by two hallmark symptoms: a decrease in hearing sensitivity (manifested in, for example, increased hearing level threshold) and difficulty understanding speech (particularly in noisy environments) (Edwards 2003). SNHL may also be accompanied by tinnitus, a condition in which a patient perceives a sound when no external sound stimulus is present. Tinnitus can have a substantial influence on patient well-being by affecting sleep, concentration or mood.

Loss of hearing sensitivity is often the direct result of damage to hair cells, particularly outer hair cells (OHCs). OHCs provide cochlear amplification on the order of 50 dB (Ryan & Dallos 1975). Thus, OHC death can lead to a decrease in sensitivity at frequency regions corresponding to the OHC loss.

Although the biological and physiological deficits driving poor speech intelligibility are less understood, synaptopathy and OHC function have been suggested as potential contributors to this deficit. Synaptopathy, or the uncoupling of hair cells from their respective nerve fibers, may lead to difficulty understanding speech in noise even in those patients with normal auditory thresholds as measured by standard audiometry (Kujawa and Liberman 2009; Wu et al. 2019). More recent findings indicate that good speech intelligibility in noise performance correlates with healthy OHC function (Hoben et al. 2017). In addition to amplification, OHCs provide sharp tuning of auditory signals (Liberman and Dodds 1984), which contributes to our ability to discern speech from a noisy background. Further, loss of sensitivity (OHCs) in the ultra-high frequencies of the cochlea (e.g., >8 kHz, beyond those tested by standard audiometry) affect the sharp cochlear tuning in the mid-frequency region, which is critically important for encoding speech (Badri et al. 2011).

The majority of patients with SNHL are managed with hearing aids or cochlear implants, but these devices do not repair the underlying biological deficit of cellular loss (see, for example, Ramakers et al., Laryngoscope 125, 2584-92, 2015; Raman et al., Effectiveness of Cochlear Implants in Adults with Sensorineural Hearing Loss. Agency for Healthcare Research and Quality (US), 2011; and Roche & Hansen, Otolaryngol. Clin. North Am. 48, 1097-116, 2015). While hearing aids and cochlear implants provide improvements in audibility, most users are still dissatisfied when listening in the presence of background noise (Gygi & Hall, 2016). When coupled with discomfort and stigma, this leads to an adoption rate of less than 25% of candidates (Lerner, 2019; Pratt, 2018; Sawyer et. al., 2019; Willink et. al., 2019). Despite improvements in implant technology, some qualified users still experience poor or declining speech recognition, poor sound quality, and complications in up to 15%-20% of cochlear implant patients (Health Quality Ontario, 2018).

In some cases, patients present to the clinic with normal pure tone audiometry thresholds but difficulty listening in the presence of background noise. This phenomenon is referred to as hidden hearing loss, and these patients do not benefit from hearing aids. Therefore, an experience of poor speech recognition may be observed both in patients with hearing deficits that are using implants, as well as patients having normal audiograms. In the case of implant users, although amplification of a sound stimulus may alleviate a hearing sensitivity issue, which may also provide improved speech recognition, failure to address the underlying biological deficit of cellular loss may mean that speech recognition remains an issue.

Thus, a regenerative treatment approach that improves hearing function, for example, by lowering hearing level thresholds, and/or by improving speech recognition, offers a major breakthrough for patients with sensorineural hearing loss or hidden hearing loss. Such a regenerative approach to treatment is in marked contrast to existing approaches that use hearing devices that manage the condition as opposed to treating the condition by restoring cochlear function.

Several approaches are being investigated to regenerate damaged or absent hair cells in mammalian inner ear sensory epithelia (reviewed in Mittal et al. Front Mol Neurosci. (2017); 10: 236). These include cell-based approaches (which aim to deliver exogenous cells to the inner ear to restore the sensory epithelia) and gene-based approaches (which aim to deliver exogenous genes to the sensory epithelia and reprogram endogenous cells to generate hair cells). For example, in animal models, adenovirus-mediated delivery of exogenous Atoh1 can stimulate cells within the sensory epithelia to differentiate into hair cells. One drawback with these approaches is the requirement to deliver cells or vectors to the inner ear of the patient. A treatment that can be delivered into, for example, the middle ear would be advantageous. A therapeutic method in which the endogenous signaling pathways of inner ear cells are modulated by exogenous agents are therefore attractive, because the delivery of such agents is likely to be more straightforward than cell-based or gene-based approaches.

Using molecular agents to initiate transdifferentiation, in which existing supporting cells of the cochlea are stimulated to differentiate into replacement hair cells, is one area of interest. Another area of interest is the activation of proliferative response in the supporting cells, in order to provide a new population of cells that could differentiate into hair cells, thereby replacing lost or damaged hair cells.

The combination of a Wnt pathway agonist (a glycogen synthase kinase 3 (GSK3) inhibitor) in combination with an agent that can have activity as an epigenetic modulator, for example, VPA, a compound that can, for example, act as a histone deacetylase complex (HDAC) inhibitor has shown promising results in stimulating the expansion of supporting cells in vitro and an in vivo animal model, as well as providing an improvement in loudness thresholds, as measured by ABR in an animal model (see McLean et al. Cell Rep. 2017 Feb. 21; 18(8): 1917-1929; WO 2017/151907). However, whether such treatments transfer to humans is unknown. It is also unknown how the severity of SNHL might impact response to a treatment. Furthermore, it is unknown whether a treatment can improve word recognition.

There remains a need for a successful treatment of sensorineural hearing loss and hidden hearing loss in humans.

### SUMMARY OF THE INVENTION

The inventors have discovered that, surprisingly, a combination treatment of a GSK3β inhibitor (e.g., CHIR99021) and an epigenetic modulator (e.g., valproate) can improve word recognition in human subjects with hearing loss. This combination treatment can also improve hearing thresholds at high frequencies. Accordingly, the present disclosure relates to treating sensorineural hearing loss in a human patient, for example, by using one or more hair cell regeneration agents and/or using a combination treatment using a Wnt agonist and an epigenetic modulator (for example, CHIR99021 and valproic acid, including their pharmaceutically acceptable salts etc.). One of the broad aspects disclosed herein is successful treatment of sensorineural hearing loss in humans by using any of the compounds or combinations of compounds disclosed herein, for example, by administration into the middle ear. Such a treatment is disclosed in combination with each and every one of the compounds, combinations of compounds and methods of therapy disclosed herein.

In one aspect or embodiment human patients may see improvements in hearing when treated in accordance with the invention, including improvements either not seen or that cannot be seen in animal models to date, as set out herein. For example, improvements are seen in the ability of patients to understand words, e.g., when those words are masked by background noise. To take another example, a limited change in a patient's ability to detect pure tones at certain frequencies can create a large improvement in their understanding of words, e.g., as defined herein. This in turn may suggest that the claimed therapies are improving hearing at higher frequencies than those generally tested to date, potentially including treatment of hidden hearing loss.

In any aspects, the improvements seen can occur rapidly, e.g., after a single dose and/or e.g., shortly after administration. In any aspects, those improvements remain long after the initial dose has been administered.

In any aspect or embodiment, particular groups of human patients with sensorineural hearing loss can be treated with certain classes of compounds as disclosed herein, based on the new human trial data presented in the examples. In addition, the invention relates to the extent to which such patients can be treated, for example, the significant improvements provided after only a single dose of the compound(s) in question, for example, into the middle ear.

In one more specific aspect, the invention relates to improvements in hearing in specific human patients, for example, those with hidden hearing loss, moderate hearing loss etc. as set out in detail herein.

The invention provides CHIR99021 or a pharmaceutically acceptable salt thereof and valproic acid (VPA) or a pharmaceutically acceptable salt thereof for use in treating sensorineural hearing loss in a human patient, wherein the sensorineural hearing loss is moderate or moderately severe sensorineural hearing loss.

The invention also provides CHIR99021 or a pharmaceutically acceptable salt thereof and valproic acid (VPA) or a pharmaceutically acceptable salt thereof for use in treating sensorineural hearing loss in a human patient, wherein the average of the patient's hearing thresholds across 0.5 kHz, 1 kHz, 2 kHz and 4 kHz is greater than 40 dB HL and no more than 70 dB HL when measured by pure tone audiometry prior to the treatment.

The invention also provides CHIR99021 or a pharmaceutically acceptable salt thereof and valproic acid (VPA) or a pharmaceutically acceptable salt thereof for use in treating sensorineural hearing loss in a human patient, wherein the patient has a standard word recognition score of 60% or less prior to the treatment.

The invention also provides CHIR99021 or a pharmaceutically acceptable salt thereof and valproic acid (VPA) or a pharmaceutically acceptable salt thereof for use in treating sensorineural hearing loss in a human patient, wherein the patient has a words-in-noise score of 50% or less prior to the treatment.

The invention also provides CHIR99021 or a pharmaceutically acceptable salt thereof and valproic acid (VPA) or a pharmaceutically acceptable salt thereof for use in treating sensorineural hearing loss in a human patient, wherein the sensorineural hearing loss is moderate or moderately severe sensorineural hearing loss; the average of the patient's hearing thresholds across 0.5 kHz, 1 kHz, 2 kHz and 4 kHz is greater than 40 dB HL and no more than 70 dB HL when measured by pure tone audiometry prior to the treatment; and the patient has a standard word recognition score of 60% or less prior to the treatment or a words-in-noise score of 50% or less prior to the treatment.

The invention also provides CHIR99021 or a pharmaceutically acceptable salt thereof and valproic acid (VPA) or a pharmaceutically acceptable salt thereof for use in treating tinnitus in a human patient with sensorineural hearing loss, wherein the tinnitus is diagnosed using one or more of the methods selected from the group consisting of: tinnitus functional index (TFI), tinnitus handicap index (THI), tinnitus reaction questionnaire (TRQ), tinnitus severity index (TSI), and tinnitus handicap questionnaire (THQ).

The invention also provides a hair cell regeneration agent(s) for use in treating sensorineural hearing loss in a human patient, wherein the sensorineural hearing loss is moderate or moderately severe sensorineural hearing loss

The invention also provides a hair cell regeneration agent(s) for use in treating sensorineural hearing loss in a human patient, wherein the average of the patient's hearing thresholds across 0.5 kHz, 1 kHz, 2 kHz and 4 kHz is greater than 40 dB HL and no more than 70 dB HL when measured by pure tone audiometry prior to the treatment.

The invention also provides a hair cell regeneration agent(s) for use in treating sensorineural hearing loss in a human patient, wherein the patient has a standard word recognition score of 60% or less prior to the treatment.

The invention also provides a hair cell regeneration agent(s) for use in treating sensorineural hearing loss in a human patient, wherein the patient has a words-in-noise score of 50% or less prior to the treatment.

The invention also provides a hair cell regeneration agent(s) for use in treating sensorineural hearing loss in a human patient, wherein said treatment provides an improved standard word recognition score for the patient, wherein said improvement, if tested, would be at least 10%, wherein said percentage improvement is calculated using the following formula:

$$1 \; 00 \times ( \frac{patient ’ s word recognition score after treatment}{patient ’ s word recognition score prior to treatment} - 1 )$$

The invention also provides hair cell regeneration agent(s) for use in treating sensorineural hearing loss in a human patient, wherein said treatment provides an improved words-in-noise score for the patient, wherein said improvement, if tested, would be at least 10%, wherein said percentage improvement is calculated using the following formula:

$$100 \times ( \frac{patient ’ s words in noise score after treatment}{patient ’ s words in noise score prior to treatment} - 1 )$$

The invention also provides a hair cell regeneration agent(s) for use in treating sensorineural hearing loss in a human patient, wherein said treatment provides an improved hearing threshold at 8 kHz, wherein said improvement, if tested, would be at least 5 dB relative to the patient's hearing threshold at 8 kHz prior to the treatment, wherein said hearing threshold is measured by pure tone audiometry.

The invention also provides a hair cell regeneration agent(s) for use in treating tinnitus in a human patient with sensorineural hearing loss, wherein the tinnitus is diagnosed using one or more of the methods selected from the group consisting of: tinnitus functional index (TFI), tinnitus handicap index (THI), tinnitus reaction questionnaire (TRQ), tinnitus severity index (TSI), and tinnitus handicap questionnaire (THQ).

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present invention, suitable methods and materials are described below. In the case of conflict, the present specification, including definitions, will control. In addition, the materials, methods and examples are illustrative only and are not intended to be limiting.

Other features and advantages of the invention will be apparent from the following detailed description and claims.

### BRIEF DESCRIPTION OF THE DRAWINGS

FIGS. 1A and 1B depict the study design of a phase 1/2 clinical study for FX-322.

FIG. 2 shows improvement in word recognition (WR) scores from a single dose of FX-322. (FIG. 2A) Individual WR performance showed improvements as early as day 15, and recoveries were sustained until the endpoint at day 90 (Dashed line=10% change from baseline). (FIG. 2B) FX-322 patients showed increased WR scores across day 15, 30, 60, and 90 whereas the placebo group did not improve; p=0.01, 2-tailed pairwise comparison of adjusted means between treatment groups. (FIG. 2C) A bivariate plot of Word Recognition in Quiet scores at Baseline and day 90. The diagonal dashed line represents equal performance between sessions. Curved dotted lines represent the 95% confidence interval based on Thornton and Raffin's binomial distribution (1978). Only FX-322 treated patients showed clinically significant improvement, as indicated by open diamonds outside the 95% confidence interval.

FIG. 3 shows improvement in words-in-noise (WIN) scores from a single dose of FX-322. (FIG. 3A) Individual WIN performance showed improvements as early as day 15, and recoveries were sustained until the endpoint at day 90 (Dashed line=10% change from baseline). (FIG. 3B) FX-322 patients showed a trend for greater overall increases from baseline in WIN tests scores across day 15, 30, 60, and 90 compared to placebo; p=0.211, 2-tailed pairwise comparison of adjusted means between treatment groups.

FIG. 4 shows the absolute change in WIN for each patient compared to baseline.

FIG. 5 shows psychometric functions for WIN data obtained at Baseline and Day 90. The point at which the horizontal line intersects with the psychometric function represents the predicted signal-to-noise ratio where 50% of the words were correctly identified. Improvement is indicated by a shift up and/or to the left in the function. FX-322 treated patients showed a statistically significant improvement from Baseline to Day 90, while placebo patients showed no improvement (p, n; ±SE).

FIG. 6 shows the difference in air audiometry at 8 kHz for each patient (FIG. 6A) and a 2-tailed, mixed model comparison of adjusted mean of the treated and placebo groups (FIG. 6B).

FIG. 7 shows the response rate for composite endpoint of ≥5 dB improvement and ≥10% improvement in WR or WIN test at day 90 post injection.

FIG. 8 shows the responder analysis for days 15, 30, 60 and 90 post injection.

FIG. 9 shows that some responders achieved 10 dB improvements (FIG. 9A) and that some responders improved at 6 and 8 kHz (FIG. 9B).

FIG. 10A, FIG. 10B, and FIG. 10C show a significant portion of responders have moderate and moderate severe hearing loss.

FIG. 11 shows the change in WR on day 90 post injection compared to baseline.

FIG. 12 shows the change from baseline at day 90 post injection for high frequency pure tone average at 4, 6 and 8 kHz.

FIG. 13 shows proportionality between dose cohorts (human) by plasma pharmacokinetics.

FIG. 14 shows an analysis of auditory brainstem responses for FX-322 treatment in a noise-damage model for induced hearing loss. Treatment with CHIR99021+VPA leads to hearing improvement in an in vivo noise damage model. (FIG. 14A) Image of injection procedure to transtympanically inject poloxamer into the middle ear of mice. (FIG. 14B) Animals designated to control and treated groups had elevated thresholds at 24 hours and 5 weeks after noise exposure compared to pre-exposure baseline. Control n=37 animals, treated n=47 animals. (FIG. 14C) At 5 weeks after injection, treated animals had significantly lower hearing thresholds relative to control animals for 4 of the 5 frequencies tested. (FIG. 14D) The distribution of individual hearing recoveries was analyzed. Values represent the change in dB needed to elicit an ABR response, with positive values representing further threshold increases (further hearing loss) and negative values representing threshold decreases (improved hearing). The fraction of animals with a given ABR change from 24 hours to 5 weeks are shown for each frequency tested. The treated group had a higher incidence of animals with hearing improvement and the greatest individual recoveries. Values are presented as means±SE; *=p<0.05, **=p<0.01, ***=p<0.001, ****=p<0.0001.

FIG. 15 shows an analysis of hair cell count for FX-322 treatment in a noise-damage model for induced hearing loss. (FIG. 15A) Low magnification view of a healthy isolated cochlear section showing complete rows if inner hair cells (IHCs) and outer hair cells (OHCs) (FIG. 15B) High magnification view of the region highlighted in a) showing intact IHCs and OHCs in mid frequency regions. (FIG. 15C) Cochleae of vehicle injected animals show widespread hair cell loss throughout the cochlea (apex and mid region shown. (FIG. 15D) High magnification view of the region highlighted in (FIG. 15C) showing substantial absence of hair cells in mid frequency regions, where a single IHC can be seen in the field of view (solid arrow). (FIG. 15E) Cochleae of CV treated animals show a greater overall population of hair cells compared to vehicle treated animals (apex and mid region shown). (FIG. 15F) High magnification view of the region highlighted in e) showing a complete row of IHCs (solid arrow) and a population of OHCs (open arrow). (FIG. 15G) CV treated cochlea (blue) show significantly more total hair cells, IHCs, and OHC relative to vehicle treated cochleae (grey). (FIG. 15H) The number of hair cells depicted as the percentage relative to an undamaged healthy cochlea. CV treated cochlea (blue) show significantly higher percentage of total hair cells, IHCs, and OHC relative to vehicle treated cochleae (grey). Scale bars, 100 μM low magnification, 20 μM high magnification. Values are presented as box-whisker plots; n=7 animals per group, *=p<0.05, **=p<0.01.

### DETAILED DESCRIPTION

### Sensorineural Hearing Loss

Provided in one aspect is a hair cell regeneration agent(s) for use in treating sensorineural hearing loss in a human patient. Also provided is a method of treating sensorineural hearing loss in a human patient comprising administering to the patient a hair cell regeneration agent(s). Also provided is a Wnt agonist and/or an epigenetic modulator for use in treating sensorineural hearing loss in a human patient, wherein said Wnt agonist and said epigenetic modulator are both administered to the patient. Also provided is a method of treating sensorineural hearing loss in a human patient comprising administering to the patient a Wnt agonist and/or an epigenetic modulator.

Sensorineural hearing loss accounts for approximately 90% of hearing loss and it often arises from damage or loss of hair cells in the cochlea. There are numerous causes of hair cell damage and loss, and the agents and treatments described herein may be used in the context of sensorineural hearing loss arising from any cause of hair cell damage or loss. For example, hair cells may be damaged and loss may be induced by noise exposure, leading to noise-induced sensorineural hearing loss. Thus, in some embodiments sensorineural hearing loss is noise-induced sensorineural hearing loss. Ototoxic drugs, for example, aminoglycosides and chemotherapy drug cisplatin, can also cause sensorineural hearing loss. In some embodiments sensorineural hearing loss is drug-induced sensorineural hearing loss. Infection may damage cochlear hair cells and may be a cause of sudden sensorineural hearing loss. In some embodiments sensorineural hearing loss is sudden sensorineural hearing loss (SSNHL). Hair cells can also be lost or damaged over time as part of the ageing process in humans. In some embodiments, sensorineural hearing loss is age-related sensorineural hearing loss (also known as presbycusis).

### Measurement of Sensorineural Hearing Loss

Hearing loss can be assessed by several different tests. Such tests may determine the audibility of a sound to a patient and/or the intelligibility of the sound to a patient prior to or after treatment. The audibility of a sound is a measure of a patient's ability to detect the sound (i.e., whether the patient can determine the presence or absence of a sound). The intelligibility of a sound is a measure of a patient's ability to correctly identify the sound. For instance, hearing may be assessed according to whether a patient can correctly identify a word or not. A patient with hearing loss may therefore neither be able to detect a sound nor correctly identify it (i.e., the sound is inaudible and unintelligible). However, audibility is not necessarily associated with intelligibility, and a patient may, for example, be able detect a sound, but not correctly identify it (i.e., the sound is audible but unintelligible).

### Pure Tone Audiometry

Assessment of a patient's audibility function is typically carried out by an audiologist using an audiometer in a hearing test known as pure tone audiometry. Pure tone audiometry is a standard test used to assess the audibility of a sounds and is described in detail elsewhere (see, for example, Katz, J., Medwetsky, L., Burkard, R., & Hood, L. (2009) Handbook of Clinical Audiology. Philadelphia, Pa.: Lippincott Williams and Wilkins). Pure tone audiometry is typically carried out in a sound-treated booth, which reduces ambient noise levels that may interfere with the detection of low-level sound stimuli.

In pure tone audiometry, a patient is exposed to pure tone stimuli at specific frequencies to determine the patient's hearing threshold at each frequency. Standard audiometry measures a patient's pure tone hearing threshold at each of the following frequencies 0.25 kHz, 0.5 kHz, 1 kHz, 2 kHz, 3 kHz, 4 kHz, 6 kHz, and 8 kHz. However, a patient's hearing threshold does not need to be determined at all of these frequencies to ascertain whether the patient has sensorineural hearing loss. For instance, a subset of frequencies, or a single frequency may be tested to identify a patient with sensorineural hearing loss.

To determine the hearing threshold, the volume of the pure tone is altered to determine the lowest level of stimuli that the patient can detect. The lowest level of stimuli (corresponding to the quietest sound) is the pure tone hearing threshold at a given frequency. The pure tone threshold is typically measured in a patient using decibels in hearing level (dB HL) on an audiometer. However, hearing thresholds may also be determined using other methods known to the person skilled in the art. For example, hearing function may be measured by Auditory Brainstem Response (ABR) testing or Auditory Steady State Response (ASSR) testing. Other tests can also be used to determine hearing function in a patient. For instance, otoacousic emission can be used to measure outer hair cell function, such as, Distortion product otoacoustic emissions (DPOAEs), Stimulus-Frequency otoacoustic emissions (SFOAEs), Transient-evoked otoacoustic emissions (TEOAEs), and Spontaneous otoacoustic emissions (SOAEs). Loss of otoacoustic emissions may be used in differential diagnosis of hearing loss arising from hair cell loss from hearing loss associated with higher level processing (e.g., auditory neuropathy).

Pure tone thresholds of a patient may be plotted on a graph to produce an audiogram representing these data.

Pure tone thresholds measured across different frequencies may also be averaged to provide a pure tone average. For instance, a patient that has pure tone hearing thresholds of 50 dB HL at 0.5 Hz, 60 dB HL at 1 kHz, 65 dB HL at 2 kHz, and 70 dB at 4 kHz would have a pure tone average of 61.25 dB HL, when measured across 0.5 kHz, 1 kHz, 2 kHz and 4 kHz.

Pure tone averages may be calculated across different frequencies. Pure tone thresholds at any subset of frequencies may be used to calculate pure tone averages. In some embodiments, the average of the patient hearing threshold is measured across 0.5 kHz, 1 kHz, and 2 kHz. In some embodiments, the average of the patient hearing threshold is measured across 0.5 kHz, 1 kHz, 2 kHz and 4 kHz. In some embodiments, pure tone average is measured across 4 kHz, 6 kHz, and 8 kHz. Measurement of pure tone average across 4 kHz, 6 kHz, and 8 kHz is useful when seeking to assess the patient's hearing function at the higher frequencies within the standard audiometric frequencies.

Sensorineural hearing loss can be categorized according to its severity. The severity of hearing loss is determined by the hearing levels at which a threshold level is obtained in a patient by pure tone audiometry. Severity of hearing loss is classified according to hearing thresholds using the following definitions:
Normal: 25 dB HL or less
Mild: at least 25 dB HL and no more than 40 dB HL, for example, greater than 25 dB HL and no more than 40 dB HL
Moderate: at least 40 dB HL and no more than 55 dB HL, for example, great than 40 dB HL and no more than 55 dB HL
Moderately Severe: at least 55 dB HL and no more than 70 dB HL, for example, greater than 55 dB HL and no more than 70 dB HL
Severe: at least 70 dB HL and no more than 90 dB HL, for example, greater than 70 dB HL and no more than 90 dB HL
Profound: at least 90 dB HL or more, for example, greater than 90 dB HL.
   These measures of severity are standard measures in the field (see Goodman, A. (1965). Reference zero levels for pure tone audiometer. ASHA, 7, 262-263). In some embodiments, the severity of hearing loss is classified according to a patient's hearing threshold at a single frequency (for example, 0.25 kHz, 0.5 kHz, 1 kHz, 2 kHz, 3 kHz, 4 kHz, 6 kHz, or 8 kHz). For instance, a patient may have mild hearing loss at 8 kHz, and normal hearing at the other standard audiometric frequencies. In some embodiments, the severity of hearing loss is classified according to pure tone average, when measured across a subset of frequencies. In certain such embodiments, the severity of hearing loss is classified according to the pure tone average across 0.5 kHz, 1 kHz, 2 kHz and 4 kHz. For example, a patient may have moderate hearing loss according to their pure tone average across 0.5 kHz, 1 kHz, 2 kHz and 4 kHz, but have moderately severe hearing loss at a single frequency (e.g., 8 kHz). In other embodiments, the severity of hearing loss is classified according to the pure tone average across 4 kHz, 6 kHz, and 8 kHz.

A patient that has hearing threshold of 25 dB HL or less at standard audiometric frequencies (i.e., 0.25 kHz, 0.5 kHz, 1 kHz, 2 kHz, 3 kHz, 4 kHz, 6 kHz, and 8 kHz) has normal hearing. The patient's audiogram is also a normal audiogram.

The inventors have found that patients with moderate or moderately severe hearing loss are particularly amenable to the treatments disclosed herein. Thus, in certain embodiments the sensorineural hearing loss is moderate sensorineural hearing loss. In other embodiments, the sensorineural hearing loss is moderately severe sensorineural hearing loss. In other embodiments, a therapeutic benefit may be provided in patient having less severe hearing loss than moderate sensorineural hearing loss. Thus, in some embodiments, sensorineural hearing loss is mild sensorineural hearing loss. In other embodiments, a therapeutic benefit may be provided in a patient having more severe sensorineural hearing loss than moderately severe hearing loss. In other embodiments, sensorineural hearing loss is severe sensorineural hearing loss. In other embodiments, sensorineural hearing loss is profound sensorineural hearing loss.

In some embodiments, the moderate or moderately severe sensorineural hearing loss is determined according to the average of the patient's hearing thresholds across 0.5 kHz, 1 kHz, 2 kHz, and 4 kHz when assessed by pure tone audiometry. In these embodiments, the average of the patient's hearing thresholds across 0.5 kHz, 1 kHz, 2 kHz, and 4 kHz when assessed by pure tone audiometry is at least 40 dB HL and no more than 70 dB HL (e.g., greater than 40 dB HL and no more than 70 dB HL). In certain such embodiments, the average of the patient's hearing thresholds across 0.5 kHz, 1 kHz, 2 kHz, and 4 kHz when assessed by pure tone audiometry is at least 40 dB HL and no more than 55 dB HL (e.g., greater than 40 dB HL and no more than 55 dB HL). In other embodiments, the average of the patient's hearing thresholds across 0.5 kHz, 1 kHz, 2 kHz, and 4 kHz when assessed by pure tone audiometry is at least 55 dB HL and no more than 70 dB HL (e.g., greater than 55 dB HL and no more than 70 dB HL).

The inventors have found that the patients with hearing loss at higher frequencies are particularly amenable to the treatments disclosed herein. Thus, in certain embodiments, the patient has more severe hearing loss at 4 kHz, and/or 6 kHz, and/or 8 kHz that at the other standard audiometric frequencies (i.e., 0.25 kHz, 0.5 kHz, 1 kHz, 2 kHz, and 3 kHz), when measured by pure tone audiometry. For example, in some embodiments the patient has moderate or moderately severe hearing loss at 4 kHz, 6 kHz, and 8 kHz and mild hearing loss at the other standard audiometric frequencies. In another embodiment, the patient has moderate hearing loss at 4 kHz, 6 kHz, and 8 kHz and mild hearing loss at the other standard audiometric frequencies. In another embodiment, the patient has mild hearing loss at 4 kHz, 6 kHz, and 8 kHz and normal hearing at the other standard audiometric frequencies.

In some embodiments, the patient has a hearing threshold of at least 40 dB HL at 4 kHz (e.g., greater than 40 dB HL at 4 kHz), when measured by pure tone audiometry. In some embodiments, the patient has a hearing threshold of at least 40 dB HL at 6 kHz (e.g., greater than 40 dB HL at 6 kHz), when measured by pure tone audiometry. In some embodiments, the patient has a hearing threshold of at least 40 dB HL at 8 kHz (e.g., greater than 40 dB HL at 8 kHz), when measured by pure tone audiometry.

In some embodiments, the patient has an audiogram with hearing thresholds in the following ranges when measured by pure tone audiometry:

8 kHz-40 dB HL to 95 dB HL; and/or

6 kHz-40 dB HL to 85 dB HL; and/or

4 kHz-40 dB HL to 80 dB HL; and/or

3 kHz-40 dB HL to 70 dB HL; and/or

2 kHz-40 dB HL to 70 dB HL; and/or

1 kHz-40 dB HL to 70 dB HL; and/or

0.5 kHz-40 dB HL to 70 dB HL; and/or

0.25 Hz-40 dB HL to 70 dB HL.

In some embodiments, the patient has an audiogram with a hearing threshold in the range of 40 dB HL to 95 dB HL at 8 kHz, when measured by pure tone audiometry. In certain such embodiments, the patient has an audiogram with a hearing threshold in the range of 40 dB HL to 70 dB HL at 8 kHz, when measured by pure tone audiometry.

In some embodiments, the patient has an audiogram with a hearing threshold in the range of 40 dB HL to 85 dB HL at 6 kHz, when measured by pure tone audiometry. In certain such embodiments, the patient has an audiogram with a hearing threshold in the range of 40 dB HL to 70 dB HL at 6 kHz, when measured by pure tone audiometry.

In some embodiments, the patient has an audiogram with a hearing threshold in the range of 40 dB HL to 80 dB HL at 4 kHz, when measured by pure tone audiometry. In certain such embodiments, the patient has an audiogram with a hearing threshold in the range of 40 dB HL to 70 dB HL at 4 kHz, when measured by pure tone audiometry.

In some embodiments, the patient has an audiogram with a hearing threshold in the range of 40 dB HL to 70 dB HL at 3 kHz, when measured by pure tone audiometry.

In some embodiments, the patient has an audiogram with a hearing threshold in the range of 40 dB HL to 70 dB HL at 2 kHz, when measured by pure tone audiometry.

In some embodiments, the patient has an audiogram with a hearing threshold in the range of 40 dB HL to 70 dB HL at 1 kHz, when measured by pure tone audiometry.

In some embodiments, the patient has an audiogram with a hearing threshold in the range of 40 dB HL to 70 dB HL at 0.5 kHz, when measured by pure tone audiometry.

In some embodiments, the patient has an audiogram with a hearing threshold in the range of 40 dB HL to 70 dB HL at 0.25 kHz, when measured by pure tone audiometry.

In some embodiments, the patient has an audiogram with hearing thresholds in the following ranges when measured by pure tone audiometry:

8 kHz-40 dB HL to 95 dB HL, and

6 kHz-40 dB HL to 85 dB HL; and

4 kHz-40 dB HL to 80 dB HL; and

3 kHz-40 dB HL to 70 dB HL; and

2 kHz-40 dB HL to 70 dB HL; and

1 kHz-40 dB HL to 70 dB HL, and

0.5 kHz-40 dB HL to 70 dB HL; and

0.25 Hz-40 dB HL to 70 dB HL.

In some embodiments, the patient has an audiogram with hearing thresholds in the following ranges when measured by pure tone audiometry:

8 kHz-40 dB HL to 70 dB HL; and

6 kHz-40 dB HL to 70 dB HL; and

4 kHz-40 dB HL to 70 dB HL; and

3 kHz-40 dB HL to 70 dB HL; and

2 kHz-40 dB HL to 70 dB HL, and

1 kHz-40 dB HL to 70 dB HL; and

0.5 kHz-40 dB HL to 70 dB HL; and

0.25 Hz-40 dB HL to 70 dB HL.

In some embodiments, mild sensorineural hearing loss is determined according to the average of the patient's hearing thresholds across 0.5 kHz, 1 kHz, 2 kHz, and 4 kHz when assessed by pure tone audiometry. In these embodiments, the average of the patient's hearing thresholds is at least 25 dB HL and no more than 40 dB HL (e.g., greater than 25 dB HL and no more than 40 dB HL).

In some embodiments, the patient has a hearing threshold of at least 25 dB HL and no more than 40 dB HL at 4 kHz (e.g., greater than 25 dB HL and no more than 40 dB HL at 4 kHz), when measured by pure tone audiometry. In some embodiments, the patient has a hearing threshold of at least 25 dB HL and no more than 40 dB HL at 6 kHz (e.g., greater than 25 dB HL and no more than 40 dB HL at 6 kHz), when measured by pure tone audiometry. In some embodiments, the patient has a hearing threshold of at least 25 dB HL and no more than 40 dB HL at 8 kHz (e.g., greater than 25 dB HL and no more than 40 dB HL at 8 kHz), when measured by pure tone audiometry.

In some embodiments, the patient has an audiogram with hearing thresholds in the following ranges when measured by pure tone audiometry:

8 kHz-25 dB HL to 40 dB HL; and/or

6 kHz-25 dB HL to 40 dB HL; and/or

4 kHz-25 dB HL to 40 dB HL; and/or

3 kHz-25 dB HL to 40 dB HL, and/or

2 kHz-25 dB HL to 40 dB HL; and/or

1 kHz-25 dB HL to 40 dB HL; and/or

0.5 kHz-25 dB HL to 40 dB HL; and/or

0.25 Hz-25 dB HL to 40 dB HL.

In some embodiments, the patient has an audiogram with a hearing threshold in the range of 25 dB HL to 40 dB HL at 8 kHz, when measured by pure tone audiometry.

In some embodiments, the patient has an audiogram with a hearing threshold in the range of 25 dB HL to 40 dB HL at 6 kHz, when measured by pure tone audiometry.

In some embodiments, the patient has an audiogram with a hearing threshold in the range of 25 dB HL to 40 dB HL at 4 kHz, when measured by pure tone audiometry.

In some embodiments, the patient has an audiogram with a hearing threshold in the range of 25 dB HL to 40 dB HL at 3 kHz, when measured by pure tone audiometry.

In some embodiments, the patient has an audiogram with a hearing threshold in the range of 25 dB HL to 40 dB HL at 2 kHz, when measured by pure tone audiometry.

In some embodiments, the patient has an audiogram with a hearing threshold in the range of 25 dB HL to 40 dB HL at 1 kHz, when measured by pure tone audiometry.

In some embodiments, the patient has an audiogram with a hearing threshold in the range of 25 dB HL to 40 dB HL at 0.5 kHz, when measured by pure tone audiometry.

In some embodiments, the patient has an audiogram with a hearing threshold in the range of 25 dB HL to 40 dB HL at 0.25 kHz, when measured by pure tone audiometry.

In some embodiments, the patient has an audiogram with hearing thresholds in the following ranges when measured by pure tone audiometry:

8 kHz-25 dB HL to 40 dB HL; and

6 kHz-25 dB HL to 40 dB HL; and

4 kHz-25 dB HL to 40 dB HL; and

3 kHz-25 dB HL to 40 dB HL; and

2 kHz-25 dB HL to 40 dB HL; and

1 kHz-25 dB HL to 40 dB HL; and

0.5 kHz-25 dB HL to 40 dB HL; and

0.25 Hz-25 dB HL to 40 dB HL.

The treatments disclosed herein are also suitable for use in patients having an audiogram with hearing thresholds at different severity levels for each of the standard audiometric frequencies. For example, a patient may have moderate hearing loss at a first frequency, mild hearing loss at a second frequency etc. These patients may therefore have an audiogram in which some hearing thresholds in the mild hearing loss range (i.e., at least 25 dB HL and no more than 40 dB HL (e.g., greater than 25 dB HL and no more than 40 dB HL)) and other hearing thresholds fall in the moderate hearing loss range (i.e., at least 40 dB HL and no more than 55 dB HL (e.g., greater than 40 dB HL and no more than 55 dB HL)). In certain embodiments, the patient has an audiogram with hearing thresholds in the moderate hearing loss range at 4 kHz, 6 kHz, and 8 kHz and hearing thresholds in the mild hearing loss range at 0.25 kHz, 0.5 kHz, 1 kHz, 2 kHz, and 3 kHz.

Hearing function in a patient can also be assessed at frequencies falling outside the standard audiometric range. For example, hearing function may be assessed in the ultra-high frequencies (also known as extended-high frequencies). Ultra-high frequencies in the context of pure tone audiometry are frequencies above 8 kHz. Hearing function in the ultra-high frequency range can be assessed by pure tone audiometry which may be performed at 10 kHz, 12 kHz, 14 kHz, and 16 kHz. Hearing function in the ultra-high frequency range can also be assessed by pure tone audiometry which may be performed at 9 kHz, 10 kHz, 11 kHz, 12 kHz, 14 kHz, and 16 kHz. The severity of hearing loss in the ultra-high frequency range may be classified according to the hearing thresholds used to classify severity of hearing loss in the standard audiometric frequency range. The severity of hearing loss in the ultra-high frequency range is classified using the following ranges:
Normal: 25 dB HL or less
Mild: at least 25 dB HL and no more than 40 dB HL, for example, greater than 25 dB HL and no more than 40 dB HL
Moderate: at least 40 dB HL and no more than 55 dB HL, for example, greater than 40 dB HL and no more than 55 dB HL
Moderately Severe: at least 55 dB HL and no more than 70 dB HL, for example, greater than 55 dB HL and no more than 70 dB HL
Severe: at least 70 dB HL and no more than 90 dB HL, for example, greater than 70 dB HL and no more than 90 dB HL
Profound: at least 90 dB HL or more, for example, greater than 90 dB HL

In some embodiments, the severity of hearing loss in the ultra-high frequency range is classified according to a patient's hearing threshold at a single ultra-high frequency (for example, 10 kHz, 12 kHz, 14 kHz, or 16 kHz). The severity of hearing loss at a single ultra-high frequency may be mild, moderate, moderately severe, severe or profound, as summarized above. For instance, in some embodiments, a patient may have mild hearing loss at 16 kHz, and normal hearing at the other ultra-high frequencies. In other embodiments, a patient may have moderate hearing loss at 16 kHz and mild hearing loss at the other ultra-high frequencies. In some embodiments, the severity of hearing loss is classified according to pure tone average, when measured across a subset of ultra-high frequencies. Any subset of ultra-high frequencies may be used to calculate pure tone average. In certain such embodiments, the severity of hearing loss is classified according to the pure tone average across 10 kHz, 12 kHz, 14 kHz, and 16 kHz. In other embodiments, the severity of hearing loss is classified according to the pure tone average across 9 kHz, 10 kHz, 11 kHz, 12 kHz, 14 kHz, and 16 kHz.

A patient having sensorineural hearing loss when assessed at standard audiometric frequencies may also have hearing loss in the ultra-high frequencies. Thus, in some embodiments the patient having sensorineural hearing loss also has a hearing threshold of between 40 dB HL to 70 dB HL at 16 kHz when measured by pure tone audiometry. In some embodiments, the patient has an audiogram with hearing thresholds in the following ranges when measured by pure tone audiometry:

16 kHz-40 dB HL to 70 dB HL; and/or

14 kHz-40 dB HL to 85 dB HL; and/or

12 kHz-40 dB HL to 95 dB HL; and/or

10 kHz-40 dB HL to 95 dB HL.

In some embodiments, the patient has an audiogram with a hearing threshold has in the range of 40 dB HL to 85 dB HL at 14 kHz, when measured by pure tone audiometry.

In some embodiments, the patient has an audiogram with a hearing threshold has in the range of 40 dB HL to 95 dB HL at 12 kHz, when measured by pure tone audiometry.

In some embodiments, the patient has an audiogram with a hearing threshold has in the range of 40 dB HL to 95 dB HL at 10 kHz, when measured by pure tone audiometry.

### Word Recognition Tests

Alternatively, or in addition to pure tone audiometry, hearing loss may be assessed using a word recognition test. A word recognition test measures the patient's ability to correctly identify a word, thereby providing a measure of sound intelligibility (in particular, speech intelligibility) that may not be provided by pure tone audiometry. In some embodiments, a word recognition score is used to determine the patient's ability to correctly identify words prior to treatment.

The inventors have found that the treatments disclosed herein may be particularly effective at improving sound intelligibility and so patients having poor word recognition scores may be particularly suitable for the disclosed treatments.

A standard word recognition in quiet test, also referred to herein as a standard word recognition test, is a test administered by an audiologist that measures a patient's speech intelligibility in recognizing words in a quiet environment. A quiet environment is an environment with little to no background noise.

A standard word recognition test may be used to determine a person's ability to recognize words selected from a word list and presented to the patient at a given decibel (dB) level. In some embodiments, the standard word recognition test is used to determine a patient's ability to recognize words at more than one decibel level.

In some embodiments, the standard word recognition test assesses the patient's ability to identify 50 words. However, the number of words presented to the patient may be more or less than 50. For example, in some embodiments, the standard word recognition test is for 25 words. In other embodiments, the standard word recognition test is for 10 words.

A standard word recognition test may be used to generate a standard word recognition (%) score which is calculated using the formula:

$$standard word recognition score ( \% ) = 100 \times ( \frac{words recognized in standard word recognition test}{total words} )$$

In some embodiments, the patient has a standard word recognition score of 90% or less, 85% or less, or 80% or less, 70% or less, 60% or less, or 50% or less prior to treatment. In some embodiments, the patient has a standard word recognition score of 60% or less prior to treatment. In any of the above embodiments, the patient may have a standard word recognition score of at least 10%, 15%, or 20% prior to the treatment. For example, in certain such embodiments, the patient has a standard word recognition score of between 10% and 90% prior to the treatment, or 15% and 90% prior to the treatment, or 20% and 90% prior to the treatment.

In some embodiments, the patient has a standard word recognition score of between 10% and 90% prior to the treatment, or 10% and 85% prior to the treatment, or 10% and 80% prior to the treatment, or 10% and 70% prior to the treatment, or 10% and 60% prior to the treatment, or 10% and 50% prior to the treatment.

In one embodiment the patient has a standard word recognition score of between 10% and 80% prior to the treatment. In another embodiment the patient has a standard word recognition score of between 10% and 60% prior to the treatment.

In another embodiment, the patient has a standard word recognition score of between 15% and 85% prior to the treatment.

In another embodiment, the patient has a standard word recognition score of between 20% and 80% prior to the treatment.

In another embodiment, the patient has a standard word recognition score of 80% or less prior to the treatment.

In some embodiments, the standard word recognition score is expressed as the number of words that are correctly recognized in the test. For example, in some embodiments the patient identifies 45 or fewer words, 42 or fewer words, 40 or fewer words, 35 or fewer words, 30 or fewer words, or 25 or fewer words correctly in a standard word recognition test for 50 words. In some embodiments, the patient identifies 30 or fewer words correctly in a standard word recognition test for 50 words.

In any of the above embodiments, the patient may correctly identify at least 5, 7, or 10 words. For instance, in certain such embodiments, the patient correctly identifies between 5 and 63 words, 7 and 63 words, or 10 and 63 words.

In some embodiments, the patient correctly identifies between 5 and 45 words, 5 and 42 words, 5 and 40 words, 5 and 35 words, 5 and 30 words, or 5 and 25 words in a standard word recognition test for 50 words.

In one embodiment, the patient correctly identifies between 5 and 40 words in a standard word recognition test for 50 words.

In another embodiment, the patient correctly identifies between 7 and 43 words in a standard word recognition test for 50 words.

In another embodiment, the patient correctly identifies between 10 and 40 words in a standard word recognition test for 50 words.

In another embodiment, the patient correctly identifies 40 or fewer words in a standard word recognition test for 50 words.

In some embodiments, a list of words is administered to each ear, and a standard word recognition score is calculated for each ear. Herein the results of the standard word recognition score refer to the ear that has been/will be treated.

A standard word recognition test may be carried out using any list of words. However, standard word lists are typically used in a standard word recognition test. In some embodiments, each test word is embedded in a carrier phrase. Example of carrier phrases are: “Say the word ______ again”, “You will say ______”, or “Say the word ______”.

In some embodiments, the standard word recognition test is the Maryland consonant-vowel nucleus-consonant (CNC) word test. The Maryland CNC word test has been described, for example, in Mendel, L. L., Mustain, W. D., & Magro, J. (2014). Normative data for the Maryland CNC Test. Journal of the American Academy of Audiology, 25, 775-781.

The Maryland CNC word test is a standard word recognition test that uses phonemically balanced word lists comprising words that are consonant-nucleus-consonant (CNC) monosyllables. These CNC lists are balanced so that each initial consonant, each vowel, and each final consonant appears with the same frequency within each list. The Maryland CNC test has 10 lists of 50 words.

In some embodiments, the Maryland CNC Test uses words from Lehiste and Peterson's phonemically balanced word lists, all of which were CNC monosyllables, for example, as described in Lehiste I, Peterson G E. (1959) Linguistic considerations in the study of speech intelligibility. Journal of the Acoustical Society of America 31(3): 280-286.

In some embodiments, the Maryland CNC Test uses words from revised CNC lists that eliminate rare literary words and proper names, for example, as described in Peterson G E, Lehiste I. (1962) Revised CNC lists for auditory tests. Journal of Speech and Hearing Disorders 27:62-70.

In some embodiments, the Maryland CNC Test uses words from modified CNC word lists that take into consideration the effects of coarticulation, where the acoustic properties of phonemes are influenced by those phonemes that immediately precede and follow them, for example, as described in Causey G D, Hood L J, Hermanson C L, Bowling L S. (1984) The Maryland CNC Test: normative studies. Audiology 23(6): 552-568. The words of the Maryland CNC test are spoken within the carrier phrase: ‘Say the ______ again,’

In some embodiments, the standard word recognition test is the C.I.D Auditory Test W-22 (CID W-22) test. The CID W-22 test has been described, for example, in Hirsh, I. J., Davis, H. Silverman, S. R., Reynolds, E. G., Eldert, E., & Benson, R. W. (1952). Development of Materials for Speech Audiometry. Journal of Speech, Language, and Hearing Research, 17(3), 321-337.

The CID W-22 test uses 200 monosyllabic words which are divided into four lists of 50 words each. Each list is phonetically balanced. The speech sounds within the list occur with the same relative frequency as they do in a representative sample of English speech. There are three criteria for the vocabulary in the phonetically balanced word lists. First, all the words must be one-syllable words with no repetition of words in the different lists. Second, any word chosen should be a familiar word. This second criterion is to minimize the effect of differences in the educational background of subjects. Third, the phonetic composition of each word list should correspond to that of English as a whole as closely as possible. The words of the CID W-22 test are spoken with the carrier phrase: “You will say ______”.

In some embodiments the standard word recognition test is the NU No. 6 test. The NU No. 6 has been described, for example, in Tillman, T. W., & Carhart, R. (1966). An expanded test for speech discrimination utilizing CNC monosyllabic words: Northwestern University Auditory Test No. 6. Northwestern Univ Evanston Il Auditory Research Lab.

In some embodiments, the NU No. 6 test uses 4 lists of 50 words, for example, as described in Table 28-2 of Tillman, T. W., & Carhart, R. (1966). The words of the NU No. 6 test are spoken with the carrier phrase: “Say the word ______”.

In some embodiments the standard word recognition test is the Maryland CNC test, using the words list and carrier phrases as defined in Causey G D, Hood L J, Hermanson C L, Bowling L S. (1984) The Maryland CNC Test: normative studies. Audiology 23(6): 552-568. In certain such embodiments, the word signal is provided to the patient at 40 dB above speech perception or recognition level. In other embodiments, the word signal is provided to the patient at 30 dB above speech perception or recognition level.

### Words-In-Noise (WIN) Test

A “Words-in-Noise (WIN) Test” is a test administered by an audiologist to measure a patient's speech intelligibility in recognizing words in the presence of background noise.

The WIN test consists of administering words to an ear at a varying signal-to-noise ratio (SNR) level. The signal-to-noise ratio is the ratio of the strength of the signal carrying information (e.g., the test word signal) relative to the signal of interference (e.g., noise), and is typically expressed in decibels. In some embodiments, the background noise is multi-talker babble at a fixed decibel level.

In some embodiments the multi-talker babble is comprised of six talkers (three female, three male) at a fixed level, for example, as described in Wilson, R. H., Abrams, H. B., & Pillion, A. L. (2003). A word-recognition task in multi-talker babble using a descending presentation mode from 24 dB to 0 dB signal to babble. Journal of Rehabilitation Research and Development, 40(4), 321-328.

In some embodiments, the background noise is maintained at a fixed decibel level, and the variation in the SNR decibel level is achieved by varying the decibel level of the test word signal. The SNR decibel level is therefore the SNR above the background noise. For example, if the level of multi-talker babble is fixed at 70 dB SPL, and the level of the test word signal varied from 70 dB SPL to 94 dB SPL, this would give a SNR decibel level variation of 0 dB to 24 dB. In another example, the level of multi-talker babble is fixed at 80 dB SPL, and the level of the test word signal varied from 80 dB SPL to 104 dB SPL.

In some embodiments, the test words that are used may be from any list described herein for the word recognition tests. In some embodiments, the word-in-noise test is for 70 words. In other embodiments, the words-in-noise test is for 35 words.

In some embodiments, the test consists of administering 35 or 70 monosyllabic words from the NU No. 6 word lists. The test words may be spoken with the carrier phrase: “Say the word ______”.

In some embodiments, the WIN test is administered in a descending-level SNR paradigm. In these embodiments, the test words at the high SNR decibel level are presented first, followed by test words at gradually lower SNR decibel levels, with words at the lowest SNR decibel level administered last. The high SNR decibel level is the easiest setting for the patient to identify the signal words. The low SNR decibel levels is the most difficult setting for the patient to identify the signal words. In other embodiments, the WIN test is administered in a randomized-level SNR paradigm. In these embodiments, the test words are presented at different SNR decibel levels in a randomized order.

In some embodiments the SNR decibel level of the test words varies from 24 dB SNR (easiest condition) to 0 dB SNR (most difficult condition) in 4 dB decrements, for a total of seven SNR levels (i.e., 24 dB SNR, 20 dB SNR, 16 dB SNR, 12 dB SNR, 8 dB SNR, 4 dB SNR, and 0 dB SNR).

In some embodiments the WIN test consists of administering 70 monosyllabic words from the NU No. 6 word lists, where the SNR decibel level of the test words varies from 24 dB SNR (easiest condition) to 0 dB SNR (most difficult condition) in 4 dB decrements, for a total of seven SNR levels (i.e., 24 dB SNR, 20 dB SNR, 16 dB SNR, 12 dB SNR, 8 dB SNR, 4 dB SNR, and 0 dB SNR). In one embodiment, the level of multi-talker babble is fixed at 70 dB SPL, and the level of the test word signal varies from 70 dB SPL to 94 dB SPL. In another embodiment, the level of multi-talker babble is fixed at 80 dB SPL, and the level of the test word signal varied from 80 dB SPL to 104 dB SPL

The ‘words-in-noise’ test may be used to generate a words-in-noise score.

In some embodiments the words-in-noise score is given as a percentage of the total correct words recognized by the patient in the test and calculated using the formula:

$$words in noise score ( \% ) = 10 \times ( \frac{words recognized in standard words in noise test}{total words} )$$

In some embodiments, the patient has a words-in-noise score of 90% or less, 80% or less, 70% or less, 60% or less, 50% or less, 40% or less, or 30% or less prior to treatment. In some embodiments, the patient has a words-in-noise score of 50% or less prior to treatment. In any of the above embodiments, the patient may have a word-in-noise score of at least 10%, at least 15%, or at least 20% prior to the treatment. For example, in certain such embodiments, the patient has a word-in-noise score of between 10% and 70% prior to the treatment, or between 15% and 70% prior to the treatment, or between 20% and 70% prior to the treatment.

In some embodiments, the patient has a word-in-noise score of between 10% and 90% prior to the treatment, or between 10% and 80% prior to the treatment, or between 10% and 70% prior to the treatment, or between 10% and 60% prior to the treatment, or between 10% and 50% prior to the treatment, or between 10% and 40% prior to the treatment, or between 10% and 30% prior to the treatment.

In one embodiment the, patient has a words-in-noise score of between 10% and 70% prior to the treatment.

In one embodiment, the patient has a words-in-noise score of 70% or less prior to the treatment.

In some embodiments the words-in-noise score is expressed as the number of words that are correctly recognized in the test. For example, in some embodiments the patient correctly identifies 63 or fewer, 56 or fewer, 49 or fewer, 42 or fewer, 35 or fewer, 28 or fewer, or 21 or fewer words in a word-in-noise test for 70 words. In some embodiments, the patient correctly identifies 35 or fewer words in a words-in-noise test for 70 words. In any of the above embodiments, the patient may correctly identify at least 7, 10, or 13 words. For instance, in certain such embodiments, the patient correctly identifies between 7 and 49 words, 10 and 49 words, or 13 and 49 words.

In some embodiments, the patient correctly identifies between 7 and 63 words, 7 and 56 words, 7 and 49 words, 7 and 42 words, 7 and 35 words, 7 and 28 words, or 7 and 21 words in words-in-noise test for 70 words.

In one embodiment, the patient correctly identifies between 7 and 49 words in a words-in-noise test for 70 words.

In one embodiment, the patient correctly identifies 49 or fewer words in a words-in-noise test for 70 words.

In other embodiments, the patient correctly identifies 32 or fewer, 28 or fewer, 24 or fewer, 21 or fewer, 17 or fewer, 14 or fewer, or 11 or fewer words in a words-in-noise test for 35 words. In any of the above embodiments, the patient may correctly identify at least 3, 5, or 7 words. For instance, in certain such embodiments, the patient correctly identifies between 3 and 24 words, 5 and 24 words, or 7 and 24 words.

In some embodiments, the patient correctly identifies between 3 and 32 words, 3 and 28 words, 3 and 24 words, 3 and 21 words, 3 and 17 words, 3 and 14 words, or 3 and 11 words in a words-in-noise test for 35 words.

In one embodiment, the patient correctly identifies between 3 and 24 words in a words-in-noise test for 35 words.

In one embodiment, the patient correctly identifies 24 or fewer words in a words-in-noise test for 35 words.

In some embodiments the patient's signal-to-noise ratio (SNR) for the predicted mean of 50% correct words in a words-in-noise test is calculated using the words-in-noise score at each SNR level and the Spearman-Karber equation. The predicted mean of 50% correct words is used to provide the mean dB SNR level at which the person is expected to identify 50% of the words correctly in a words-in-noise test. In some embodiments, the patient's SNR for a predicted mean of 50% correct words in a words-in-noise test is about 25 dB, about 24 dB, about 23 dB, about 22 dB, about 21 dB, about 20 dB, about 19 dB, 18 dB, about 17 dB, about 16 dB, about 15 dB, about 14 dB, about 13 dB, about 12 dB, about 11 dB, about 10 dB, about 9 dB, about 8 dB, about 7 dB, or about 6 dB. In some embodiments, the patient's signal-to-noise ratio (SNR) for the predicted mean of 50% correct words in a words-in-noise test is about 21 dB, for example, 20.8 dB, about 20 dB, about 19 dB, for example, 18.8 dB, about 18 dB, for example, 17.6 dB, about 17 dB, for example, 16.8 dB, or about 16 dB, for example, 16.4 dB.

For patients whose preferred language is not English, comparable tests provided in a preferred language can be used, and are intended to be encompassed by terms such as ‘standard word recognition’ and “words-in-noise” test. For instance, in German the Freiburg Speech Intelligibility Test may be used (see, for example, Hoth, HNO 2016 64:540-548). In Spanish, the Castilian Spanish Hearing in Noise Test (HINT) may be used (for example, as described in Huarte, International Journal of Audiology 2008 47:369037). When a standardized test is not established, e.g., for a particular language, a suitable test used in the art may be used.

### Hidden Hearing Loss

In some embodiments, the patient has hidden hearing loss.

In the context of this disclosure, a patient with “hidden hearing loss” has a difficulty hearing in noisy environments but does not have sensorineural hearing loss when assessed at standard audiometric frequencies (and so has a normal audiogram). A patient with hidden hearing loss therefore has normal hearing function in terms of audibility but reduced intelligibility function. The reduced intelligibility function may become apparent when the patient is presented with background noise. Recent work has suggested that hidden hearing loss may arise from damage at the synapses between hair cells and cochlear neurons (Lieberman et al. PLoS One 2016 11(9):e0162726). In this study, hidden hearing loss was associated with elevated hearing thresholds at ultra-high frequencies and reduced performance in a words-in-noise test.

In some embodiments, a patient with hidden hearing loss has hearing thresholds of less than 25 dB HL at 0.25 kHz, 0.5 kHz, 1 kHz, 2 kHz, 3 kHz, 4 kHz, 6 kHz, and 8 kHz and a words-in-noise score of 90% or less, 80% or less, 70% or less, 60% or less, or 50% or less prior to the treatment. In some embodiments, a patient has hearing thresholds of less than 25 dB HL at 0.25 kHz, 0.5 kHz, 1 kHz, 2 kHz, 3 kHz, 4 kHz, 6 kHz, and 8 kHz; and a words-in-noise score of 90% or less, 80% or less, 70% or less, 60% or less, or 50% or less prior to the treatment, but has not necessarily been diagnosed with hidden hearing loss. In some embodiments, the patient has hearing thresholds of less than 25 dB HL at 0.25 kHz, 0.5 kHz, 1 kHz, 2 kHz, 3 kHz, 4 kHz, 6 kHz, and 8 kHz and a words-in-noise score of 60% or less prior to the treatment.

A patient with hidden hearing loss may have hearing thresholds that are higher than normal in the ultra-high frequency range. Thus, in some embodiments, the patient with hidden hearing loss also has a hearing threshold of between 40 dB HL to 70 dB HL at 16 kHz when measured by pure tone audiometry. In some embodiments, the patient with hidden hearing loss has an audiogram with hearing thresholds in the following ranges when measured by pure tone audiometry:

16 kHz-40 dB HL to 70 dB HL; and/or

14 kHz-40 dB HL to 85 dB HL; and/or

12 kHz-40 dB HL to 95 dB HL; and/or

10 kHz-40 dB HL to 95 dB HL.

In some embodiments, the patient with hidden hearing loss has an audiogram with a hearing threshold has in the range of 40 dB HL to 85 dB HL at 14 kHz, when measured by pure tone audiometry.

In some embodiments, the patient with hidden hearing loss has an audiogram with a hearing threshold in the range of 40 dB HL to 95 dB HL at 12 kHz, when measured by pure tone audiometry.

In some embodiments, the patient with hidden hearing loss has an audiogram with a hearing threshold in the range of 40 dB HL to 95 dB HL at 10 kHz, when measured by pure tone audiometry.

A patient that has hearing thresholds of less than 25 dB HL at 0.25 kHz, 0.5 kHz, 1 kHz, 2 kHz, 3 kHz, 4 kHz, 6 kHz and 8 kHz and a words-in-noise score of 90% or less, 80% or less, 70% or less, 60% or less, or 50% or less prior to the treatment, but has not necessarily been diagnosed with hidden hearing loss, may also have hearing thresholds that are higher than normal in the ultra-high frequency range. In certain such embodiments, the patient also has a hearing threshold of between 40 dB HL to 70 dB HL at 16 kHz when measured by pure tone audiometry. In certain such embodiments, the patient has an audiogram with hearing thresholds in the following ranges when measured by pure tone audiometry:

16 kHz-40 dB HL to 70 dB HL; and/or

14 kHz-40 dB HL to 85 dB HL; and/or

12 kHz-40 dB HL to 95 dB HL; and/or

10 kHz-40 dB HL to 95 dB HL.

In some embodiments, the patient has an audiogram with a hearing threshold in the range of 40 dB HL to 85 dB HL at 14 kHz, when measured by pure tone audiometry.

In some embodiments, the patient has an audiogram with a hearing threshold in the range of 40 dB HL to 95 dB HL at 12 kHz, when measured by pure tone audiometry.

In some embodiments, the patient has an audiogram with a hearing threshold in the range of 40 dB HL to 95 dB HL at 10 kHz, when measured by pure tone audiometry.

A patient with hidden hearing loss will typically have poor performance in a words-in-noise test. The words-in-noise test described herein may be used to identify a patient amenable to the treatment as described herein.

In some embodiments, the patient with hidden hearing loss has a words-in-noise score of 90% or less, 80% or less, 70% or less, 60% or less, or 50% or less prior to treatment. In some embodiments, the patient with hidden hearing loss has a words-in-noise score of 60% or less prior to treatment.

In some embodiments, the patient with hidden hearing loss correctly identifies 63 or fewer, 54 or fewer, 49 or fewer, 42 or fewer, or 35 or fewer words in a word-in-noise test for 70 words. In other embodiments, the patient correctly identifies 32 or fewer, 28 or fewer, 24 or fewer, 21 or fewer, or 17 or fewer words in a words-in-noise test for 35 words.

In some embodiments the patient with hidden hearing loss has a signal-to-noise ratio for a predicted mean of 50% correct words in a words-in-noise test is about 18 dB, about 17 dB, about 16 dB, about 15 dB, about 14 dB, about 13 dB, about 12 dB, about 11 dB, about 10 dB, about 9 dB, about 8 dB, about 7 dB, about 6 dB, about 5 dB, or about 4 dB. In some embodiments, the patient's signal-to-noise ratio (SNR) for the predicted mean of 50% correct words in a words-in-noise test is about 11 dB, about 10 dB, or about 9 dB.

### Tinnitus

In some embodiments, the patient has tinnitus. In certain such embodiments, the patient has sensorineural hearing loss as defined elsewhere herein and tinnitus. In other embodiments, the patient has hidden hearing loss as defined elsewhere herein and tinnitus.

Tinnitus may be assessed or diagnosed using one or more measures selected from the group consisting of: tinnitus functional index (TFI), tinnitus handicap index (THI), tinnitus reaction questionnaire (TRQ), tinnitus severity index (TSI), tinnitus handicap questionnaire (THQ), and tinnitus questionnaire (TQ). In some embodiments, tinnitus is assessed or diagnosed using the tinnitus functional index (TFI).

For example, the Tinnitus Functional Index (TFI) and Tinnitus Handicap Inventory (THI) may be used to assess the burden of tinnitus.

### Tinnitus Function Index (TFI)

The TFI has eight subscales which assess (i) the intrusiveness of tinnitus, (ii) the sense of control the patient has, (iii) cognitive interference, (iv) sleep disturbance, (v) auditory issues, (vi) relaxation issues, (vii) quality of life (QOL), and (viii) emotional distress (Henry et al. 2014 and Meikle et al. 2012). The patient fills in a questionnaire of 25 questions which asks the patient to quantify the impact of tinnitus in different areas of their life. The patient's answers to these questions are then used to calculate a TFI score, which indicates the how severe the problem of tinnitus is to the patient. TFI scores are as follows:

Mean score of 14 (range 0-17) not a problem;

Mean score of 21 (range 18-31) small problem;

Mean score of 42 (range 32-53) moderate problem;

Mean score of 65 (range 54-72) big problem;

Mean score of 78 (range 73-100) very big problem.

### Tinnitus Handicap Inventory (THI)

The THI has 23 questions for a patient to answer, which allows identification, quantification, and evaluation of the difficulty of tinnitus experience (as described in Noble 1998). The answers to the questions are used to calculate a THI score out of 100. THI scores are graded as follows:

1-16: Slight or no handicap (Grade 1);

18-36: Mild handicap (Grade 2);

38-56: Moderate handicap (Grade 3);

58-76: Severe handicap (Grade 4);

78-100: Catastrophic handicap (Grade 5)

### Tinnitus Reaction Questionnaire (TRQ)

The TRQ is a 26-item questionnaire to quantify the psychological distress associated with tinnitus stemming from four general symptom categories: general distress, interference, severity, and avoidance. Each item is scored on a 5 point scale (0: not al all, 4 points: almost all of the time) (see, for example, Wilson et al. Journal of Speech and Hearing Research (1991) 34: 197-201)

### Tinnitus Severity Index (TSI):

The TSI is a 12-item questionnaire and measures how much tinnitus negatively affects a patient's life, and how bothersome patients perceive their tinnitus to be. Questions 1-9 deal with interference and are rated from 1 (Never) to 5 (Always). Questions 10, 11, and 12 probe sleep, effort, and discomfort.

### Tinnitus Handicap Questionnaire (THQ)

The THQ is a 27-item questionnaire designed to assess perceived attitudes and reactions of others in the areas of life quality, concentration difficulties, discomfort in quiet environments, ability to suppress tinnitus, anxiety and worry, and tense or irritable feelings. It is the only questionnaire that has been designed to assess the influence of significant others in the overall management process (see, for example, Kuk et al. Ear and Hearing (1990) 11(6):434-445).

### Tinnitus Questionnaire (TQ)

The TQ is a 52-item questionnaire which assesses five dimensions of tinnitus complaint: emotional distress, auditory perceptual difficulties, intrusiveness, sleep disturbance, and somatic complaints. Each question relates directly to the “noises” in the ear as the major cause or source of distress and reflect inappropriate or lack of coping skills. Subjects indicate their agreement to each statement using one of three response alternatives: true (2 points), partly true (1 point), or not true (0 points). (see, for example. Baguley et al. The Journal of Laryngology & Otology (2000) 114:840-843).

Tinnitus may also be assessed using other means established in the field (see, for example, Newman, C. W., Sandridge, S. A., & Snow, J. B. (2004). Tinnitus questionnaires. Tinnitus: Theory and management, 237-254)

The different measurements of tinnitus have been compared. For instance, measurement of TFI and THI have good agreement in assessing the severity of tinnitus. TFI showed exceptionally high internal consistency (α≥0.95), high construct validity with the THI (r=0.80) and high test-retest reliability (ICC=0.87) (Fackrell et al. 2018). TFI and TQ have also been shown to have good agreement with one another (Jacquemin et al. 2019).

### Therapeutic Effect

Effective treatment of sensorineural hearing loss may be determined using different criteria. These criteria can be categorized as either improvements in sound audibility or improvements in sound intelligibility or both. An improvement in audibility function means that the patient has an improved ability to detect when a sound is present or absent. In other words, an improvement in audibility means that the patient is able to detect the presence of a quieter sound. An improvement in sound intelligibility means that the patient has improved ability to correctly identify a sound. In some embodiments, the treatment provides the patient with improved audibility function. In some embodiments, the treatment provides the patient with improved intelligibility function. In some embodiments, the treatment provides the patient with improved audibility function and improved intelligibility function.

An improvement in audibility function may be associated with an improvement in intelligibility function. For example, in these situations, the patient may be able to detect the sound of a word more easily, and correctly identify the word. However, in other situations, an improvement in audibility may not be associated with an improvement in intelligibility. In these situations, a patient may now be able to hear a word, but unable to correctly identify the word. An improvement in audibility is nevertheless advantageous as it may allow a patient to hear sounds that were previously inaudible to the patient.

In other situations, a patient may experience little or no change in audibility function as measured by standard audiometry tests yet nonetheless experience an improvement in intelligibility function following treatment. For example, in these situations a patient may be able to detect the presence of a word stimulus at the same sound level as prior to the treatment, but is now able to correctly identify the word, whereas prior to the treatment the word was incorrectly identified. An improvement in intelligibility is an important therapeutic benefit because as a result a patient may be able to understand more sounds in a real world situation. Thus, in some embodiments, the treatment provides the patient with improved intelligibility function. In some situations, a patient may experience little or no change in audibility function as measured by standard audiometry tests but nonetheless an improvement in audibility function is observed at the ultra-high frequencies.

Improvements in audibility may be measured using pure tone audiometry as described herein. However, an improvement in audibility does not necessarily need to be measured in order for an improvement to be provided by the treatment. Similarly, an improvement in intelligibility may be measured using word recognition tests as described herein. However, an improvement in intelligibility does not necessarily need to be measured in order for an improvement to be provided by the treatment. The treatments described herein may be used to provide an improvement in hearing function without measurement of hearing function before and after the treatment.

The inventors have found that the treatments described herein may be particularly effective at improving audibility function at high frequencies. Thus, in some embodiments, the treatment provides an improved hearing threshold at 4 kHz, 6 kHz, and/or 8 kHz. This improvement may be observed as a reduced pure tone threshold at 4 kHz, 6 kHz, and/or 8 kHz, when measured by pure tone audiometry. In some embodiments, the patient has a reduced pure tone threshold at 4 kHz after treatment relative to the patient's pure tone threshold prior to treatment. In some embodiments, the patient has a reduced pure tone threshold at 6 kHz after treatment relative to the patient's pure tone threshold prior to treatment. In some embodiments, the patient has a reduced pure tone threshold at 8 kHz after treatment relative to the patient's pure tone threshold prior to treatment.

In some embodiments the improved hearing threshold at 4 kHz, 6 kHz, and/or 8 kHz is at least 5 dB relative to the patient's hearing threshold at 4 kHz, 6 kHz and/or 8 kHz prior to the treatment. In some embodiments the improved hearing threshold at 4 kHz, 6 kHz, and/or 8 kHz is at least 10 dB relative to the patient's hearing threshold at 4 kHz, 6 kHz, and/or 8 kHz prior to the treatment. In some embodiments the improved hearing threshold at 4 kHz, 6 kHz, and/or 8 kHz is at least 20 dB relative to the patient's hearing threshold at 4 kHz, 6 kHz, and/or 8 kHz prior to the treatment. In some embodiments the improved hearing threshold at 4 kHz, 6 kHz, and/or 8 kHz is at least 30 dB relative to the patient's hearing threshold at 4 kHz, 6 kHz, and/or 8 kHz prior to the treatment.

In some embodiments, the treatment provides an improved hearing threshold of at least 5 dB at 8 kHz relative to a patient's hearing threshold at 8 kHz prior to the treatment, when measured by pure tone audiometry.

In some embodiments, the treatment provides an improved hearing threshold of at least 5 dB at 6 kHz relative to a patient's hearing threshold at 6 kHz prior to the treatment, when measured by pure tone audiometry.

In a particular embodiment, the treatment provides an improved hearing threshold of at least 5 dB at 6 kHz and 8 kHz relative to a patient's hearing threshold at 6 kHz and 8 kHz prior to the treatment, when measured by pure tone audiometry.

In some embodiments, the improvement in audibility is assessed using the average of the patient's pure tone thresholds when measured across 4 kHz, 6 kHz, and 8 kHz. In certain embodiments, the treatment provides an improvement to the average of the patient hearing thresholds across 4 kHz, 6 kHz, and 8 kHz when measured by pure tone audiometry, wherein said improvement is at least 1, 2, 3, 4, 5, 6, 7, 8, 9, 10, 12, 15, 20, 25, or 30 dB relative to the average of the patient hearing thresholds across 4 kHz, 6 kHz, and 8 kHz when measured by pure tone audiometry prior to the treatment.

Improvements in audibility function may be observed in an individual patient, or as an average across a population of patients.

Improvements in intelligibility may be measured using word recognition tests as described herein. Improvements in intelligibility function may be observed in an individual patient, or as an average across a population of patients.

Improvements in audibility and/or intelligibility may be observed in a percentage of patients in a population of patients. In some embodiments, an improvement in audibility, as assessed according to any of the methods described herein, is observed in at least 20%, at least 30%, at least 40% or at least 50% of a patient population. In some embodiments, an improvement in intelligibility, as assessed according to any of the methods described herein, is observed in at least 20%, at least 30%, at least 40%, or at least 50% of a patient population.

In some embodiments, improvement in intelligibility is measured using a standard word recognition score, as described herein. Alternatively, or in addition to, improvement in intelligibility may be measured using a words-in-noise test, as described herein.

The inventors have found that the treatments described herein are effective at improving intelligibility of words when assessed using a standard word recognition test. Accordingly, in some embodiments, the treatment provides an improved standard word recognition score, wherein said improvement is at least 10%, at least 20%, at least 30%, at least 50%, at least 70%, at least 100%, at least 200%, at least 300%, at least 400%, at least 500%, at least 600%, at least 700%, at least 800%, or at least 900% wherein said percentage improvement is calculated using the following formula:

$$100 \times ( \frac{patient ’ s word recognition score after treatment}{patient ’ s word recognition score prior to treatment} - 1 )$$

In some embodiments, the improved word recognition score is at least 10% wherein said percentage improvement is calculated using the following formula:

$$100 \times ( \frac{patient ’ s word recognition score after treatment}{patient ’ s word recognition score prior to treatment} - 1 )$$

A standard word recognition test of 50 words may be used to assess hearing function. In some embodiments, the treatment provides an improved standard word recognition for the patient, wherein said improvement, if tested, would be at least 5, at least 10, or at least 15 words relative to the number of words recognized by the patient in a standard word recognition test of 50 words prior to the treatment.

In some embodiments, the treatment provides an improved standard word recognition for the patient, wherein said improvement, if tested, would be at least 5 words relative to the number of words recognized by the patient in a standard word recognition test of 50 words prior to the treatment.

An improvement in the number of words recognized by a patient in a standard word recognition test may also be expressed as a percentage of the number of words in the standard word recognition test. Accordingly, in some embodiments, the treatment provides an improved standard word recognition score for the patient, wherein said improvement, if tested, would be at least 6%, at least 10%, at least 20%, at least 25%, at least 30%, at least 35%, at least 40%, or at least 50%, wherein said percentage improvement is calculated using the following formula:

$$100 \times ( \frac{WR score post treatment - WR score prior to treatment}{number of words in standard word recognition test} )$$

In some embodiments, the criteria set out in Thornton and Raffin (1978) for determining whether a change in standard word recognition score represents a significant change in a patient's ability to recognize words is used to assess word recognition scores before and after treatment. In some embodiments, the treatment provides an improved standard word recognition score for the patient, wherein said improvement, if tested, is a word recognition score falling outside the 95% confidence interval for the patient's word recognition score prior to the treatment as defined by Thornton and Raffin (1978). In other embodiments, a 99% confidence interval is used. In other embodiments a 97.5% confidence interval is used. In other embodiments a 90% confidence interval is used. In other embodiments an 85% confidence interval is used. These confidence intervals may be calculated in radians using the arcsine transformation for proportions of words recognized as detailed by Studebaker (1985). In these embodiments, radian confidence intervals are converted back to word recognition proportions per the iterative procedure detailed by Thornton and Raffin

Confidence intervals for changes in word recognition scores may also be calculated using other established methods. For instance, Carney and Schalch (2007) describe a refinement of the Thornton and Raffin framework which may also be used to determine whether a change in standard word recognition score represents a significant change in a patient's ability to recognize words.

In another embodiment, the 95% confidence interval bounds may be calculated in radians using the arcsine transformation for proportions of words recognized as detailed by Studebaker (1985). In this embodiment, radian confidence intervals are converted back to word recognition proportions per the iterative procedure detailed by Thornton and Raffin.

The inventors have found that the treatments described herein are also effective at improving intelligibility of sounds in background noise. Thus, in some embodiments, the treatment provides an improved words-in-noise score for the patient, wherein said improvement is at least 10%, at least 20%, at least 30%, at least 50%, at least 70%, at least 100%, at least 200%, at least 300%, at least 400%, at least 500%, at least 600%, at least 700%, at least 800%, or at least 900% wherein said percentage improvement is calculated using the following formula:

$$100 \times ( \frac{patient ’ s words in noise score after treatment}{patient ’ s words in noise score prior to treatment} - 1 )$$

In some embodiments, the improved words-in-noise score is at least 10% wherein said percentage improvement is calculated using the following formula:

$$100 \times ( \frac{patient ’ s words in noise score after treatment}{patient ’ s words in noise score prior to treatment} - 1 )$$

A words-in-noise test of 70 words may be used to assess hearing function. Accordingly, in some embodiments, the treatment provides improved words-in-noise recognition for the patient, wherein said improvement, if tested, would be at least 5, at least 7, or at least 10 words relative to the number of words recognized by the patient in a words-in-noise test of 70 words prior to the treatment.

In one embodiment, the treatment provides an improved words-in-noise recognition for the patient, wherein said improvement, if tested, would be at least 5 words relative to the number of words recognized by the patient in a words-in-noise test of 70 words prior to the treatment.

A words-in-noise test of 35 words may be used to assess hearing function. Thus, in some embodiments, the treatment provides improved words-in-noise recognition for the patient, wherein said improvement, if tested, would be at least 2, at least 3, or at least 5 words relative to the number of words recognized by the patient in a words-in-noise test of 35 words prior to the treatment.

In some embodiments, the treatment provides improved words-in-noise recognition for the patient, wherein said improvement, if tested, would be at least 2 words relative to the number of words recognized by the patient in a words-in-noise test of 35 words prior to the treatment.

An improvement in the number of words recognized by a patient in words-in-noise test may also be expressed as a percentage of the number of words in the words-in-noise test. Accordingly, in some embodiments, the treatment provides an improved words-in-noise score for the patient, wherein said improvement, if tested, would be at least 6%, at least 10%, at least 20%, at least 25%, at least 30%, at least 35%, at least 40%, or at least 50%, wherein said percentage improvement is calculated using the following formula:

$$100 \times ( \frac{WIN score post treatment - WIN score prior to treatment}{number of words in words in noise test} )$$

An improvement in words-in-noise recognition may also be assessed using the Signal-to-Noise ratio (SNR) for the predicted mean of 50% correct words in a words-in-noise test for the patient. In some embodiments, the criteria set out in Wilson & McArdle, 2007 for determining whether a change in SNR represents a significant change in a patient's ability to recognize words is used to assess words-in-noise scores.

In some embodiments, the treatment provides an improved Signal-to-Noise ratio (SNR) for the predicted mean of 50% correct words in a words-in-noise test for the patient, wherein said improvement, if tested, would be at least 3 dB, relative to the patient's SNR for the predicted mean of 50% correct words in a words-in-noise test prior to the treatment, wherein the SNR is calculated using the Spearman-Kärber equation.

An improvement in words-in-noise score may be seen without a corresponding improvement in audibility function. Accordingly, in some embodiments, the treatment provides an improved words-in-noise score without a change in audibility function when measured by pure tone audiometry. In certain such embodiments, the average of the patient's hearing thresholds across 0.5 kHz, 1 kHz, 2 kHz and 4 kHz after the treatment is no more than 5 dB increased or decreased to the average of the patient's hearing thresholds across 0.5 kHz, 1 kHz, 2 kHz, and 4 kHz prior to the treatment, wherein said hearing thresholds are measured by pure tone audiometry.

In some embodiments, the treatment provides (i) an improved hearing threshold at 8 kHz, wherein said improvement, if tested, would be at least 5 dB relative to the patient's hearing threshold at 8 kHz prior to the treatment, wherein said hearing threshold is measured by pure tone audiometry and (ii) an improved standard word recognition score for the patient or an improved words-in-noise score for the patient, wherein said improvement in standard word recognition score, if tested, would be at least 10%, wherein said percentage improvement is calculated using the following formula:

$$100 \times ( \frac{patient ’ s word recognition score after treatment}{patient ’ s word recognition score prior to treatment} - 1 )$$

wherein said improvement in words-in-noise score, if tested, would be at least 10%, wherein said percentage improvement is calculated using the following formula:

$$100 \times ( \frac{patient ’ s words in noise score after treatment}{patient ’ s words in noise score prior to treatment} - 1 )$$

In certain such embodiments, the treatment also provides an improved hearing threshold at 6 kHz, wherein said improvement, if tested, would be at least 5 dB relative to the patient's hearing threshold at 6 kHz prior to the treatment.

Improvement in sound intelligibility may be particularly relevant in the context of treating two patient groups that have normal audibility function but reduced intelligibility function. These two groups are (i) patients with hidden hearing loss, and (ii) patients having hearing thresholds within normal ranges (i.e., up to 25 dB) at standard audiometric frequencies (0.25 kHz-8 kHz) yet have difficulty in perceiving sound correctly. These patients typically show reduced function in a words-in-noise test. Thus, for patients in either of these patient groups, an effective treatment manifests in an improved intelligibility function. An improvement in audibility function may also be observed. Without wishing to be bound by theory, the improvement in words-in-noise score may arise due to the treatment providing an improvement in the ultra-high frequency range.

The inventors have found that an improvement in audibility function and/or intelligibility function may be observed shortly after treatment. In some embodiments, the treatment provides an improvement in audibility function and/or intelligibility function within 15, 30, 60, or 90 days after treatment (e.g., after initial treatment or after the completion of treatment). In some embodiments, an improvement in audibility function and/or intelligibility function is provided within 90 days.

The improvement in audibility function and/or intelligibility function may be maintained following treatment.

In some embodiments, the improvement is maintained until at least 90, 120, 180 or 365 days.

In certain embodiments, the improvement is maintained until at least 90 days.

In certain embodiments, the improvement is maintained until at least 120 days.

In certain embodiments, the improvement is maintained until at least 180 days.

In certain embodiments, the improvement is maintained until at least 365 days.

An improvement in audibility function and/or intelligibility function may be provided by a single administration of a hair cell regeneration agent(s) and/or Wnt agonist and/or epigenetic modulator as described herein. In some embodiments, the treatment comprises no more than a single administration.

In some embodiments, the treatment provides for treatment of tinnitus. Without wishing to be bound by theory, the improvement in tinnitus may arise through restoration or repair of a damaged or disrupted auditory pathway in the cochlea. For instance, regeneration of outer or inner hair cells may alleviate disruption of the auditory pathway in the cochlea by providing lost input, as is often seen by increasing auditory input using hearing aids.

In certain such embodiments, the patient has sensorineural hearing loss as defined elsewhere herein and tinnitus. In other embodiments, the patient has hidden hearing loss as defined elsewhere herein and tinnitus. In some embodiments, the treatment provides for treatment of tinnitus and sensorineural hearing loss as defined elsewhere herein. In other embodiments, the treatment provides for treatment of tinnitus and hidden hearing loss as defined elsewhere herein.

Tinnitus may be assessed using one or more measures selected from the group consisting of: tinnitus functional index (TFI), tinnitus handicap index (THI), tinnitus reaction questionnaire (TRQ), tinnitus severity index (TSI), tinnitus handicap questionnaire (THQ), and tinnitus questionnaire (TQ). In some embodiments, tinnitus is assessed using the tinnitus functional index (TFI).

In some embodiments, the treatment provides for treatment of tinnitus as assessed by TFI or TQ. Clinical significance may be defined as a decrease on the TFI (for example, a decrease of ≥13 points) and/or the TQ (for example, a decrease of ≥12 points) (Henry et al. 2015). Typically, an assessment of tinnitus is made at least three times before treatment, and three times after treatment.

In some embodiments, the treatment provides for treatment of tinnitus as assessed by TFI, wherein, if tested, the TFI score would be at least a decrease of 13, 14, 15, 16, 17, 18, 20, 25, or 30 points relative to the patient's TFI score prior to the treatment. In some embodiments, the treatment provides for treatment of tinnitus as assessed by TFI, wherein, if tested, the TFI score would be at least a decrease of 13 points relative to the patient's TFI score prior to the treatment.

In some embodiments, the treatment provides for treatment of tinnitus as assessed by TQ, wherein, if tested, the TQ score would be at least a decrease of 12, 13, 14, 15, 17, 19, 21, 26, or 31 points relative to the patient's TQ score prior to the treatment. In some embodiments, the treatment provides for treatment of tinnitus as assessed by TQ, wherein, if tested, the TQ score would be at least a decrease of 12 points relative to the patient's TQ score prior to the treatment.

Improvements in tinnitus may be observed in an individual patient, or as an average across a population of patients.

### Hair Cell Regeneration Agents

A hair cell regeneration agent is an agent that promotes regeneration of hair cells. A single agent may be used as a hair cell regeneration agent or a combination of agents may provide the hair cell regenerative function. Thus, in some embodiments, the hair cell regeneration agent is a single agent. In other embodiments the hair cell regeneration agent is a combination of agents. In certain such embodiments, the combination of agents may be formulated together in a single composition. In other embodiments, the combination of agents may be provided to a patient separately.

A hair cell regeneration agent may promote regeneration of hair cells by stimulating transdifferentiation of supporting cells within the sensory epithelium of cochlea into replacement hair cells. Alternatively, or additionally, a hair cell regeneration agent may activate a proliferative response in the sensory epithelium of the cochlea, thereby providing a new population of cells that can subsequently differentiate into supporting cells.

In some embodiments, the hair cell regeneration agent stimulates proliferation of cochlear supporting cells in which proliferation is stimulated expresses Lgr5 (Leucine-rich repeat-containing G-protein coupled receptor 5). However, the hair cell regeneration agent may also stimulate proliferation of supporting cells with little or no Lgr5 expression. In some embodiments, the hair cell regeneration agent produces an expanded population of cochlea cells. In some embodiments, the expanded cells are enriched for Lgr5 expression (i.e., a greater percentage of the expanded cell population express Lgr5 compared to the starting cell population).

Lgr5 is a member of GPCR class A receptor proteins that is expressed across a diverse range of tissues such as in the muscle, placenta, spinal cord and brain, and particularly as a biomarker of adult stem cells in certain tissues. Lgr5+ stem cells are the precursors for sensory hair cells that are present in the cochlea. Increasing the population of Lgr5+ cochlear cells is therefore beneficial because it increases the population of precursor cells which may differentiate into sensory hair cells.

In some embodiments, the hair cell regeneration agent is a Wnt agonist and an epigenetic modulator. Any Wnt agonist and epigenetic modulator described herein may be used.

In some embodiments, the hair cell regeneration agent is a Wnt agonist and two or more epigenetic modulators. Any Wnt agonist and epigenetic modulator described herein may be used.

In some embodiments, the hair cell regeneration agent is a Wnt agonist alone. A Wnt agonist may be used alone in line with any of the treatments disclosed herein that relate to Wnt agonists and/or epigenetic modulators in which both the Wnt agonist and epigenetic modulator are administered to the patient. In these embodiments, the epigenetic modulator is not included. Any Wnt agonist described herein may be used. In certain such embodiments, the hair cell regeneration agent is a GSK3 inhibitor. Any GSK3 inhibitor described herein may be used.

In some embodiments, the hair cell regeneration agent is gamma secretase inhibitor. Suitable gamma secretase inhibitors are described in WO 2018007331 A1; WO 2018111926 A2; WO 2018065340 A1; WO 2018060300 A1; WO 2018011164 A1; WO 2018087018 A1; WO 2018001918 A1; WO 2018118791 A2; WO 2018118782 A2 and WO 2014045156 A1, each of which is incorporated by reference. Any gamma secretase inhibitor described herein may be used.

In some embodiments, the hair cell regeneration agent is an Atoh1 activator. Suitable Atoh1 activators are described in US 20160030445 A1; WO 2018172997 A1; WO 2016022776 A2; WO 2014145205 A2 and WO 2009100438 A2, each of which is incorporated by reference.

In some embodiments, the hair cell regeneration agent is a Notch inhibitor. Suitable Notch inhibitors are described in WO2017007702-A1; WO2016056999-A1; WO2014039781A1; WO2014047369A1; WO2014047372A1; WO2014047390A1; WO2014047391A1; WO2014047397A1; WO2014047392A1; WO2014047370A1; WO2014047374A1; WO2013093885A1; WO2013178821A1 and WO2013016081A1, each of which is incorporated by reference.

In some embodiments, the hair cell regeneration agent is a Wnt agonist and a Notch inhibitor. Any Wnt agonist and Notch inhibitor may be used as described herein. In certain such embodiments the Wnt agonist is a GSK3 inhibitor. Any GSK3 inhibitor described herein may be used.

In some embodiments, the hair cell regeneration agent is a Wnt agonist and a gamma secretase inhibitor. Any Wnt agonist and gamma secretase inhibitor may be used as described herein. In certain such embodiments, the Wnt agonist is a GSK inhibitor. Any GSK3 inhibitor described herein may be used.

### WNT Agonists

Provided in one aspect is a Wnt agonist and/or an epigenetic modulator for use in treating sensorineural hearing loss in a human patient, wherein said Wnt agonist and said epigenetic modulator are administered to a human patient. Also provided is a method of treating sensorineural hearing loss in a human patient comprising administering to the patient a Wnt agonist and an epigenetic modulator. A Wnt agonist and/or an epigenetic modulator may be used for treating a patient as described elsewhere herein.

A Wnt agonist refers to an agent that increases the expression, levels, and/or activity of a Wnt gene, protein, or signaling pathway (e.g., TCF/LEF, Frizzled receptor family, Wif1, Lef1, Axin2, and/or β-catenin) in a cell, for example, a cochlear cell. A Wnt agonist includes a GSK3 inhibitor, such as a GSK3-α or a GSK3-β inhibitor. In some embodiments the Wnt agonist is a GSK inhibitor that inhibits both GSK3-α and GSK3-β.

The TCF/LEF family is a group of transcription factors that bind to DNA through a high mobility group domain, and which are involved in the Wnt signaling pathway where they recruit the coactivator β-catenin to enhancer elements of targeted genes. Frizzled is a family of G protein-coupled receptor proteins that serves as receptors in the Wnt signaling pathway. Frizzled receptors inhibit intracellular β-catenin degradation and activate TCF/LEF-mediated transcription.

In some embodiments, the Wnt agonist increases Wnt signaling in a cochlear cell by about or at least about 10, 20, 30, 40, 50, 60, 70, 80, 90, 100, 200, 300, 400, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more) or more relative to a control, for example, relative to a baseline level of activity.

In some embodiments, the Wnt agonist increases TCF/LEF-mediated transcription in a cochlear cell, for example, by about or at least about 10, 20, 30, 40, 50, 60, 70, 80, 90, 100, 200, 300, 400, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more) or more relative to a control, for example, relative to a baseline level of activity.

In some embodiments, the Wnt agonist binds and activates a Frizzled receptor family member, for example, by about or at least about 10, 20, 30, 40, 50, 60, 70, 80, 90, 100, 200, 300, 400, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more) or more relative to a control, for example, relative to a baseline level of activity.

In some embodiments, the Wnt agonist inhibits GSK3 for example, by about or at least about 10, 20, 30, 40, 50, 60, 70, 80, 90, 100, 200, 300, 400, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more or more relative to a control, for example, relative to a baseline level of activity.

In some embodiments, the Wnt agonist preferentially upregulates Jag-1, Deltex-1, or Hif-1 more than the Wnt agonist upregulates Hes or Hey. In some embodiments, the Wnt agonist increases the expression of Jag-1, Deltex-1, and/or Hif-1 10%, 25%, 50%, 75%, 100%, 125%, 150%, 175%, 200%, 250% or more than it increases the expression or activity of Hes and Hey.

Exemplary agents having activity as a Wnt agonist are provided in Table 1 and 2 below, including pharmaceutically acceptable salts thereof.

TABLE 1

| | | GSK- | | | | Formul. |
|---|---|---|---|---|---|---|
| | | 3 | GSK-3 | Lgr5+ | Perilym | Conc. |
| Agent | CAS | alpha | alpha | Assay | ph Conc. | Intraymp |
| CHIR9902 | 252917- | 4.4 | 6.6 nM | 2-6 | 2-6 μM | 4 mM |
| 1 | 06-9 | nM | | μM | | |
| AZD 1080 | 612487- | 6.9 | 31 nM | 1-5 | 1-5 μM | 1-5 mM |
| | 72-6 | nM | | μM | | |
| GSK XXII | 1195901- | 2.3 | 2.0 nM | 0.2-1 | 0.2-1 μM | 0.2-1 mM |
| | 31-5 | nM | | μM | | |
| LY2090314 | 603288- | 2.1 | 0.9 nM | 5-20 | 5-20 nM | 5-20 μM |
| | 22-8 | nM | | nM | | |

TABLE 2

| Class | Agent | CAS |
|---|---|---|
| WNT | | |
| ARFGAP1 | QS 11 | 944328-88-5 |
| ARFGAP1 | WASP-1, ZINC00087877 | 352328-6 |
| Axin | Cpd1 | 1357473-75-6 |
| Axin | Cpd2 | 1228659-47-9 |
| Axin | HLY78 | 854847-61-3 |
| Axin | SKL2001 | 909089-13-0 |
| beta-catenin | DCA | 56-47-3 |
| Disrupts the Axin | Compound 2 | 1360540-82-4 |
| Complex | | |
| Disrupts the Axin | Compound 71 | 1622429-71-3 |
| Complex | | |
| Disrupts the Axin | ISX 9 | 832115-62-5 |
| Complex | | |
| DKK1 inhibitor | WAY-262611 | 1123231-07-1 |
| MEK | Radicicol | 12772-57-5 |
| MEK | Selumetinib (AZD6244) | 606143-52-6 |
| PP2A | IQ 1 | 331001-62-8 |
| sFRP-1 inhibitor | (Dimethylamino)propyl)- | 915754-88-0 |
| | 2-ethyl-5- | |
| | (phenylsulfonyl) | |
| | benzenesulfonamide | |
| sFRP-1 inhibitor | Cyclosporine A (CsA) | 59865-13-3 |
| sFRP-1 inhibitor | Cyclosporine analogues | |
| sFRP-1 inhibitor | PSC833 (Valspodar) | 121584-18-7 |
| sFRP-1 inhibitor | WAY 316606 | 915759-45-4 |
| Target Undetermined | Diketones | WO 2016029021 A1; |
| | | WO 2012024404 A1 |
| Target Undetermined | Diketones | 1622429-56-4 |
| Target Undetermined | Diketones | 1360540-88-0 |
| Target Undetermined | Diketones | 1360540-89-1 |
| Target Undetermined | Diketones | 1622429-79-1 |
| Target Undetermined | Diketones | 1622429-75-7 |
| Target Undetermined | Diketones | 1622429-74-6 |
| Target Undetermined | Diketones | 1622430-76-5 |
| Target Undetermined | Diketones | 1622430-31-2 |
| Target Undetermined | Diketones | 1622430-52-7 |
| Target Undetermined | Diketones | 1622429-67-7 |
| Target Undetermined | Diketones | 1622429-65-5 |
| Target Undetermined | Diketones | 1622429-69-9 |
| van-Gogh-like | Compound 109 | 1314885-81-8 |
| receptor proteins | | |
| (Vangl) | | |
| Wnt Ligand | Wnt-1 | Protein |
| Wnt Ligand | Wnt-10a | Protein |
| Wnt Ligand | Wnt-10b/12 | Protein |
| Wnt Ligand | Wnt-11 | Protein |
| Wnt Ligand | Wnt-16 | Protein |
| Wnt Ligand | Wnt-2/Irp | Protein |
| | (Int-I-related protein) | |
| Wnt Ligand | Wnt-2b/13 | Protein |
| Wnt Ligand | Wnt-3/Int-4 | Protein |
| Wnt Ligand | Wnt-3a | Protein |
| Wnt Ligand | Wnt-4 | Protein |
| Wnt Ligand | Wnt-5a | Protein |
| Wnt Ligand | Wnt-5b | Protein |
| Wnt Ligand | Wnt-6 | Protein |
| Wnt Ligand | Wnt-7a | Protein |
| Wnt Ligand | Wnt-7b | Protein |
| Wnt Ligand | Wnt-8a/8d | Protein |
| Wnt Ligand | Wnt-8b | Protein |
| Wnt Ligand | Wnt-9a/14 | Protein |
| Wnt Ligand | Wnt-9b/14b/15 | Protein |
| Wnt Related Protein | Norrin | Protein |
| Wnt Related Protein | R-Spondin 1/2/3/4 | Protein |
| Wnt-3a/Dkk-1 | BML-284 | 853220-52-7 |
| Wnt-3a/Dkk-1 | Compound 1 | 1084833-94-2 |
| Wnt-3a/Dkk-1 | Compound 25 | 1084834-05-8 |
| GSK3 alpha | | |
| CREB knockdown | 666-15 | 1433286-70-4 |
| Isonicotinamides | Compound 29 | 1772823-37-6 |
| Isonicotinamides | Compound 33 | 1772823-64-9 |
| Isonicotinamides | Compound 39 | 1772824-10-8 |
| Maleimide | I5 | 264217-24-5 |
| Maleimide | Tivantinib | 905854-02-6 |
| Organometallic | Compound (R)-DW12 | 1047684-07-0 |
| Organometallic | Compound 3 | 1498285-39-4 |
| | | 1498285-48-5 |
| Organometallic | Compound lambda-OS1 | 1291104-51-2 |
| | | 1292843-11-8 |
| Oxadiazoles | Compound 14d | 1374671-64-3 |
| Oxadiazoles | Compound 15b | 1374671-66-5 |
| Oxadiazoles | Compound 27 | 1820758-44-8 |
| Oxindole | AZD1080 | 612487-12-6 |
| Pyrazole | AT 7519 | 844442-38-2 |
| Pyrazole | Compound 4a | 1627557-91-8 |
| Pyrazole | Compound 4t | 1627558-10-4 |
| Pyrazole | Compound 4z | 1627558-16-0 |
| Pyrazole | GSK-3b XXII | 1195901-31-5 |
| Pyrazolopyridazines | Compound 18 | 405223-20-3 |
| Pyrazolopyridazines | Compound 19 | 405223-71-4 |
| Pyrazolopyridines | Compound 14 | 583038-63-5 |
| Pyrazolopyridines | Compound 23 | 583038-76-0 |
| Pyrazolopyridines | Pyrazolopyridine 34 | 583039-27-4 |
| Pyrazolo- | BRD1172 | 1597438-86-2 |
| tetrahydroquinolinone | | |
| Pyrazolo- | BRD1652 | 1597438-93-1 |
| tetrahydroquinolinone | | |
| Pyrazolo- | BRD4003 chiral | 1597439-60-5 |
| tetrahydroquinolinone | | |
| Pyrazolo- | BRD4003 chiral | 1597439-59-2 |
| tetrahydroquinolinone | | |
| Pyrazolo- | Compound 11 | 1597439-12-7 |
| tetrahydroquinolinone | | |
| Pyrazolo- | Compound 16 | 1597440-17-9 |
| tetrahydroquinolinone | | |
| Pyrazolo- | Compound 8 | 1597439-01-4 |
| tetrahydroquinolinone | | |
| Pyrazolo- | Compound 9 | 1597439-02-5 |
| tetrahydroquinolinone | | |
| Triazolpyrimidine | Compound 90 | 91322-11-1 |
| Triazolpyrimidine | Compound 92 | 1043429-30-6 |
| Urea | AR-A014418 | 487021-52-3 |
| GSK3-beta | | |
| Acid | Bikinin | 188011-69-0 |
| Acid | Valproic Acid, Sodium Salt | 99-66-1 |
| Aloisines | Aloisine A | 496864-16-5 |
| Aloisines | Aloisine B | 496864-14-3 |
| Aloisines | TWS119 | 1507095-58-0 |
| Aminopyrimidine | CHIR98014 (CT98014) | 252935-94-7 |
| Aminopyrimidine | CHIR98023 (CT98023) | 252904-84-0 |
| Aminopyrimidine | CHIR98024 (CT98024) | 556813-39-9 |
| Aminopyrimidine | CHIR99021 (CT99021) | 252917-06-9 |
| Aminopyrimidine | CT20026 | 403808-63-9 |
| Aminopyrimidinyl | CGP60474 | 164658-13-3 |
| Aminopyrimidinyl | GSK-3β inhibitor XVIII | 1139875-74-3 |
| Azaindolylinaleimide | Compound 29 | 436866-61-4 |
| Azaindolylmaleimide | Compound 46 | 682807-74-5 |
| Bisindolylmaleimide | Bisindolylmaleimide X HCl | 131848-97-0 |
| Bisindolylmaleimide | Compound 5a | 436866-54-5 |
| Bisindolylmaleimide | Enzastaurin (LY317615) | 170364-57-5 |
| Bisindolylmaleimide | GF109203x | 176504-36-2 |
| Bisindolylmaleimide | Ro318220 | 125314-64-9 |
| Dihydropyridine | ML320 | 1597438-84-0 |
| Flavone | Flavopiridol | 146426-40-6 |
| Furanosesquiterpenes | Palinurin | 254901-27-4 |
| Furanosesquiterpenes | Tricantin | 853885-55-9 |
| Furopyrimidine | Compound 100 | 744255-19-4 |
| Halomethylketones | Compound 17 | 62673-69-2 |
| Halomethylketones | GSK-3β Inhibitor VI | 62673-69-2 |
| Halomethylketones | GSK-3β Inhibitor VII | 99-73-0 |
| Hymenidin | Hymenidin | 107019-95-4 |
| Indirubins | 5-Iodo-indirubin-3′-monoxime | 331467-03-9 |
| Indirubins | 6-Bromoindirubin-3-acetoxime | 667463-85-6 |
| Indirubins | GSK-3 Inhibitor IX | 667463-62-9 |
| Indirubins | GSK-3 Inhibitor X | 740841-15-0 |
| Indirubins | Indirubin | 479-41-4 |
| Indirubins | Indirubin-3′-monoxime | 160807-49-8 |
| Indirubins | Indirubin-5-sulfonic acid | 331467-05-1 |
| | sodium salt | |
| Inorganic atom | Beryllium | |
| Inorganic atom | Lithium Chloride | |
| Inorganic atom | Tungstate | |
| Inorganic atom | Zinc | |
| Isoindolone | Staurosporine | 62996-74-1 |
| Isonicotinamides | Compound 29 | 1772823-37-6 |
| Isonicotinamides | Compound 33 | 1772823-64-9 |
| Isonicotinamides | Compound 39 | 1772824-10-8 |
| Maleimide | 3F8 | 159109-11-2 |
| Maleimide | 603281-31-8 | 603281-31-8 |
| Maleimide | BIP-135 | 941575-71-9 |
| Maleimide | Compound 34 | 396091-16-0 |
| Maleimide | CP21R7 | 125314-13-8 |
| Maleimide | GSK-3 inhibitor 1 | 603272-51-1 |
| Maleimide | GSK-3β Inhibitor XI | 626604-39-5 |
| Maleimide | I5 | 264217-24-5 |
| Maleimide | IM-12 | 1129669-05-1 |
| Maleimide | Isogranulatimide | 244148-46-7 |
| Maleimide | KT 5720 | 108068-98-0 |
| Maleimide | LY2090314 | 603288-22-8 |
| Maleimide | SB-216763 | 280744-09-4 |
| Maleimide | SB-415286 (SB-41528) | 264218-23-7 |
| Maleimide | TCS 21311 | 1260181-14-3 |
| Maleimide | Tivantinib | 905854-02-6 |
| Manzamines | Manzamine A | 104196-68-1 |
| Miscellaneous | AZD2858 (AR28) | 486424-20-8 |
| Miscellaneous | CID 755673 | 521937-07-5 |
| Miscellaneous | Dibromocantharelline | 101481-34-9 |
| Miscellaneous | TCS 2002 | 1005201-24-0 |
| Organometallic | (RRu)-HB1229 | |
| Organometallic | (RRu)-NP549 | |
| Organometallic | Compound (R)-DW12 | 1047684-07-0 |
| Organometallic | Compound 3 | 1498285-39-4, |
| | | 1498285-48-5 |
| Organometallic | Compound lambda-OS1 | 1291104-51-2, |
| | | 1292843-11-8 |
| Organometallic | DW12 | 861251-33-4 |
| Organometallic | HB12 | 800384-87-6 |
| Organometallic | NP309 | 937810-13-4 |
| Oxadiazol | Compound 14d | 1374671-64-3 |
| Oxadiazol | Compound 15b | 1374671-66-5 |
| Oxadiazol | Compound 20x | 1005201-80-8 |
| Oxadiazol | GSK-3 Inhibitor II | 478482-75-6 |
| Oxadiazol | GSK3 Inhibitor, 2 | 1377154-01-2 |
| Oxadiazol | TC-G 24 | 1257256-44-2 |
| Oxindole | AZD1080 | 612487-72-6 |
| Oxindole | SU9516 | 77090-84-1 |
| Patent | CN 101341138 B | |
| Patent | CN 1319968 C | |
| Patent | CP-70949 | |
| Patent | CT118637 | |
| Patent | EP 1739087 A1 | |
| Patent | EP 1961748 A2 | |
| Patent | EP 2765188 A1 | |
| Patent | GI179186X | |
| Patent | GW784752X | |
| Patent | GW784775X | |
| Patent | US 20070088080 A1 | |
| Patent | US 20100292205 A1 | |
| Patent | US 7514445 B2 | |
| Patent | US 8071591 B2 | |
| Patent | US 8207216 B2 | |
| Patent | US 8686042 B2 | |
| Patent | US 8771754 B2 | |
| Patent | WO 2001085685 A1 | |
| Patent | WO 2003037891 A1 | |
| Patent | WO 2006018633 A1 | |
| Patent | WO 2007102770 A1 | |
| Patent | WO 2008077138 A1 | |
| Patent | WO 2007106537 A2 | |
| Patent | WO 2009017453 A1 | |
| Patent | WO 2010075551 A1 | |
| Patent | WO 2010104205 A1 | |
| Patent | WO 2011089416 A1 | |
| Patent | WO 2013124413 A1 | |
| Patent | WO 2014003098 A1 | |
| Patent | WO 2014013255 A1 | |
| Patent | WO 2014050779 A1 | |
| Patent | WO 2014059383 A1 | |
| Patent | WO 2014083132 A1 | |
| Patent | WO2006100490A1/ | |
| | EP 1863904 A1 | |
| Patent | WO2009017455 A1 | |
| Paullone | Cmpd 17b | 408532-42-3 |
| Paullone | Kenpaullone | 142273-20-9 |
| Paullones | Alsterpaullone | 237430-03-4 |
| Paullones | Alsterpaullone CN Ethyl | 852529-97-0 |
| Pautiones | Azakenpaullone | 676596-65-9 |
| Paullones | Cazpaullone | 914088-64-5 |
| Peptide | FRATtide | |
| Peptide | L803 | |
| Peptides | L803-mts | |
| Publication | 705701 | |
| Publication | 708244 | |
| Publication | 709125 | |
| Publication | AR79 | |
| Publication | AZ13282107 | No Structure |
| Publication | AZ13282107 | |
| Publication | CEP-16805 | No Structure |
| Publication | CG-301338 | No Structure |
| Publication | CT73911 | |
| Publication | LY2064827 | |
| Publication | NP-103 | No Structure |
| Publication | SAR 502250 | No Structure |
| Publication | SAR 502250 (Sanofi) | 1073653-58-3 |
| Publication | XD-4241 | No Structure |
| Pyrazole | AT 7519 | 844442-38-2 |
| Pyrazole | Compound 4a | 1627557-91-8 |
| Pyrazole | Compound 4t | 1627558-10-4 |
| Pyrazole | Compound 4z | 1627558-16-0 |
| Pyrazole | GSK-3 Inhibitor XXII | 1195901-31-5 |
| Pyrazolone | GSK-3beta Inhibitor XXVI | 871843-09-3 |
| Pyrazolopyridazines | Compound 18 | 405223-20-3 |
| Pyrazolopyridazines | Compound 19 | 405223-71-4 |
| Pyrazolopyridine | Pyrazolopyridine 18 | 405221-39-8 |
| Pyrazolopyridine | Pyrazolopyridine 34 | 583039-27-4 |
| Pyrazolopyridine | Pyrazolopyridine 9 | 923029-74-7 |
| Pyrazolopyridines | Compound 14 | 583038-63-5 |
| Pyrazolopyridines | Compound 14 | 583038-63-5 |
| Pyrazolopyridines | Compound 23 | 583038-76-0 |
| Pyrazoloquinoxaline | NSC 693868 (Compound 1) | 40254-90-8 |
| Pyrazoloquinoxaline | NSC 693868 (Compound 1) | 40254-90-8 |
| Pyridinone | Compound 150 | 1282042-18-5 |
| Pyrrolopyridinyl | Compound 12 | 2025388-10-5 |
| Pyrrolopyridinyl | Compound 27 | 2025388-25-2 |
| Pyrroloazepine | Hymenialdisine | 82005-12-7 |
| Quinazolin | GSK-3 Inhibitor XIII | 404828-08-6 |
| Quinolinecarb | VP0.7 | 331963-23-6 |
| Quinoline | | 1132813-46-7 |
| Quinoline | | 1132812-98-6 |
| Quinoline | | 950727-66-9 |
| Quinolinc | | 950727-04-5 |
| Quinoline | | 1132812-98-6 |
| Thiadiazolidindiones | GSK-3β Inhibitor I | 327036-89-5 |
| Thiadiazolidindiones | NP031112 (Tideglusib) | 865854-05-3 |
| Thiadiazolidindiones | NP031115 | 1400575-57-6 |
| Triazolpyrimidine | Compound 90 | 91322-11-1 |
| Triazolpyrimidine | Compound 92 | 1043429-30-6 |
| Urea | GSK-3β Inh. VIII | 487021-52-3 |
| | AR-A014418 | |
| Urea | A-1070722 | 1384424-80-9 |

In some embodiments, an agent of having activity as a Wnt agonist is a GSK3 inhibitor. In some embodiments, the GSK3 inhibitor is AZD1080, GSK3 inhibitor XXII, CHIR99021, or LY2090314. In some embodiments, the Wnt agonist is CHIR99021. In other embodiments, Wnt agonist and/or GSK3 inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione. (Formula A.)

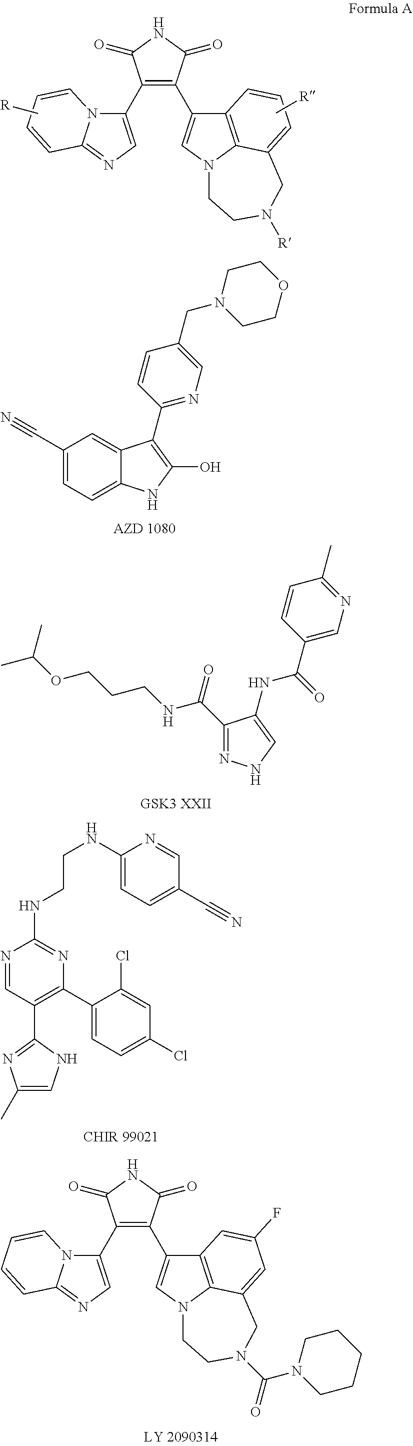

The Wnt agonist can be any selected from WO 2018/125746, which is hereby incorporated by reference. In some embodiments, the Wnt agonist can be the compound as defined in claim 1 of WO 2018/125746. In some embodiments, the Wnt agonist can be the compound as defined in claim 12 of WO 2018/125746.”

Exemplary, substituted 3-Imidazo[1,2-a]pyridin-3-yl-4-(1,2,3,4-tetrahydro-[1,4]diazepino-[6,7,1-hi]indol-7-yl)pyrrole-2,5-dione include: 3-(imidazo[1,2-a]pyridin-3-yl)-4-(2-(piperidine-1-carbonyl)-9-(trifluoromethyl)-1,2,3,4-tetrahydro-[1,4]diazepino[6,7,1-hi]indol-7-yl)-1H-pyrrole-2,5-dione; 7-(4-(imidazo[1,2-a]pyridin-3-yl)-2,5-dioxo-2,5-dihydro-1H-pyrrol-3-yl)-2-(piperidine-1-carbonyl)-1,2,3,4-tetrahydro-[1,4]diazepino[6,7,1-hi]indole-9-carbonitrile; 3-(9-ethynyl-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-amino-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 1-(9-fluoro-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-2-carbonyl)piperidine-4-carbaldehyde; 3-(9-fluoro-2-(4-(hydroxymethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4,4-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(8-oxa-3-azabicyclo[3.2.1]octane-3-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(benzo[d]isoxazol-3-yl)-4-(9-fluoro-2-(piperidine-1-carbonyl)-1,2,3,4-tetrahydro-[1,4]diazepino[6,7,1-hi]indol-7-yl)-1H-pyrrole-2,5-dione; N-(7-(4-(imidazo[1,2-a]pyridin-3-yl)-2,5-dioxo-2,5-dihydro-1H-pyrrol-3-yl)-2-(piperidine-1-carbonyl)-1,2,3,4-tetrahydro-[1,4]diazepino[6,7,1-hi]indol-9-yl)acetamide; 3-(9-(difluoromethyl)-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(3,3-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-((1R,4R)-2,5-diazabicyclo[2.2.1]heptane-2-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 2-(8-oxa-3-azabicyclo[3.2.1]octane-3-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 2-(3,3-difluoropiperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 2-(4,4-difluoropiperidine-1-carbonyl)-7-(4-(imidazo[1,2-a]pyridin-3-yl)-2,5-dioxo-2,5-dihydro-H-pyrrol-3-yl)-1,2,3,4-tetrahydro-[1,4]diazepino[6,7,1-hi]indole-9-carbonitrile; 3-(2-(4,4-difluoropiperidine-1-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(8-oxa-3-azabicyclo[3.2.1]octane-3-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(aminomethyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(hydroxymethyl)piperidine-1-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 2-(4-(hydroxymethyl)piperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 3-(9-fluoro-2-(3,3,4,4,5,5-hexafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(3,3,5,5-tetrafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(2,2,6,6-tetrafluoromorpholine-4-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4,4-difluoro-3-hydroxypiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(difluoro(hydroxy)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(6,6-difluoro-1,4-oxazepane-4-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-([1,2,4]triazolo[4,3-a]pyridin-3-yl)-4-(9-fluoro-2-(piperidine-1-carbonyl)-1,2,3,4-tetrahydro-[1,4]diazepino[6,7,1-hi]indol-7-yl)-1H-pyrrole-2,5-dione; 3-(9-fluoro-2-(piperidine-1-carbonyl-d10)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(piperidine-1-carbonyl)-1,2,3,4-tetrahydro-[1,4]diazepino[6,7,1-hi]indol-7-yl-3,3,4,4-d4)-4-(imidazo[1,2-a]pyridin-3-yl)-1H-pyrrole-2,5-dione; 3-(9-fluoro-2-(4-(2,2,2-trifluoro-1-hydroxyethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(4-((methylamino)methyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-((dimethylamino)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-aminopiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(4-(methylamino)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(dimethylamino)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 9-fluoro-7-(4-(imidazo[1,2-a]pyridin-3-yl)-2,5-dioxo-2,5-dihydro-1H-pyrrol-3-yl)-N-(piperidin-4-ylmethyl)-3,4-dihydro-[1,4]diazepino[6,7,1-hi]indole-2(1H)-carboxamide; 9-fluoro-7-(4-(imidazo[1,2-a]pyridin-3-yl)-2,5-dioxo-2,5-dihydro-1H-pyrrol-3-yl)-N-methyl-N-(piperidin-4-ylmethyl)-3,4-dihydro-[1,4]diazepino[6,7,1-hi]indole-2(1H)-carboxamide; 9-fluoro-7-(4-(imidazo[1,2-a]pyridin-3-yl)-2,5-dioxo-2,5-dihydro-1H-pyrrol-3-yl)-N-methyl-N-((1-methylpiperidin-4-yl)methyl)-3,4-dihydro-[1,4]diazepino[6,7,1-hi]indole-2(1H)-carboxamide; 3-(9-fluoro-2-((1R,4R)-5-methyl-2,5-diazabicyclo[2.2.1]heptane-2-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(2-methyl-2,8-diazaspiro[4.5]decane-8-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(8-methyl-2,8-diazaspiro[4.5]decane-2-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(imidazo[1,2-a]pyridin-3-yl)-4-(2-(2,2,6,6-tetrafluoromorpholine-4-carbonyl)-9-(trifluoromethyl)-1,2,3,4-tetrahydro-[1,4]diazepino[6,7,1-hi]indol-7-yl)-1H-pyrrole-2,5-dione; 3-(2-(6,6-difluoro-1,4-oxazepane-4-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 2-(4-(dimethylamino)piperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 9-cyano-7-(4-(imidazo[1,2-a]pyridin-3-yl)-2,5-dioxo-2,5-dihydro-1H-pyrrol-3-yl)-N-methyl-N-((1-methylpiperidin-4-yl)methyl)-3,4-dihydro-[1,4]diazepino[6,7,1-hi]indole-2(1H)-carboxamide; 7-(4-(imidazo[1,2-a]pyridin-3-yl)-2,5-dioxo-2,5-dihydro-1H-pyrrol-3-yl)-2-(8-methyl-2,8-diazaspiro[4.5]decane-2-carbonyl)-1,2,3,4-tetrahydro-[1,4]diazepino[6,7,1-hi]indole-9-carbonitrile; 3-(8,9-difluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; or 3-(9-fluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione (LY20900314).

In some embodiments, the substituted 3-Imidazo[1,2-a]pyridin-3-yl-4-(1,2,3,4-tetrahydro-[1,4]diazepino-[6,7,1-hi]indol-7-yl)pyrrole-2,5-dione is: 3-(imidazo[1,2-a]pyridin-3-yl)-4-(2-(piperidine-1-carbonyl)-9-(trifluoromethyl)-1,2,3,4-tetrahydro-[1,4]diazepino[6,7,1-hi]indol-7-yl)-1H-pyrrole-2,5-dione; 7-(4-(imidazo[1,2-a]pyridin-3-yl)-2,5-dioxo-2,5-dihydro-1H-pyrrol-3-yl)-2-(piperidine-1-carbonyl)-1,2,3,4-tetrahydro-[1,4]diazepino[6,7,1-hi]indole-9-carbonitrile; 3-(9-ethynyl-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(4-(hydroxymethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4,4-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(8-oxa-3-azabicyclo[3.2.1]octane-3-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-(difluoromethyl)-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(3,3-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 2-(4,4-difluoropiperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 3-(2-(8-oxa-3-azabicyclo[3.2.1]octane-3-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(hydroxymethyl)piperidine-1-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(3,3,4,4,5,5-hexafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(3,3,5,5-tetrafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(2,2,6,6-tetrafluoromorpholine-4-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4,4-difluoro-3-hydroxypiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(difluoro(hydroxy)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(6,6-difluoro-1,4-oxazepane-4-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(piperidine-1-carbonyl-d10)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(piperidine-1-carbonyl)-1,2,3,4-tetrahydro-[1,4]diazepino[6,7,1-hi]indol-7-yl-3,3,4,4-d4)-4-(imidazo[1,2-a]pyridin-3-yl)-1H-pyrrole-2,5-dione; 3-(9-fluoro-2-(4-(2,2,2-trifluoro-1-hydroxyethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-((dimethylamino)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(dimethylamino)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 9-fluoro-7-(4-(imidazo[1,2-a]pyridin-3-yl)-2,5-dioxo-2,5-dihydro-1H-pyrrol-3-yl)-N-methyl-N-((1-methylpiperidin-4-yl)methyl)-3,4-dihydro-[1,4]diazepino[6,7,1-hi]indole-2(1H)-carboxamide; 3-(imidazo[1,2-a]pyridin-3-yl)-4-(2-(2,2,6,6-tetrafluoromorpholine-4-carbonyl)-9-(trifluoromethyl)-1,2,3,4-tetrahydro-[1,4]diazepino[6,7,1-hi]indol-7-yl)-1H-pyrrole-2,5-dione; 3-(2-(6,6-difluoro-1,4-oxazepane-4-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(8,9-difluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; or 3-(9-fluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione. (LY2090314).

In certain embodiments, the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione. (LY2090314).

The structures of the substituted 3-Imidazo[1,2-a]pyridin-3-yl-4-(1,2,3,4-tetrahydro-[1,4]diazepino-[6,7,1-hi]indol-7-yl)pyrrole-2,5-dione are shown below in Table 3.

TABLE 3

| | | |
|---|---|---|
| Compound I-1 | 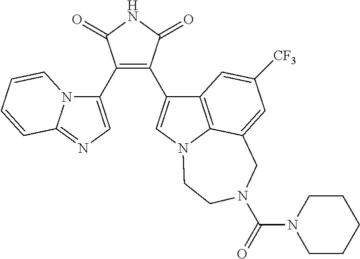 [SMILES: O=C1NC(=O)C(c2cnc3ccccn23)=C1c1cn2c3c(cc(C(F)(F)F)cc13)CN(C(=O)N1CCCCC1)CC2] | 3-(imidazo[1,2-a]pyridin-3-yl)-4-(2- (piperidine-1-carbonyl)-9-(trifluoromethyl)- 1,2,3,4-tetrahydro-[1,4]diazepino[6,7,1- hi]indol-7-yl)-1H-pyrrole-2,5-dione |
| Compound I-2 | 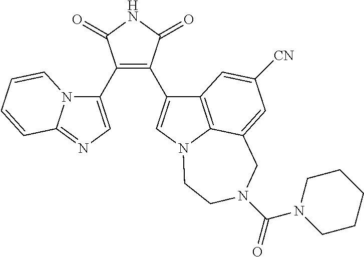 [SMILES: N#Cc1cc2c3c(c1)c(C1=C(c4cnc5ccccn45)C(=O)NC1=O)cn3CCN(C(=O)N1CCCCC1)C2] | 7-(4-(imidazo[1,2-a]pyridin-3-yl)-2,5-dioxo- 2,5-dihydro-1H-pyrrol-3-yl)-2-(piperidine-1- carbonyl)-1,2,3,4-tetrahydro- [1,4]diazepino[6,7,1-hi]indole-9-carbonitrile |
| Compound I-3 | 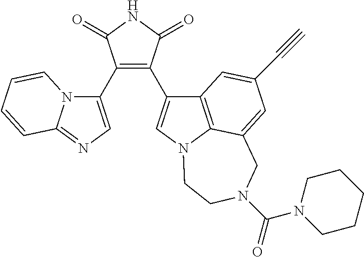 [SMILES: C#Cc1cc2c3c(c1)c(C1=C(c4cnc5ccccn45)C(=O)NC1=O)cn3CCN(C(=O)N1CCCCC1)C2] | 3-(9-ethynyl-2-(piperidine-1-carbonyl)- 1,2,3,4-tetrahydro-[1,4]diazepino[6,7,1- hi]indol-7-yl)-4-(imidazo[1,2-a]pyridin-3-yl)- 1H-pyrrole-2,5-dione |
| Compound I-4 | 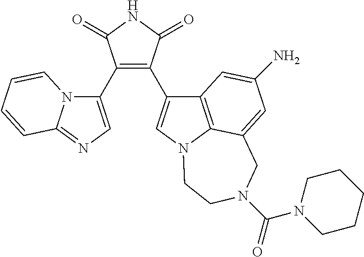 [SMILES: Nc1cc2c3c(c1)c(C1=C(c4cnc5ccccn45)C(=O)NC1=O)cn3CCN(C(=O)N1CCCCC1)C2] | 3-(9-amino-2-(piperidine-1-carbonyl)-1,2,3,4- tetrahydro-[1,4]diazepino[6,7,1-hi]indol-7-yl)- 4-(imidazo[1,2-a]pyridin-3-yl)-1H-pyrrole- 2,5-dione |
| Compound I-5 | 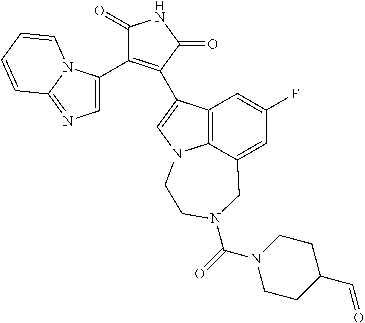 [SMILES: O=CC1CCN(C(=O)N2CCn3cc(C4=C(c5cnc6ccccn56)C(=O)NC4=O)c4cc(F)cc(c43)C2)CC1] | 1-(9-fluoro-7-(4-(imidazo[1,2-a]pyridin-3-yl)- 2,5-dioxo-2,5-dihydro-1H-pyrrol-3-yl)- 1,2,3,4-tetrahydro-[1,4]diazepino[6,7,1- hi]indole-2-carbonyl)piperidine-4- carbaldehyde |
| Compound I-6 | 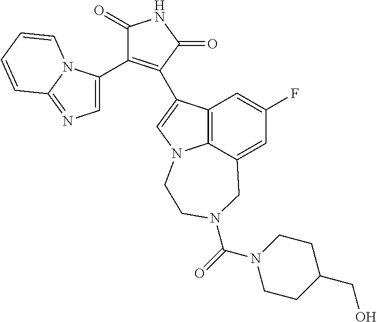 [SMILES: O=C1NC(=O)C(c2cnc3ccccn23)=C1c1cn2c3c(cc(F)cc13)CN(C(=O)N1CCC(CO)CC1)CC2] | 3-(9-fluoro-2-(4-(hydroxymethyl)piperidine-1- carbonyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-7 | 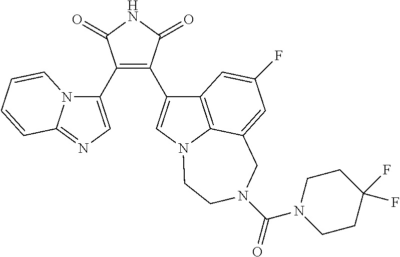 [SMILES: O=C1NC(=O)C(c2cnc3ccccn23)=C1c1cn2c3c(cc(F)cc13)CN(C(=O)N1CCC(F)(F)CC1)CC2] | 3-(2-(4,4-difluoropiperidine-1-carbonyl)-9- fluoro-1,2,3,4-tetrahydro-[1,4]diazepino[6,7,1- hi]indol-7-yl)-4-(imidazo[1,2-a]pyridin-3-yl)- 1H-pyrrole-2,5-dione |
| Compound I-8 | 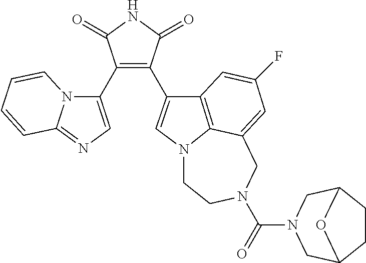 [SMILES: O=C1NC(=O)C(c2cnc3ccccn23)=C1c1cn2c3c(cc(F)cc13)CN(C(=O)N1CC3CCC(C1)O3)CC2] | 3-(2-(8-oxa-3-azabicyclo[3.2.1]octane-3- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-9 | 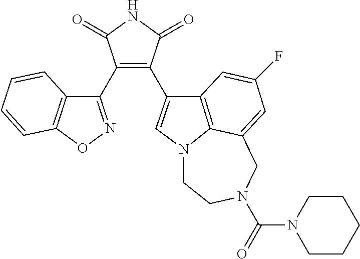 [SMILES: O=C1NC(=O)C(c2cn3c4c(cc(F)cc24)CN(C(=O)N2CCCCC2)CC3)=C1c1noc2ccccc12] | 3-(benzo[d]isoxazol-3-yl)-4-(9-fluoro-2- (piperidine-1-carbonyl)-1,2,3,4-tetrahydro- [1,4]diazepino[6,7,1-hi]indol-7-yl)-1H- pyrrole-2,5-dione |
| Compound I- 10 | 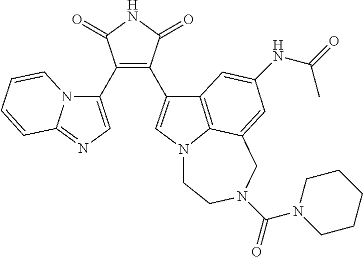 [SMILES: CC(=O)Nc1cc2c3c(c1)c(C1=C(c4cnc5ccccn45)C(=O)NC1=O)cn3CCN(C(=O)N1CCCCC1)C2] | N-(7-(4-(imidazo[1,2-a]pyridin-3-yl)-2,5- dioxo-2,5-dihydro-1H-pyrrolo-3-yl)-2- (piperidine-1-carbonyl)-1,2,3,4-tetrahydro- [1,4]diazepino[6,7,1-hi]indol-9-yl)acetamide |
| Compound I- 11 | 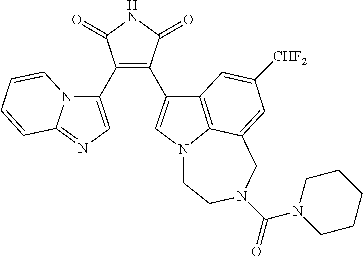 [SMILES: O=C1NC(=O)C(c2cnc3ccccn23)=C1c1cn2c3c(cc(C(F)F)cc13)CN(C(=O)N1CCCCC1)CC2] | 3-(9-(difluoromethyl)-2-(piperidine-1- carbonyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 12 | 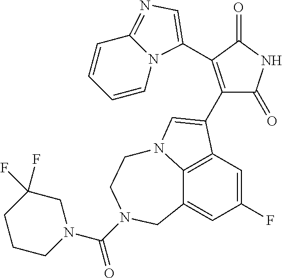 [SMILES: O=C1NC(=O)C(c2cnc3ccccn23)=C1c1cn2c3c(cc(F)cc13)CN(C(=O)N1CCCC(F)(F)C1)CC2] | 3-(2-(3,3-difluoropiperidine-1-carbonyl)-9- fluoro-1,2,3,4-tetrahydro-[1,4]diazepino[6,7,1- hi]indol-7-yl)-4-(imidazo[1,2-a]pyridin-3-yl)- 1H-pyrrole-2,5-dione |
| Compound I- 13 | 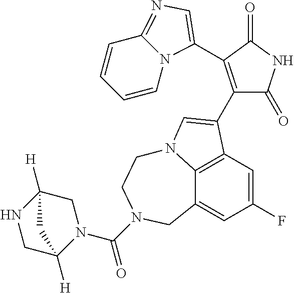 [SMILES: O=C1NC(=O)C(c2cnc3ccccn23)=C1c1cn2c3c(cc(F)cc13)CN(C(=O)N1C[C@H]3C[C@@H]1CN3)CC2] | 3-(2-((1R,4R)-2,5-diazabicyclo[2.2.1]heptane- 2-carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 14 | 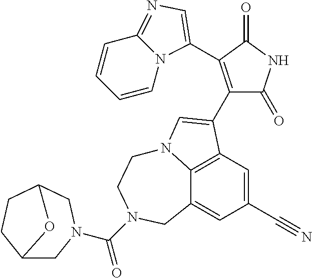 [SMILES: N#Cc1cc2c3c(c1)c(C1=C(c4cnc5ccccn45)C(=O)NC1=O)cn3CCN(C(=O)N1CC3CCC(C1)O3)C2] | 2-(8-oxa-3-azabicyclo[3.2.1]octane-3- carbonyl)-7-(4-(imidazo[1,2-a]pyridin-3-yl)- 2,5-dioxo-2,5-dihydro-1H-pyrrol-3-yl)- 1,2,3,4-tetrahydro-[1,4]diazepino[6,7,1- hi]indole-9-carbonitrile |
| Compound I- 15 | 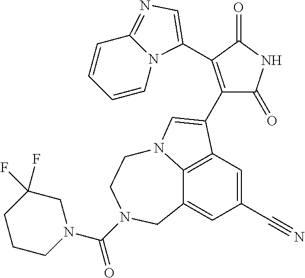 [SMILES: N#Cc1cc2c3c(c1)c(C1=C(c4cnc5ccccn45)C(=O)NC1=O)cn3CCN(C(=O)N1CCCC(F)(F)C1)C2] | 2-(3,3-difluoropiperidine-1-carbonyl)-7-(4- (imidazo[1,2-a]pyridin-3-yl)-2,5-dioxo-2,5- dihydro-1H-pyrrol-3-yl)-1,2,3,4-tetrahydro- [1,4]diazepino[6,7,1-hi]indole-9-carbonitrile |
| Compound I- 16 | 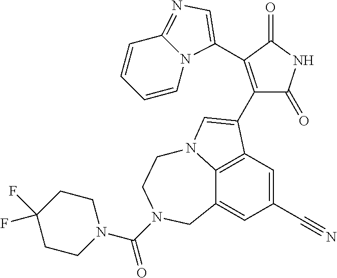 [SMILES: N#Cc1cc2c3c(c1)c(C1=C(c4cnc5ccccn45)C(=O)NC1=O)cn3CCN(C(=O)N1CCC(F)(F)CC1)C2] | 2-(4,4-difluoropiperidine-1-carbonyl)-7-(4- (imidazo[1,2-a]pyridin-3-yl)-2,5-dioxo-2,5- dihydro-1H-pyrrol-3-yl)-1,2,3,4-tetrahydro- [1,4]diazepino[6,7,1-hi]indole-9-carbonitrile |
| Compound I- 17 | 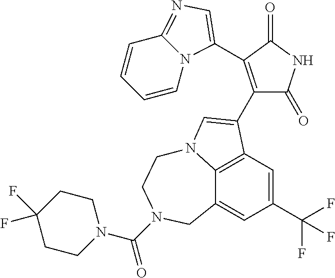 [SMILES: O=C1NC(=O)C(c2cnc3ccccn23)=C1c1cn2c3c(cc(C(F)(F)F)cc13)CN(C(=O)N1CCC(F)(F)CC1)CC2] | 3-(2-(4,4-difluoropiperidine-1-carbonyl)-9- (trifluoromethyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 18 | 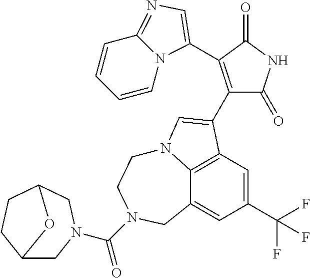 [SMILES: O=C1NC(=O)C(c2cnc3ccccn23)=C1c1cn2c3c(cc(C(F)(F)F)cc13)CN(C(=O)N1CC3CCC(C1)O3)CC2] | 3-(2-(8-oxa-3-azabicyclo[3.2.1]octane-3- carbonyl)-9-(trifluoromethyl)-1,2,3,4- tetrahydro-[1,4]diazepino[6,7,1-hi]indol-7-yl)- 4-(imidazo[1,2-a]pyridin-3-yl)-1H-pyrrole- 2,5-dione |
| Compound I- 19 | 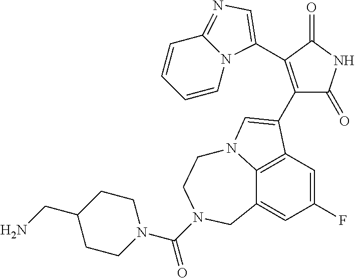 [SMILES: NCC1CCN(C(=O)N2CCn3cc(C4=C(c5cnc6ccccn56)C(=O)NC4=O)c4cc(F)cc(c43)C2)CC1] | 3-(2-(4-(aminomethyl)piperidine-1-carbonyl)- 9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 20 | 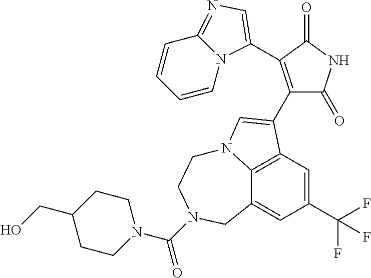 [SMILES: O=C1NC(=O)C(c2cnc3ccccn23)=C1c1cn2c3c(cc(C(F)(F)F)cc13)CN(C(=O)N1CCC(CO)CC1)CC2] | 3-(2-(4-(hydroxymethyl)piperidine-1- carbonyl)-9-(trifluoromethyl)-1,2,3,4- tetrahydro-[1,4]diazepino[6,7,1-hi]indol-7-yl)- 4-(imidazo[1,2-a]pyridin-3-yl)-1H-pyrrole- dione |
| Compound I- 21 | 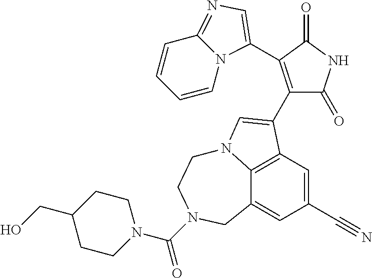 [SMILES: N#Cc1cc2c3c(c1)c(C1=C(c4cnc5ccccn45)C(=O)NC1=O)cn3CCN(C(=O)N1CCC(CO)CC1)C2] | 2-(4-(hydroxymethyl)piperidine-1-carbonyl)- 7-(4-(imidazo[1,2-a]pyridin-3-yl)-2,5-dioxo- 2,5-dihydro-1H-pyrrol-3-yl)-1,2,3,4- tetrahydro-[1,4]diazepino[6,7,1-hi]indole-9- carbonitrile |
| Compound I- 22 | 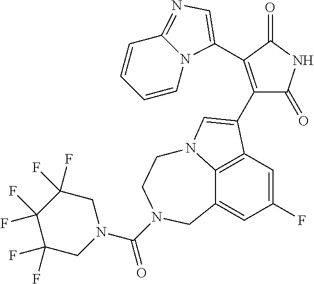 [SMILES: O=C1NC(=O)C(c2cnc3ccccn23)=C1c1cn2c3c(cc(F)cc13)CN(C(=O)N1CC(F)(F)C(F)(F)C(F)(F)C1)CC2] | 3-(9-fluoro-2-(3,3,4,4,5,5- hexafluoropiperidine-1-carbonyl)-1,2,3,4- tetrahydro-[1,4]diazepino[6,7,1-hi]indol-7-yl)- 4-(imidazo[1,2-a]pyridin-3-yl)-1H-pyrrole- 2,5-dione |
| Compound I- 23 | 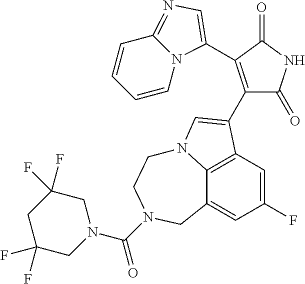 [SMILES: O=C1NC(=O)C(c2cnc3ccccn23)=C1c1cn2c3c(cc(F)cc13)CN(C(=O)N1CC(F)(F)CC(F)(F)C1)CC2] | 3-(9-fluoro-2-(3,3,5,5-tetrafluoropiperidine-1- carbonyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| 1Compound I- 24 | 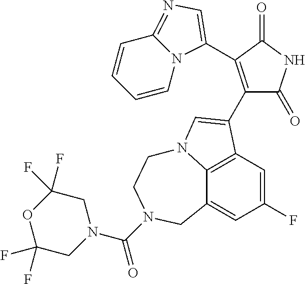 [SMILES: O=C1NC(=O)C(c2cnc3ccccn23)=C1c1cn2c3c(cc(F)cc13)CN(C(=O)N1CC(F)(F)OC(F)(F)C1)CC2] | 3-(9-fluoro-2-(2,2,6,6-tetrafluoromorpholine- 4-carbonyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 25 | 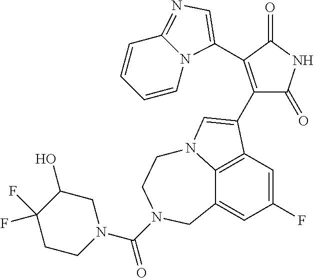 [SMILES: O=C1NC(=O)C(c2cnc3ccccn23)=C1c1cn2c3c(cc(F)cc13)CN(C(=O)N1CCC(F)(F)C(O)C1)CC2] | 3-(2-(4,4-difluoro-3-hydroxypiperidine-1- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 26 | 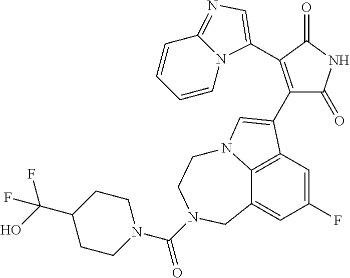 [SMILES: O=C1NC(=O)C(c2cnc3ccccn23)=C1c1cn2c3c(cc(F)cc13)CN(C(=O)N1CCC(C(O)(F)F)CC1)CC2] | 3-(2-(4-(difluoro(hydroxy)methyl)piperidine- 1-carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 27 | 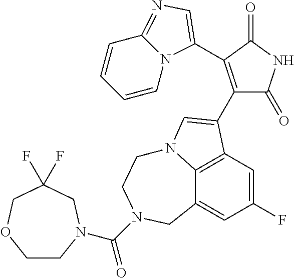 [SMILES: O=C1NC(=O)C(c2cnc3ccccn23)=C1c1cn2c3c(cc(F)cc13)CN(C(=O)N1CCOCC(F)(F)C1)CC2] | 3-(2-(6,6-difluoro-1,4-oxazepane-4-carbonyl)- 9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 28 | 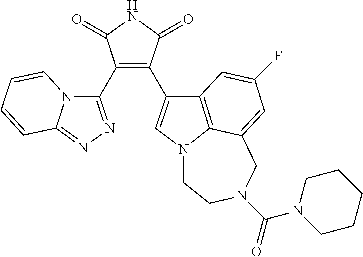 [SMILES: O=C1NC(=O)C(c2nnc3ccccn23)=C1c1cn2c3c(cc(F)cc13)CN(C(=O)N1CCCCC1)CC2] | 3-([1,2,4]triazolo[4,3-a]pyridin-3-yl)-4-(9- fluoro-2-(piperidine-1-carbonyl)-1,2,3,4- tetrahydro-[1,4]diazepino[6,7,1-hi]indol-7-yl)- 1H-pyrrole-2,5-dione |
| Compound I- 29 | 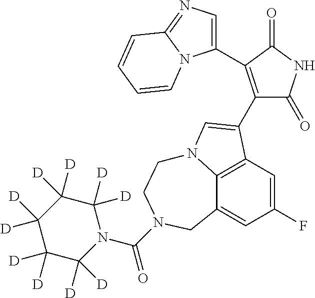 [SMILES: [2H]C1([2H])N(C(=O)N2CCn3cc(C4=C(c5cnc6ccccn56)C(=O)NC4=O)c4cc(F)cc(c43)C2)C([2H])([2H])C([2H])([2H])C([2H])([2H])C1([2H])[2H]] | 3-(9-fluoro-2-(piperidine-1-carbonyl-d10)- 1,2,3,4-tetrahydro-[1,4]diazepino[6,7,1- hi]indol-7-yl)-4-(imidazo[1,2-a]pyridin-3-yl)- 1H-pyrrole-2,5-dione |
| Compound I- 30 | 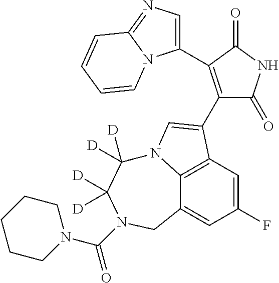 [SMILES: [2H]C1([2H])N(C(=O)N2CCCCC2)Cc2cc(F)cc3c(C4=C(c5cnc6ccccn56)C(=O)NC4=O)cn(c23)C1([2H])[2H]] | 3-(9-fluoro-2-(piperidine-1-carbonyl)-1,2,3,4- tetrahydro-[1,4]diazepino[6,7,1-hi]indol-7-yl- 3,3,4,4-d4)-4-(imidazo[1,2-a]pyridin-3-yl)- 1H-pyrrole-2,5-dione |
| Compound I- 31 | 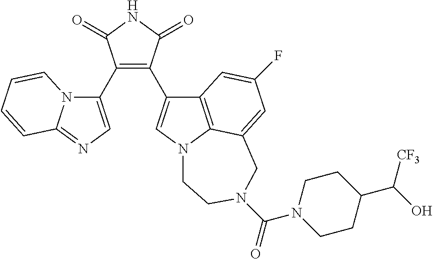 [SMILES: O=C1NC(=O)C(c2cnc3ccccn23)=C1c1cn2c3c(cc(F)cc13)CN(C(=O)N1CCC(C(O)C(F)(F)F)CC1)CC2] | 3-(9-fluoro-2-(4-(2,2,2-trifluoro-1- hydroxyethyl)piperidine-1-carbonyl)-1,2,3,4- tetrahydro-[1,4]diazepino[6,7,1-hi]indol-7-yl)- 4-(imidazo[1,2-a]pyridin-3-yl)-1H-pyrrole- 2,5-dione |
| Compound I- 32 | 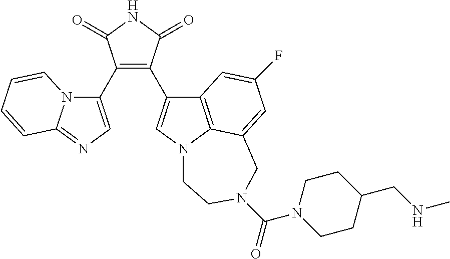 [SMILES: CNCC1CCN(C(=O)N2CCn3cc(C4=C(c5cnc6ccccn56)C(=O)NC4=O)c4cc(F)cc(c43)C2)CC1] | 3-(9-fluoro-2-(4- ((methylamino)methyl)piperidine-1-carbonyl)- 1,2,3,4-tetrahydro-[1,4]diazepino[6,7,1- hi]indol-7-yl)-4-(imidazo[1,2-a]pyridin-3-yl)- 1H-pyrrole-2,5-dione |
| Compund I- 33 | 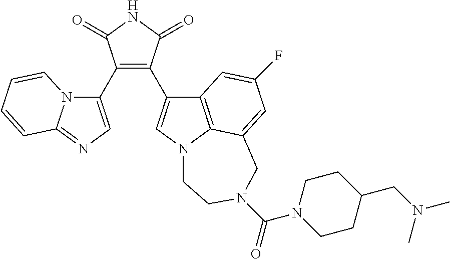 [SMILES: CN(C)CC1CCN(C(=O)N2CCn3cc(C4=C(c5cnc6ccccn56)C(=O)NC4=O)c4cc(F)cc(c43)C2)CC1] | 3-(2-(4-((dimethylamino)methyl)piperidine-1- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 34 | 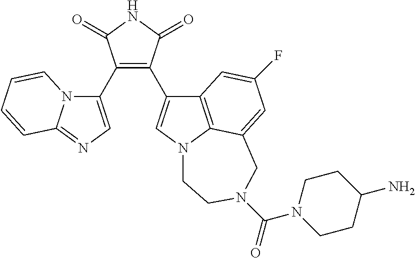 [SMILES: NC1CCN(C(=O)N2CCn3cc(C4=C(c5cnc6ccccn56)C(=O)NC4=O)c4cc(F)cc(c43)C2)CC1] | 3-(2-(4-aminopiperidine-1-carbonyl)-9-fluoro- 1,2,3,4-tetrahydro-[1,4]diazepino[6,7,1- hi]indol-7-yl)-4-(imidazo[1,2-a]pyridine-3-yl)- 1H-pyrrole-2,5-dione |
| Compound I- 35 | 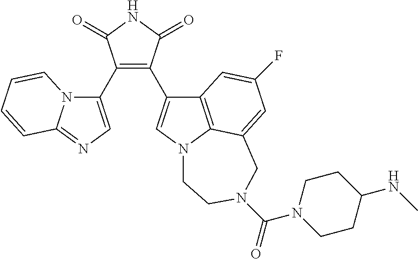 [SMILES: CNC1CCN(C(=O)N2CCn3cc(C4=C(c5cnc6ccccn56)C(=O)NC4=O)c4cc(F)cc(c43)C2)CC1] | 3-(9-fluoro-2-(4-(methylamino)piperidine-1- carbonyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 36 | 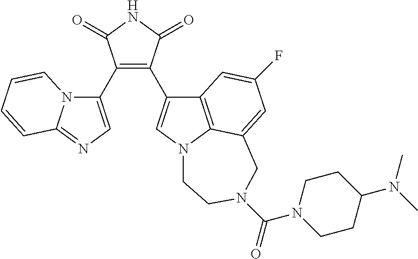 [SMILES: CN(C)C1CCN(C(=O)N2CCn3cc(C4=C(c5cnc6ccccn56)C(=O)NC4=O)c4cc(F)cc(c43)C2)CC1] | 3-(2-(4-(dimethylamino)piperidine-1- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 37 | 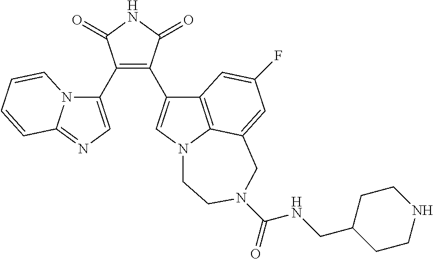 [SMILES: O=C1NC(=O)C(c2cnc3ccccn23)=C1c1cn2c3c(cc(F)cc13)CN(C(=O)NCC1CCNCC1)CC2] | 9-fluoro-7-(4-(imidazo[1,2-a]pyridin-3-yl)- 2,5-dioxo-2,5-dihydro-1H-pyrrol-3-yl)-N- (piperidin-4-ylmethyl)-3,4-dihydro- [1,4]diazepino[6,7,1-hi]indole-2(1H)- carboxamide |
| Compound I- 38 | 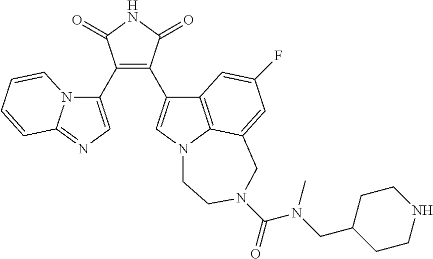 [SMILES: CN(CC1CCNCC1)C(=O)N1CCn2cc(C3=C(c4cnc5ccccn45)C(=O)NC3=O)c3cc(F)cc(c32)C1] | 9-fluoro-7-(4-(imidazo[1,2-a]pyridin-3-yl)- 2,5-dioxo-2,5-dihydro-1H-pyrrol-3-yl)-N- methyl-N-(piperidin-4-ylmethyl)-3,4-dihydro- [1,4]diazepino[6,7,1-hi]indole-2(1H)- carboxamide |
| Compound I- 39 | 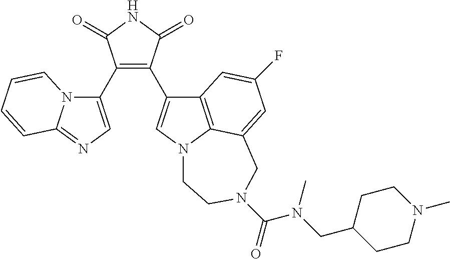 [SMILES: CN1CCC(CN(C)C(=O)N2CCn3cc(C4=C(c5cnc6ccccn56)C(=O)NC4=O)c4cc(F)cc(c43)C2)CC1] | 9-fluoro-7-(4-(imidazo[1,2-a]pyridin-3-yl)- 2,5-dioxo-2,5-dihydro-1H-pyrrol-3-yl)-N- methyl-N-((1-methylpiperidin-4-yl)methyl)- 3,4-dihydro-[1,4]diazepino[6,7,1-hi]indole- 2(1H)-carboxamide |
| Compound I- 40 | 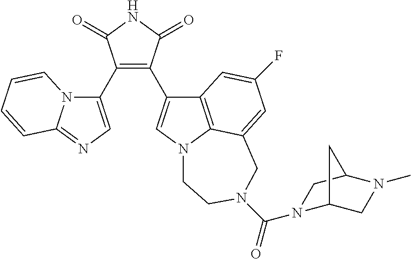 [SMILES: CN1CC2CC1CN2C(=O)N1CCn2cc(C3=C(c4cnc5ccccn45)C(=O)NC3=O)c3cc(F)cc(c32)C1] | 3-(9-fluoro-2-((1R,4R)-5-methyl-2,5- diazabicyclo[2.2.1]heptane-2-carbonyl)- 1,2,3,4-tetrahydro-[1,4]diazepino[6,7,1- hi]indol-7-yl)-4-(imidazo[1,2-a]pyridin-3-yl)- 1H-pyrrole-2,5-dione |
| Compound I- 41 | 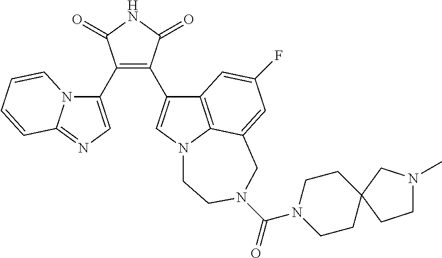 [SMILES: CN1CCC2(CCN(C(=O)N3CCn4cc(C5=C(c6cnc7ccccn67)C(=O)NC5=O)c5cc(F)cc(c54)C3)CC2)C1] | 3-(9-fluoro-2-(2-methyl-2,8- diazaspiro[4.5]decane-8-carbonyl)-1,2,3,4- tetrahydro-[1,4]diazepino[6,7,1-hi]indol-7-yl)- 4-(imidazo[1,2,a]pyridin-3-yl)-1H-pyrrole- 2,5-dione |
| Compound I- 42 | 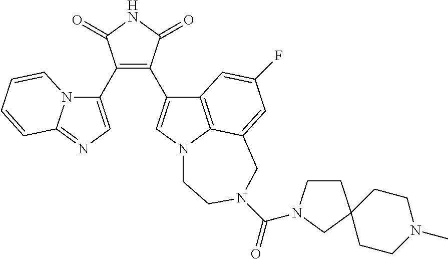 [SMILES: CN1CCC2(CC1)CCN(C(=O)N1CCn3cc(C4=C(c5cnc6ccccn56)C(=O)NC4=O)c4cc(F)cc(c43)C1)C2] | 3-(9-fluoro-2-(8-methyl-2,8- diazaspiro[4.5]decane-2-carbonyl)-1,2,3,4- tetrahydro-[1,4]diazepino[6,7,1-hi]indol-7-yl)- 4-(imidazo[1,2-a]pyridin-3-yl)-1H-pyrrole- 2,5-dione |
| Compound I- 43 | 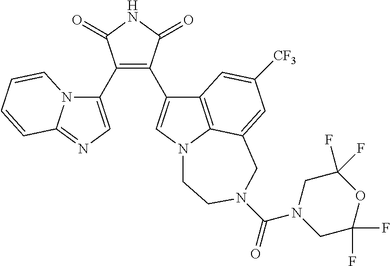 [SMILES: O=C1NC(=O)C(c2cnc3ccccn23)=C1c1cn2c3c(cc(C(F)(F)F)cc13)CN(C(=O)N1CC(F)(F)OC(F)(F)C1)CC2] | 3-(imidazo[1,2-a]pyridin-3-yl)-4-(2-(2,2,6,6- tetrafluoromorpholine-4-carbonyl)-9- (trifluoromethyl)-1,2,3,4-tetrahydro- [1,4]diazepino[6,7,1-hi]indol-7-yl)-1H- pyrrole-2,5-dione |
| Compound I- 44 | 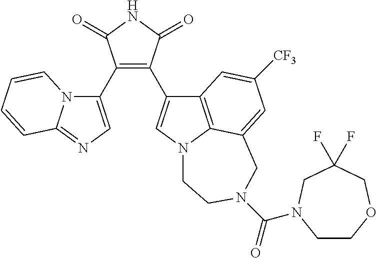 [SMILES: O=C1NC(=O)C(c2cnc3ccccn23)=C1c1cn2c3c(cc(C(F)(F)F)cc13)CN(C(=O)N1CCOCC(F)(F)C1)CC2] | 3-(2-(6,6-difluoro-1,4-oxazepane-4-carbonyl)- 9-(trifluoromethyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 45 | 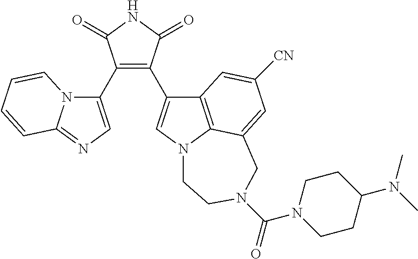 [SMILES: CN(C)C1CCN(C(=O)N2CCn3cc(C4=C(c5cnc6ccccn56)C(=O)NC4=O)c4cc(C#N)cc(c43)C2)CC1] | 2-(4-(dimethylamino)piperidine-1-carbonyl)- 7-(4-(imidazo[1,2-a]pyridin-3-yl)-2,5-dioxo- 2,5-dihydro-1H-pyrrol-3-yl)-1,2,3,4- tetrahydro-[1,4]diazepino[6,7,1-hi]indole-9- carbonitrile |
| Compound I- 46 | 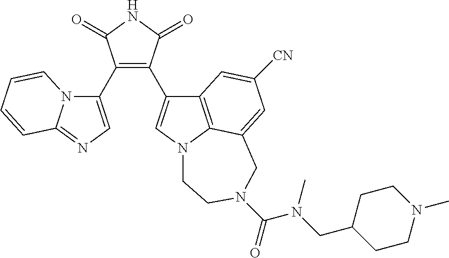 [SMILES: CN1CCC(CN(C)C(=O)N2CCn3cc(C4=C(c5cnc6ccccn56)C(=O)NC4=O)c4cc(C#N)cc(c43)C2)CC1] | 9-cyano-7-(4-(imidazo[1,2-a]pyridin-3-yl)- 2,5-dioxo-2,5-dihydro-1H-pyrrol-3-yl)-N- methyl-N-((1-methylpiperidin-4-yl)methyl)- 3,4-dihydro-[1,4]diazepino[6,7,1-hi]indole- 2(1H)-carboxamide |
| Compound I- 47 | 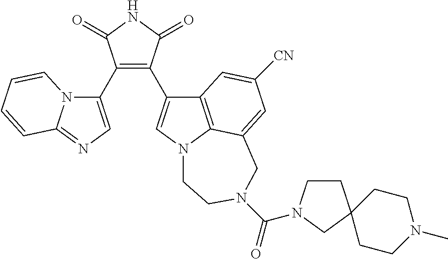 [SMILES: CN1CCC2(CC1)CCN(C(=O)N1CCn3cc(C4=C(c5cnc6ccccn56)C(=O)NC4=O)c4cc(C#N)cc(c43)C1)C2] | 7-(4-(imidazo[1,2-a]pyridin-3-yl)-2,5-dioxo- 2,5-dihydro-1H-pyrrol-3-yl)-2-(8-methyl-2,8- diazaspiro[4.5]decane-2-carbonyl)-1,2,3,4- tetrahydro-[1,4]diazepino[6,7,1-hi]indole-9- carbonitrile |
| Compound I- 48 | 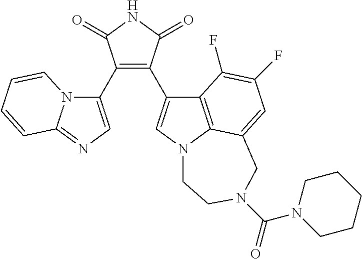 [SMILES: O=C1NC(=O)C(c2cnc3ccccn23)=C1c1cn2c3c(cc(F)c(F)c13)CN(C(=O)N1CCCCC1)CC2] | 3-(8,9-difluoro-2-(piperidine-1-carbonyl)- 1,2,3,4-tetrahydro-[1,4]diazepino[6,7,1- hi]indol-7-yl)-4-(imidazo[1,2-a]pyridin-3-yl)- 1H-pyrrole-2,5-dione |

In other embodiments, Wnt agonist and/or GSK3 inhibitor as described in WO 2018/125746, US 20180214458 and U.S. Ser. No. 62/608,663 the contents of which are each incorporated by reference in their entireties.

### Epigenetic Modulators

Epigenetic modulators include epigenetic modifiers, mediators and modulators. Epigenetic modifiers are genes whose products modify the epigenome directly through DNA methylation, the post-translational modification of chromatin or the alteration of the structure of chromatin. The epigenetic mediators are often the target of epigenetic modification, although they are rarely mutated themselves. The epigenetic mediators largely overlap with the genes involved in stem cell reprogramming and their role in cancer followed directly from the discovery of their reprogramming role. Epigenetic mediators are those genes whose products are the targets of the epigenetic modifiers. Epigenetic modulators are the as genes lying upstream of the modifiers and mediators in signaling and metabolic pathways

In some embodiments, an agent of having activity as an epigenetic modulator is selected from the group consisting of an HDAC inhibitor, a LSD-1 inhibitor, an EZH2 inhibitor, a DOT1L inhibitor, and KDM inhibitor.

As used herein, the term epigenetic modulator defines an agent that is capable of having activity as an epigenetic modifiers, mediators or modulators, when tested, for example, in an in vitro assay.

### HDAC Inhibitors

Histone deacetylases (HDAC) are a class of enzymes that remove acetyl groups (O═C—CH3) from an ε-N-acetyl lysine amino acid on a histone, allowing the histones to wrap the DNA more tightly. This is important because DNA is wrapped around histones, and DNA expression is regulated by acetylation and de-acetylation.

HDACs are classified in four classes depending on sequence homology to the yeast original enzymes and domain organization. The HDAC classes include HDACI, HDAC IIA, HDAC IIB, HDAC III, and HDAC IV.

Histone deacetylase (HDAC) inhibitors (HDACi, HDIs) are chemical compounds that inhibit histone deacetylases.

Thus, “HDAC inhibitor” refers to an agent capable of the decreasing the expression or enzymatic activity of an HDAC. For example, administration of an HDAC inhibitor results in a decrease in histone deacetylation of a target gene in a cell.

In certain embodiments, the HDAC inhibitor decreases the expression or enzymatic activity of HDAC by at least 5, 10, 20, 30, 40, 50, 60, 70, 80, 90, or 100% relative to a control, for example, relative to a baseline level of activity.

In certain embodiments, the HDAC inhibitor decreases histone deacetylation of a target gene by at least 5, 10, 20, 30, 40, 50, 60, 70, 80, 90, or 100% relative to a control, for example, relative to a baseline level of activity.

In some embodiments, the HDAC inhibitor increases expression or activity of a target gene by at least 5, 10, 20, 30, 40, 50, 60, 70, 80, 90, or 100% relative to a control, for example, relative to a baseline level of activity.

In some embodiments, the HDAC inhibitor decreases expression or enzymatic activity of HDAC by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example, relative to a baseline level of activity.

In some embodiments, the HDAC inhibitor decreases histone deacetylation of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example, relative to a baseline level of activity.

In some embodiments, the HDAC inhibitor increases expression or activity of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example, relative to a baseline level of activity.

TABLE 4

| | | | Mechanism | | | | | |
|---|---|---|---|---|---|---|---|---|
| | | Chemo- | HDAC | Class | HDAC | Lgr5+ | Perilymph | Formulation |
| Agent | CAS | type | Inhib | selectivity | Potenc | Assay | Cone | Cone |
| Sodium | 1069- | Acid | 1, 2, 3, 8 | Class I | 39- | 100 | 100 μM- | 100 mM- |
| Valproate | 66-5 | | | | 161 μM | μM-4 | 4 mM | 4000 mM |
| | | | | | | mM | | |
| 2-hexyl-4- | 96017- | Acid | 1,2,3,8 | Class I | 13 μM | 100 | 100 μM- | 100 mM- |
| pentynoic acid | 59-3 | | | | | μM-4 | 4 mM | 4000 mM |
| | | | | | | mM | | |
| Na | 1716 - | Acid | 1, 2, 3, 8 | Class I > | 9- | 100 | 100 μM- | 100 mM- |
| phenylbutyrate | 12-7 | | | Class IIb | 16 μM | μM-4 | 4 mM | 4000 mM |
| | | | | | | 1 nm | | |

In various embodiments, the treatments disclosed herein include use an HDAC inhibitor. Exemplary HDAC inhibitors are provide in Table 5

TABLE 5

| Class | Agent | CAS |
|---|---|---|
| Aliphatic Acid | Butyrate | 107-92-6 |
| Aliphatic Acid | Phenyl butyrate | 1821-12-1 |
| Aliphatic Acid | Valproic Acid | 99-66-1 |
| Aliphatic Acid Ester | AN-9 | 122110-53-6 |
| Amine | 932718-22-4 | 932718-22-4 |
| Benzamide | 4SC-202 | 1186222-89-8 |
| Benzamide | BML-210 | 537034-17-6 |
| Benzamide | Chidamide | 743438-4440 |
| Benzamide | Entinostat (MS-275) | 209783-80-2 |
| Benzamide | HDAC Inhibitor IV | 537034-15-4 |
| Benzamide | Mocetinostat (MGCD0103) | 726169-73-9 |
| Benzamide | NKL 22 | 537034-15-4 |
| Benzamide | RGFP109 | 1215493-56-3 |
| Benzamide | RGFP136 | 1215493-97-2 |
| Benzamide | RGFP966 | 1357389-11-7 |
| Benzamide | Tacedinaline | 112522-64-2 |
| Benzamide | TC-H 106, HDAC Inhibitor VII | 937039-45-7 |
| Cyclic peptide | Apicidin | 183506-66-3 |
| Cyclic peptide | Dihydrochlamydocin | 52574-64-8 |
| Cyclic peptide | HC Toxin | 83709-65-8 |
| Cyclic peptide | Romidepsin | 128517-07-7 |
| Cyclic Peptide | Thailandepsin A | 1269219-30-8 |
| Cyclic peptide | Trapoxin A | 133155-89-2 |
| Epoxide | (−)-Depudecin | 139508-73-9 |
| Epoxide | Parthenolide | 20554-84-1 |
| Hydroxamate | (S)-HDAC-42 | 935881-37-1 |
| Hydroxamate | 4-(dimethylamino)-N-[6- | 193551-00-7 |
| | (hydroxyamino)-6-oxohexyl]- | |
| | benzamide | |
| Hydroxamate | 4-iodo-SAHA | 1219807-87-0 |
| Hydroxamate | 4SC-201 (Resminostat) | 864814-88-0 |
| Hydroxamate | ACY1215 | 1316214-52-4 |
| Hydroxamate | APHA Compound 8 | 676599-90-9 |
| Hydroxamate | BRD9757 | 1423058-85-8 |
| Hydroxamate | Bufexamac | 2438-72-4 |
| Hydroxamate | Butyrylhydroxamic acid | 4312-91-8 |
| Hydroxamate | CAY10603 | 1045792-66-2 |
| Hydroxamate | CBHA | 174664-65-4 |
| Hydroxamate | CG200745 | 936221-33-9 |
| Hydroxamate | CHR-3996 | 1256448-47-1 |
| Hydroxamate | CUDC-101 | 1012054-59-9 |
| Hydroxamate | Droxinostat | 99873-43-5 |
| Hydroxamate | HDAC Inhibitor II | 174664-65-4 |
| Hydroxamate | HDAC Inhibitor VI | 926908-04-5 |
| Hydroxamate | HDAC Inhibitor XXIV | 854779-95-6 |
| Hydroxamate | HDAC6 Inhibitor III | 1450618-49-1 |
| Hydroxamate | HDAC-IN-1 | 1239610-44-6 |
| Hydroxamate | HNHA | 926908-04-5 |
| Hydroxamate | HPOB | 1479651-50-2 |
| Hydroxamate | ITF2357 | 497833-27-9 |
| Hydroxamate | ITF2357 (Givinostat) | 497833-27-9 |
| Hydroxamate | LAQ-824 | 591207-53-3 |
| Hydroxamate | LBH-589 (panobinostat) | 404950-80-7 |
| Hydroxamate | LMK235 | 1418033-25-6 |
| Hydroxamate | M 344 | 251456-60-7 |
| Hydroxamate | MC 1568 | 852475-26-4 |
| Hydroxamate | Nexturastat A | 1403783-31-2 |
| Hydroxamate | NSC 57457 | 6953-61-3 |
| Hydroxamate | Oxamflatin | 151720-43-3 |
| Hydroxamate | PCI-24781 (Abexinostat) | 783355-60-2 |
| Hydroxamate | PCI-34051 | 950762-95-5 |
| Hydroxamate | PDX-101 (belinostat) | 866323-14-0 |
| Hydroxamate | Pyroxamide | 382180-17-8 |
| Hydroxamate | SAHA (Zolinza, vorinostat) | 149647-78-9 |
| Hydroxamate | SB939 (Pracinostat) | 929016-96-6 |
| Hydroxamate | SBHA | 38937-66-5 |
| Hydroxamate | Scriptaid | 287383-59-9 |
| Hydroxamate | Tefinostat (CHR-2845) | 914382-60-8 |
| Hydroxamate | Trichostatin A (TSA) | 58880-19-6 |
| Hydroxamate | Tubacin | 537049-40-4 |
| Hydroxamate | Tubastatin A | 1252003-15-8 |
| Hydroxamate | VAHA | 106132-78-9 |
| Ketone | Compound 43 | 891259-76-0 |
| Ketone-a-ketoamides | 436150-82-2 | 436150-82-2 |
| Ketone-CF3 | Compound 27 | 946499-86-1 |
| Ketone-CF3 | Compound 6e | 946500-31-8 |
| Ketone-CF3 | Compound 6H | 946500-39-6 |
| Non classical | Tasquinimod | 254964-60-8 |
| Non classical | TMP269 | 1314890-29-3 |
| Polyketide | Ratjadone A | 163564-92-9 |
| Silylalcohol | 1587636-32-5 | 1587636-32-5 |
| Sulphonamide | 1587636-33-6 | 1587636-33-6 |
| Sulphonamide | 329967-25-1 | 329967-25-1 |
| Sulphonyl Urea | 960130-17-0 | 960130-17-0 |
| Thioester | HDAC Inhibitor XXII | 848354-66-5 |
| Thioester | KD 5170 | 940943-37-3 |
| Thioester | PTACH | 848354-66-5 |
| Thioester | TCS HDAC6 20b | 956154-63-5 |
| Thioketone | SIRT1/2 Inhibitor VII | 143034-06-4 |
| Thiol | 1368806-68-1 | 1368806-68-1 |
| Thiol | 1428536-05-3 | 1428536-05-3 |
| Thiol | 827036-76-0 | 827036-76-0 |
| Thiol | 828920-13-4 | 828920-13-4 |
| Thiol | 908860-21-9 | 908860-21-9 |
| Tropones | 1411673-95-4 | 1411673-95-4 |
| Tropones | 46189-88-2 | 46189-88-2 |

In some embodiments the HDAC inhibitor is a class I HDAC inhibitor. In these embodiments, the class I HDAC inhibitor may be a short chain carboxylic acid. In some embodiments, the HDAC inhibitor is valproic acid (VPA), 2-hexyl-4-pentynoic acid, or Na phenylbutyrate. In certain embodiments, the HDAC inhibitor is valproic acid (VPA). In certain such embodiments, the HDAC inhibitor is sodium valproate.

As used herein the terms “valproic acid” and “VPA” are used interchangeably to refer to the same compound. Moreover, as used herein the terms “valproic acid” and “VPA” also refer any pharmaceutically acceptable salts thereof.

### LSD1 Inhibitors

LSD1 mediated H3K4 demethylation can result in a repressive chromatin environment that silences gene expression. LSD1 has been shown to play a role in development in various contexts. LSD1 can interact with pluripotency factors in human embryonic stem cells and is important for decommissioning enhancers in stem cell differentiation. Beyond embryonic settings, LSD1 is also critical for hematopoietic differentiation. LSD1 is overexpressed in multiple cancer types and recent studies suggest inhibition of LSD1 reactivates the all-trans retinoic acid receptor pathway in acute myeloid leukemia (AML). These studies implicate LSD1 as a key regulator of the epigenome that modulates gene expression through post-translational modification of histones and through its presence in transcriptional complexes.

Thus, an “LSD1 inhibitor” refers to an agent capable of the decreasing the expression or enzymatic activity of LSD1. For example, an LSD1 inhibitor results in a decrease in H3K4 demethylation of a target gene in a cell, for instance, in a cochlear cell or a vestibular. cell

In certain embodiments, an LSD1 inhibitor decreases the expression or enzymatic activity of LSD1 by at least 5, 10, 20, 30, 40, 50, 60, 70, 80, 90, or 100% relative to a control, for example, relative to a baseline level of activity.

In certain embodiments, an LSD1 inhibitor decreases H3K4 demethylation by at least 5, 10, 20, 30, 40, 50, 60, 70, 80, 90, or 100% relative to a control, for example, relative to a baseline level of activity.

In some instances, an LSD1 inhibitor decreases H3K4 demethylation by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example, relative to a baseline level of activity.

In some instances, a LSD1 inhibitor modulates (i.e., increases or decreases) expression or activity of a target gene by at least 5, 10, 20, 30, 40, 50, 60, 70, 80, 90, or 100% relative to a control, for example, relative to a baseline level of activity.

In some instances, a LSD1 inhibitor modulates (i.e., increases or decreases) expression or enzymatic activity of LSD1 by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example, relative to a baseline level of activity.

In some instances, an LSD1 inhibitor is reversible. In other instances, the LSD1 inhibitor is irreversible.

Exemplary agents having activity as an LSD1 inhibitor are provided in Table 6 below, including pharmaceutically acceptable salts thereof.

TABLE 6

| | | | | | | Select | | | | | | |
|---|---|---|---|---|---|---|---|---|---|---|---|---|
| | | pKi | Reversible | | Select | MAOs | Liter | | | Formul. | Human | |
| | | or | or | Chemo- | KDM | A and | at. | Lgr5+ | Perilymph | Conc. | Plasma | Human |
| Agent | CAS | IC50 | Irreversible | type | 1b | B | Cell | Assay | Conc. | Intratymp. | Conc | Dossage |
| GSK- | 1401966- | 1.7 | Irreversible | Cyproylamine | | 20 μM | EC50 = | 40 nM- | 40 nM- | 40 μM to | 1-100 | 1 or 2 |
| 2879552 | 69-5 | μM | | | | | 2-240 | 30 μM | 30 □M | 30 mM | nM | mg |
| | | (0.11 | | | | | nM | | | | | QD |
| | | μM) | | | | | | | | | | PO |
| GSK- | 1431368- | 16 | Irreversible | Cyproylamine | >1000X | >1000X | | 4 nM- | 4 nM-50 | 4 μM to | 1-100 | 10-100 |
| LSD1 | 48-7 | nM | | | | | | 50 μM | μM | 50 mM | nM | mg |
| | | | | | | | | | | | | PO |
| Phenelzine | 51-71-8 | 5.6 | Irreversible | Hydrazine | | MAO | 900 | | 0.1-10 | 0.1-10 | Cmax | 15-90 |
| sulfate | | μM | | | | inhibit | nM in | | μM | mM | 10 to | mg/day |
| | | | | | | or 900 | Cell | | | | 60 | PO |
| | | | | | | nM in | | | | | ng/mL | |
| | | | | | | Cell | | | | | (73- | |
| | | | | | | | | | | | 440 | |
| | | | | | | | | | | | nM) | |
| TCP | 155-09-9 | 11- | Irreversible | Cyproylamine | 186 | 1 μM, | | 0.1-20 | 0.1-20 | 0.1-20 | Cmax | 15- |
| (tranylcypro- | | 477 | | | μM | | | μM | μM | mM | 30-200 | 150 |
| mine) | | μM | | | | | | | | | ng/ml | mg/day |
| | | | | | | | | | | | (255- | PO |
| | | | | | | | | | | | 1500 | |
| | | | | | | | | | | | nM) | |
| ORY-1001 | 1431326- | <20 | Irreversible | Cyproylamine | >100 | >100 | 0.5-3 | 41 nM | 4 nM-50 | 4 uM to | 1-100 | 1-100 |
| (RG6016, | 61-2 | nM | | | μM | □M | nM | | μM | 50 mM | nM | mg |
| RO7051790, | | | | | | >100 | | | | | | PO |
| Iadademstat) | | | | | | □M | | | | | | |
| RN-1 | 1781835- | 70 | Irreversible | Cyproylamine | | 0.51 | | 41 nM | 4 nM-50 | 4 uM to | 1-100 | 10-100 |
| | 13-9 | nM | | | | μM | | | μM | 50 mM | nM | mg |
| | | | | | | 2.78 | | | | | | PO |
| | | | | | | μM | | | | | | |
| CC-90011 | 2179319- | | Reversible | Likely | | | | | | | | |
| | 65-2 | | | pyrimidinyl | | | | | | | | |
| GCG-11047 | 308145- | | Reversible | Polyamine | | | | | | | | |
| (PG-11047) | 19-9 | | | | | | | | | | | |
| IMG-7289 | 2229826- | | Irreversible | Likely | | | | | | | Cmax | 80 mg |
| | 41-7 | | | Cyproylamine | | | | | | | 63 | QD |
| | | | | | | | | | | | ng/ml | PO |
| INCB059872 | 1802909- | | Irreversible | Likely | | | | | | | | |
| | 49-4 | | | Cyproylamine | | | | | | | | |
| ORY-1001 | 1431326- | <20 | Irreversible | Cyproylamine | >100 | >100 | 0.5-3 | Activity | | | | Up to |
| (RG6016, | 61-2 | nM | | | μM | μM | nM | | | | | 2 mg |
| RO7051790, | | | | | | >100 | | | | | | |
| Iadademstat) | | | | | | μM | | | | | | |
| ORY-2001 | 1357362- | | Irreversible | Cyproylamine | | | | | | | | |
| (Vafidemstat) | 02-7 | | | | | | | | | | | |
| Osimertinib | 1421373- | 3.98 | Reversible | Pyrimidinyl | | | 43 | | | | | 10-80 |
| (AZD9291) | 65-0 | μM | | | | | nM | | | | | mg |
| SP-2577 | 1423715- | | Irreversible | Hydrazone | | | | | | | | |
| (Seclidemstat) | 37-0 | | | | | | | | | | | |
| | 1821307- | | | | | | | | | | | |
| | 10-1 | | | | | | | | | | | |
| TCP Trans | 3721- | 284 | Irreversible | Cyproylamine | 137 | B: 4.4 | | | | | | |
| Chiral | 28-6 | μM | | | μM | μM | | | | | | |
| TCP Trans | 3721- | 168 | Irreversible | Cyproylamine | 127 | B: 89 | | | | | | |
| Chiral | 26-4 | μM | | | μM | μM | | | | | | |
| TCP Cis | 13531- | | Irreversible | Cyproylamine | | 11 μM | | | | | | |
| | 35-6 | | | | | 19 μM | | | | | | |
| TCP Cis | 69684- | | Irreversible | Cyproylamine | | | | | | | | |
| Chiral | 88-4 | | | | | | | | | | | |
| TCP Cis | 69684- | | Irreversible | Cyproylamine | | | | | | | | |
| Chiral | 89-5 | | | | | | | | | | | |
| RN-1 | 1781835- | 70 | Irreversible | Cyproylamine | 0.51 | | FHZ- | | | | | |
| | 13-9 | nM | | | μM | | 455 | | | | | |
| | | | | | 2.78 | | | | | | | |
| | | | | | μM | | | | | | | |
| Compound 1 | 1221595- | 10 | Irreversible | Cyproylamine | | | | | | | | |
| | 26-1 | nM | | | | | | | | | | |
| Compound 45 | 1667721- | 9 | Irreversible | Cyproylamine | | 15 μM | | | | | | |
| | 01-8 | nM | | | | >40 | | | | | | |
| | | | | | | μM | | | | | | |
| RN-7 | 1352345- | 31 | Irreversible | Cyproylamine | | | | | | | | |
| | 82-4 | nM | | | | | | | | | | |
| Compound 5A | 1613476- | 12 | Irreversible | Cyproylamine | | | | | | | | |
| | 09-7 | nM | | | | | | | | | | |
| Compound 2 | 1235863- | 67 | Irreversible | Cyproylamine | | >37 | | | | | | |
| | 51-0 | nM | | | | μM | | | | | | |
| Compound 43 | 1784703- | 610 | Irreversible | Cyproylamine | | | | | | | | |
| | 61-2 | nM | | | | | | | | | | |
| Compound 12f | 1802319- | 86 | Irreversible | Cyproylamine | | 460 | | | | | | |
| | 25-0 | nM | | | | nM, | | | | | | |
| | | | | | | >70 | | | | | | |
| | | | | | | μM | | | | | | |
| T-3775440 | 1422620- | 2.1 | Irreversible | Cyproylamine | | 110 | | | | | | |
| | 34-5 | nM | | | | μM, 17 | | | | | | |
| | | | | | | μM | | | | | | |
| OG-L002 | 1357299- | 20 | Irreversible | Cyproylamine | | | | | | | | |
| | 45-6 | nM | | | | | | | | | | |
| S-2101 | 1239262- | 990 | Irreversible | Cyproylamine | | | | | | | | |
| | 36-2 | nM | | | | | | | | | | |
| NCL-1 | 1196119- | 1.6 | Irreversible | Cyproylamine | | | | | | | | |
| | 03-5 | μM | | | | | | | | | | |
| Compound 9A | 2095849- | 1.2 | Irreversible | Cyproylamine | | | | | | | | |
| | 74-2 | μM | | | | | | | | | | |
| Compound 19l | 2173543- | 0.97 | Irreversible | Cyproylamine | | | | | | | | |
| | 81-0 | μM | | | | | | | | | | |
| NCD-25 | 1456972- | 480 | Irreversible | Cyproylamine | | | | | | | | |
| | 46-5 | nM | | | | | | | | | | |
| NCD-38 | 2078047- | 590 | Irreversible | Cyproylamine | | | | | | | | |
| | 42-2 | nM | | | | | | | | | | |
| Compound 14A | 2247939- | 2.2 | Irreversible | Cyproylamine | | | | | | | | |
| | 53-1 | nM | | | | | | | | | | |
| Compound 15A | 2247939- | 70 | Irreversible | Cyproylamine | | | | | | | | |
| | 55-3 | nM | | | | | | | | | | |
| Compound 15B | 2247939- | 11 | Irreversible | Cyproylamine | | | | | | | | |
| | 56-4 | nM | | | | | | | | | | |
| Compound 4 | 2226461- | 43 | Irreversible | Cyproylamine | | | 3.8 | | | | | |
| | 60-3 | nM | | | | | μM | | | | | |
| Pargyline | 555-57- | 1000 | Irreversible | Amino-propyne | | | | | | | | |
| | 7 | μM | | | | | | | | | | |
| Peptide | 945548- | | Irreversible | Amino-propyne | | | | | | | | |
| | 35-6 | | | | | | | | | | | |
| Bizine | 1591932- | 59 | Irreversible | Hydrazine | | | | FHZ- | | | | |
| | 50-1 | nM | | | | | | 457 | | | | |
| Compound 5a | 1990536- | 1.4 | Reversible | Hydrazone | | | | | | | | |
| | 90-7 | nM | | | | | | | | | | |
| Compound 5n | 1990537- | 1.7 | Reversible | Hydrazone | | | | | | | | |
| | 03-5 | nM | | | | | | | | | | |
| SP-2509 | 1423715- | 13 | Reversible | Hydrazone | | >300 | 350- | Activity | | | | |
| (HCI-2509) | 09-6 | nM | | | | μM | 650 | | | | | |
| | | | | | | | nM | | | | | |
| LSD1-IN-32 | 2137044- | 83 | Reversible | Amide | | | 670 | | | | | |
| | 49-4 | nM | | | | | nM | | | | | |
| LSD1-IN-11p | 2101951- | 20- | Reversible | Pyrazole | | | 0.52 | | | | | |
| | 67-9 | 80 | | | | | μM | | | | | |
| | | nM | | | | | | | | | | |
| Resveratrol | 501-36- | 15 | Reversible | Resveratrol | | | | | | | | |
| | 0 | μM | | | | | | | | | | |
| Hydroxylamine | 2035912- | 121 | Reversible | Resveratrol | | | | | | | | |
| | 55-9 | nM | | | | | | | | | | |
| Compound 8c | 2170023- | 283 | Reversible | Resveratrol | | | 5 to 9 | | | | | |
| | 28-4 | nM | | | | | μM | | | | | |
| CBB-1007 | 1379573- | 2.1 | Reversible | Polyamine | | | IC50 | Activity | | | | |
| | 92-8 | μM | | | | | ≤5 | | | | | |
| | | | | | | | μM | | | | | |
| Namoline | 342795- | 51 | Reversible | Benzopyran-4- | | | | | | | | |
| | 11-3 | μM | | one | | | | | | | | |
| GSK-354 | 1841508- | 29- | Reversible | Diphpyridine | | A >50 | 1.3 | | | | | |
| | 96-0 | 80 | | | | μM | μM | | | | | |
| | | nM | | | | | | | | | | |
| | | | | | | B = 19 | | | | | | |
| | | | | | | μM | | | | | | |
| GSK-690 | 2101305- | 37 | Reversible | Diphpyridine | | | | | | | | |
| | 84-2 | nM | | | | | | | | | | |
| E11 | 1239589- | 243 | Reversible | 2,4- | | | | | | | | |
| | 91-3 | nM | | Quinazolinediamine | | | | | | | | |
| MC2694 | 1435055- | 1 | Reversible | 2,4- | | | | | | | | |
| | 66-5 | μM | | Quinazolinediamine | | | | | | | | |
| Alpha- | 11/11/6147 | 2.8 | Reversible | mangostin | | | | | | | | |
| mangostin | | μM | | | | | | | | | | |
| Compound | 1923750- | 0.41 | Reversible | Barbituate | | | | | | | | |
| 12 A | 07-5 | μM | | | | | | | | | | |
| Compound 4 | 126118- | 6.4 | Reversible | Purine-2,6-dione | | | | | | | | |
| | 57-8 | μM | | | | | | | | | | |
| Compound 10d | 2226997- | 4 | Reversible | Carboxamide | | | | | | | | |
| | 31-3 | μM | | | | | | | | | | |
| Compound 90 | 1884266- | 162 | Reversible | Carboxamide | | | | | | | | |
| | 15-2 | nM | | | | | | | | | | |
| Compound 46 | 1884266- | 8 | Reversible | Carboxamide | | | 1-4 | | | | | |
| | 36-7 | nM | | | | | μM | | | | | |
| Compound 49 | 1884266- | 7 | Reversible | Carboxamide | | | 1-4 | | | | | |
| | 49-2 | nM | | | | | μM | | | | | |
| Compound 50 | 1884266- | 8 | Reversible | Carboxamide | | | 1-4 | | | | | |
| | 48-1 | nM | | | | | μM | | | | | |
| Polymyxin B | 1404- | 157 | Reversible | Polymyxin B | | | | | | | | |
| | 26-8 | nM | | | | | | | | | | |
| Polymyxin E | 1066- | 193 | Reversible | Polymyxin E | | | | | | | | |
| | 17-7 | nM | | | | | | | | | | |
| Baicalin | 21967- | 3.0 | Reversible | Baicalin | | | | | | | | |
| | 41-9 | μM | | | | | | | | | | |
| Compound 16Q | 1612870- | 9.5 | Reversible | Benzenesulfonamide | | >500 | | | | | | |
| | 90-2 | μM | | | | μM | | | | | | |
| LSD1 | 1853269- | 1 | Reversible | Imidazole | | | | | | | | |
| inhibitor 24 | 07-4 | nM | | | | | | | | | | |
| geranylgeranoic | 35750- | 120 | Reversible | Geranyl | | | | | | | | |
| acid | 48-2 | μM | | | | | | | | | | |
| Geranylgeraniol | 24034- | 80 | Reversible | Geranyl | | | | | | | | |
| | 73-9 | μM | | | | | | | | | | |
| Thiocarbamate | 1430852- | 390 | Reversible | Thiocarbamate | | >1250 | | | | | | |
| | 56-4 | nM | | | | μM | | | | | | |
| Thiourea | 1637373- | 650 | Reversible | Thiourea | | >1250 | | | | | | |
| | 61-5 | nM | | | | μM | | | | | | |
| Thiourea | 2035417- | 154 | Reversible | Thiourea | | | | | | | | |
| | 23-1 | nM | | | | | | | | | | |
| Thienopyrrole | 1206028- | 2.9 | Reversible | Thienopyrrole | >100 | >100 | | | | | | |
| | 57-0 | μM | | | μM | μM, | | | | | | |
| | | | | | | 57 μM | | | | | | |
| Thienopyrrole | 1884266- | 162 | Reversible | Thienopyrrole | | | | | | | | |
| | 15-2 | nM | | | | | | | | | | |
| Thienopyrrole | 1884266- | 7.8 | Reversible | Thienopyrrole | 13 μM | 41 μM, | | | | | | |
| | 48-1 | nM | | | | 100 | | | | | | |
| | | | | | | μM | | | | | | |
| 4SC-202 | 910462- | 1-10 | Reversible | o-aminoph | | | | | | | | 25-400 |
| | 43-0 | μM | | | | | | | | | | mg/Day |
| ORY-3001 | 2179325- | | | | | | | | | | | |
| | 30-3 | | | | | | | | | | | |
| JL1037 | | | | | | | | | | | | |
| FLI-06 | 313967- | 92 | Inhibits | Dihydropyridine | | | | | | | | |
| | 18-9 | nM | expression | | | | | | | | | |
| | | | of LSD1 | | | | | | | | | |
| Rhodium | | 40 | | Rhodium | | | | | | | | |
| Complex 1 | | nM | | | | | | | | | | |

In some embodiments, an agent of having activity as a LSD1 inhibitor is GSK-2879552, GSK-LSD1, osimertinib (AZD9291), phenelzine sulfate, tranylcypromine (TCP), ORY-1001, seclidemstat (SP-2577), vafidemstat (ORY-2001), CC-90011, IMG-7289 or, INCB059872. In certain embodiments, the LSD1 inhibitor is GSK-2879552, GSK-LSD1, phenelzine sulfate or tranylcypromine (TCP).

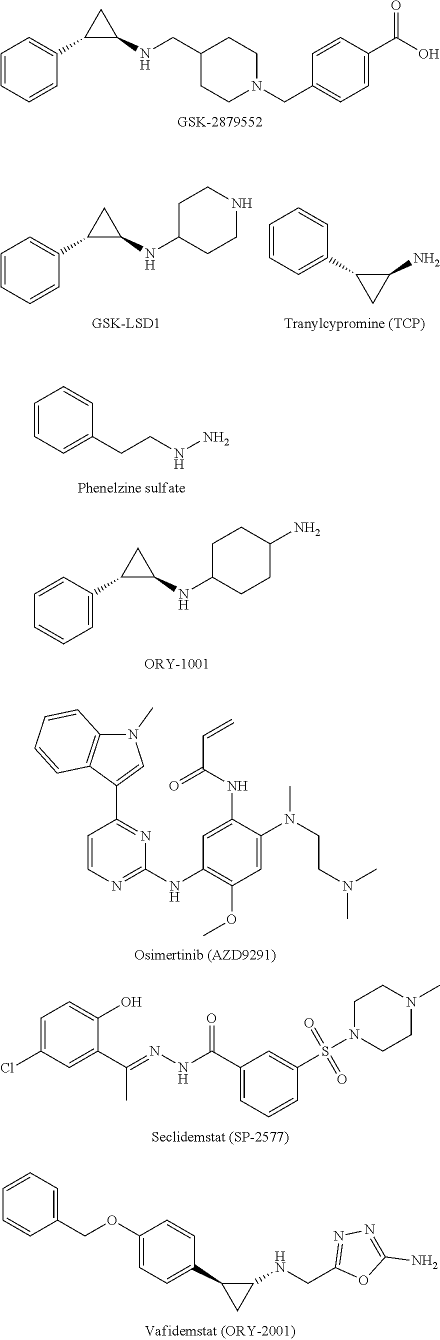

In some cases, the LSD1 inhibitor is GSK-2879552, GSK-LSD-1, or tranylcypromine (TCP).

### EZH2 Inhibitors

Enhancer of zeste homolog 2 (EZH2) is a histone-lysine N-methyltransferase enzyme encoded by EZH2 gene, that participates in histone methylation and, ultimately, transcriptional repression. EZH2 catalyzes the addition of methyl groups to histone H3 at lysine 27, by using the cofactor S-adenosyl-L-methionine. Methylation activity of EZH2 facilitates heterochromatin formation thereby silences gene function. Remodeling of chromosomal heterochromatin by EZH2 is also required during cell mitosis.

EZH2 is the functional enzymatic component of the Polycomb Repressive Complex 2 (PRC2), which is responsible for healthy embryonic development through the epigenetic maintenance of genes responsible for regulating development and differentiation EZH2 is responsible for the methylation activity of PRC2, and the complex also contains proteins required for optimal function (EED, SUZ12, JARID2, AEBP2, RbAp46/48, and PCL).

EZH2 inhibitors are chemical compounds that inhibit histone-lysine N-methyltransferase enzyme encoded by EZH2 gene

Thus, “EZH2 inhibitor” refers to an agent capable of the decreasing the expression or enzymatic activity of EZH2. For example, an EZH2 inhibitor results in a decrease in histone methylation of a target gene in a cell.

In certain embodiments, the EZH2 inhibitor decreases the expression or enzymatic activity of EZH2 by at least 5, 10, 20, 30, 40, 50, 60, 70, 80, 90, or 100% relative to a control, for example, relative to a baseline level of activity.

In certain embodiments, the EZH2 inhibitor decreases histone methylation of a target gene by at least 5, 10, 20, 30, 40, 50, 60, 70, 80, 90, or 100% relative to a control, for example, relative to a baseline level of activity.

In some embodiments, the EZH2 inhibitor increases expression or activity of a target gene by at least 5, 10, 20, 30, 40, 50, 60, 70, 80, 90, or 100% relative to a control, for example, relative to a baseline level of activity.

In some embodiments, the EZH2 inhibitor decreases expression or enzymatic activity of EZH2 by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example, relative to a baseline level of activity.

In some embodiments, the EZH2 inhibitor decreases histone methylation of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example, relative to a baseline level of activity.

In some embodiments, the EZH2 inhibitor increases expression or activity of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example, relative to a baseline level of activity.

Exemplary EZH2 inhibitors are provide in Table 7

TABLE 7

| | | | | | | | | | | Human | |
|---|---|---|---|---|---|---|---|---|---|---|---|
| | | pKi | | | Select | Lit | | | Formul. | In | |
| | | or | Enzymatic/ | | vs | Cell | Lgr5+ | Perilymph | Conc. | Vivo | Human |
| Agent | CAS | IC50 | Non-enzymatic | Chemo-type | EZH-1 | Poten | Assay | Conc | Intratymp | Conc | Dosage |
| PF-06821497 | 1844849- | <1 | enzymatic | 2-Pyridone | 70 | 4-6 | | 1-100 nM | 1-100 | 5-50 | 75 mg to |
| | 10-0 | nM | | | nM | nM | | | μM | nM | 625 mg |
| | | | | | | | | | | | BID PO |
| PF-06726304 | 1616287- | 1.1 | enzymatic | 2-Pyridone | | 10-25 | 100 | 100 nM- | 100 □M- | 100 | 100 to 1000/ |
| | 82-1 | nM | | | | nM | nM-3 | 3 □M | 3 mM | nM- | day mg |
| | | | | | | | □M | | | 30 | PO |
| | | | | | | | | | | □M | |
| CPI-1205 | 1621862- | 2.2 | enzymatic | 2-Pyridone | 24x | 32 | | 10-1000 | 10-1000 | 25-250 | 800 mg |
| | 70-1 | nM | | | | nM | | nM | μM | nM | BID and |
| | | | | | | | | | | | subsequently |
| | | | | | | | | | | | TID - PO |
| Valemetostat | 1809336- | 2.5 | enzymatic | 2-Pyridone | 8.4 | 25- | | 10-1000 | 10-1000 | 25-250 | PO starting |
| (DS-3201b, | 39-7 | nM | | | nM | 250 | | nM | μM | nM | dose of 100 |
| (R)-OR-S2) | | | | | | nM | | | | | mg QD |
| | | | | | | | | | | | with dose |
| | | | | | | | | | | | escal dep on |
| | | | | | | | | | | | tox |
| Tazemetostat | 1403254- | 2.5 | enzymatic | 2-Pyridone | 35x | | 0.37- | 0.37-1.1 | 0.1-1 | 100- | PO 100 |
| (EPZ-6438) | 99-8 | nM | | | | | 1.1 μM | μM | mM | 800 | BID to 800 |
| | | | | | | | | | | ng/ml | mg BID. |
| | | | | | | | | | | (200- | |
| | | | | | | | | | | 1600 | |
| | | | | | | | | | | nM) | |
| E11 | 1418308- | 13 | enzymatic | 2-Pyridone | 90x | | 5 μM | 1-10 μM | 1-10 mM | 1-10 | (100 to |
| | 27-6 | nM | | | | | | | | μM | 1000/ |
| | | | | | | | | | | | day mg PO) |
| CPI-169 | | 0.24 | enzymatic | 2-Pyridone | 6 nM | | | 1-10 μM | 1-10 mM | 1-10 | 100 to |
| | | nM | | | | | | | | μM | 1000/day |
| | | | | | | | | | | | mg PO |
| CPI-360 | 1802175- | 0.5 | enzymatic | 2-Pyridone | ~50 | | 0.1-10 | 0.1-10 | 0.1-10 | 1-10 | 100 to |
| | 06-9 | nM | | | nM | | □M | □M | mM | □M | 1000/day |
| | | | | | | | | | | | mg PO |
| EPZ-011989 | 1598383- | <3 | enzymatic | 2-Pyridone | | 94 | 100 | 0.10-30 | 100- | 0.10- | 100 to 1000/ |
| | 40-4 | nM | | | | nM | nM- | □M | 30,000 | 30 | day mg |
| | | | | | | | 30 | | □M | □M | PO |
| | | | | | | | □M | | | | |
| UNC 2399 | 1433200- | | enzymatic | 2-Pyridone | | | 0.1-10 | 0.1-10 | 0.1-10 | 0.1-100 | 100 to 1000/ |
| | 53-3 | | | | | | □M | □M | mM | □M | day mg |
| | | | | | | | | | | | PO |
| (R)-OR-S1 | 1809336- | 10 | enzymatic | 2-Pyridone | 7.4 | | | | | | IV 50 mg- |
| | 19-3 | nM | | | nM | | | | | | poor oral |
| | | | | | | | | | | | bio. |
| A-395 | 2089148- | 0.3 | EED | Amino | | 90 | | | | | 50 mg and |
| | 72-9 | nM | Inhibit | pyrrolidines | | nM | | | | | 200 mg PO |
| Astemizole | 68844- | 94 | EED | Benzimidazole | | | | | | | |
| | 77-9 | μM | Inhibit | | | | | | | | |
| Compound 19 | 2079895- | 1.3 | EED | Imidazole | | 1.9 | | | | | |
| | 22-8 | μM | Inhibit | | | μM | | | | | |
| Compound 22 | 1802175- | 2 | enzymatic | 2-Pyridone | | | | | | | |
| | 07-0 | nM | | | | | | | | | |
| Compound 24 | 1659298- | 40 | enzymatic | 2-Pyridone | | | | | | | |
| | 29-9 | nM | | | | | | | | | |
| Compound 34 | 2055347- | 29 | enzymatic | 2-Pyridone | >100x | | | | | | |
| | 72-1 | nM | | | | | | | | | |
| Compound 41 | 2055347- | 11 | enzymatic | 2-Pyridone | >100x | | | | | | |
| | 94-7 | nM | | | | | | | | | |
| CPI-0169 | 1450655- | 0.24 | enzymatic | 2-Pyridone | 6 nM | | | | | | |
| | 76-1 | nM | | | | | | | | | |
| CPI-0169 | 1802175- | 0.24 | enzymatic | 2-Pyridone | 6 nM | | 1.1 μM | | | | |
| | 07-0 | nM | | | | | | | | | |
| CPI-0169 | | | | | | | | | | | |
| CPI-360 | 1802175- | 0.5 | enzymatic | 2-Pyridone | ~50 | | | | | | |
| | 06-9 | nM | | | nM | | | | | | |
| EBI-2511 | 2098546- | 4 | enzymatic | 2-Pyridone | | | | | | | |
| | 05-3 | nM | | | | | | | | | |
| EED162 | 1010897- | 30 | EED | Triazo | | 80 | | | | | |
| | 73-0 | nM | Inhibit | | | nM | | | | | |
| EED226 | 2083627- | | EED | Triazo | | | Activity | | | | |
| | 02-3 | | Inhibit | | | | | | | | |
| EPZ-005687 | 1396772- | 24 | enzymatic | 2-Pyridone | 50X | | | | | | |
| | 26-1 | nM | | | | | | | | | |
| EPZ-011989 | 1598383- | <3 | enzymatic | 2-Pyridone | | 94 | | | | | |
| | 40-4 | nM | | | | nM | | | | | |
| GSK126 | 1346574- | <3 | enzymatic | 2-Pyridone | 150x | | | | | | |
| | 57-9 | nM | | | | | | | | | |
| GSK343 | 1346704- | 1.2 | enzymatic | 2-Pyridone | 60x | 174 | | | | | |
| | 33-3 | nM | | | | nM | | | | | |
| GSK503 | 1346572- | <10 | enzymatic | 2-Pyridone | | | | | | | |
| | 63-1 | nM | | | | | | | | | |
| GSK926 | 1346704- | 7.9 | enzymatic | 2-Pyridone | | 324 | | | | | |
| | 13-9 | nM | | | | nM | | | | | |
| MAK683 | 1951408- | | EED | Triazo | | | | | | | |
| (EED162) | 58-4 | | inhibitor | | | | | | | | |
| | (likely | | | | | | | | | | |
| | patent) | | | | | | | | | | |
| SHR2554 | 2098545- | | enzymatic | 2-Pyridone | | | | | | | |
| | 98-1 | | | | | | | | | | |
| SKLB1049 | 1826865- | 7.2 | enzymatic | 2-Pyridone | | 12 | | | | | |
| | 42-2 | nM | | | | μM | | | | | |
| ZLD1039 | 1826865- | <15 | enzymatic | 2-Pyridone | | | | | | | |
| | 46-6 | nM | | | | | | | | | |
| ZLD1122 | 1826865- | <15 | enzymatic | 2-Pyridone | | | | | | | |
| | 51-3 | nM | | | | | | | | | |
| | 1404094- | 74 | enzymatic | 2-Pyridone | | 2510 | | | | | |
| | 15-0 | nM | | | | nM | | | | | |
| | 1404094- | 14 | enzymatic | 2-Pyridone | | 1995 | | | | | |
| | 16-1 | nM | | | | nM | | | | | |
| DZNep | 102052- | | SAH- | SAH derived | | 1 μM | Activity | | | | |
| | 95-9 | | hydrolase | | | | | | | | |
| | | | inhibitor | | | | | | | | |
| Cmpd 44 | 1378002- | 32 | SAM | Benzamide | | 9 μM | | | | | |
| | 93-7 | nM | Comp | | | | | | | | |
| Compound 27 | 1676100- | 270 | SAH- | SAH derived | | | | | | | |
| | 59-6 | nM | hydrolase | | | | | | | | |
| | | | inhibitor | | | | | | | | |
| Sinefungin | 58944- | 20 | SAH- | SAH derived | | 33 | | | | | |
| | 73-3 | nM | hydrolase | | | nM | | | | | |
| | | | inhibitor | | | | | | | | |
| Tanshindiol B | 97465- | 520 | enzymatic | Tanshindiols | | | | | | | |
| | 70-8 | nM | | | | | | | | | |
| Tanshindiol C | 97465- | 550 | enzymatic | Tanshindiols | | | | | | | |
| | 71-9 | nM | | | | | | | | | |
| UNC1999 | 1431612- | 10 | enzymatic | 2-Pyridone | 10x | 124 | Activity | | | | |
| | 23-5 | nM | | | | nM | | | | | |
| (−)- | 989-51-5 | | enzymatic | a,b-unsat | | | | | | | |
| Epigallocatechin- | | | | | | | | | | | |
| 3-gallate | | | | | | | | | | | |
| (EGCG) | | | | | | | | | | | |
| Curcumin | 458-37-7 | | enzymatic | a,b-unsat | | | | | | | |
| MC1945 | 169903- | | enzymatic | a,b-unsat | | | | | | | |
| | 68-8 | | | | | | | | | | |
| MC1947 | 949090- | | Non- | | | | | | | | |
| | 12-4 | | enzymatic | | | | | | | | |
| MC1948 | 949090- | | Non- | | | | | | | | |
| | 20-4 | | enzymatic | | | | | | | | |
| SAH-EZH2 | | | Non- | reactive | | | | | | | |
| | | | enzymatic | | | | | | | | |
| Sulforaphane | 4478-93- | | EED | Stapled | | | | | | | |
| | 7 | | Inhibit | Peptide | | | | | | | |

In some embodiments the EZH2 inhibitor is PF-06821497, CPI-120, Valemetostat, Tazemetostat or El1.

### DOT1L Inhibitors

DOT1-like (Disruptor of telomeric silencing 1-like), histone H3K79 methyltransferase (S. cerevisiae), also known as DOT1L, is a protein found in humans, as well as other eukaryotes. The methylation of histone H3 lysine 79 (H3K79) by DOT1L which is a conserved epigenetic mark in many eukaryotic epigenomes, increases progressively along the aging process.

DOT1L inhibitors are chemical compounds that inhibits histone H3K79 methyltransferase.

Thus, “DOT1L inhibitor” refers to an agent capable of the decreasing the expression or enzymatic activity of DOT1L. For example, an EZH2 inhibitor results in a decrease in histone methylation of a target gene in a cell.

In certain embodiments, the DOT1L inhibitor decreases the expression or enzymatic activity of DOT1L by at least 5, 10, 20, 30, 40, 50, 60, 70, 80, 90, or 100% relative to a control, for example, relative to a baseline level of activity.

In certain embodiments, the DOT1L inhibitor decreases histone methylation of a target gene by at least 5, 10, 20, 30, 40, 50, 60, 70, 80, 90, or 100% relative to a control, for example, relative to a baseline level of activity.

In some embodiments, the DOT1L inhibitor increases expression or activity of a target gene by at least 5, 10, 20, 30, 40, 50, 60, 70, 80, 90, or 100% relative to a control, for example, relative to a baseline level of activity.

In some embodiments, the DOT1L inhibitor decreases expression or enzymatic activity of DOT1L by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example, relative to a baseline level of activity.

In some embodiments, the DOT1L inhibitor decreases histone methylation of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example, relative to a baseline level of activity.

In some embodiments, the DOT1L inhibitor increases expression or activity of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example, relative to a baseline level of activity.

Exemplary DOT1L inhibitors are provide in Table 8.

TABLE 8

| | | | | | | | | Human | |
|---|---|---|---|---|---|---|---|---|---|
| | | pKi | | | | | Formulation | In | |
| | | or | | Lit | Lgr5+ | Perilymph | Conc. | Vivo | Human |
| Agent | CAS | IC50 | Chemo-type | Cell | Assay | Conc | Intratymp | Conc | Dossage |
| EPZ004777 | 1338466- | 0.3 | Adenosine | 11 | 0.6- | 0.6-45 | 0.1-45 | 0.1- | 10-100 |
| | 77-5 | nM | | nM | 45 μM | μM | nM | 45 μM | mg/m2 |
| | | | | | | | | | per day |
| | | | | | | | | | IV |
| Pinometostat | 1380288- | 0.08 | Adenosine | 2.7 | | 0.1-10 | 0.1-10 | Total | 54-90 |
| (EPZ-5676) | 87-8 | nM | | nM | | μM | mM | plasma | mg/m2 |
| | | | | | | | | Css | per day |
| | | | | | | | | 800- | by |
| | | | | | | | | 1600 | continuous |
| | | | | | | | | ng/mL | IV, |
| | | | | | | | | (1.42- | Potential |
| | | | | | | | | 2.94 | for SC |
| | | | | | | | | μM) | dosing |
| | | | | | | | | (1-10 | |
| | | | | | | | | μM) | |
| SGC0946 | 1561178- | 0.3 | Adenosine | 10 | 0.6- | 0.6-5 | 0.6-5 mM | 0.1-5 | 10-100 |
| | 78-17-3 | nM | | nM | 5 μM | μM | | μM | mg/m2 |
| | | | | | | | | | per day |
| | | | | | | | | | IV |
| Bromo- | 1428254- | 77 | Adenosine | | | | | | |
| deaza- | 21-0 | nM | | | | | | | |
| SAH | | | | | | | | | |
| CN SAH | 1985669- | 13 | Adenosine | | | | | | |
| | 27-9 | nM | | | | | | | |
| Compound 10 | 1645266- | 29 | Adenosine | | | | | | |
| | 99-4 | nM | | | | | | | |
| Compound 13 | 1940206- | 0.4 | Aminopyrimidine | | | | | | |
| | 71-2 | nM | | | | | | | |
| Compound 7 | 2088518- | <1 | pyrrolopyrimidine | | | | | | |
| | 50-5 | nM | | | | | | | |
| Compound 8 | 1940224- | 14 | Acetylene | | | | | | |
| | 84-9 | nM | | | | | | | |
| EPZ002696 | 1381760- | 13 | Adenosine | | | | | | |
| | 94-6 | nM | | | | | | | |
| EPZ004450 | 1380315- | 4 | Adenosine | | | | | | |
| | 97-8 | nM | | | | | | | |
| SAH | 979- | 60 | Adenosine | | | | | | |
| | 92-0 | nM | | | | | | | |
| SYC-522 | 1381761- | 0.76 | Adenosine | 6 | | | | | |
| | 52-9 | nM | | μM | | | | | |
| SYC-687 | 1440509- | 1.1 | Non-Ribose | 200 | | | | | |
| | 94-3 | nM | | nM | | | | | |
| | 1440510- | 1.1 | Adenosine | 200 | | | | | |
| | 30-1, | nM | | nM | | | | | |
| | 1440509- | | | | | | | | |
| | 94-3 | | | | | | | | |
| Compound 21 | | | Peptides | | | | | | |
| Compound 28 | | | Peptides | | | | | | |
| Compound 6 | 167558- | 8.3 | triazolothiadiazol | | | | | | |
| | 34-1 | μM | | | | | | | |
| Compound 8H | | | pyrimidylamino- | | | | | | |
| | | | quinoline | | | | | | |
| | 1163729- | 1.5 | pyrimidine | | | | | | |
| | 79-0 | μM | | | | | | | |

In some embodiments the DOT1L inhibitor is EPZ004777, Pinometostat or SGC0946.

### KDM Inhibitors

About 30 JmjC domain-containing proteins have been identified as lysine demethylases in the human genome. Based on histone lysine sites and demethylation states, the JmjC domain-containing protein family is divided into six subfamilies: KDM2, KDM3, KDM4, KDM5, KDM6, and PHF. The JmjC domain-containing proteins belong to the Fe(II) and 2-oxoglutarate (2-OG)-dependent dioxygenases, which demethylate a variety of targets, including histones (H3K4, H3K9, H3K27, H3K36 as well as H1K26) and non-histone proteins. Unlike the LSD family, the JmjC-domain-containing histone demethylases (JHDMs) are able to erase all three kinds of histone lysine-methylation states since the JHDMs do not require protonated nitrogen for demethylation.

The KDM2 (also named FBXL) subfamily includes two members: KDM2A and KDM2B, KDM4 gene family, first identified in silico, consists of six members, including KDM4A, KDM4B, KDM4C, KDM4D, KDM4E and KDM4F. The KDM5 subfamily contains four enzymes: KDM5A, KDM5B, KDM5C and KDM5D, which specifically remove methyl marks from H3K4me2/3. In the human genome, the KDM6 subfamily is comprised of KDM6A, KDM6B and UTY, which share a well-conserved JmjC histone catalytic domain.

KDM inhibitors are chemical compounds that inhibit lysine demethylases.

Thus, “KDM inhibitor” refers to an agent capable of the decreasing the expression or enzymatic activity of KDM. For example, an KDM inhibitor results in a decrease in histone demethylation of a target gene in a cell.

In certain embodiments, the KDM inhibitor decreases the expression or enzymatic activity of KDM by at least 5, 10, 20, 30, 40, 50, 60, 70, 80, 90, or 100% relative to a control, for example, relative to a baseline level of activity.

In certain embodiments, the KDM inhibitor decreases histone demethylation of a target gene by at least 5, 10, 20, 30, 40, 50, 60, 70, 80, 90, or 100% relative to a control, for example, relative to a baseline level of activity.

In some embodiments, the KDM inhibitor increases expression or activity of a target gene by at least 5, 10, 20, 30, 40, 50, 60, 70, 80, 90, or 1000% relative to a control, for example, relative to a baseline level of activity.

In some embodiments, the KDM inhibitor decreases expression or enzymatic activity of KDM by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example, relative to a baseline level of activity.

In some embodiments, the KDM inhibitor decreases histone demethylation of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example, relative to a baseline level of activity.

In some embodiments, the KDM inhibitor increases expression or activity of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example, relative to a baseline level of activity.

Exemplary KDM inhibitors are provide in Table 9.

TABLE 9

| | | | Cova- | Se- | Se- | Se- | Se- | Se- | Se- | Se- | Se- | | | | | Human | |
|---|---|---|---|---|---|---|---|---|---|---|---|---|---|---|---|---|---|
| | | | lent | lect | lect | lect | lect | lect | lect | lect | lect | | | Peri- | Formu- | In | |
| | | | or | KD | KD | KD | KD | KD | KD | KD | KD | Lit | Lgr5+ | lymph | lation | Vivo | Human |
| Agent | CAS | Chemo-type | not | M1 | M2 | M3 | M4 | M5 | M6 | M7 | M8 | Cell | Assay | Conc | Conc | Conc | Dosage |
| AS 8351 | 796-42-9 | Hydrazone | | | | | | | | | | | 1-3 | 1-3 μM | 1-3 mM | 1-3 | 100- |
| | | | | | | | | | | | | | μM | | | μM | 2000 |
| | | | | | | | | | | | | | | | | | mg/day |
| TC-E | 1453071- | Hydroamate | No | | 6.8 | | 83 | 55 | >100 | 200, | | 16- | 0.12- | 0.12-10 | 0.12-10 | 0.12- | 100- |
| 5002 | 47-0 | | | | μM | | μM | μM | μM | 1200 | | 40 | 10 | μM | mM | 10 | 1000 |
| | | | | | | | | | | nM | | μM | μM | | | μM | mg/day |
| EPT- | | | | | | | 20- | <1 | 3K | | | 1.8 | 1- | 1-100 | 1-100 | 5-50 | 10 mg |
| 103182 | | | | | | | 50 | nM | | | | nM | 100 | nM | μM | nM | to |
| | | | | | | | | | | | | | nM | | | | 1000 |
| | | | | | | | | | | | | | | | | | mg/day |
| Compound | 1844064- | pyrimidin-4- | No | | | | 102/ | 23 | | | | | | | | | |
| 54k | 06-7 | one | | | | | 31 | nM | | | | | | | | | |
| | | | | | | | nM | | | | | | | | | | |
| Cmpd 1 | 1516899- | Cyproamine | Yes | | 220 | | | 190 | | 220 | | | | | | | |
| | 38-9 | isonicotinic | | | nM | | | nM | | nM | | | | | | | |
| | | acid | | | | | | | | | | | | | | | |
| Cmpd | 1613514- | Isonicotinic | No | | | | | <100 | | | | 0.1- | | | | | |
| 105 | 89-8 | acid | | | | | | nM | | | | 1 | | | | | |
| | | | | | | | | | | | | μM | | | | | |
| Compound | 1461602- | Isonicotinic | No | | | | <100 | <100 | | | | | | | | | |
| 34 | 86-7 | acid | | | | | nM | nM | | | | | | | | | |
| Compound | 1628332- | pyrido- | No | | | | | <100 | | | | | | | | | |
| 41 | 52-4 | pyrimidinone | | | | | | nM | | | | | | | | | |
| compound | 1628210- | cyanopyrazole | No | | | | | 15 | | | | 340 | | | | | |
| 48 | 26-3 | | | | | | | nM | | | | nM | | | | | |
| Compound | 1993438- | naphthyridones | No | | | | | 206 | | | | >10 | | | | | |
| 18 | 65-5 | | | | | | | nM | | | | μM | | | | | |
| Compound | 1613410- | pyrazolyl- | No | | | | | 10 | | | | ~1 | | | | | |
| 33 | 75-5 | pyridines | | | | | | nM | | | | μM | | | | | |
| Compound | 1905482- | Amide | No | | | | | | | | | 1-10 | | | | | |
| 48 | 57-6 | | | | | | | | | | | μM | | | | | |
| Compound | 1905482- | Pyrazole | No | | | | | | | | | | | | | | |
| 48 | 57-6 | | | | | | | | | | | | | | | | |
| Compound | 1905481- | Pyrazole | No | | | | | | | | | | | | | | |
| 49 | 35-7 | | | | | | | | | | | | | | | | |
| Compound | 1905481- | Pyrazole | No | | | | | | | | | | | | | | |
| 50 | 36-8 | | | | | | | | | | | | | | | | |
| Compound | 2169272- | 1-H-Indole | No | | | | | 50 | | | | | | | | | |
| 6 | 46-0 | | | | | | | nM | | | | | | | | | |
| Compound | 1807514- | Triazole | No | | | | | 65 | | | | 1.5 | | | | | |
| R-35 | 47-1 | | | | | | | nM | | | | μM | | | | | |
| CPI-455 | 1628208- | cyanopyrazole | No | | | | 41 | 13, 2 | | | | 90 | | | | | |
| | 23-0 | | | | | | nM | nM | | | | nM | | | | | |
| CPI- | 1628214- | cyanopyrazol | No | | | | | 1.1 | | | | | | | | | |
| 4203 | 07-2 | | | | | | | μM | | | | | | | | | |
| E67-2 | 1364914- | Quinazoline | No | | | | | | | | | | | | | | |
| | 62-4 | | | | | | | | | | | | | | | | |
| GSK- | 1628332- | Pyrazole | No | | | | | 14 | | | | | | | | | |
| 467 | 52-4 | | | | | | | nM | | | | | | | | | |
| GSK-J1 | 1373422- | Acid | No | | | | | | | | | 50 | | | | | |
| | 53-7 | | | | | | | | | | | μM | | | | | |
| GSK-J4 | 1373423- | Ethyl Ester | No | | | | | 170 | 28 | | | | | | | | |
| | 53-0 | | | | | | | nM | nM | | | | | | | | |
| KDM5- | 1596em- | Pyridine | No | | | | | | | | | | | | | | |
| C49 | bodi- | | | | | | | | | | | | | | | | |
| | ment-16-1 | | | | | | | | | | | | | | | | |
| KDM5- | 1596348- | Pyridine | No | | | | | | | | | | | | | | |
| C50 | 32-1 | | | | | | | | | | | | | | | | |
| KDOAM | 2230731- | Amide | No | | | | | | | | | | | | | | |
| 25 | 99-2 | | | | | | | | | | | | | | | | |
| N11 | 1613515- | isonicotinic | No | | | | | 65 | | | | 1600 | | | | | |
| | 45-9 | | | | | | | nM | | | | | | | | | |
| | 1807514- | Amide | No | | | | | 90 | | | | | | | | | |
| | 47-1 | | | | | | | nM | | | | | | | | | |
| | 1844064- | Pyrido | No | | | | | 45 | | | | 960 | | | | | |
| | 07-8 | pyrimidinone | | | | | | nM | | | | | | | | | |
| | — | | | | | | | | | | | | | | | | |
| Compound | | Rh Complex | | | | | | | | | | | | | | | |
| 1 | | | | | | | | | | | | | | | | | |
| Compound | 1498996- | Hydrazine | | | | | | X | | | | | | | | | |
| 15e | 89-6 | | | | | | | | | | | | | | | | |
| Daminozide | 1596-84-5 | Hydrazine | | | X | | | | | X | | | | | | | |
| JIB-04 | 99596-05- | Hydrazine | | | | | | 220 | | | | | | | | | |
| | 9 | | | | | | | nM | | | | | | | | | |
| Methylstat | 1310877- | Unsat amide | | | | | | | | | | | | | | | |
| | 95-2 | | | | | | | | | | | | | | | | |
| Compound | 2098902- | cyanopyrazole | No | | | | | | | | | | | | | | |
| 10r | 68-0 | | | | | | | | | | | | | | | | |
| N71 | | | Yes | | | | X | | | | | | | | | | |
| NSC | | | | | | | 410 | | | | | | | | | | |
| 6369819 | | | | | | | nM | | | | | | | | | | |

In some embodiments the KDM inhibitor is AS 8351 or TC-E 5002.

### TAZ Activators

TAZ motif (also called WWTR1) a transcriptional coactivator with a PDZ-binding was identified as a 14-3-3-binding protein. It is similar to Yes-associated protein 1 (YAP1) in its molecular structure, which consists of an N-terminal TEAD binding domain, one or two WW domains, and a transcriptional activation domain.

TAZ is phosphorylated at four sites by large tumor suppressor kinase 1 (LATS1) and LATS2, which are core kinases of the Hippo pathway. Phosphorylated TAZ is trapped by 14-3-3, is recruited from the nucleus to the cytoplasm, and undergoes protein degradation. In this way, the Hippo pathway negatively regulates TAZ.

In addition to the Hippo pathway, TAZ is regulated by cell junction proteins such as ZO-1, ZO-2, and angiomotin. Recent studies have revealed that TAZ is under the control of the actin cytoskeleton and the mechanical stretch. Moreover, Wnt signaling stabilizes. Conversely, cytoplasmic TAZ binds-catenin and Dishevelled (DVL) and inhibits-catenin nuclear localization and DVL phosphorylation to negatively regulate the Wnt pathway.

TAZ activator are chemical compounds that stabilizes and increases unphosphorylated TAZ level.

Thus, “TAZ activator” refers to an agent capable of the increasing the stability or activity of TAZ. For example, an TAZ activator results in a decrease in TAZ phosphorylation and/or TAX protein degradation.

In certain embodiments, the TAZ activator increases the stability or activity of TAZ by at least 5, 10, 20, 30, 40, 50, 60, 70, 80, 90, or 100% relative to a control, for example, relative to a baseline level of activity.

In certain embodiments, the TAZ activator increases the expression of a target gene by at least 5, 10, 20, 30, 40, 50, 60, 70, 80, 90, or 100% relative to a control, for example, relative to a baseline level of activity.

In some embodiments, the TAZ activator increases the stability or activity of TAZ by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example, relative to a baseline level of activity.

In some embodiments, increases the expression of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example, relative to a baseline level of activity.

Exemplary TAZ Activators are provide in Table 10.

TABLE 10

| | | Chemo- | | Lit | Lgr5+ | Perilymph | Formulation | Human |
|---|---|---|---|---|---|---|---|---|
| Agent | CAS | type | Mechanism | Cell | Assay | Conc | Conc | Dosage |
| IBS008738 | 371128- | Hydrazone | TAZ | | 1.1-30 μM | 1.1-30 μM | 1.1-30 mM | 25-500 mg |
| | 48-2 | | Activ. | | | | | |
| TM-25659 | 260553- | AT II | TAZ | 10-100 μM | 10-100 μM | 10-100 μM | 10-100 mM | 25-500 mg |
| | 97-7 | | Activ. | | | | | |
| TT10 | 2230640- | Thiazole | TAZ | 1 μM | 1-10 μM | 1-10 μM | 1-10 mM | 25-500 mg |
| | 94-3 | | Activ. | | | | | |
| IBS003031 | 381177- | Acridine | YAP | | | | | |
| | 81-7 | | Activ. | | | | | |
| TAZ12 | 371128- | Thiazole | TAZ | | | | | |
| | 48-2 | | Activ. | | | | | |
| TM-53 | 1257247- | AT II | TAZ | 10-100 μM | | | | |
| | 76-9 | | Activ. | | | | | |
| TM-54 | 1257247- | AT II | TAZ | 10-100 μM | | | | |
| | 77-0 | | Activ. | | | | | |
| (−)- | 1257-08- | Nat Prod | | | | | | |
| epicatechin | 5 | | | | | | | |
| gallate | | | | | | | | |
| Ethacridine | 1837-57- | Acridine | | | Activity | | | |
| | 6 | | | | | | | |
| Ethacridine | 1837-57- | Acridine | | | Activity | | | |
| | 6 | | | | | | | |
| kaempferol | 520-18-3 | Nat Prod | | | | | | |
| KR 62980 | 867187- | N-Oxide | | | | | | |
| | 61-9 | | | | | | | |
| phorbaketal | 1196507- | Nat Prod | | | | | | |
| A | 03-5 | | | | | | | |

In some embodiments the TAZ activator is IBS008738, TM-25659 or TT10.

In some embodiments the agents are a gamma-secretase inhibitor, a Taz activator, a Notch inhibitor, or an ErbB3/HER3 inhibitor.

### Gamma Secretase Inhibitors

Gamma secretase is an internal protease that cleaves within the membrane-spanning domain of its substrate proteins, including amyloid precursor protein (APP) and Notch.

Sequential cleavages of the APP by β- and γ-secretases generates Aβ. First, APP is proteolytically processed by β-secretase (BACE1) and generates a 12 kDa C-terminal stub of APP (C99); second, C99 is cleaved by γ-secretase to yield two major species of Aβ ending at residue 40 (Aβ40) or 42 (Aβ42).

Gamma secretase inhibitors may target γ-secretase and reduce Aβ production.

Exemplary gamma secretase inhibitors are provided in Table 11

TABLE 11

| | | Chemo- | | | |
|---|---|---|---|---|---|
| Agent | CAS | type | Lit cell conc | | Human Dosage |
| Semagacestat | 425386-60-3 | Amide | Aβ38, Aβ40, and Aβ42 with | | 60 mg-140 mg |
| LY 450139 | | | IC50 = 12.0, 12.1, 10.9 nM, | | |
| Begacestat/ | 769169-27-9 | Sulphon- | Lowers Aβ42, Aβ40 (EC50 = 12.4, | | 10 and 50-mg |
| GSI-953 | | amide | 14.8 nM in cells | | |
| Avagacestat/ | 1146699-66-2 | Sulphon- | IC50 = 0.27 and 0.30 nM for Aβ42 | | 25 to 125 mg |
| BMS-708163 | | amide | and Aβ40, | | |
| EVP-0962 | | | | | 10, 50, 100 or 200 mg |
| Crenigacestat | 1421438-81-4 | Amide | IC50 of ~1nM in most of the tumor | | 2.5 mg-100 mg |
| LY 3039478 | | | cell lines | | |
| (JSMD194) | | | | | |
| MK-0572 | 471905-41-6 | Acid | SH-SY5Y cells with an IC50 value | | |
| | | | of 5 nM | | |
| NIC5-15 | | | | | 800-2000 mg |
| NGP 555 | 1304630-27-0 | Hetero- | 10 | nM | 100 mg, 200 mg, |
| | | cycle | | | or 400 mg |
| Nirogacestat PF | 1290543-63-3 | Amide | (IC50 values are 1.2 and 6.2 nM in | | 150 mg |
| 03084014 | | | whole cell and cell-free assays | | |
| PF-06648671 | 1587727-31-8 | Amide | | | 300 mg |
| RO4929097 | 847925-91-1 | Amide | | | 20 mg, 30 mg, 45 mg, |
| | | | | | 90 mg or 140 mg |
| BMS-905024 | 1401066-79-2 | Amide | | | |
| BMS-932481 | 1263871-36-8 | Hetero- | IC50 at 6.6 to reduce Aβ42 | | |
| | | cycle | | | |
| BMS-986133 | | | IC50 3.5 nM to reduce Aβ42 | | |
| BMS 299897 | 290315-45-6 | Sulphon- | Inhibits Aβ40 and Aβ42 in | | |
| | | amide | vitro (IC50 7.4 and 7.9 nM | | |
| BPN-15606 | 1914989-49-3 | Hetero- | IC50 of 7 nM and 17 nM to reduce | | |
| | | cycle | Aβ42 and Aβ40 cells | | |
| Carprofen | 53716-49-7 | Acid | 76 | μM | |
| CHF5022 | 749269-77-0 | Acid | | | |
| CHF5074 | 749269-83-8 | Acid | reduces Aβ42 and Aβ40 secretion, | | |
| | | | IC50 3.6, 18.4 μM | | |
| Compound E | 209986-17-4 | Amide | | | |
| Compound W | 173550-33-9 | Acid | neuronal culture (IC50 115, 200 | | |
| | | | nM for total Aβ, Aβ42 | | |
| DAPT | 208255-80-5 | Amide | neuronal culture (IC50 115, 200 | | |
| | | | nM for total Aβ, Aβ42 | | |
| DBZ | 209984-56-5 | Amide | | | |
| E-2012 | 870843-42-8 | Unsatur- | | | |
| | | amide | | | |
| EVP-A | | | IC50 reduction of Aβ40 and Aβ42 | | |
| | | | 0.24 μM and 0.14 μM, | | |
| EVP-B | | | IC50 reduction of Aβ40 and Aβ42 | | |
| | | | 0.24 μM and 0.14 μM, | | |
| EVP-0015962 | 1447811-26-8 | Acid | | | |
| Flurizan | 51543-40-9 | Acid | | | |
| GSI-136 | 443989-01-3 | Sulphon- | | | |
| | | amide | | | |
| Indomethacin | 53-86-1 | Acid | 25-50 | μM | |
| JLK 6 | 62252-26-0 | Aniline | 30 | μM | |
| JNJ-40418677 | 1146594-87-7 | Acid | 0.18-0.2 | μM, | |
| L-685, 458 | 292632-98-5 | Peptide | 48-67 | nM | |
| LY 411575 | 209984-57-6 | Amide | | | |
| Deshydroxy | 209984-56-5 | Amide | | | |
| LY-411575 | | | | | |
| MDL 28170 | 88191-84-8 | Amide | | | |
| MRK 560 | 677772-84-8 | Sulphon- | 0.65 | nM | |
| | | amide | | | |
| MW167 | | | | | |
| NMK-T-057 | | | | | |
| Suldinac sulfide | 32004-67-4 | Acid | 34 | μM | |

### Notch Inhibitors

Exemplary Notch inhibitors are provided in Table 12

TABLE 12

| | CAS |
|---|---|
| Agent | |
| 3H4MB | 1958071-88-9 |
| BMS-871 | 15894631-89-9 |
| EDD3 | 25279-15-6 |
| ELN-46719 | 1576239-16-1 |
| FLI-06 | 313967-28-9 |
| IMR-1 | 310456-65-6 |
| FLI-06 | 62252-26-0 |
| TAPI-1 | 171235-71-5 |
| Natural Products | |
| Honokiol | |
| epigallocatechin-3-gallate (EGCG) | |
| 3,5-bis(2,4-difluorobenzylidene)-4-piperidone | |
| (DiFiD) | |
| curcumin | |
| 3,3'-diindolylmethane (DIM) | |
| resveratrol | |
| Antibodies | |
| MEDI0639 | |

### ERBB3/HER3 Inhibitors

Exemplary ErbB3/HER3 inhibitors are provided in Table 13.

TABLE 13

| | | HER 3 pKi | | | |
|---|---|---|---|---|---|
| | CAS | or IC50 | HER 1 | HER 2 | HER 4 |
| Agent | | | | | |
| Bosutinib/ | 380843-75-4 | 0.77 nM | | 2500 nM | 26 nM |
| SKI-606 | | | | | |
| Dasatinib/KIN001-5 | 302962-49-8 | 18 nM | | 1400 nM | 55 nM |
| Sapitinib/AZD8931/ | 848942-61-0 | 4 nM | 4 nM | 3 nM | |
| Vandetanib | | 180 nM | | 2600 nM | 480 nM |
| WS3 | 1421227-52-2 | 74 nM | | | |
| WS6 | 1421227-53-3 | 280 nM | | | |
| Afatinib | 850140-72-6 | | | 14 nM | |
| Erlotinib | 183321-74-6 | 1100 nM | | 2900 nM | 230 nM |
| Gefitinib | 184475-35-2 | 790 nM | | 3500 nM | 410 nM |
| KIN001-51 | | | | | |
| KIN001-111 | | | | | |
| Lapatinib | 231277-92-2 | 5500 nM | | 7 nM | 54 nM |
| Neratinib | 698387-09-6 | | | 59 nM | |
| poziotinib | 1092364-38-9 | | 3 nM | 5 nM | 23 nM |
| 1X2-21-1 | 1603845-42-6 | | | | |
| WS1 | 936099-44-4 | 3.8 μM | | | |
| Antibodies | | | | | |
| AV-203 | | | | | |
| Duligotuzumab | | | | | |
| Elgemtumab | | | | | |
| LJM716/ | | | | | |
| GSK2849330 | | | | | |
| KTN3379/ | | | | | |
| CDX-3379 | | | | | |
| Lumretuzumab | | | | | |
| RG7116 | | | | | |
| Patritumab/ | | | | | |
| U3-128 7 | | | | | |
| Seribantumab/MM-121 | | | | | |
| U3-1402 | | | | | |
| MEHD7945A/Duligotumab | | | | | |
| MCLA-128 | | | | | |
| MM-111 | | | | | |
| MM-141/Istiratumab | | | | | |

In some embodiments the ErbB3/HER3 inhibitors is WS3 or WS6.

### Pharmaceutical Compositions/Formulations and Routes of Administration

Certain embodiments relate to pharmaceutical, prophylactic, and/or therapeutic compositions, comprising a pharmaceutically-acceptable carrier and an epigenetic modulator and a Wnt agonist (and optionally a second epigenetic modulator), a pharmaceutically-acceptable salt thereof, or combinations thereof as described herein (collectively referred to herein as the “compound(s)”).

Certain embodiments relate to pharmaceutical, prophylactic, and/or therapeutic compositions, comprising a pharmaceutically-acceptable carrier and an epigenetic modulator and a Wnt agonist (and optionally a second epigenetic modulator), a pharmaceutically-acceptable salt thereof or combinations thereof as described herein (collectively referred to herein as the “compound(s)”).

In some embodiments, the concentration of the compound(s) in the pharmaceutical compositions are at the “formulation effective concentration” as described supra.

In some embodiments, the pharmaceutical composition comprises an epigenetic modulator that is an HDAC inhibitor at a concentration about 10 μM to 1,000,000 mM, about 1000 μM to 100,000 mM, about 10,000 μM to 10,000 mM, about 1000 μM to 10,000 μM, about 10,000 μM to 100,000 μM, about 100,000 μM to 1,000,000 μM, about 1,000 mM to 10,000 mM, or about 10,000 mM to 100,000 mM.

In some embodiments, the pharmaceutical composition comprises a HDAC inhibitor that is VPA at a concentration about 100 mM to 4,000 mM.

In some embodiments, the pharmaceutical composition comprises VPA at a unit dose of about 50 mg, about 100 mg, about 125 mg, about 250 mg, about 500 mg, 1000 mg, 2000 mg, 3000 mg, 4000 mg, or about 5000 mg

In some embodiments, the pharmaceutical composition comprises an oral dosage form of VPA at a unit dose of about 50 mg, about 100 mg, about 125 mg, about 250 mg, about 500 mg, 1000 mg, 2000 mg, 3000 mg, 4000 mg, or about 5000 mg

In some embodiments, the pharmaceutical composition comprises a HDAC inhibitor that is 2-hexyl-4-pentynoic acid at concentration about 100 mM to 4,000 mM.

In some embodiments, the pharmaceutical composition comprises 2-hexyl-4-pentynoic acid at a unit dose of 50 mg, about 100 mg, about 125 mg, about 250 mg, about 500 mg, 1000 mg, 2000 mg, 3000 mg, 4000 mg, or about 5000 mg

In some embodiments, the pharmaceutical composition comprises an oral dosage form of 2-hexyl-4-pentynoic acid at a unit dose of about 50 mg, about 100 mg, about 125 mg, about 250 mg, about 500 mg, 1000 mg, 2000 mg, 3000 mg, 4000 mg, or about 5000 mg

In some embodiments, the pharmaceutical composition comprises, Na phenylbutyrate that is at a concentration about 100 mM to 4,000 mM.

In some embodiments, the pharmaceutical composition comprises Na phenylbutyrate at a unit dose of about 50 mg, about 100 mg, about 125 mg, about 250 mg, about 500 mg, 1000 mg, 2000 mg, 3000 mg, 4000 mg, or about 5000 mg

In some embodiments, the pharmaceutical composition comprises an oral dosage form of the Na phenylbutyrate at a unit dose of about 50 mg, about 100 mg, about 125 mg, about 250 mg, about 500 mg, 1000 mg, 2000 mg, 3000 mg, 4000 mg, or about 5000 mg

In some embodiments, the pharmaceutical composition comprises a LSD-1 inhibitor at a concentration of about 0.01 nM to 1000 μM, about 1 nM to 100 μM, about 10 nM to 10 μM, about 1 nM to 10 μM, about 10 μM to 100 μM, about 100 μM to 1000 μM, about 1 μM to 10 μM, 0.01 mM to 1000 mM, about 1 mM to 100 mM, or about 10 mM to 100 mM.

In some embodiments, the pharmaceutical composition comprises a LSD1-inhibitor that is GSK-2879552 at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM.

In some embodiments, the pharmaceutical composition comprises a LSD1-inhibitor that is GSK-2879552 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM.

In some embodiments, the pharmaceutical composition comprises GSK-2879552 at a unit dose of about 0.01 mg to 500 mg about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, or about 5-10 mg.

In some embodiments, the pharmaceutical composition comprises a LSD1-inhibitor that is GSK-LSD1 at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM.

In some embodiments, the pharmaceutical composition comprises a LSD1-inhibitor that is GSK-LSD1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the pharmaceutical composition comprises GSK-LSD1 at a unit dose of about of about 0.01 mg to 500 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, about 5-10 mg, about 10-25 mg, about 25-50 mg, or about 50-100 mg.

In some embodiments, the pharmaceutical composition comprises a LSD1-inhibitor that is tranylcypromine at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the pharmaceutical composition comprises a LSD1-inhibitor that is tranylcypromine at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM.

In some embodiments, the pharmaceutical composition comprises tranylcypromine at a unit dose of about 1.5 mg to 750 mg, about 5 mg to 500 mg, about 10 mg to 250 mg, about 15 mg to 150 mg, about 1.5 mg to 10 mg, about 10 mg to 20 mg, about 20 mg to 30 mg, about 30 mg to 40 mg, about 40 mg to 50 mg, about 50 mg to 60 mg, about 60 mg to 70 mg, about 70 mg to 80 mg, about 90 mg to 100 mg, about 100 mg to 120 mg, or about 120 mg to 150 mg.

In some embodiments, the pharmaceutical composition comprises a LSD1-inhibitor that is phenelzine sulfate at a concentration of about 0.1 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 M, about 100 mM to 1,000 mM, or about 1,000 mM to 10.000 mM.

In some embodiments, the pharmaceutical composition comprises a LSD1-inhibitor that is phenelzine sulfate at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM.

In some embodiments, the pharmaceutical composition comprises phenelzine sulfate at a unit dose of about 1.5 mg to 750 mg, about 5 mg to 500 mg, about 10 mg to 250 mg, about 15 mg to 150 mg, about 1.5 mg to 10 mg, about 10 mg to 20 mg, about 20 mg to 30 mg; about 30 mg to 40 mg; about 40 mg to 50 mg about 50 mg to 60 mg; about 60 mg to 70 mg; about 70 mg to 80 mg; or about 90 mg to 100 mg.

In some embodiments, the pharmaceutical composition comprises a GSK3 Inhibitor that is CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1.000 mM, or about 1,000 mM to 10,000 mM. In certain such embodiments, the CHIR99021 is at a concentration of about 1 mM, 2 mM, 3 mM, 4 mM, 5 mM, 6 mM, 7 mM, 8 mM, 9 mM or 10 mM.

In some embodiments, the pharmaceutical composition comprises a GSK3 Inhibitor that is AZD1080, at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1.000 mM, or about 1,000 mM to 10,000 mM. In certain such embodiments, the AZD1080 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a GSK3 Inhibitor that is LY2090314 at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM. In certain such embodiments, LY2090314 the is at a concentration of about 1 μM, 5 μM, 10 μM, 15 μM, 20 μM, or 40 μM.

In some embodiments, the pharmaceutical composition comprises a GSK3 Inhibitor that is a substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM. In certain such embodiments, the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM, or 500 μM.

In some embodiments, the pharmaceutical composition comprises a GSK3 Inhibitor that is GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM. In certain such embodiments, the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises an epigenetic modulator that is an EZH2 inhibitor

In some embodiments, the pharmaceutical composition comprises an EZH2 inhibitor that is PF-06821497 at a concentration of 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1 mM.

In some embodiments, the pharmaceutical composition comprises an EZH2 inhibitor that is PF-06821497 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM.

In some embodiments, the pharmaceutical composition comprises PF-06821497 at a daily dose of about 50 mg to 5,000 mg, about 50 mg to 4000 mg, about 50 mg to 3000 mg, about 50 mg to 200) mg, about 50 mg to 1000 mg, about 50 mg to 500 mg, about 100 mg to 2500 mg, about 100 mg to 2000 mg, about 100 mg to 1500 mg, about 100 mg to 1000 mg, about 100 mg to 500 mg, about 150 mg to 2500 mg, about 150 mg to 2000 mg, about 150 mg to 1500 mg, about 150 mg to 1250 mg, about 75 mg, about 100 mg, about 150 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg.

In some embodiments, the pharmaceutical composition comprises an EZH2 inhibitor that is CPI-1205 at a concentration of 0.001 μM to 100 mM, about 0.01 μM to 10 mM, about 0.1 μM to 1 mM, about 1 μM to 100 μM, about 1 μM to 10 μM, about 10 μM to 100 μM, or about 100 μM to 1000 μM.

In some embodiments, the pharmaceutical composition comprises CPI-1205 is that is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM.

In some embodiments, the pharmaceutical composition comprises an EZH2 inhibitor that is CPI-1205 ay a unit dose about 100 to 5,000 mg, about 100 mg to 4000 mg, about 100 mg to 3000 mg, about 100 mg to 2000 mg, about 500 to 5,000 mg, about 500 mg to 4000 mg, about 500 mg to 3000 mg, about 750 to 5,000 mg, about 750 mg to 4000 mg, about 750 mg to 3000 mg, about 800 mg to 2400 mg, about 400 mg, about 600 mg, about 800 mg, about 1000 mg, about 1200 mg, about 1400 mg, about 1600 mg, about 1800 mg, about 2000 mg, about 2200 mg, about 2400 mg, about 2600 mg, about 2800 mg, about 3000 mg, about 3250 mg, about 3500 mg, about 4000 mg, about 4500 mg, or about 5000 mg.

In some embodiments, the pharmaceutical composition comprises an EZH2 inhibitor that is Valemetostat at a concentration of about 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1000 μM.

In some embodiments, the pharmaceutical composition comprises Valemetost that is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or 1 mM.

In some embodiments, the pharmaceutical composition comprises an EZH2 inhibitor is Valemetostat at a unit dose of about 50 mg to 5,000 mg, about 50 mg to 4000 mg, about 50 mg to 3000 mg, about 50 mg to 2000 mg, about 50 mg to 1000 mg, about 50 mg to 500 mg, about 100 mg to 2000 mg, about 100 mg to 1500 mg, about 100 mg to 1000 mg, about 100 mg to 500 mg, about 100 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg.

In some embodiments, the pharmaceutical composition comprises an EZH2 inhibitor that is Tazemetostat at a concentration of about 0.001 μM to 100 mM, about 0.01 μM to 10 mM, about 0.1 μM to 1 mM, about 1 μM to 100 μM, about 1 μM to 10 μM, 10 μM to 100 μM, about 100 μM to 1000 μM or about 1 mM to 10 mM.

In some embodiments, the pharmaceutical composition comprises Tazemetostat t at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is Tazemetostat at a unit dose of about 50 mg to 5,000 mg, about 50 mg to 4000 mg, about 50 mg to 3000 mg, about 50 mg to 2000 mg, about 50 mg to 1000 mg, about 50 mg to 500 mg, about 100 mg to 2500 mg, about 100 mg to 2000 mg, about 100 mg to 1500 mg, about 100 mg to 1000 mg, about 100 mg to 500 mg, about 200 mg to 2500 mg, about 200 mg to 2000 mg, about 200 mg to 1600 mg, about 200 mg to 1000 mg, about 100 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg.

In some embodiments, the pharmaceutical composition comprises an EZH2 inhibitor that is El1 at a concentration of about 0.1 μM to 1000 mM, about 1 μM to 100 mM, about 10 μM to 10 mM, about 100 μM to 10 mM, about 1 μM to 10 μM, 10 μM to 100 μM, about 100 μM to 1000 μM, 1 mM to 10 mM, or about 10 mM to 100 mM.

In some embodiments, the pharmaceutical composition comprises El1 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, or about 50 mM.

In some embodiments, the pharmaceutical composition comprises an EZH2 inhibitor is El1 at a unit dose of about 50 mg to 5,000 mg, about 50 mg to 4000 mg, about 50 mg to 3000 mg, about 50 mg to 2000 mg, about 50 mg to 1000 mg, about 50 mg to 500 mg, about 100 mg to 2500 mg, about 100 mg to 2000 mg, about 100 mg to 1500 mg, about 100 mg to 1000 mg, about 100 mg to 500 mg, about 200 mg to 2500 mg, about 200 mg to 2000 mg, about 200 mg to 1500 mg, about 200 mg to 1000 mg, about 100 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg.

In some embodiments, the pharmaceutical composition comprises an EZH2 inhibitor that is CPI-169 at a concentration of about 0.1 μM to 1000 mM, about 1 μM to 100 mM, about 10 μM to 10 mM, about 100 μM to 10 mM, about 1 μM to 10 μM, 10 μM to 100 μM, about 100 μM to 1000 μM, 1 mM to 10 mM, or about 10 mM to 100 mM.

In some embodiments, the pharmaceutical composition comprises CPI-169 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, or about 50 mM.

In some embodiments, the pharmaceutical composition comprises an EZH2 inhibitor that is CPI-169 at a unit dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 200 mg to 2500 mg/day, about 200 mg to 2000 mg/day, about 200 mg to 1500 mg/day, about 200 mg to 1000 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the pharmaceutical composition comprises a DOT1L inhibitor that is EPZ004777 at a unit dose of about 1-1000 mg, about 10-100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises EPZ004777 at a concentration of about 0.01 μM to 1000 mM, about 0.1 μM to 100 mM, about 1 μM to 10 mM, about 10 μM to 1 mM, 10 μM to 100 μM, about 100 μM to 1000 μM, about 1 mM to 10 mM, or about 10 mM to 100 mM.

In some embodiments, the pharmaceutical composition comprises EPZ004777 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments the additional epigenetic modulator is a DOT1L inhibitor.

In some embodiments, the pharmaceutical composition comprises a DOT1L inhibitor is EPZ004777 at a unit dose of about 1-1000 mg, about 10-100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a DOT1L inhibitor is EPZ004777 formulated for IV administration at a unit dose of 1-1000 mg, about 10-100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a DOT1L inhibitor that is SGC0946 at a concentration of about 0.01 μM to 1000 mM, about 0.1 μM to 100 mM, about 1 μM to 10 mM, about 10 μM to 1 mM, 10 μM to 100 μM, about 100 μM to 1000 μM, about 1 mM to 10 mM, or about 10 mM to 100 mM.

In some embodiments, the pharmaceutical composition comprises SGC0946 that is at a concentration of about 1.0 μM, 2.0 μM, 3.0 μM, 4.0 μM, 5.0 μM 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400n μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments, the pharmaceutical composition comprises a DOT1L inhibitor is SGC0946 at a unit dose of 1-1000 mg, about 10-100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a DOT1L inhibitor is SGC0946 formulated for IV administration at a unit dose of 1-1000 mg, about 10-100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a DOT1L inhibitor that is Pinometostat at a concentration of about 0.01 μM to 1000 mM, about 0.1 μM to 100 mM, about 1 μM to 10 mM, about 10 μM to 1 mM, 10 μM to 100 μM, about 100 μM to 1000 μM, about 1 mM to 10 mM, or about 10 mM to 100 mM.

In some embodiments, the pharmaceutical composition comprises a Pinometostat a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments, the pharmaceutical composition comprises a DOT1L inhibitor that is pinometostat at a unit dose of about 1-1000 mg, about 10-100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a DOT1L inhibitor that is formulated for IV administration at a unit dose of 1-1000 mg, about 10-100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments the additional epigenetic modulator is a KDM inhibitor.

In some embodiments, the pharmaceutical composition comprises a KDM inhibitor that is AS 8351 at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM.

In some embodiments, the pharmaceutical composition comprises a AS 8351 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM.

In some embodiments, the pharmaceutical composition comprises a KDM inhibitor that is AS 8351 at a unit dose of about 50 mg to 5,000 mg, about 50 mg to 4000 mg, about 50 mg to 3000 mg, about 50 mg to 2000 mg, about 50 mg to 1000 mg, about 50 mg to 500 mg, about 100 mg to 2500 mg, about 100 mg to 2000 mg, about 100 mg to 1500 mg, about 100 mg to 1000 mg, about 100 mg to 500 mg, about 200 mg to 2500 mg, about 200 mg to 2000 mg, about 200 mg to 1600 mg, about 200 mg to 1000 mg, about 100 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg.

In some embodiments, the pharmaceutical composition comprises a KDM inhibitor that is TC-E 5002 at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM.

In some embodiments, the pharmaceutical composition comprises a AS TC-E 5002 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM.

In some embodiments, the pharmaceutical composition comprises a KDM inhibitor is TC-E 5002 at a unit dose of about 50 mg to 5,000 mg, about 50 mg to 4000 mg, about 50 mg to 3000 mg, about 50 mg to 2000 mg, about 50 mg to 1000 mg, about 50 mg to 500 mg, about 100 mg to 2500 mg, about 100 mg to 2000 mg, about 100 mg to 1500 mg, about 100 mg to 1000 mg, about 100 mg to 500 mg, about 200 mg to 2500 mg, about 200 mg to 2000 mg, about 200 mg to 1600 mg, about 200 mg to 1000 mg, about 100 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg

In some embodiments, the pharmaceutical composition comprises a KDM inhibitor that is EPT-103182 at a concentration of 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1 mM.

In some embodiments, the pharmaceutical composition comprises EPT-103182 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM.

In some embodiments, the pharmaceutical composition comprises a KDM inhibitor is EPT-103182 at a unit dose of about 50 mg to 5,000 mg, about 50 mg to 4000 mg, about 50 mg to 3000 mg, about 50 mg to 2000 mg, about 50 mg to 1000 mg, about 50 mg to 500 mg, about 100 mg to 2500 mg, about 100 mg to 2000 mg, about 100 mg to 1500 mg, about 100 mg to 1000 mg, about 100 mg to 500 mg, about 150 mg to 2500 mg, about 150 mg to 2000 mg, about 150 mg to 1500 mg, about 150 mg to 1250 mg, about 75 mg, about 100 mg, about 150 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg.

In some embodiments, the pharmaceutical composition comprises a IBS008738 that is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM.

In some embodiments, the pharmaceutical composition comprises a IBS008738 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments, the pharmaceutical composition comprises a TAZ activator that is IBS008738 at a unit dose of about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a TAZ activator that is TT-10 at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM.

In some embodiments, the pharmaceutical composition comprises a TT-10 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments the additional epigenetic modulator is a TAZ activator.

In some embodiments, the pharmaceutical composition comprises a TAZ activator is TT-10 at a unit dose of about 10 mg to 5,000n mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a TAZ activator is TM-25659 at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM.

In some embodiments, the pharmaceutical composition comprises a TM-25659 at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, the pharmaceutical composition comprises a TAZ activator is TM-25659 at a unit dose of about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg. In certain embodiments, the pharmaceutical composition comprises a GSK3 Inhibitor that is AZD1080 and a HDAC inhibitor that is VPA. The AZD1080 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the AZD1080 is at a concentration of 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the AZ1080 is at a concentration of about 6.75 mM and the VPA is sodium valproate at a concentration of about 533 mM. In certain such embodiments, the AZD1080 is at a concentration of 3.14 mg/ml and the VPA is at a concentration of 88.6 mg/ml.

In certain embodiments, the pharmaceutical composition comprises a GSK3 Inhibitor that is LY2090314 and a HDAC inhibitor that is VPA. The LY2090314 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the LY2090314 is at a concentration of 1 μM, 5 μM, 10 μM, 15 μM, 20 μM, or 40 μM and the VPA at a concentration about 100 mM to 4,000 mM.

In some embodiments, the LY2090314 is at a concentration of about 6.75 mM and the VPA is sodium valproate at a concentration of about 533 mM. In certain such embodiments, the LY2090314 is at a concentration of 3.14 mg/ml and the VPA is at a concentration of 88.6 mg/ml.

In certain embodiments, the pharmaceutical composition comprises a GSK3 Inhibitor that is 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of 1 μM, 5 μM, 10 μM, 15 μM, 20 μM, 50 μM, 100 μM, 250 μM, or 500 μM and the VPA at a concentration about 100 mM to 4,000 mM.

In some embodiments, the 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about 6.75 mM and the VPA is sodium valproate at a concentration of about 533 mM. In certain such embodiments, the 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of 3.14 mg/ml and the VPA is at a concentration of 88.6 mg/ml.

In certain embodiments, the pharmaceutical composition comprises a GSK3 Inhibitor that GSK3-inhibitor XXII and a HDAC inhibitor that is VPA. The GSK3-inhibitor XXII is at a concentration of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA at a concentration about 100 mM to 4,000 mM.

In some embodiments, the GSK3-inhibitor XXII is at a concentration of 0.1 mM, 0.2 mM, 0.3 mM, 0.4 mM, 0.5 mM, 0.6 mM, 0.7 mM, 0.8 mM, 0.9 mM, or 1.0 mM and the VPA at a concentration about 100 mM to 4,000 mM.

In some embodiments, GSK3-inhibitor XXII is at a concentration of about 6.75 mM and the VPA is sodium valproate at a concentration of about 533 mM. In certain such embodiments, GSK3-inhibitor XXII and the VPA is at a concentration of 88.6 mg/ml.

In certain embodiments, the pharmaceutical composition comprises a GSK3 Inhibitor that is CHIR99021 and a HDAC inhibitor that is VPA. The CHIR99021 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the CHIR99021 is at a concentration of about 1 mM, 2 mM, 3 mM, 4 mM, 5 mM, 6 mM, 7 mM, 8 mM, 9 mM or 10 mM and the VPA at a concentration about 100 mM to 4,000 mM.

In some embodiments, the CHIR99021 is at a concentration of about 6.75 mM and the VPA is sodium valproate at a concentration of about 533 mM. In certain such embodiments, the CHIR99021 is at a concentration of 3.14 mg/ml and the VPA is at a concentration of 88.6 mg/ml.

In some embodiments, the pharmaceutical composition comprises a LSD1-inhibitor that is GSK-2879552 and a GSK3 Inhibitor that is AZD1080. The GSK-2879552 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM, and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the GSK-2879552 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a LSD1-inhibitor that is GSK-2879552 and a GSK3 Inhibitor that is LY2090314. The GSK-2879552 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM, and the LY2090314 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the GSK-2879552 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400n μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 is at a concentration of about 1 μM, 5 μM, 10 μM, 15 μM, 20 μM, or 40 μM.

In some embodiments, the pharmaceutical composition comprises a LSD1-inhibitor that is GSK-2879552 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The GSK-2879552 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM, and the substituted 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the GSK-2879552 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM or about 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about 1 μM, 5 μM, 10 μM, 15 μM, 20 μM, 50 μM, 100 μM, 250 μM or 500 μM.

In some embodiments, the pharmaceutical composition comprises a LSD1-inhibitor that is GSK-2879552 and a GSK3 Inhibitor that is GSK3-inhibitor XXII. The GSK-2879552 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM, and the GSK3-inhibitor XXII is at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, the GSK-2879552 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises a LSD1-inhibitor that is GSK-2879552 and a GSK3 Inhibitor that is CHIR99021. The GSK-2879552 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM, and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,00) mM, or about 1,000 mM to 10,000 mM.

In some embodiments, the GSK-2879552 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a LSD1-inhibitor that is GSK-LSD1 and a GSK3 Inhibitor that is AZD1080. The GSK-LSD1 at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the GSK-LSD1 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the AZ1090 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a LSD1-inhibitor that is GSK-LSD1 and a GSK3 Inhibitor that is LY2090314. The GSK-LSD1 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM and the LY2090314 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the GSK-LSD1 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the LY2090314 is at a concentration of about 1 μM, 5 μM, 10 μM, 15 μM, 20 μM, or 30 μM.

In some embodiments, the pharmaceutical composition comprises a LSD1-inhibitor that is GSK-LSD1 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The GSK-LSD1 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM and the substituted 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the GSK-LSD1 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about 1 μM, 5 μM, 10 μM, 15 μM, 20 μM, 50 μM, 100 μM, 250 μM or 500 μM.

In some embodiments, the pharmaceutical composition comprises a LSD1-inhibitor that is GSK-LSD1 and a GSK3 Inhibitor that is GSK3-inhibitor XXII. The GSK-LSD1 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM. and the GSK3-inhibitor XXII is at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, the GSK-LSD1 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises a LSD1-inhibitor that is GSK-LSD1 and a GSK3 Inhibitor that is CHIR99021. The GSK-LSD1 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM and the CHIR99021 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the GSK-LSD1 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a LSD1-inhibitor that is tranylcypromine and a GSK3 Inhibitor that is AZD1080. The tranylcypromine is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM, and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the tranylcypromine is at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the AZ1090 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a LSD1-inhibitor that is tranylcypromine and a GSK3 Inhibitor that is LY2090314. The tranylcypromine is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM about 10 mM to 100 mM, about 100 mM to 1,000 mM, or about 1,000 mM to 10,000 mM and the LY2090314 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the tranylcypromine is at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the LY2090314 is at a concentration of about 1 μM, 5 μM, 10 μM, 15 μM, 20 μM, or 40 μM.

In some embodiments, the pharmaceutical composition comprises a LSD1-inhibitor that is tranylcypromine and a GSK3 Inhibitor that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The tranylcypromine is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments the tranylcypromine is at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about 1 μM, 5 μM, 10 μM, 15 μM, 20 μM, 50 μM, 100 μM, 250 μM or 500 μM.

In some embodiments, the pharmaceutical composition comprises a LSD1-inhibitor that is tranylcypromine and a GSK3 Inhibitor that is GSK3-inhibitor XXII. tranylcypromine is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the GSK3-inhibitor XXII is at a concentration of about of about 0.1 μM to 1,000 mM, about 1 μM to 100 mM, about 10 μM to 10 mM, about 0.1 μM to 1 μM about 1 μM to 10 μM, about 10 μM to 100 μM, about 100 μM to 1 mM, about 1 mM to 10 mM, about 10 mM to 100 mM, or about 100 mM to 1000 mM.

In some embodiments, tranylcypromine is at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises a LSD1-inhibitor that is tranylcypromine and a GSK3 inhibitor that is CHIR99021. The tranylcypromine is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the CHIR99021 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the tranylcypromine is at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises an LSD1-inhibitor that is phenelzine sulfate and a GSK3 Inhibitor that is AZD1080. The phenelzine sulfate is at a concentration of about 0.1 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the phenelzine sulfate is at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the AZ1090 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises an LSD1-inhibitor that is phenelzine sulfate and a GSK3 Inhibitor that is LY2090314. The phenelzine sulfate is at a concentration of about 0.1 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the LY2090314 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments the phenelzine sulfate is at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the LY2090314 the is at a concentration of about 1 μM, 5 μM, 10 μM, 15 μM, 20 μM, or 40 μM.

In some embodiments, the pharmaceutical composition comprises an LSD1-inhibitor that is phenelzine sulfate and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The phenelzine sulfate is at a concentration of about 0.1 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments the phenelzine sulfate is at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about 1 μM, 5 μM, 10 μM, 15 μM, 20 μM, 50 μM, 100 μM, 250 μM or 500 μM.

In some embodiments, the pharmaceutical composition comprises a LSD1-inhibitor that is phenelzine sulfate and a GSK3 Inhibitor that is GSK3-inhibitor XXII. The phenelzine sulfate is at a concentration of about 0.1 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the GSK3-inhibitor XXII is at a concentration of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments the phenelzine sulfate is at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises a LSD1-inhibitor that is phenelzine sulfate and a GSK3 Inhibitor that is CHIR99021. The phenelzine sulfate is at a concentration of about 0.1 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the CHIR99021 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments the phenelzine sulfate is at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises an LSD1-inhibitor that is GSK-2879552 and a GSK3 Inhibitor that is AZD1080 and a HDAC inhibitor that is VPA. The GSK-2879552 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA is at a concentration about 100 mM to 4,000 mM.

In some embodiments the GSK-2879552 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a concentration of about 1 mM, 2 mM, 3 mM, 4 mM, 5 mM, 6 mM, 7 mM, 8 mM, 9 mM, or 10 mM and the VPA is at a concentration about 100 mM to 4.000 mM.

In some embodiments, the pharmaceutical composition comprises a LSD1-inhibitor that is GSK-2879552 and a GSK3 Inhibitor that is LY2090314 and a HDAC inhibitor that is VPA. The GSK-2879552 is at a concentration of about 0.001 μM to 1,000 mM, about 0.01 μM to 100.000 μM, about 0.1 μM to 10,000 μM, about 1 μM to 1,000 μM, about 1 μM to 10 μM, about 10 μM to 100 μM, about 100 μM to 1 mM, or about 1 mM to 10 mM and the LY2090314 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA is at a concentration about 100 mM to 4,000 mM.

In some embodiments, the GSK-2879552 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM or 40 μM and the VPA is at a concentration about 100 mM to 4,000 mM.

In some embodiments, the pharmaceutical composition comprises a LSD1-inhibitor that is GSK-2879552 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The GSK-2879552 is at a concentration of about 0.001 μM to 1,000 mM, about 0.01 μM to 100.000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM. and the VPA is at a concentration about 100 mM to 4,000 mM.

In some embodiments, the GSK-2879552 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about 1 μM, 5 μM, 10 μM, 15 μM, 20 μM, 50 μM, 100 μM, 250 μM, or 500 μM and the VPA is at a concentration about 100 mM to 4,000 mM.

In some embodiments, the pharmaceutical composition comprises a LSD1-inhibitor that is GSK-2879552 and a GSK3 Inhibitor that is GSK3-inhibitor XXII and a HDAC inhibitor that is VPA. The GSK-2879552 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII is at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA is at a concentration about 100 mM to 4.000 mM.

In some embodiments, the GSK-2879552 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM. and the VPA is at a concentration about 100 mM to 4,000 mM.

In some embodiments, the pharmaceutical composition comprises a LSD1-inhibitor that is GSK-2879552 and a GSK3 Inhibitor that is CHIR99021 and a HDAC inhibitor that is VPA. The GSK-2879552 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CHIR99021 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1.000 mM, or about 1,000 mM to 10,000 mM and the VPA is at a concentration about 100 mM to 4,000 mM.

In some embodiments, the GSK-2879552 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM and the VPA is at a concentration about 100 mM to 4,000 mM.

In some embodiments, the pharmaceutical composition comprises a LSD1-inhibitor that is tranylcypromine and a GSK3 Inhibitor that is AZD1080 and a HDAC inhibitor that is VPA. The tranylcypromine is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the AZD1080 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA is at a concentration about 100 mM to 4,000 mM.

In some embodiments, the tranylcypromine is at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the AZD1080 is at a concentration of about 1 mM, 2 mM 3 mM, 4 mM, 5 mM, 6 mM, 7 mM, 8 mM, 9 mM, or 10 mM and the VPA is at a concentration about 100 mM to 4,000 mM.

In some embodiments, the pharmaceutical composition comprises a LSD1-inhibitor that is tranylcypromine and a GSK3 Inhibitor that is LY2090314 and a HDAC inhibitor that is VPA. The tranylcypromine is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the LY2090314 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA is at a concentration about 100 mM to 4,000 mM.

In some embodiments, the tranylcypromine is at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the LY2090314 is at a concentration of about 1 μM, 5 μM, 10 μM, 15 μM, 20 μM, or 40 μM and the VPA is at a concentration about 100 mM to 4,000 mM.

In some embodiments, the pharmaceutical composition comprises a LSD1-inhibitor that is tranylcypromine and a GSK3 Inhibitor that is 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The tranylcypromine is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA is at a concentration about 100 mM to 4,000 mM.

In some embodiments, the tranylcypromine is at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about 1 μM, 5 μM, 10 μM, 15 μM, 20 μM, 50 μM, 100 μM, 250 μM, or 500 μM and the VPA is at a concentration about 100 mM to 4,000 mM.

In some embodiments, the pharmaceutical composition comprises a LSD1-inhibitor that is tranylcypromine and a GSK3 Inhibitor that is GSK3-inhibitor XXII and a HDAC inhibitor that is VPA. The tranylcypromine is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the GSK3-inhibitor XXII is at a concentration of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA is at a concentration about 100 mM to 4,000 mM.

In some embodiments, the tranylcypromine is at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM and the VPA is at a concentration about 100 mM to 4,000 mM.

In some embodiments, the pharmaceutical composition comprises a LSD1-inhibitor that is tranylcypromine and a GSK3 Inhibitor that is CHIR99021 and a HDAC inhibitor that is VPA. The tranylcypromine is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM about 10 mM to 100 mM, about 100 mM to 1,000 mM, or about 1.000 mM to 10.000 mM and the CHIR99021 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA is at a concentration about 100 mM to 4,000 mM.

In some embodiments, the tranylcypromine is at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the CHIR99021 is at a concentration of about 1 mM, 2 mM, 3 mM, 4 mM, 5 mM, 6 mM, 7 mM, 8 mM, 9 mM, or 10 mM and the VPA is at a concentration about 100 mM to 4,000 mM.

In some embodiments, the pharmaceutical composition comprises a LSD1-inhibitor that is GSK-LSD1 and a GSK3 Inhibitor that is AZD1080 and a HDAC inhibitor that is VPA. The GSK-LSD1 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM and the AZD1080 is at a concentration of about 0.001 mM to 10.000 mM, about 0.01 mM to 1.000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA is at a concentration about 100 mM to 4,000 mM.

In some embodiments, the GSK-LSD1 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the AZD1080 is at a concentration of about 1 mM, 2 mM, 3 mM, 4 mM, 5 mM, 6 mM, 7 mM, 8 mM, 9 mM, or 10 mM and the VPA is at a concentration about 100 mM to 4,000 mM.

In some embodiments, the pharmaceutical composition comprises a LSD1-inhibitor that is GSK-LSD1 and a GSK3 Inhibitor that is LY2090314 and a HDAC inhibitor that is VPA. The GSK-LSD1 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 M, about 10 μM to 100 μM, or about 100 μM to 1,000 μM and the LY2090314 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA is at a concentration about 100 mM to 4,000 mM.

In some embodiments, the GSK-LSD1 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the LY2090314 is at a concentration of about 1 μM, 5 μM, 10 μM, 15 μM, 20 μM, or 40 μM and the VPA is at a concentration about 100 mM to 4,000 mM.

In some embodiments, the pharmaceutical composition comprises a LSD1-inhibitor that is GSK-LSD1 and a GSK3 Inhibitor that is 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The GSK-LSD1 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM and the A3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA is at a concentration about 100 mM to 4,000 mM.

In some embodiments, the GSK-LSD1 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about 1 μM, 5 μM, 10 μM, 15 μM, 20 μM, 50 μM, 100 μM, 250 μM, or 500 μM and the VPA is at a concentration about 100 mM to 4,000 mM.

In some embodiments, the pharmaceutical composition comprises a LSD1-inhibitor that is GSK-LSD1 and a GSK3 Inhibitor that is GSK3-inhibitor XXII and a HDAC inhibitor that is VPA. The GSK-LSD1 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM and the GSK3-inhibitor XXII is at a concentration of about 0.1 μM to 1,000 mM, about 1 μM to 100 mM about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA is at a concentration about 100 mM to 4,000 mM.

In some embodiments, the GSK-LSD1 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM and the VPA is at a concentration about 100 mM to 4,000 mM.

In some embodiments, the pharmaceutical composition comprises a LSD1-inhibitor that is GSK-LSD1 and a GSK3 Inhibitor that is CHIR99021 and a HDAC inhibitor that is VPA. The GSK-LSD1 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM and the CHIR99021 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA is at a concentration about 100 mM to 4,000 mM.

In some embodiments, GSK-LSD1 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the CHIR99021 is at a concentration of about 1 mM, 2 mM, 3 mM, 4 mM, 5 mM, 6 mM, 7 mM, 8 mM, 9 mM, or 10 mM and the VPA is at a concentration about 100 mM to 4,000 mM.

In some embodiments, the pharmaceutical composition comprises a LSD1-inhibitor that is tranylcypromine and a GSK3 Inhibitor that is AZD1080 and a HDAC inhibitor that is VPA. The tranylcypromine is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to mM, about 1 mM to 10 mM about 10 mM to 100 mM, about 100 mM to 1,000 mM, or about 1,000 mM to 10,000 mM and the AZD1080 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA is at a concentration about 100 mM to 4,000 mM.

In some embodiments, the tranylcypromine is at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the AZD1080 is at a concentration of about 1 mM, 2 mM, 3 mM, 4 mM, 5 mM, 6 mM, 7 mM, 8 mM, 9 mM, or 10 mM and the VPA is at a concentration about 100 mM to 4,000 mM.

In some embodiments, the pharmaceutical composition comprises a LSD1-inhibitor that is tranylcypromine and a GSK3 Inhibitor that is LY2090314 and a HDAC inhibitor that is VPA. The tranylcypromine is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the LY2090314 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA is at a concentration about 100 mM to 4,000 mM.

In some embodiments, the tranylcypromine is at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the LY2090314 is at a concentration of about 1 μM, 5 μM, 10 μM, 15 μM, 20 μM, or 40 μM and the VPA is at a concentration about 100 mM to 4,000 mM.

In some embodiments, the pharmaceutical composition comprises a LSD1-inhibitor that is tranylcypromine and a GSK3 Inhibitor that is 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The tranylcypromine is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA is at a concentration about 100 mM to 4,000 mM.

In some embodiments, the tranylcypromine is at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about 1 μM, 5 μM, 10 μM, 15 μM, 20 μM, 50 μM, 100 μM, 250 μM, or 500 μM and the VPA is at a concentration about 100 mM to 4,000 mM.

In some embodiments, the pharmaceutical composition comprises a LSD1-inhibitor that is tranylcypromine and a GSK3 Inhibitor that is GSK3-inhibitor XXII and a HDAC inhibitor that is VPA. The tranylcypromine is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the GSK3-inhibitor XXII is at a concentration of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA is at a concentration about 100 mM to 4,000 mM.

In some embodiments, the tranylcypromine is at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM and the VPA is at a concentration about 100 mM to 4,000 mM.

In some embodiments, the pharmaceutical composition comprises an LSD1-inhibitor that is tranylcypromine and a GSK3 Inhibitor that is CHIR99021 and a HDAC inhibitor that is VPA. The tranylcypromine is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the CHIR99021 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA is at a concentration about 100 mM to 4,000 mM.

In some embodiments, the tranylcypromine is at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the CHIR99021 is at a concentration of about 1 mM, 2 mM, 3 mM, 4 mM, 5 mM, 6 mM, 7 mM, 8 mM, 9 mM, or 10 mM and the VPA is at a concentration about 100 mM to 4,000 mM.

In some embodiments, the pharmaceutical composition comprises a LSD1-inhibitor that is GSK-LSD1 and a GSK3 Inhibitor that is AZD1080 and a HDAC inhibitor that is VPA. The GSK-LSD1 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM and the AZD1080 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA is at a concentration about 100 mM to 4,000 mM.

In some embodiments, the GSK-LSD1 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the AZD1080 is at a concentration of about 1 mM, 2 mM, 3 mM, 4 mM, 5 mM, 6 mM, 7 mM, 8 mM, 9 mM, or 10 mM and the VPA is at a concentration about 100 mM to 4,000 mM.

In some embodiments, the pharmaceutical composition comprises a LSD1-inhibitor that is GSK-LSD1 and a GSK3 Inhibitor that is LY2090314 and a HDAC inhibitor that is VPA. The GSK-LSD1 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM and the LY2090314 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA is at a concentration about 100 mM to 4,000 mM.

In some embodiments, the GSK-LSD1 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the LY2090314 is at a concentration of about 1 μM, 5 μM, 10 μM, 15 μM, 20 μM, or 40 μM and the VPA is at a concentration about 100 mM to 4,000 mM.

In some embodiments, the pharmaceutical composition comprises a LSD1-inhibitor that is GSK-LSD1 and a GSK3 Inhibitor that is 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The GSK-LSD1 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM and the A3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA is at a concentration about 100 mM to 4,000 mM.

In some embodiments, the GSK-LSD1 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about 1 μM, 5 μM, 10 μM, 15 μM, 20 μM, 50 μM, 100 μM, 250 μM, or 500 μM and the VPA is at a concentration about 100 mM to 4.000 mM.

In some embodiments, the pharmaceutical composition comprises a LSD1-inhibitor that is GSK-LSD1 and a GSK3 Inhibitor that is GSK3-inhibitor XXII and a HDAC inhibitor that is VPA. The GSK-LSD1 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM and the GSK3-inhibitor XXII is at a concentration of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA is at a concentration about 100 mM to 4,000 mM.

In some embodiments, the GSK-LSD1 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM and the VPA is at a concentration about 100 mM to 4,000 mM.

In some embodiments, the pharmaceutical composition comprises a LSD1-inhibitor that is GSK-LSD1 and a GSK3 Inhibitor that is CHIR99021 and a HDAC inhibitor that is VPA. The GSK-LSD1 is at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM and the CHIR99021 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA is at a concentration about 100 mM to 4,000 mM.

In some embodiments, GSK-LSD1 is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the CHIR99021 is at a concentration of about 1 mM, 2 mM, 3 mM, 4 mM, 5 mM, 6 mM, 7 mM, 8 mM, 9 mM, or 10 mM and the VPA is at a concentration about 100 mM to 4.000 mM.

In some embodiments, as noted above, a composition is adapted for administration to the inner ear and/or middle ear, for example, local administration to the round window membrane or intratympanic or transtympanic administration, for example, to cochlear tissue. Alternatively, as noted above, a composition is adapted for administration systemically for example, orally or parentally.

When administered locally, for example, to the inner and/or middle ear, the compounds (s) are administered at a unit dose of about 25 μl to 500 μl, or about 50 μl to 200 μl.

The phrase “pharmaceutically-acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically-acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically-acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

Certain compositions comprise at least one biocompatible matrix. The term “biocompatible matrix” as used herein is a polymeric carrier that is acceptable for administration to humans for the release of therapeutic agents. In some instances, a biocompatible matrix may be a biocompatible gel, foam, fiber, film, or mats. In some embodiments the biocompatible matrix is derived from silk.

In some embodiments the biocompatible matrix comprises hyaluronic acid, hyaluronates, lecithin gels, pluronics, poly(ethyleneglycol), polymers, poloxamers, chitosans, xyloglucans, collagens, fibrins, polyesters, poly(lactides), poly(glycolide), poly(lactic-co-glycolic acid (PLGA), sucrose acetate isobutyrate, glycerol monooleate, poly anhydrides, poly caprolactone sucrose, glycerol monooleate or a combination thereof.

Exemplary polymers suitable for formulating the biologically active compositions of the present disclosure include, but are not limited to polyamides, polycarbonates, polyalkylenes (polyethylene glycol (PEG)), polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

In some embodiments, the polymer is in a concentration between about 5 wt % and about 25 wt % relative to the composition, or about 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, or 25 wt % relative to the composition. In certain embodiments, the polymer is in a concentration between about 10 wt % and about 23 wt % relative to the composition. In some embodiments the polymer is in a concentration between about 15 wt % and about 20 wt % relative to the composition. In particular embodiments, the polymer is in a concentration is approximately 17 wt % relative to the composition.

In one embodiment, a biologically active composition of the present disclosure is formulated in a ABA-type or BAB-type triblock copolymer or a mixture thereof, wherein the A-blocks are relatively hydrophobic and comprise biodegradable polyesters or poly(orthoester), and the B-blocks are relatively hydrophilic and comprise polyethylene glycol (PEG). The biodegradable, hydrophobic A polymer block comprises a polyester or poly(ortho ester), in which the polyester is synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxyhexanoic acid, γ-butyrolactone, γ-hydroxybutyric acid, δ-valerolactone, δ-hydroxyvaleric acid, hydroxybutyric acids, malic acid, and copolymers thereof.

In some embodiments, the copolymer is in a concentration between about 5 wt % and about 25 wt % relative to the composition, or about 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, or 25 wt % relative to the composition. In certain embodiments, the copolymer is in a concentration between about 10 wt % and about 23 wt % relative to the composition. In some embodiments the copolymer is in a concentration between about 15 wt % and about 20 wt % relative to the composition. In particular embodiments, the copolymer is in a concentration is approximately 17 wt % relative to the composition.

Certain compositions comprise at least one poloxamer. Poloxamers are triblock copolymers formed of (i.e., hydrophilic poly(oxyethylene) blocks and hydrophobic poly(oxypropylene) blocks) configured as a triblock of poly(oxyethylene)-poly(oxypropylene)-poly(oxyethylene). Poloxamers are one class of block copolymer surfactants having a propylene oxide block hydrophobe and an ethylene oxide hydrophile. Poloxamers are commercially available (e.g., Pluronic® polyols are available from BASF Corporation). Alternatively, poloxamers can be synthesized by known techniques.

Exemplary poloxamers include Poloxamer 124, Poloxamer 188, Poloxamer 237. Poloxamer 338, and Poloxamer 407. In some embodiments, the poloxamer comprises mixtures of two or more of Poloxamer 124, Poloxamer 188, Poloxamer 237, Poloxamer 338 or Poloxamer 407. In some embodiments, the mixture of two or more poloxamers comprise Poloxamer 407 and Poloxamer 124. In certain embodiments the poloxamer comprises at least one of Poloxamer 188 and Poloxamer 407 or mixtures thereof. In some embodiments, the poloxamer is Poloxamer 407.

In some embodiments, the poloxamer is in a concentration between about 5 wt % and about 25 wt % relative to the composition, or about 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, or 25 wt % relative to the composition. In certain embodiments, the poloxamer is in a concentration between about 10 wt % and about 23 wt % relative to the composition. In some embodiments the poloxamer is in a concentration between about 15 wt % and about 20 wt % relative to the composition. In particular embodiments, the poloxamer is in a concentration is approximately 17 wt % relative to the composition.

In some embodiments, wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Certain compositions comprise at least one antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In specific embodiments, the viscosity of the composition at about body temperature is substantially different (e.g., lesser, greater) than the viscosity of the composition at room temperature.

In some embodiments, the composition comprises a buffer. For example, in certain instances, the buffer is physiological saline or phosphate-buffered saline (PBS).

In some embodiments, the composition is at or near physiological pH. For instance, in some embodiments, the composition has a pH of between about 6 and about 8, including all integers, decimals, and ranges in between, for example, about 6 to about 6.5 to about 7 to about 7.5 to about 8. In specific embodiments, the composition has a pH of about 7.4 (±0.2).

In some aspects, the present disclosure the pharmaceutical compositions are lyophilized. Comprising one or more agents described herein and a gelling agent.

In some embodiments, the lyophilized pharmaceutical composition is in the form of a lyophilized cake.

In some embodiments, the lyophilized pharmaceutical composition has a higher stability to oxygen and/or light as compared to a comparable pharmaceutical composition comprising one or more solvents.

In some embodiments, the present disclosure provides a reconstituted solution of the lyophilized pharmaceutical compositions.

As used herein, the term “gelling agent” refers to an agent capable of imparting a gel-like or thickening quality to the pharmaceutical composition or reconstituted solution of the present disclosure upon being subjected to a gelling condition (e.g., a particular temperature or temperature range, the presence of an ion, a pH value or range, or a concentration of gelling agent that causes the gelling agent to undergoing a change or transition from low viscosity to high viscosity, or the reverse). In some embodiments, the gelling condition is a particular temperature (e.g., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C.). In some embodiments, the gelling condition is a particular temperature range (e.g., about 26° C., or higher, about 27° C., or higher, about 28° C., or higher, about 29° C., or higher, about 30° C., or higher, about 31° C., or higher, about 32° C., or higher, about 33° C., or higher, about 34° C., or higher, about 35° C., or higher, about 36° C., or higher, about 37° C., or higher, about 38° C., or higher, about 39° C., or higher, or about 40° C., or higher). In some embodiments, the gelling agent provides a viscosity of between about 1,000 and 10,000,000 centipoise, between about 5,000 and 5,000,000 centipoise, or between about 100.000 and 4,000.000 centipoise, to the pharmaceutical composition or reconstituted solution of the present disclosure. In some embodiments, the gelling agent provides a viscosity of between about 50,000 and 2,000,000 centipoise to the pharmaceutical composition or reconstituted solution of the present disclosure.

In some embodiments, prior to gelling (e.g., at ambient temperature (e.g., between about 20° C. and about 26° C.)), the gelling agent provides a viscosity of less than about 100,000 centipoise, less than about 50,000 centipoise, 20,000 centipoise, less than about 10,000 centipoise, less than about 8,000 centipoise, less than about 7,000 centipoise, less than about 6,000 centipoise, less than about 5,000 centipoise, less than about 4,000 centipoise, less than about 3,000 centipoise, less than about 2,000 centipoise, or less than about 1,000 centipoise to the pharmaceutical composition or reconstituted solution of the present disclosure.

In some embodiments, upon gelling (e.g., at the temperature of a human body (e.g., between about 35° C. to about 39° C., between about 36° C. to about 38° C., or at about 37° C.)), the gelling agent provides a viscosity of greater than about 1,000 centipoise, greater than about 5,000 centipoise, greater than about 10,000 centipoise, greater than about 20,000 centipoise, greater than about 50,000 centipoise, greater than about 60,000 centipoise, greater than about 70,000 centipoise, greater than about 80,000 centipoise, greater than about 90,000 centipoise, or greater than about 100,000 centipoise.

In some embodiments, upon gelling (e.g., at the temperature of a human body (e.g., between about 36° C. to about 39° C., or at about 37° C.)), the viscosity of the pharmaceutical composition or reconstituted solution of the present disclosure, as measured in units of centipoise, being about 2 fold or greater, about 5 fold or greater, about 10 fold or greater, about 20 fold or greater, about 50 fold or greater, about 60 fold or greater, about 7 fold or greater, about 80 fold or greater, about 90 fold or greater, about 100 fold or greater as compared to the viscosity of the pharmaceutical composition or reconstituted solution prior to gelling (e.g., at ambient temperature (e.g., at about 25° C.)).

It is understood that the gelling condition (e.g., gelling temperature) of the pharmaceutical composition or reconstituted solution of the present disclosure may be measured with a variety of techniques in the art. In some embodiment, the gelling temperature is determined using a commercially available rheometer having a parallel plate geometry (e.g., with plate distance ranging from 0.5 mm to 1.0 mm). In some embodiments, the analysis is performed over a continuous temperature range (e.g., 15° C. to 40° C.) at a constant rate (e.g., 2 to 3° C./min) and a deformation frequency of 0.74 Hz to 1 Hz. The gelation temperature is determined at the temperature whereby the shear storage modulus (G′) and the shear loss modulus (G″) are equal.

In some embodiments, the gelling agent comprises acacia, alginic acid, bentonite, poly(acrylic acid) (Carbomer), carboxymethyl cellulose, ethylcellulose, gelatin, hydroxyethyl cellulose, hydroxypropyl cellulose, magnesium aluminum silicate (Veegum), methylcellulose, poloxamer, hyaluronic acid sodium, polylacticglycolic acid sodium, chitosan, polyvinyl alcohol, sodium alginate, tragacanth, xanthan gum, or any combination thereof. In some embodiment, the gelling agent comprises poloxamer.

In some embodiments, the gelling agent is a thermoreversible gelling agent.

As used herein, the term “thermoreversible” refers to a capability of being reversible by the application of heat. The “thermoreversible gelling agent” refers to an agent capable of reversibly imparting a gel-like or thickening quality to the pharmaceutical composition or reconstituted solution of the present disclosure upon application of heat.

In some embodiments, the thermoreversible gelling agent comprises a poloxamer.

It is understood that the gelling agent (e.g., the thermoreversible gelling agent) may also be a bulking agent of the pharmaceutical composition or reconstituted solution of the present disclosure. In some embodiments, a poloxamer (e.g., poloxamer 407) is the gelling agent and/or the bulking agent of the pharmaceutical composition or reconstituted solution of the present disclosure. Poloxamers are a general class of commercially available and pharmaceutically acceptable triblock copolymers of polyethylene oxide-polypropylene oxide-polyethylene oxide which exhibit relatively low viscosity at low temperatures (e.g., room temperature or below) but much high viscosities at elevated temperatures (e.g., body temperatures of approximately 37° C.) whereby compositions containing such thermoreversible gelling agents effectively solidify in place. Other thermoreversible gelling agents such as polyethylene oxide—polylactic acid-polyethylene oxide polymers are also suitable in various embodiments.

In some embodiments, the poloxamer (e.g., poloxamer 407) is the gelling agent and the bulking agent of the pharmaceutical composition or reconstituted solution of the present disclosure. In some embodiments, the presence of the poloxamer (e.g., poloxamer 407) in the pharmaceutical composition (e.g., the lyophilized pharmaceutical composition) alleviates the need for any other excipient (e.g., additional bulking agent). Such alleviation may provide one or more advantages to the pharmaceutical composition (e.g., enhanced stability and/or reduced reconstitution time).

In some embodiments, the poloxamer is selected from the group consisting of Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, and Poloxamer 407.

In some embodiments, the poloxamer is Poloxamer 188 or Poloxamer 407.

In some embodiments, the poloxamer is Poloxamer 407.

In some embodiments, the poloxamer is a purified poloxamer (e.g., purified Poloxamer 407).

In some embodiments, the purified poloxamer (e.g., purified Poloxamer 407) has an average molecular weight of about 9 kDa or greater, about 9.2 kDa or greater, about 9.4 kDa or greater, about 9.6 kDa or greater, about 9.8 kDa or greater, about 10 kDa or greater, about 10.2 kDa or greater, about 10.4 kDa or greater, about 10.6 kDa or greater, about 10.8 kDa or greater, about 11 kDa or greater, about 11.2 kDa or greater, about 11.4 kDa or greater, about 11.6 kDa or greater, about 11.8 kDa or greater, about 12 kDa or greater, or about 12.1 kDa or greater.

In some embodiments, the purified poloxamer (e.g., purified Poloxamer 407) has a reduced level of polymer chains with molecular weight below 9 kDa as compared to the unpurified poloxamer (e.g., unpurified Poloxamer 407).

In some embodiments, the purified poloxamer (e.g., purified Poloxamer 407) has about 99% or less, about 98% or less, about 95% or less, about 90% or less, about 80% or less, about 70% or less, about 60% or less, about 50% or less, about 40% or less, about 30% or less, about 20% or less, or about 10% or less of polymer chains with molecular weight below 9 kDa as compared to the unpurified poloxamer (e.g., unpurified Poloxamer 407).

In some embodiments, the purified poloxamer (e.g., purified Poloxamer 407) is prepared by liquid-liquid extraction or size exclusion chromatography.

In some embodiments, about 10% or more, about 20% or more, about 30% or more, about 40% or more, about 50% or more, about 60% or more, about 70% or more, about 80% or more, about 90% % or more, about 95% or more, about 98% or more, or about 99% or more of the one or more impurities having molecular weights below 9 kDa are removed from the poloxamer (e.g., Poloxamer 407) during the purification.

In some embodiments, about 10% or more, about 20% or more, about 30% or more, about 40% or more, about 50% or more, about 60% or more, about 70% or more, about 80% or more, about 90% or more, about 95% or more, about 98% or more, or about 99% or more of the one or more diblock copolymers (e.g., PEO-PPO), single block polymers (e.g., PEO), and/or aldehydes are removed from the poloxamer (e.g., Poloxamer 407) during the purification.

In some embodiments, the pharmaceutical composition, pharmaceutical composition, the lyophilized pharmaceutical composition or reconstituted solution of the present disclosure comprises a buffering agent. The buffer controls the pH of the reconstituted solution to a range of from about 4 to about 13, from about 5 to about 12, from about 6 to about 11, from about 6.5 to about 10.5, or from about 7 to about 10.

Examples of the buffering agent include, but are not limited to, citrate buffering agents, acetate buffering agents, phosphate buffering agents, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. Lubricating agents may be selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

In some embodiments, the buffering agent comprises phosphate buffered saline, TRIS, tris acetate, tris HCl-65, sodium citrate, histidine, arginine, sodium phosphate, tris base-65, hydroxyethyl starch, or any combination thereof.

In some embodiments, the pharmaceutical composition, pharmaceutical composition, the lyophilized pharmaceutical composition or reconstituted solution of the present disclosure comprises a bulking agent.

In some embodiments, the bulking agent comprises poloxamer (e.g., poloxamer 407), mannitol, sucrose, maltose, trehalose, dextrose, sorbitol, glucose, raffinose, glycine, histidine, polyvinylpyrrolidone (e.g., polyvinylpyrrolidone K12 or polyvinylpyrrolidone K17), lactose, or any combination thereof.

In some embodiments, the pharmaceutical composition, pharmaceutical composition, the lyophilized pharmaceutical composition or reconstituted solution of the present disclosure comprises a stabilizing agent.

In some embodiments, the stabilizing agent comprises a cryoprotectant. In some embodiments, the cryoprotectant is a polyol (e.g., a diol or a triol such as propylene glycol (i.e., 1,2-propanediol), 1,3-propanediol, glycerol, (+/−)-2-methyl-2,4-pentanediol, 1,6-hexanediol, 1,2-butanediol, 2,3-butanediol, ethylene glycol, or diethylene glycol), a nondetergent sulfobetaine (e.g., NDSB-201 (3-(1-pyridino)-1-propane sulfonate), an osmolyte (e.g., L-proline or trimethylamine N-oxide dihydrate), a polymer (e.g., polyethylene glycol 200 (PEG 200), PEG 400, PEG 600, PEG 1000, PEG 3350, PEG 4000, PEG 8000, PEG 10000, PEG 20000, polyethylene glycol monomethyl ether 550 (mPEG 550), mPEG 600, mPEG 2000, mPEG 3350, mPEG 4000, mPEG 5000, polyvinylpyrrolidone (e.g., polyvinylpyrrolidone K 15), pentaerythritol propoxylate, or polypropylene glycol P 400), an organic solvent (e.g., dimethyl sulfoxide (DMSO) or ethanol), a sugar (e.g., D-(+)-sucrose, D-sorbitol, trehalose, D-(+)-maltose monohydrate, meso-erythritol, xylitol, myo-inositol, D-(+)-raffinose pentahydrate, D-(+)-trehalose dihydrate, or D-(+)-glucose monohydrate), or a salt (e.g., lithium acetate, lithium chloride, lithium formate, lithium nitrate, lithium sulfate, magnesium acetate, sodium chloride, sodium formate, sodium malonate, sodium nitrate, sodium sulfate, or any hydrate thereof) or any combination thereof.

In some embodiments, the stabilizing agent comprises a salt. In some embodiment, the salt is selected from the group consisting of lithium salts (e.g., lithium acetate, lithium chloride, lithium formate, lithium nitrate, lithium sulfate, or any hydrate thereof), magnesium salts (e.g., magnesium acetate or a hydrate thereof), and sodium salts (e.g., sodium chloride, sodium formate, sodium malonate, sodium nitrate, sodium sulfate, or any hydrate thereof). For another example, the formulation comprises one or more sodium salts. For yet another example, the formulation comprises sodium chloride.

In some embodiment, the stabilizing agent comprises a surfactant. In some embodiments, the surfactant comprises one or more anionic surfactants (e.g., 2-acrylamido-2-methylpropane sulfonic acid, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium dodecylbenzenesulfonate, sodium laurate, sodium laureth sulfate, sodium lauroyl sarcosinate, sodium myreth sulfate, sodium nonanoyloxybenzenesulfonate, sodium pareth sulfate, sodium stearate, or sulfolipid), one or more cationic surfactants (e.g., behentrimonium chloride, benzalkonium chloride, benzethonium chloride, benzododecinium bromide, bronidox, carbethopendecinium bromide, cetalkonium chloride, cetrimonium bromide, cetrimonium chloride, cetylpyridinium chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, octenidine dihydrochloride, olaflur, n-oleyl-1,3-propanediamine, pahutoxin, stearalkonium chloride, tetramethylammonium hydroxide, or thonzonium bromide), one or more zwitterionic surfactants (e.g., cocamidopropyl betaine, cocamidopropyl hydroxysultaine, dipalmitoylphosphatidylcholine, egg lecithin, hydroxysultaine, lecithin, myristamine oxide, peptitergents, or sodium lauroamphoacetate), and/or one or more non-ionic surfactants (e.g., alkyl polyglycoside, cetomacrogol 1000, cetostearyl alcohol, cetyl alcohol, cocamide dea, cocamide mea, decyl glucoside, decyl polyglucose, glycerol monostearate, igepal ca-630, isoceteth-20, lauryl glucoside, maltosides, monolaurin, mycosubtilin, narrow-range ethoxylate, nonidet p-40, nonoxynol-9, nonoxynols, np-40, octaethylene glycol monododecyl ether, n-octyl beta-d-thioglucopyranoside, octyl glucoside, oleyl alcohol, peg-10 sunflower glycerides, pentaethylene glycol monododecyl ether, polidocanol, α-tocopheryl polyethylene glycol succinate (TPGS), poloxamer (e.g., poloxamer 407), polyethoxylated tallow amine, polyglycerol polyricinoleate, polysorbate (e.g., polysorbate 20, polysorbate 40, polysorbate 60, or polysorbate 80), sorbitan, sorbitan monolaurate, sorbitan monostearate, sorbitan tristearate, stearyl alcohol, surfactin, Triton® x-100).

In some embodiments, the pharmaceutical composition, pharmaceutical composition, the lyophilized pharmaceutical composition or reconstituted solution of the present disclosure comprises a tonicity-adjusting agent.

In some embodiments, the tonicity-adjusting agent comprises NaCl, dextrose, dextran, Ficoll®, gelatin, mannitol, sucrose, glycine, glycerol, or any combination thereof.

In some embodiments, the pharmaceutical composition or reconstituted solution of the present disclosure comprises a soothing agent. In some embodiments, the soothing agent comprises lidocaine

In addition to these components, the pharmaceutical composition, pharmaceutical composition, the lyophilized pharmaceutical composition or reconstituted solution of the present disclosure includes any substance useful in pharmaceutical compositions.

In some embodiments, the pharmaceutical composition, pharmaceutical composition, the lyophilized pharmaceutical composition or reconstituted solution of the present disclosure includes one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are known in the art (see for example, Remington's The Science and Practice of Pharmacy, 21st Edition, A. R. Gennaro; Lippincott, Williams & Wilkins, Baltimore, Md., 2006 and 22nd Edition 2012 (ISBN: 978-0-85711-062-6)).

Examples of diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN® 20], polyoxyethylene sorbitan [TWEEN® 60], polyoxyethylene sorbitan monooleate [TWEEN® 80], sorbitan monopalmitate [SPAN® 40], sorbitan monostearate [SPAN® 60], sorbitan tristearate [SPAN® 965], glyceryl monooleate, sorbitan monooleate [SPAN® 80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers. (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC® F 68, POLOXAMER® 188, cetrimonium bromide, cetylpyridinium chloride, benzalkonium chloride, docusate sodium, and/or combinations thereof.

A binding agent may be starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN® II, NEOLONE™, KATHON™, and/or EUXYL®.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, an/or silicone oil.

Compounds or compositions described herein can be formulated in any manner suitable for a desired delivery route, e.g., transtympanic injection, intratympanic injection, transtympanic wicks and catheters, cochlear implants, and injectable depots. In some instances, compositions or formulations include one or more physiologically acceptable components, including derivatives or prodrugs, solvates, stereoisomers, racemates, or tautomers thereof with any physiologically acceptable carriers, diluents, and/or excipients.

As noted above, certain compositions are adapted for, and certain methods employ, administration to the middle ear or inner ear, for example, by local administration to the round window membrane. The membrane of the round window is the biological barrier to the inner ear space and represents the major obstacle for the local treatment of hearing impairment. The administered drug must overcome this membrane to reach the inner ear space. The drug can operatively (e.g., injection through the tympanic membrane) be placed locally to the round window membrane and can then penetrate through the round window membrane. Substances that penetrate the round window typically distribute in the perilymph and thus reach the hair cells and supporting cells.

The pharmaceutical compositions or formulations may also contain a membrane penetration enhancer, which supports the passage of the agents mentioned herein through the round window membrane. Accordingly, liquid, gel or foam formulations may be used. It is also possible to apply the active ingredient orally or to employ a combination of delivery approaches.

Certain compositions are adapted for, and certain methods employ, administration to the middle ear or inner ear, for example, by intratympanic or transtympanic administration. Intratympanic (IT) delivery of drugs to the ear is increasingly used for both clinical and research purposes. Some groups have applied drugs in a sustained manner using microcatheters and microwicks, while the majority have applied them as single or as repeated IT injections (up to 8 injections over periods of up to 2 weeks).

Intratympanically applied drugs are thought to enter the fluids of the inner ear primarily by crossing the round window (RW) membrane. Calculations show that a major factor controlling both the amount of drug entering the ear and the distribution of drug along the length of the ear is the duration the drug remains in the middle ear space. Single, ‘one-shot’ applications or applications of aqueous solutions for few hours' duration result in steep drug gradients for the applied substance along the length of the cochlea and rapidly declining concentration in the basal turn of the cochlea as the drug subsequently becomes distributed throughout the ear.

Other injection approaches include by osmotic pump, or, by combination with implanted biomaterial, and by injection or infusion. Biomaterials that can aid in controlling release kinetics and distribution of drug include hydrogel materials, degradable materials. One class of materials that is used includes in situ gelling materials. All potential materials and methodologies mentioned in references (Almeida H, Amaral M H, Lobao P, Lobo J M, Drug Discov Today 2014; 19:400-12; Wise A K, Gillespie L N, J Neural Eng 2012; 9:065002; Surovtseva E V, Johnston A H, Zhang W, et al., Int J Pharmaceut 2012; 424:121-7; Roy S, Glueckert R, Johnston A H, et al., Nanomedicine 2012; 7:55-63; Rivera T, Sanz L. Camarero G, Varela-Nieto I,. Curr Drug Deliv 2012; 9:231-42; Pararas E E, Borkholder D A, Borenstein J T, Adv Drug Deliv Rev 2012; 64:1650-60; Li M L, Lee L C, Cheng Y R, et al., IEEE T Bio-Med Eng 2013; 60:2450-60; Lajud S A, Han Z, Chi F L. et al., J Control Release 2013; 166:268-76; Kim D K, Park S N, Park K H, et al., Drug Deliv 2014; Engleder E, Honeder C, Klobasa J, Wirth M, Amoldner C, Gabor F, Int J Pharmaceut 2014; 471:297-302; Bohl A, Rohm H W, Ceschi P, et al., J Mater Sci Mater Med 2012; 23:2151-62; Hoskison E, Daniel M, Al-Zahid S, Shakesheff K M, Bayston R, Birchall J P, Ther Deliv 2013; 4:115-24; Staecker H, Rodgers B, Expert Opin Drug Deliv 2013; 10:639-50; Pritz C O, Dudas J, Rask-Andersen H, Schrott-Fischer A, Glueckert R. Nanomedicine 2013; 8:1155-72), which are included herein by reference in their entirety. Other materials include collagen or other natural materials including fibrin, gelatin, and decellularized tissues. Gelfoam may also be suitable.

Delivery may also be enhanced via alternate means including but not limited to agents added to the delivered composition such as penetration enhancers, or could be through devices via ultrasound, electroporation, or high-speed jet.

Methods described herein can also be used for inner ear cell types that may be produced using a variety of methods know to those skilled in the art including those cell types described in PCT Application No. WO2012103012 A1.

With regard to human treatment, the amount of a particular agent(s) that is administered may be dependent on a variety of factors, including the disorder being treated and the severity of the disorder; activity of the specific agent(s) employed; the age, body weight, general health, sex and diet of the patient; the time of administration, route of administration, and rate of excretion of the specific agent(s) employed; the duration of the treatment; drugs used in combination or coincidental with the specific agent(s) employed; the judgment of the prescribing physician; and like factors known in the medical arts.

The agents described herein may be administered in a therapeutically effective amount to a subject in need of treatment. Administration of compositions described herein can be via any of suitable route of administration, for example, by intratympanic administration. Other routes include ingestion, or alternatively parenterally, for example, intravenously, intra-arterially, intraperitoneally, intrathecally, intraventricularly, intraurethrally, intrasternally, intracranially, intramuscularly, intranasally, subcutaneously, sublingually, transdermally, or by inhalation or insufflations, or topical by ear instillation for absorption through the skin of the ear canal and membranes of the eardrum. Such administration may be as a single or multiple oral dose, defined number of ear drops, or a bolus injection, multiple injections, or as a short- or long-duration infusion. Implantable devices (e.g., implantable infusion pumps) may also be employed for the periodic parenteral delivery over time of equivalent or varying dosages of the particular formulation. For such parenteral administration, the compounds may be formulated as a sterile solution in water or another suitable solvent or mixture of solvents. The solution may contain other substances such as salts, sugars (particularly glucose or mannitol), to make the solution isotonic with blood, buffering agents such as acetic, citric, and/or phosphoric acids and their sodium salts, and preservatives.

Compositions described herein can be administered by several methods sufficient to deliver the composition to the inner ear. Delivering a composition to the inner ear includes administering the composition to the middle ear, such that the composition may diffuse across the round window to the inner ear. It also includes administering a composition to the inner ear by direct injection through the round window membrane. Such methods include, but are not limited to auricular administration, by transtympanic wicks or catheters, or parenteral administration, for example, by intraauricular, transtympanic, or intracochlear injection.

In particular embodiments, the compounds, compositions and formulations of the disclosure are locally administered, meaning that they are not administered systemically.

In one embodiment, a syringe and needle apparatus is used to administer compounds or compositions to a subject using auricular administration. A suitably sized needle is used to pierce the tympanic membrane and a wick or catheter comprising the composition is inserted through the pierced tympanic membrane and into the middle ear of the subject. The device may be inserted such that it is in contact with the round window or immediately adjacent to the round window. Exemplary devices used for auricular administration include, but are not limited to, transtympanic wicks, transtympanic catheters, round window microcatheters (small catheters that deliver medicine to the round window), and Silverstein Microwicks™ (small tube with a “wick” through the tube to the round window, allowing regulation by subject or medical professional).

In some embodiments, a syringe and needle apparatus is used to administer compounds or compositions to a subject using transtympanic injection, injection behind the tympanic membrane into the middle and/or inner ear. The formulation may be administered directly onto the round window membrane via transtympanic injection or may be administered directly to the cochlea via intracochlear injection.

In some embodiments, a compound or composition disclosed herein is administered to a subject in a single intratympanic injection.

In some embodiments, the delivery device is an apparatus designed for administration of compounds or compositions to the middle and/or inner ear. By way of example only: GYRUS Medical GmbH offers micro-otoscopes for visualization of and drug delivery to the round window niche; Arenberg has described a medical treatment device to deliver fluids to inner ear structures in U.S. Pat. Nos. 5,421,818; 5,474,529; and 5,476,446, each of which is incorporated by reference herein for such disclosure. U.S. patent application Ser. No. 08/874,208, which is incorporated herein by reference for such disclosure, describes a surgical method for implanting a fluid transfer conduit to deliver compositions to the inner ear. U.S. Patent Application Publication 2007/0167918, which is incorporated herein by reference for such disclosure, further describes a combined otic aspirator and medication dispenser for transtympanic fluid sampling and medicament application.

In some embodiments, a compound or composition disclosed herein is administered to a subject in need thereof once. In some embodiments, a compound or composition disclosed herein is administered to a subject in need thereof more than once. In some embodiments, a first administration of a compound or composition disclosed herein is followed by a second, third, fourth, or fifth administration of a compound or composition disclosed herein.

The number of times a compound or composition is administered to a subject in need thereof depends on the discretion of a medical professional, the disorder, the severity of the disorder, and the subject's response to the formulation. In some embodiments, the compound or composition disclosed herein is administered once to a subject in need thereof with a mild acute condition. In some embodiments, a compound or composition disclosed herein is administered more than once to a subject in need thereof with a moderate or severe acute condition. In the case wherein the subject's condition does not improve, upon the doctor's discretion the compound or composition may be administered chronically, that is, for an extended period of time, including throughout the duration of the subject's life in order to ameliorate or otherwise control or limit the symptoms of the subject's disease or condition.

In the case wherein the subject's status does improve, upon the doctor's discretion the compound or composition may administered continuously; alternatively, the dose of drug being administered may be temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday may be from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once the subject's hearing has improved, a maintenance dose can be administered, if necessary. Subsequently, the dosage or the frequency of administration, or both, is optionally reduced, as a function of the symptoms, to a level at which the improved disease, disorder or condition is retained. In certain embodiments, subjects require intermittent treatment on a long-term basis upon any recurrence of symptoms.

Certain embodiments include is a pharmaceutical product comprising a sealed packaging and the compound(s) in the container. The container size can be optimized to reduce head space in the container after packaging and any head space may be filled with an inert gas such as nitrogen. Furthermore, container material of construction can be chosen to minimize the moisture and oxygen ingress inside the container after packaging.

### CHIR99021 and VPA

The inventors have found that a combination treatment of a CHIR99021 or a pharmaceutically acceptable salt thereof and valproic acid or a pharmaceutically acceptable salt thereof may be particularly effective at treating sensorineural hearing loss. Provided in one aspect is CHIR99021 or a pharmaceutically acceptable salt thereof and valproic acid (VPA) or a pharmaceutically acceptable salt thereof for use in treating sensorineural hearing loss in a human patient.

The structure of CHIR99021 is as follows:

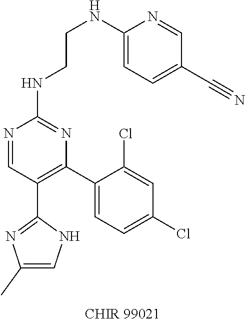 [SMILES: Cc1c[nH]c(-c2cnc(NCCNc3ccc(C#N)cn3)nc2-c2ccc(Cl)cc2Cl)n1]

The structure of VPA is as follows:

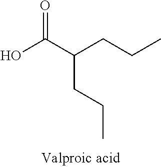

In some embodiments, e pharmaceutically acceptable salt of VPA is sodium valproate.

In some embodiments, the CHIR99021 is at a concentration of about 1 mM, 2 mM, 3 mM, 4 mM, 5 mM, 6 mM, 7 mM, 8 mM, 9 mM, or 10 mM and the VPA or a pharmaceutically acceptable salt thereof at a concentration about 100 mM to 4,000 mM.

In some embodiments, the CHIR99021 is at a concentration of about 5.5 mM, 5.6 mM, 5.7 mM, 5.8 mM, 5.9 mM, 6.0 mM, 6.1 mM, 6.2 mM, 6.3 mM, 6.4 mM, 6.5 mM, 6.6 mM, 6.7 mM, 6.8 mM, 6.9 mM or 7.0 mM and the VPA or a pharmaceutically acceptable salt thereof at a concentration about 100 mM, 200 mM, 300 mM, 400 mM, 500 mM, 600 mM, 700 mM, 800 mM, 900 mM, or 1000 mM.

In some embodiments, the CHIR99021 is at a concentration of about 5.5 mM, 5.6 mM, 5.7 mM, 5.8 mM, 5.9 mM, 6.0 mM, 6.1 mM, 6.2 mM, 6.3 mM, 6.4 mM, 6.5 mM, 6.6 mM, 6.7 mM, 6.8 mM, 6.9 mM or 7.0 mM and the VPA or a pharmaceutically acceptable salt thereof at a concentration about 100 mM, 200 mM, 300 mM, 400 mM, 500 mM, 600 mM, 700 mM, 800 mM, 900 mM, or 1000 mM.

In some embodiments, the CHIR99021 is at a concentration of about 6.1 mM, about 6.2 mM, about 6.3 mM, about 6.4 mM, about 6.5 mM, about 6.6 mM, about 6.7 mM, about 6.8 mM, about 6.9 mM, about 7.0 mM, about 7.1 mM, about 7.2 mM, about 7.3 mM, about 7.4 mM or about 7.5 mM and the VPA or a pharmaceutically acceptable salt thereof at a concentration about 465 mM, about 475 mM, about 485 mM, about 495 mM, about 505 mM, about 515 mM, about 525 mM, about 535 mM, about 545 mM or about 555 mM, about 565 mM, about 575 mM, about 585 mM, about 595 mM, or about 605 mM.

In some embodiments, the CHIR99021 or a pharmaceutically acceptable salt thereof is at a concentration of about 6.75 mM and the VPA is sodium valproate at a concentration of about 533 mM. In certain such embodiments, the CHIR99021 or a pharmaceutically acceptable salt thereof is at a concentration of 3.14 mg/ml and the VPA is sodium valproate at a concentration of 88.6 mg/ml.

In some embodiments, the CHIR99021 or a pharmaceutically acceptable salt thereof is at a concentration of about 0.3% (w/w) and the VPA is sodium valproate at a concentration of about 8% (w/w), for example, as described in Example 5. In some embodiments, the CHIR99021 or a pharmaceutically acceptable salt thereof is at a concentration of between 0.27% and 0.33% (w/w) and the sodium valproate is at a concentration of between 7.2% and 8.8% (w/w).

In some embodiments, the CHIR99021 or a pharmaceutically acceptable salt thereof and VPA or a pharmaceutically acceptable salt thereof are formulated as a single composition. In other embodiments, the CHIR99021 or a pharmaceutically acceptable salt thereof and VPA or a pharmaceutically acceptable salt thereof are formulated as separate compositions.

In some embodiments, as noted above, a composition is adapted for administration to the inner ear and/or middle ear, for example, local administration to the round window membrane or intratympanic or transtympanic administration, for example, to cochlear tissue.

In some embodiments, the composition comprises a poloxamer. In some embodiments, the poloxamer is Poloxamer 407.

In some embodiments, CHIR99021 or a pharmaceutically acceptable salt thereof and VPA or a pharmaceutically acceptable salt thereof as described herein are administered to the middle ear. In some embodiments, administration to the middle ear is by intratympanic injection.

When administered locally, for example, to the inner and/or middle ear, the compounds (s) are administered at a unit dose of about 25 μl to 500 μl, or about 50 μl to 200 μl. In some cases, CHIR99021 or a pharmaceutically acceptable salt thereof and VPA or a pharmaceutically acceptable salt is formulated in a single composition as is administered locally, to the inner and/or middle ear, at a unit dose of about is 50 μl to 200 μl.

Patients having moderate hearing loss when assessed by pure tone audiometry may be particularly suitable for treatment with CHIR99021 or a pharmaceutically acceptable salt thereof and VPA or a pharmaceutically acceptable salt thereof as described herein. In certain embodiments, the average of the patient's hearing thresholds across 0.5 kHz, 1 kHz, 2 kHz and 4 kHz is at least 40 dB HL and no more than 70 dB HL when measured by pure tone audiometry prior to the treatment.

Patients having poor intelligibility may be particularly suitable for treatment with CHIR99021 or a pharmaceutically acceptable salt thereof and VPA or a pharmaceutically acceptable salt thereof as described herein. Accordingly, in some embodiments, the patient has a standard word recognition score of 60% or less prior to the treatment. In certain such embodiments the standard word recognition test is the Maryland CNC test, using the words list and carrier phrases as defined in Causey G D, Hood L J, Hermanson C L, Bowling L S. (1984) The Maryland CNC Test: normative studies. Audiology 23(6): 552-568. In this embodiment, the word signal is provided to the patient at 40 dB above speech perception level.

In some embodiments, the patient has a words-in-noise score of 50% or less prior to the treatment. In certain such embodiments the WIN test consists of administering 70 monosyllabic words from the NU No. 6 word lists, where the SNR decibel level of the test words varies from 24 dB SNR (easiest condition) to 0 dB SNR (most difficult condition) in 4 dB decrements, for a total of seven SNR levels (i.e., 24 dB SNR, 20 dB SNR, 16 dB SNR, 12 dB SNR, 8 dB SNR, 4 dB SNR and 0 dB SNR). In one embodiment, the level of multi-talker babble is fixed at 70 dB SPL, and the level of the test word signal varies from 70 dB SPL to 94 dB SPL. In another embodiment, the level of multi-talker babble is fixed at 80 dB SPL, and the level of the test word signal varies from 80 dB SPL to 104 dB SPL

In some embodiments, the average of the patient's hearing thresholds across 0.5 kHz, 1 kHz, 2 kHz and 4 kHz is at least 40 dB HL and no more than 70 dB HL when measured by pure tone audiometry prior to the treatment, the patient has a standard word recognition score of 60% or less prior to the treatment and the patient has a words-in-noise score of 50% or less prior to the treatment.

The inventors have found that treatment with CHIR99021 or a pharmaceutically acceptable salt thereof and VPA or a pharmaceutically acceptable salt thereof as described herein provides particular improvements in a patient's sound intelligibility as assessed using word recognition tests. Accordingly, in some embodiments, treatment provides an improved standard word recognition score for the patient, wherein said improvement, if tested, would be at least 100%, wherein said percentage improvement is calculated using the following formula:

$$100 \times ( \frac{patient ’ s word recognition score after treatment}{patient ’ s word recognition score prior to treatment} - 1 )$$

In some embodiments, treatment provides an improved words-in-noise score for the patient, wherein said improvement, if tested, would be at least 10%, wherein said percentage improvement is calculated using the following formula:

$$100 \times ( \frac{patient ’ s words in noise score after treatment}{patient ’ s words in noise score prior to treatment} - 1 )$$

An improvement in a patient's sound intelligibility as assessed using a standard word recognition test or a words-in-noise test is not necessarily accompanied by an improvement in a patient's sound audibility. Accordingly, in some embodiments, the average of the patient's hearing thresholds across 0.5 kHz, 1 kHz, 2 kHz and 4 kHz after the treatment, if tested, would be no more than 5 dB increased or decreased to the average of the patient's hearing thresholds across 0.5 kHz, 1 kHz, 2 kHz and 4 kHz prior to the treatment, wherein said hearing thresholds are measured by pure tone audiometry.

The inventors have found that treatment with CHIR99021 or a pharmaceutically acceptable salt thereof and VPA or a pharmaceutically acceptable salt thereof as described herein may provide an improvement in audibility function at the higher frequencies of the standard audiometric frequencies. Accordingly, in some embodiments the treatment provides an improved hearing threshold at 8 kHz, wherein said improvement, if tested, would be at least 5 dB relative to the patient's hearing threshold at 8 kHz prior to the treatment, wherein said hearing threshold is measured by pure tone audiometry.

In some embodiments, the treatment provides (i) an improved hearing threshold at 8 kHz, wherein said improvement, if tested, would be at least 5 dB relative to the patient's hearing threshold at 8 kHz prior to the treatment, wherein said hearing threshold is measured by pure tone audiometry and (ii) an improved standard word recognition score for the patient or an improved words-in-noise score for the patient, wherein said improvement in standard word recognition score, if tested, would be at least 10%, wherein said percentage improvement is calculated using the following formula:

$$100 \times ( \frac{patient ’ s word recognition score after treatment}{patient ’ s word recognition score prior to treatment} - 1 )$$

wherein said improvement in words-in-noise score, if tested, would be at least 10%, wherein said percentage improvement is calculated using the following formula:

$$100 \times ( \frac{patient ’ s words in noise score after treatment}{patient ’ s words in noise score prior to treatment} - 1 )$$

In certain such embodiments, the treatment also provides an improved hearing threshold at 6 kHz, wherein said improvement, if tested, would be at least 5 dB relative to the patient's hearing threshold at 6 kHz prior to the treatment.

The inventors have found that treatment with CHIR99021 or a pharmaceutically acceptable salt thereof and VPA or a pharmaceutically acceptable salt thereof as described herein are able to provide an improvement in hearing function following a single administration. Accordingly, in some embodiments, an improvement in hearing is provided by a single administration.

The inventors have found that treatment with CHIR99021 or a pharmaceutically acceptable salt thereof and VPA or a pharmaceutically acceptable salt thereof as described herein are able to provide an improvement in hearing function within 90 days of a single administration. Thus, in some embodiments, an improvement in hearing function, for example, in threshold and/or word recognition, is provided within 90 days.

### Definitions

All references to a specific compound also encompass all equivalents of that compound. For example, all references to a specific compound also encompass and disclose salts, deuterates, solid forms (e.g., solvates, polymorphs, hydrates, etc.), prodrugs, metabolites, and combinations thereof.

### EXAMPLES

The invention now being generally described will be more readily understood by reference to the following examples, which are included merely for the purpose of illustrating certain aspects and embodiments of the present invention, and are not intended to limit the invention.

The examples use a specific form of FX-322, as defined below, which are included merely for purposes of illustration of certain aspects and embodiments of the present invention, and are not intended to limit the invention.

FX-322, as used herein, unless otherwise noted comprises the following:

| FX-322 | mg/ml |
|---|---|
| CHIR99021 | 3.14 |
| Valproate Na | 88.63 |
| Poloxamer 407 | 161.5 |
| DMSO | 55 |
| Water | q.s. |
| pH | 9.0 |
| Osmolality | 2500 |
| (mOsm/kg) | |

### Example 1

### In Vivo Mouse Hearing Loss Model

The effects of CHIR99021 and valproic acid (VPA) on hearing in mice with SNHL were examined. Ten-week-old CBA/CaJ mice were deafened using an established method in which mice were exposed to 8-16 kHz octave band noise for 2 hours at ≥116 dB (Wang et al., 2002). This model was shown to cause immediate and extensive hair cell loss, but also causes damage to other structures, such as the lateral wall, supporting cells, and spiral ganglion, all of which could limit the extent of possible hearing recovery (Wang et al., 2002). Auditory brainstem responses (ABRs) were obtained using tone-burst stimuli for frequencies spanning ˜80% of the cochlea 24 hours after noise administration to establish a baseline for recovery. Animals were dosed once following the 24 hour ABR. Distortion product otoacoustic emissions (DPOAEs) were not routinely analyzed since thresholds after treatment were above DPOAE detection levels. CHIR99021 and VPA were delivered locally by intratympanic injection into the middle ear using a pulled glass pipette, a technique that mimics the standard clinical middle ear injection technique used in humans. The delivery vehicle was adapted from previous work using thermo-reversible poloxamer gels to deliver drugs into the middle ear for diffusion into the cochlea (Salt et al, 2011; Wang et al, 2009). Doses of CHIR99021 and VPA were scaled several hundred-fold above the active in vitro concentration to account for the gradient of drug entry through the round window membrane described in previous studies (Plontke et al., 2008). Specifically, mice were administered 10 □L of a composition containing 87.6 mg/ml NaVPA (527 mM) and 1.39 mg/ml CHIR99021 (approximately 3 mM) (CHIR99021/VPA). Using established techniques (Hirose et al., 2014), perilymph was sampled from 7 animals and analyzed using mass spectrometry to determine entry of CHIR99021 and VPA into the cochlea. Within 0.5 hours, CHIR99021 was detected at 3.5 μM+1.5 μM and VPA was detected at 310.3 μM+51.8 μM. Thus, concentrations that were active in the in vitro Lgr5+ cell proliferation assay (as described in McLean et. al., 2017) were achieved within the cochlea using an intratympanic injection.

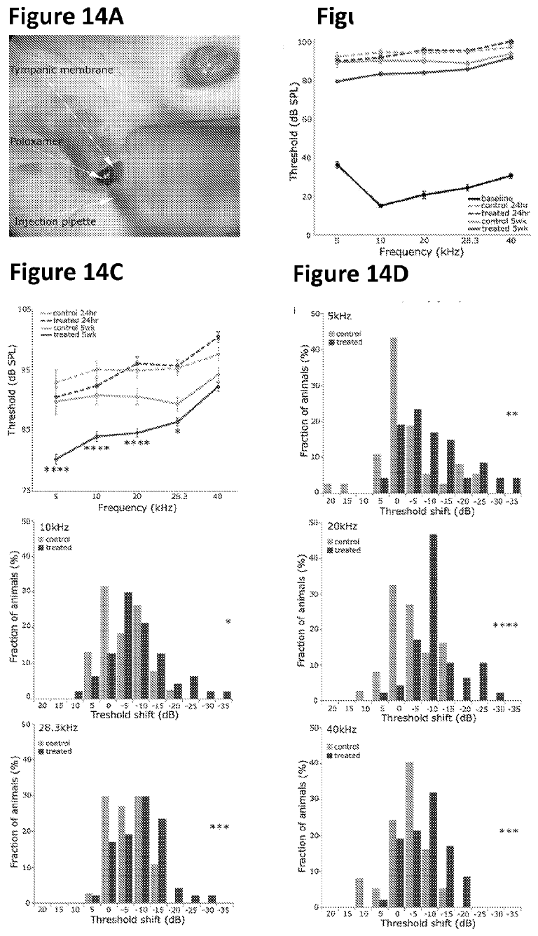

Consistent with previous reports of rapid HC death using this noise-damage model (Wang et al., 2002), total HC numbers observed prior to injection (24 hours after damage) did not significantly differ from those observed in vehicle-injected animals at 5 weeks (data not shown, n=6, p=0.11). This confirms prior work demonstrating a lack of spontaneous hair cell regeneration in post-natal mammals (Cox et al., 2014; Bramhall et al., 2014). Five weeks after injection, animals that received CHIR99021/VPA showed significantly lower absolute ABR thresholds relative to vehicle-injected animals at 5, 10, 20 (p<0.0001), and 28.3 (p<0.05) kHz (FIG. 14). Average threshold changes from post-damage to 5 weeks were significantly greater across all frequencies tested in treated animals, with some demonstrating threshold recoveries up to 35 dB (FIG. 14).

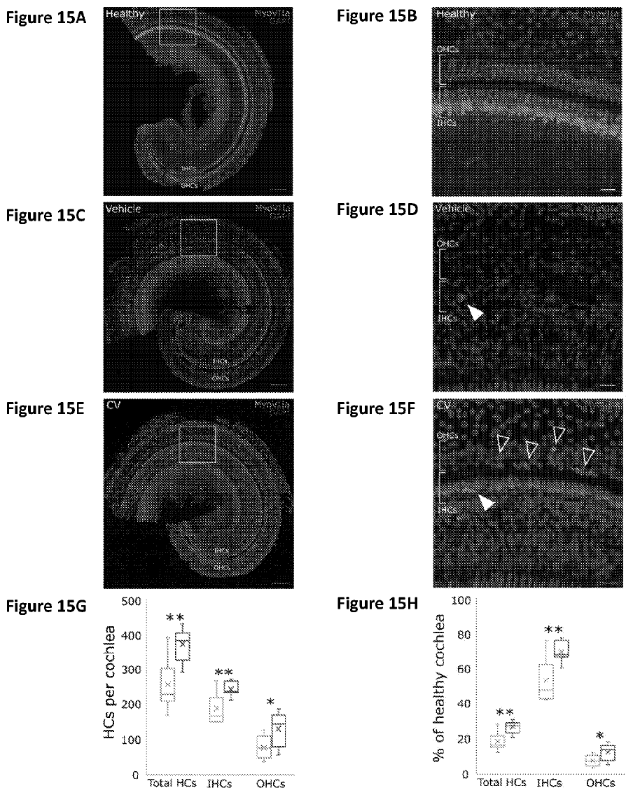

These data demonstrate that a Wnt activator combined with an epigenetic modulator can be used to improve hearing. After final physiological testing, histologic analyses were performed. It was found that total hair cell (total HC), inner hair cell (IHC), and outer hair cell (OHC) numbers increased in CHIR99021/VPA-treated animals relative to vehicle-treated animals (Total HCs=376.0±18.5, IHCs=245.9±7.9, OHCs=130.1±18.8; mean±SE) vs (Total HCs=259.3±29.0 [p<0.01], IHCs=188.6±16.5 [p<0.01], OHCs=75.3±12.4 [p<0.05]) (FIG. 15).

These data demonstrate that the combination of a Wnt activator (CHIR99021) and an epigenetic modulator (VPA), the components of FX-322 can improve auditory thresholds in a model of hearing loss. Furthermore, these data demonstrate that the composition can restore hair cells in vivo.

### Example 2

### Study Design and Oversight

Although FX-322 demonstrated positive effects in rodent models of noise-induced hearing loss, it was unknown if the effects would translate to humans, not only because of inherent differences between rodents and humans, but also because human SNHL can be caused by a number of different factors. Accordingly, a Phase 1/2, double-blind, randomized, placebo-controlled study was conducted at four study sites in the United States between July 2018 and October 2018. The study was conducted according to International Conference on Harmonisation guidelines, Good Clinical Practices, and the Declaration of Helsinki. The protocol and amendments were approved by the Institutional Review Board for participating investigators.

### Patient Population and Treatments

Adults aged 18-65 years were eligible for participation in the clinical trial if they had an established diagnosis of stable (no documented changes of >10 dB at any frequency for >6 months) noise-induced or idiopathic sudden SNHL. Patients were excluded for 1) current use of VPA in any form; 2) tympanic membrane perforation or other disorders of the tympanic membrane; 3) conductive hearing loss of >10 dB in either ear at two or more frequencies; 4) pure tone average of >70 dB at 500, 1000, 2000, and 4000 Hz in the ear to be injected; 5) active chronic middle ear disease; 6) history of major middle ear surgery as an adult; 7) receipt of an intratympanic injection within 6 months; 8) history of clinically significant vestibular symptoms; 9) clinically significant systemic autoimmune disease; or 10) history of head or neck radiation treatment.

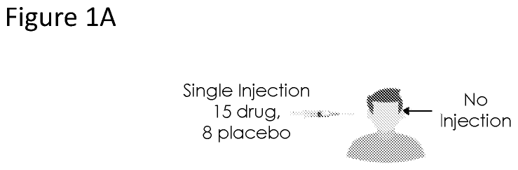

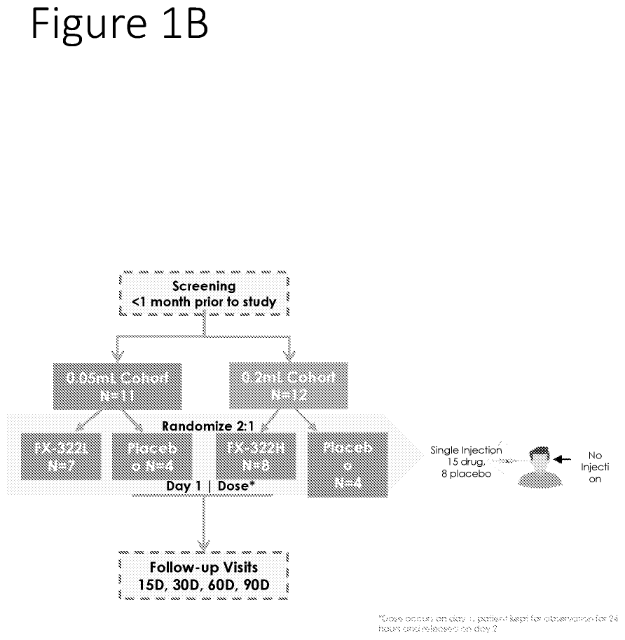

Patients were randomized to one of four treatment groups (FIG. 1): FX-322 low volume (50 μL), placebo low volume. FX-322 high volume (200 μL), or placebo high volume using a 1:1 allocation ratio for dose cohort (12 per cohort) and within each cohort using a 2:1 allocation ratio for study drug (8 FX-322: 4 placebo). Since the volume in the syringe could potentially result in unblinding, cohort assignment may not have been blinded to the otolaryngologist delivering the treatment, although randomization to FX-322 or placebo remained double-blind to all other study staff and patients. Pharmacy staff who prepared study drug and the independent statistician and/or independent statistical programmer were unblinded.

Twenty-three patients with stable, noise-induced (n=12) or idiopathic sudden SNHL (n=11) were randomized to one of four treatment groups: FX-322 low volume (50 μL; n=7), placebo low volume (n=4), FX-322 high volume (200 μL; n=8), or placebo high volume (n=4) (FIG. 1). Patients were balanced across treatment groups for baseline demographic and clinical characteristics although the placebo group had a somewhat longer duration of hearing loss (Table 14).

TABLE 14
Summary of baseline characteristics

| | FX-322 | FX-322 | Pooled | |
|---|---|---|---|---|
| | Low | High | Placebo | Total |
| | (n = 7) | (n = 8) | (n = 8 | (n = 23) |
| Age, years ^{a} | 59.1 ± 5.7 | 50.5 ± 9.2 | 57.0 ± 5.8 | 55.4 ± 7.8 |
| Age range, years | 48-64 | 33-62 | 45-63 | 33-64 |
| Male, n (%) | 5 (71.4) | 5 (62.5) | 4 (50.0) | 14 (64.3) |
| Race, n (%) | | | | |
| White | 6 (85.7) | 7 (87.5) | 8 (100) | 21 (91.4) |
| American Indian/Alaska | 0 | 1 (12.5) | 0 | 1 (4.3) |
| Native | | | | |
| Asian | 1 (14.3) | 0 | 0 | 1 (4.3) |
| Etiology, n (%) | | | | |
| Noise-induced hearing loss | 4 | 3 | 5 | 12 |
| Sudden sensorineural | 3 | 5 | 3 | 11 |
| hearing loss | | | | |
| Median duration, years | 3 | 4 | 5 | 4 |
| Mean duration, years | 5.4 | 7.6 | 11.9 | 8.4 |
| Duration range, years | 1-20 | 1-21 | 1-43 | 1-43 |

### Study Assessments and Endpoints

Exploratory endpoints included measures of audibility and speech intelligibility. Comprehensive audiometric assessment including otoscopy, tympanometry, pure-tone audiometry, word recognition in quiet (WR), and words-in-noise (WIN) were performed at screening and on days 15, 30, 60, and 90.

Word recognition (WR) was performed at screening and on days 15, 30, 60, and 90 to quantify the ability of listeners to understand monosyllabic English words at a suprathreshold level. The WR test involved the administration of 50 monosyllabic words in a quiet background at a loud, but comfortable level. The words were played from a CD player at 40 dB above speech perception threshold. The Maryland consonant-vowel nucleus-consonant (CNC) list was used. One list was administered in each ear. Randomized lists were used across the study. WR is a validated test used widely by audiologists. The total number of words repeated correctly was recorded.

The Words-in-Noise Test (WIN) was performed at screening and days 15, 30, 60, and 90 to quantify the ability of listeners to understand monosyllabic words in background noise (Wilson, 2003). The WIN test involved the administration of 70 monosyllabic words in a multitalker background. The level of the multitalker babble is fixed at 40 dB above the patient's speech reception threshold, with 3 females and 3 males talking about various topics. Words are played from 24 dB signal-to-noise ratio (SNR) to 0 dB SNR in a descending manner. The results were analyzed for the SNR (dB) at which 50% correct word performance was achieved.

A responder definition was created while blinded that required both an improvement in audiometry (≥5 dB at 8 kHz) and a functional hearing improvement in either WR or WIN (≥10%) compared to baseline. Measurements at 8 kHz were specifically evaluated because an effective concentration of the drug in the base of the cochlea was anticipated. Once established, these improvements needed to be sustained for all subsequent visits in the study for the subject to be classified as a responder in this example.

Plasma samples were obtained pre-dose and up to 24 hours post injection to assess the systemic exposure to the active pharmaceutical ingredients of FX-322, CHIR99021 and VPA. Patients underwent physical examination of weight and height, vital signs (body temperature, pulse rate, blood pressure), 12-lead electrocardiogram (ECG), drug screen, clinical laboratory testing (hematology, serum chemistry, urinalysis), urine pregnancy test, and hepatitis B and C antibody tests.

### Statistical Analysis

The selected sample size was considered adequate for an initial assessment of safety and tolerability and was not based on formal statistical considerations. The statistical analyses consisted of descriptive statistics: mean, standard deviation (SD), median, minimum, and maximum statistics for continuous endpoints and numbers and percent for categorical endpoints. Comparisons between groups for WR, WIN, and audiometry were done with a 2-tailed, mixed model comparison of adjusted mean (standard error) percent change from baseline on each study day. For each comparison, 95% confidence intervals (CIs) were calculated for the mean (SE) difference between treatments. For response, treatment groups were compared with Fisher's Exact Test.

### Efficacy

The performance of two individual responders is provided in Table 15 as an example of results. These patients had asymmetric hearing loss with one ear performing significantly better on measures of speech understanding. Both patients showed marked improvements after single injection.

TABLE 15
Examples of individual patient treatment effect.

| | | | | % increase in |
|---|---|---|---|---|
| | | | Performance 90 | performance |
| | | | days after single | over baseline 90 |
| | | Baseline | injection | days after single |
| Patient | Endpoint | (words) | (words) | injection |
| 936 | Word recognition | 20/50 | 39/50 | 95 |
| | Words in noise | 18/70 | 28/70 | 55 |
| | dB SNR in WIN | 18.8 | 14.8 | 21 |
| 916 | Word recognition | 14/50 | 34/50 | 140 |
| | Words in noise | 13/70 | 23/70 | 76 |
| | dB SNR in WIN | 20.8 | 16.8 | 19 |

Patient 936 was a 58 year old male with stable, moderate noise-induced hearing loss. The first patient entered the study scoring 20/50 (40%) on WR in quiet in the poorer ear and 38/50 (76%) in the better ear. By day 90, the performance of the poorer ear (39/50, 78%) improved significantly to match that of the better ear (41/50; 82%). Looking at performance over baseline measures, the treated ear showed a 95% increase in performance for WR and a 55% increase in performance for WIN at 90 days after injection. In terms of absolute percentage change, patient 916 improved 40% in WR and 14% in WIN. Patient 936 improved 38% WR and 14% WIN. Patient 936 also had a 5 dB improvement in 8 kHz audiometry, which represents a near doubling in sound pressure. Taken together, this means improved hearing for patient 936.

Patient 916 was a 55 year old male with stable sudden sensorineural hearing loss. Patient 916 entered the study scoring 14/50 (28%) in the poorer ear, and 46/50 (92%) in the better ear for WR testing. WR in the poorer ear improved to 34/50 (68%), and 50/50 (100%) in the better ear. For patient 916, WIN improved from 13/70 (19%) to 23/70 (33%), which represents a 76% improvement over baseline measures. Patient 916 also had a 10 dB improvement in 8 kHz audiometry, which represents more than a tripling in sound pressure. Taken together, this means improved hearing for patient 916. In addition, patients 916 and 936 both showed clinically meaningful improvement in WIN from baseline to day 90, with SNR improvements that exceeded the 3.1 dB 95% confidence interval established by Wilson and McArdle, 2007.

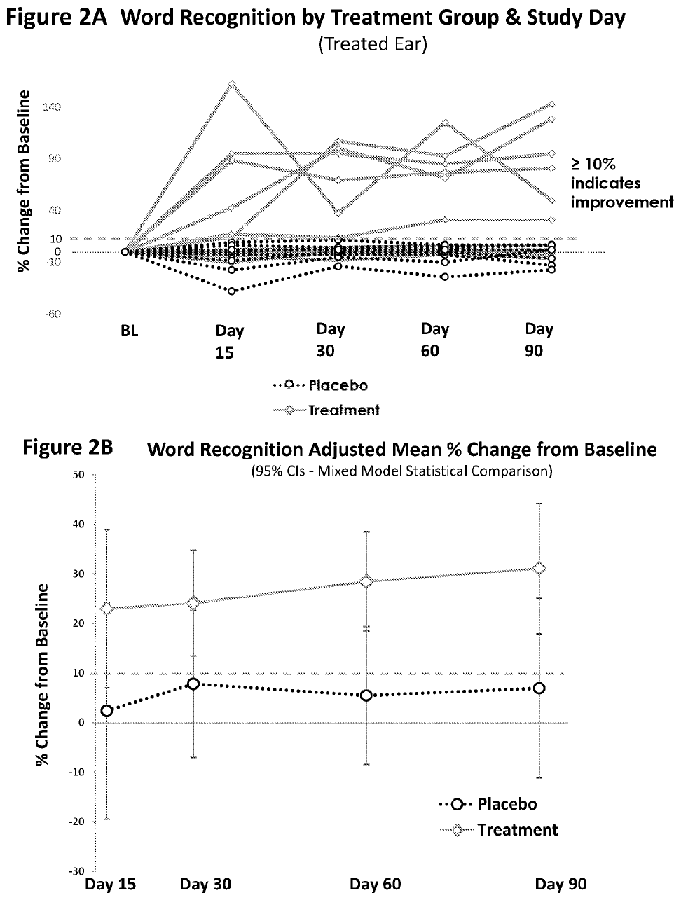

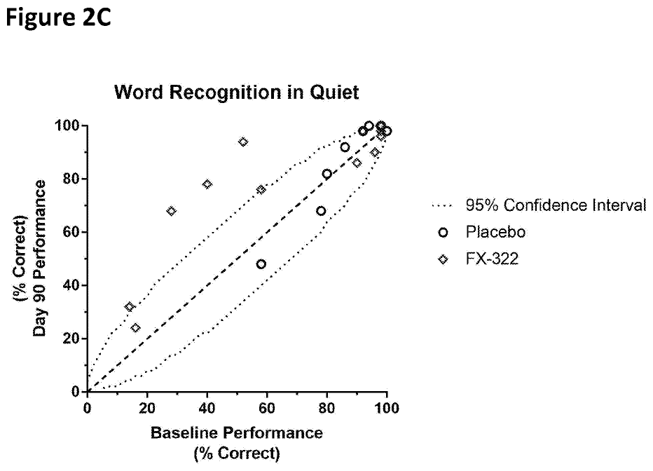

Overall, significant changes were seen in the cohort of FX-322-treated patients on measures of speech intelligibility. For WR, significant improvements were seen at Day 15 and were sustained for the duration of the study (p=0.01) (FIG. 2B). On average, FX-322-treated patients show a 30% improvement (FIG. 2B), and only FX-322-treated patients showed improvements of >10% (FIG. 2A). Four (27%) of the FX-322 patients, but none of the placebo patients showed a clinically significant improvement from baseline to Day 90 in the WR test according to the definition and criteria established by Thornton and Raffin, 1978 (FIG. 2C). Specifically, the 95% CI bounds were calculated in radians using the arcsine transformation for proportions of words recognized as detailed by Studebaker. Radian Cis were then converted back to word recognition proportions per the iterative procedure detailed by Thornton and Raffin. For individual patients, changes from baseline falling outside the 95% CIs were for individual patients were determined to be significant. Therefore FX-322 treatment resulted in an increase in intelligibility and speech perception.

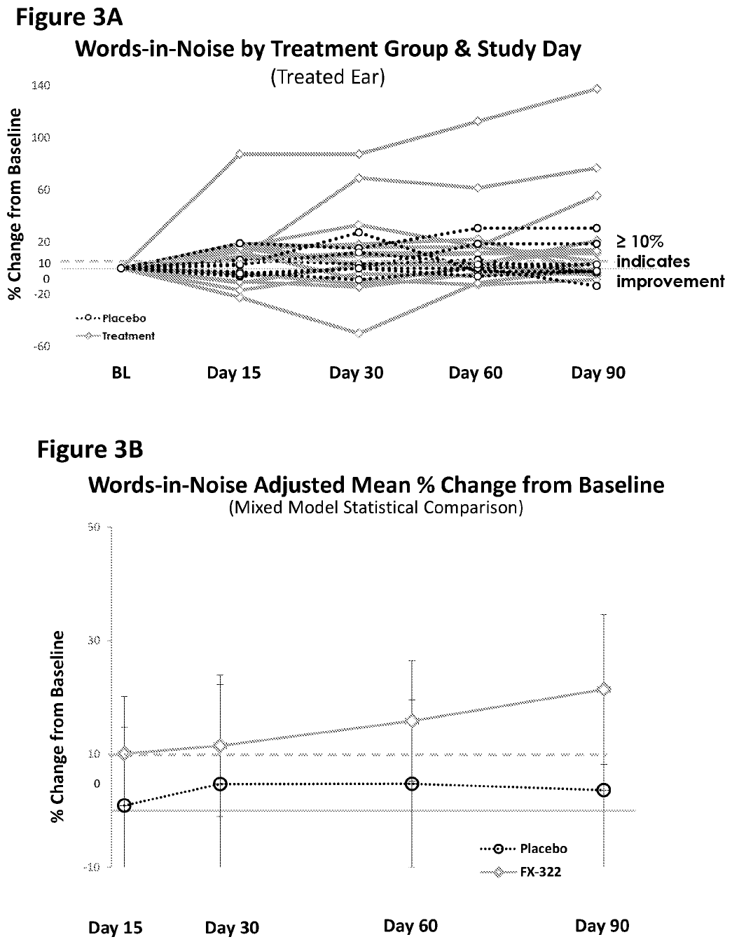

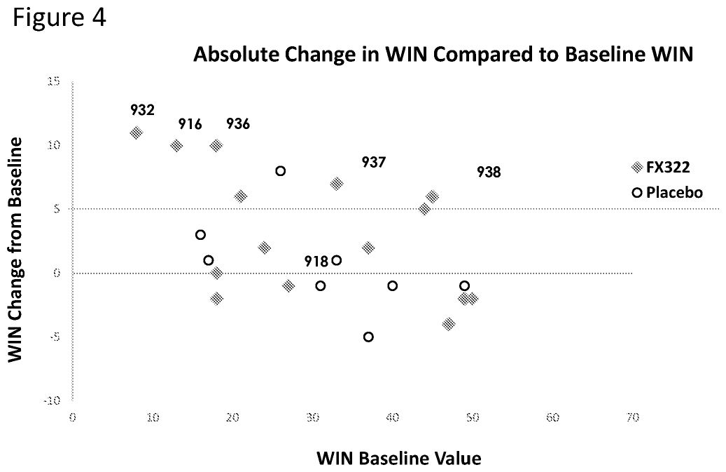

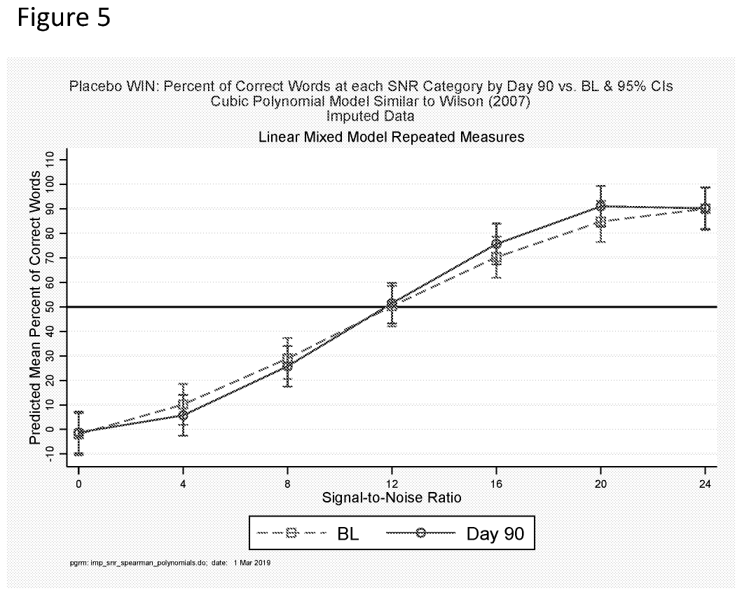

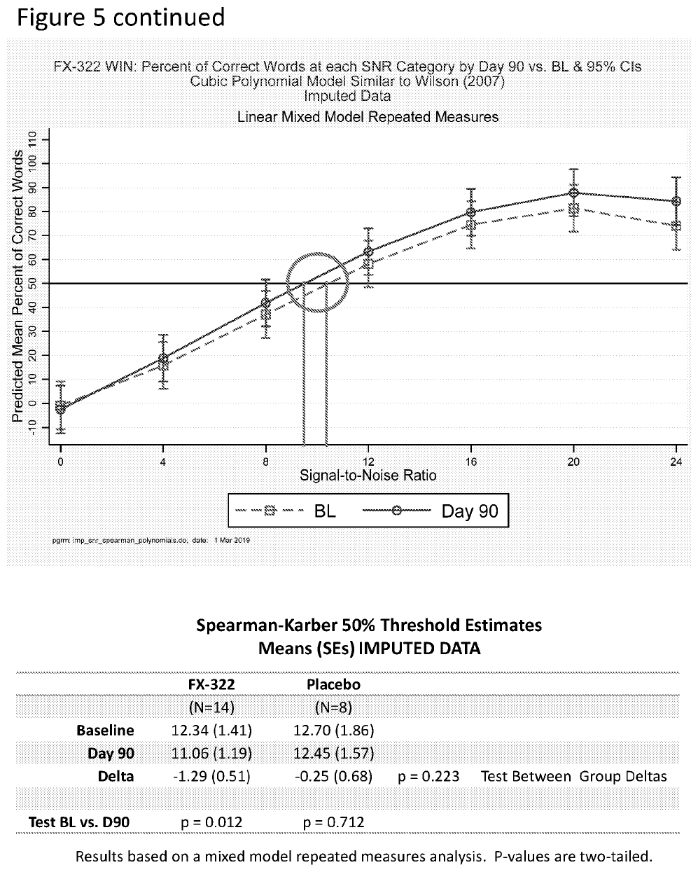

FX-322 patients showed a clear trend in favor of improvement in WIN testing compared to the placebo group (FIG. 3). Four subjects were tested at four visits with only 35 words. After confirming that correlation between first and second 35 word list scores, similar values were imputed for the first and second 35 word lists scores for these subjects. FX-322 and placebo groups showed no statistical difference in improvement above baseline scores in the WIN test (FIG. 3B). However, FX-322-treated patients had a greater absolute change in WIN compared to placebo-treated patients (FIG. 4). FX-322-treated patients also showed significant improvements in signal-to-noise ratio (SNR) from baseline to day 90 on the WIN test (p=0.012) whereas the placebo group did not (FIG. 5). Therefore FX-322 treatment resulted in an increase in intelligibility and speech perception.

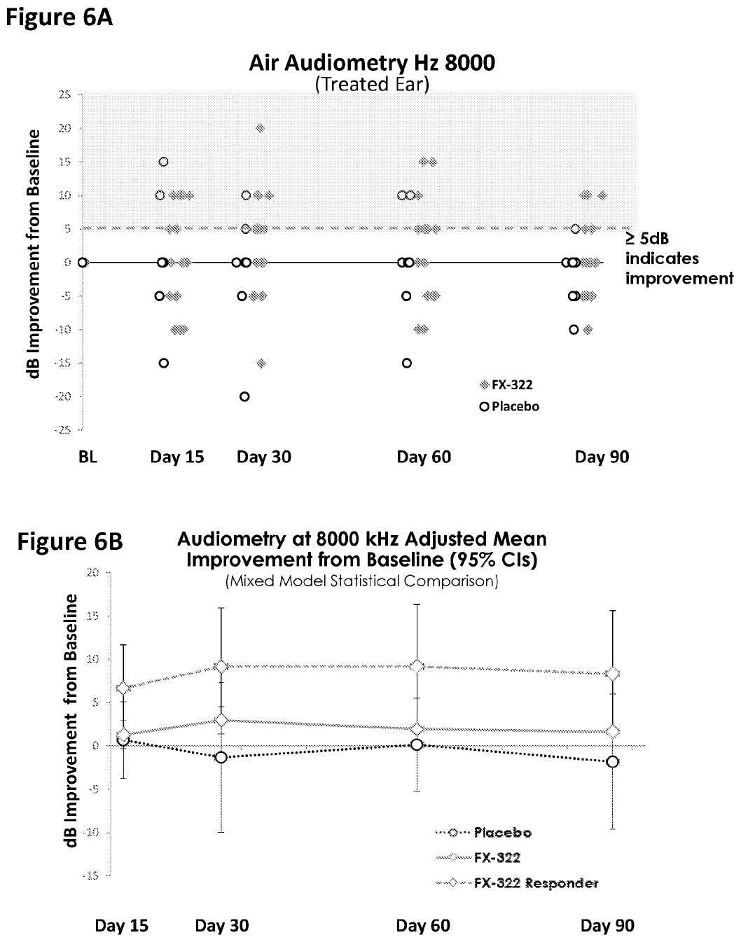

A total of 6 FX-322-treated patients and 1 placebo-treated patient demonstrated an improvement in air audiometry of ≥5 dB at 8 kHz on day 90 after injection (FIG. 6). The FX-322-treated patients showed a trend of improvement across the study compared to the placebo group (FIG. 6B). Although no group differences were seen in pure-tone audiometry, further analyses showed that a subset of FX-322 patients exhibited threshold improvements at 8 kHz. More specifically, four FX-322 patients had 10 dB recoveries and two FX-322 patients had 5 dB recoveries at 8 kHz. One placebo patient showed a 5 dB improvement at 8 kHz. When we analyzed 8 kHz responses for the five FX-322 patients that had clinically meaningful responses in WR, we found that there was a trend for threshold improvement over placebo (FIG. 6B). FX-322 treatment therefore resulted in a trend of increased audibility.

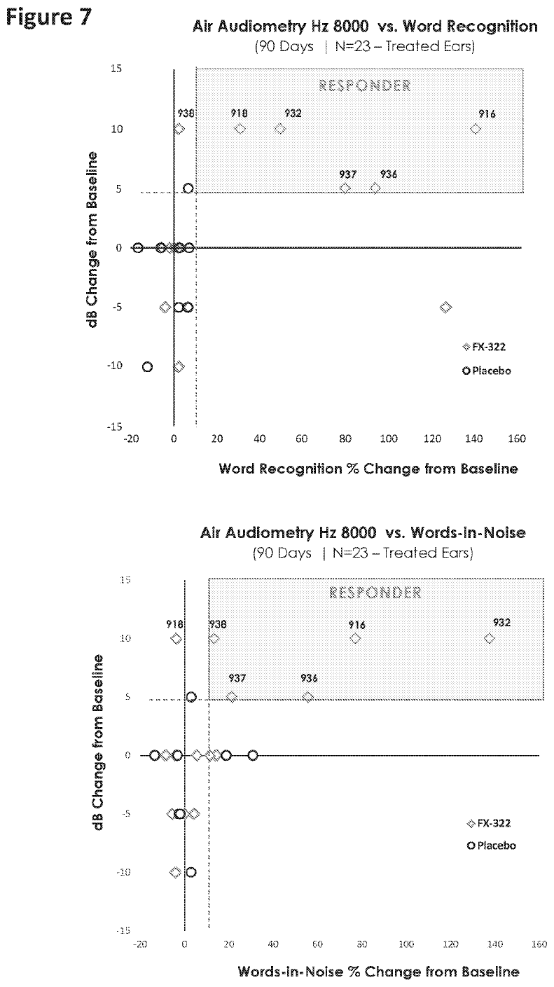

Responders were defined as patients who demonstrate a ≥5 dB improvement in hearing at 8 kHz in pure tone audiometry and ≥10% improvement in WR or WIN. These improvements must then have been observed on all subsequent test days. Overall, 6 of 15 (40%) FX-322-treated patients and 0 of 8 placebo-treated patients met the composite endpoint for response of ≥5 dB improvement in hearing at 8 kHz and ≥10% improvement in WR/WIN on day 90 post injection (Fisher's Exact Test, one-tailed, p=0.05) (FIG. 7). Therefore, only FX-322-treat patients met the requirements for the composite endpoint demonstrating improved audibility and intelligibility.

Age may be considered an acceptable proxy of hearing loss. There was no difference in age between the responder and non-responder groups (Table 16), i.e., response is not influenced by age.

TABLE 16
Analysis of the age of responders and non-responders

| Age Influence | Responders (N = 6) | Non-responders (N = 17) |
|---|---|---|
| Mean | 54.6 | 57.5 |
| Median | 57 | 57.5 |
| Standard deviation | 8.6 | 4.8 |
| Minimum, Maximum | 33, 64 | 50, 63 |

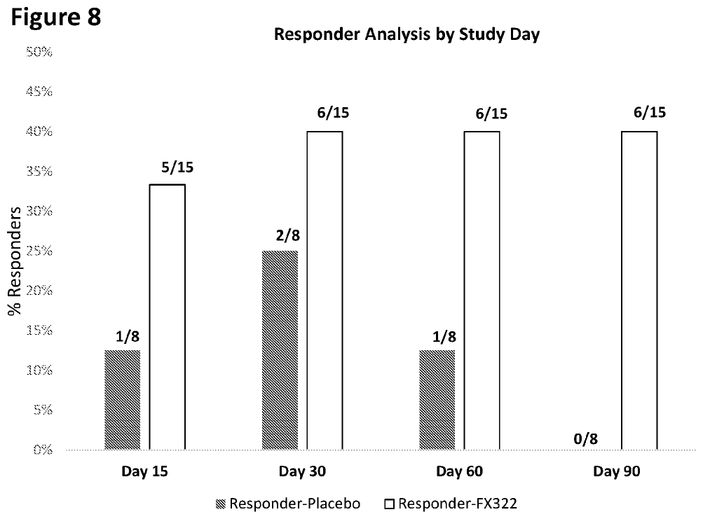

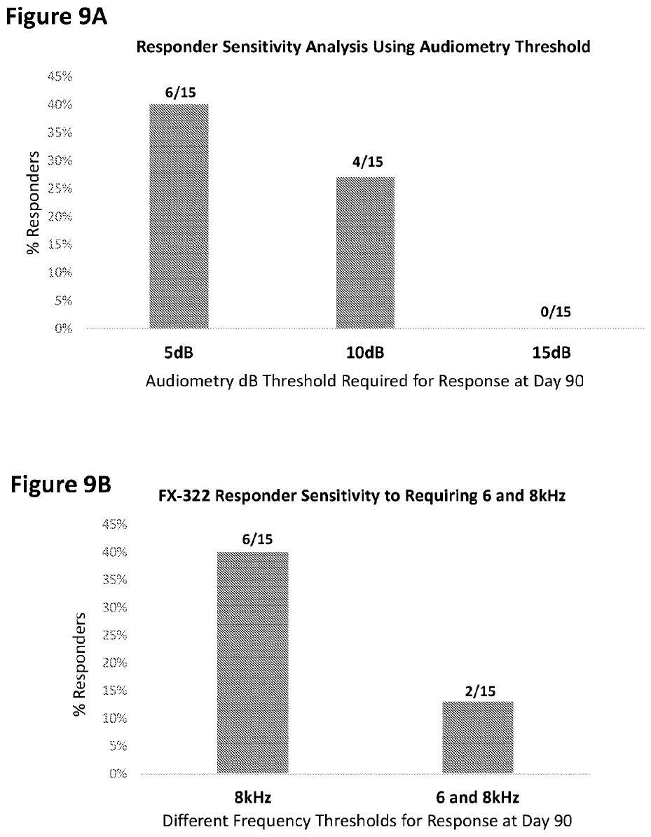

Responders were identified in both the FX-322-treated and placebo-treated groups at days 15, 30 and 60 after the single injection (FIG. 8). On each of these days there were more responders in the FX-322-treated group than the placebo-treated group. However, the only responders on day 90 post injection were FX-322-treated patients (6/15; 40%). The 40% response rate for the FX-322-treated group was sustained from day 30 to day 90 post injection with a high response rate (33%) observed as early as 15 days post injection (FIG. 8). Taking this analysis further to look at sensitivity, 4 of the 6 FX-322-treated responders demonstrated an improvement of ≥10 dB at 8 kHz on day 90 post injection (FIG. 9A). These four responders demonstrate an even more rigorous and clinically meaningful improvement. Furthermore, 2 of the 6 FX-322-treated responders demonstrated a ≥5 dB at both 6 and 8 kHz on day 90 post injection (FIG. 9B). FX-322 treatment provided a rigorous and clinically meaningful improvement in hearing.

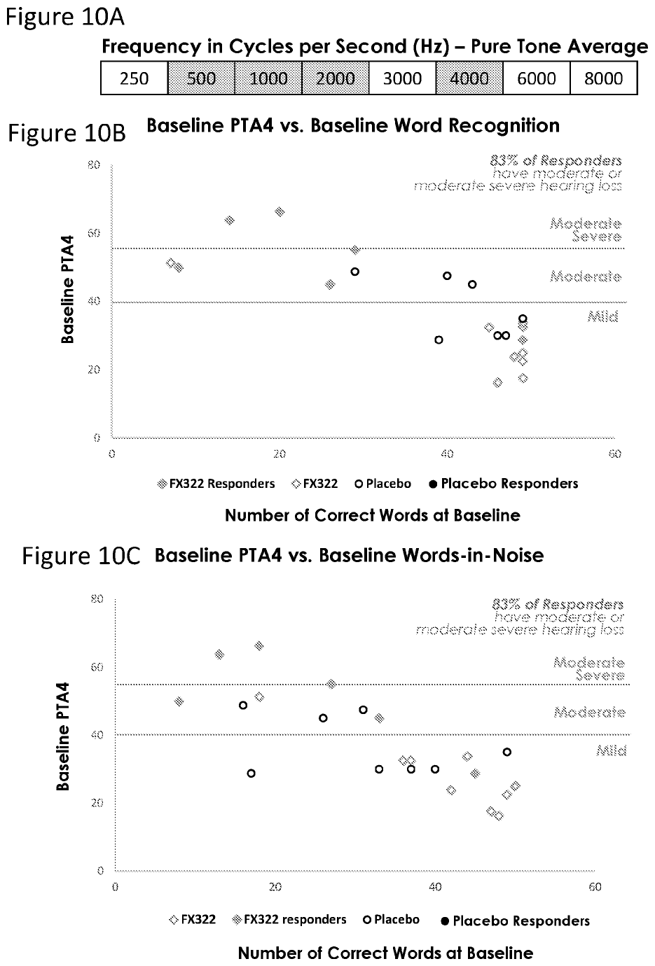

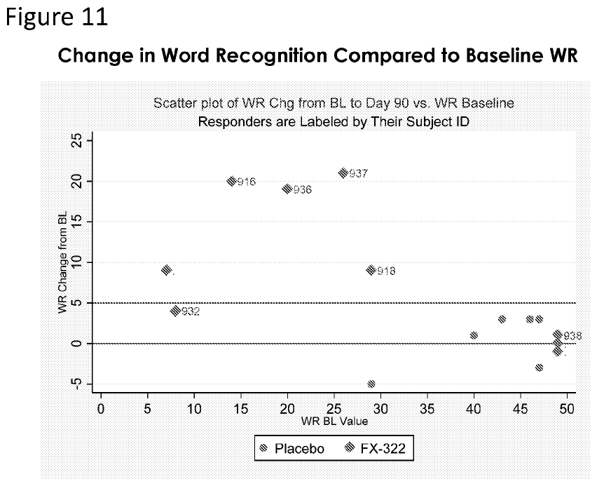

The pure tone average (PTA) was determined from thresholds measured at 500, 1000, 2000 and 4000 Hz to provide the PTA4 for each patient (FIG. 10A). Moderately severe hearing loss was defined as a PTA4 above 55 dB, moderate hearing loss was defined as a PTA4 above 40 dB and mild hearing loss was defines as a PTA4 below 40 dB. Predictably, patients with mild hearing loss had more correct words at baseline than patients with moderate or moderately severe hearing loss in both WR and WIN tests (FIGS. 10B and 10C). On day 90 post infection, 83% of responders, all of which were FX-322-treated patients, had moderate or moderately severe hearing loss, i.e., a PTA4 above 40 dB. It was also found that patients with a lower baseline for either the WR or WIN tests showed greater improvements with FX-322 treatment (FIG. 11). FX-322 treatment is therefore particularly effectively for patients with moderate hearing loss.

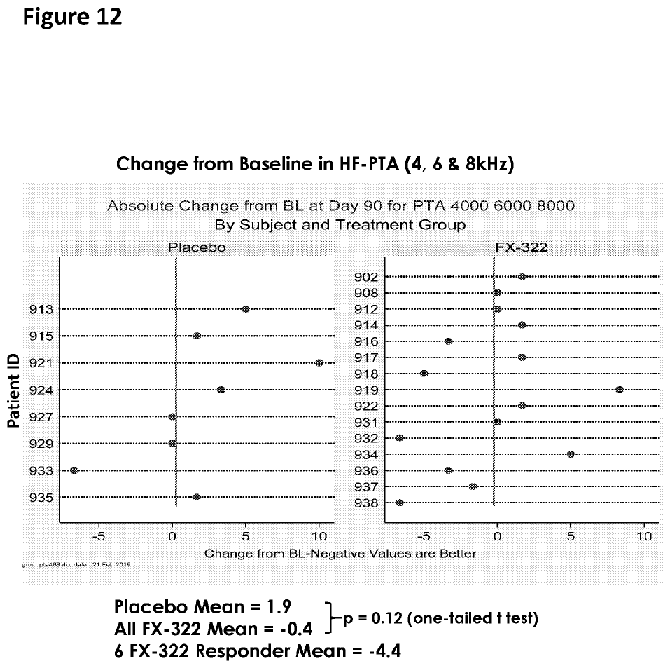

The high frequency pure tone average (HF-PTA) was determined for thresholds measured at 4, 6 and 8 kHz at baseline and day 90 post injection. The change in HF-PTA was calculated for each patient and revealed an enhanced improvement in audibility in the FX-322-treated patients compared to the placebo-treated patients (FIG. 12). Each FX-322-treated responder displayed a decreased HF-PTA further demonstrating improved hearing. FX-322 treatment resulted in increased audibility at high frequencies.

In addition, three subjects reported an improvement in tinnitus. All three of these subjects were in the cohort that had improved word recognition scores.

### Safety and Tolerability

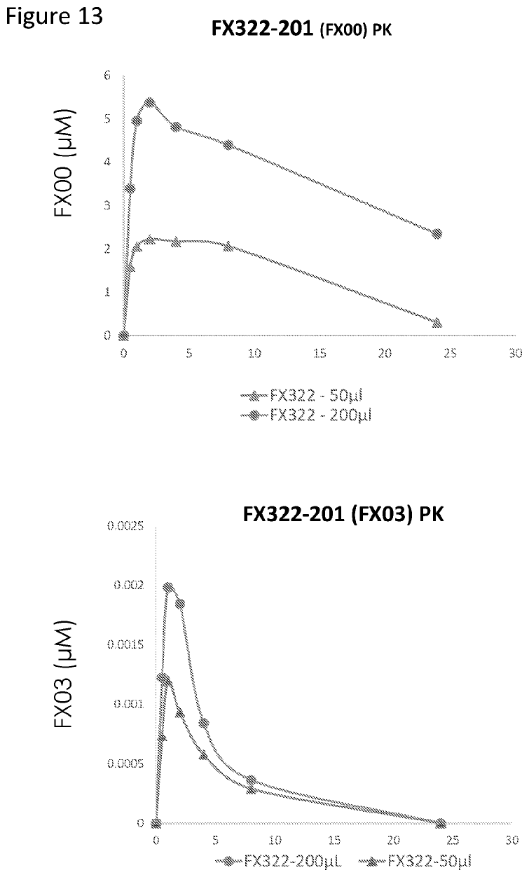

Mean plasma concentrations of CHIR99021 and VPA were approximately dose proportional over 24 hours for the two doses of FX-322 with peak plasma concentrations achieved within 2 hours and cleared from the systemic circulation within 24 hours (FIG. 13).

The majority of treatment-related adverse effects (AEs) were associated with intratympanic injection, including pain, discomfort, and itching of the treated ear (Table 17). Most AEs were mild, transient, and resolved within minutes of dosing. One patient experienced tympanic membrane perforation that resolved within 30 days. No systemic AE occurred in more than one patient. No serious AEs occurred, and no clinically relevant changes were observed for clinical laboratory values, vital signs, ECG, otoscopy or tympanometry.

TABLE 17
Incidence of treatment-related adverse events.

| | Number (%) of Events | | | |
|---|---|---|---|---|
| | FX-322 | FX-322 | Pooled | |
| | Low | High | Placebo | Total |
| | (n = 7) | (n = 8) | (n = 8) | (n = 23) |
| Ear discomfort | 5 (71.4) | 4 (50.0) | 2 (25.0) | 11 (47.8) |
| Ear pain | 1 (14.3) | 3 (37.5) | 3 (37.5) | 7 (30.4) |
| Ear pruritis | 1 (14.3) | 0 | 1 (12.5) | 2 (8.7) |
| Paraesthesia ear | 0 | 1 (12.5) | 0 | 1 (4.3) |
| Tinnitus | 0 | 1 (12.5) | 0 | 1 (4.3) |
| Tympanic membrane perforation | 0 | 1 (12.5) | 0 | 1 (4.3) |
| Dizziness | 1 (14.3) | 0 | 0 | 1 (4.3) |
| Headache | 0 | 1 (12.5) | 1 (12.5) | 2 (8.7) |
| Parosmia | 0 | 1 (12.5) | 0 | 1 (4.3) |
| Oropharyngeal pain | 1 (14.3) | 0 | 0 | 1 (4.3) |
| Throat irritation | 0 | 1 (12.5) | 0 | 1 (4.3) |

### Discussion

FX-322 has demonstrated the ability to induce regeneration of hair cells in cochlear tissue across a number of preclinical studies (McLean et. al., 2017). The Phase 1/2 clinical study extended these findings and showed statistically significant and clinically meaningful evidence of restoration of hearing in patients with stable SNHL. In a clinically meaningful composite endpoint of improvement in both audiometry and word recognition, 40% of patients with FX-322 responded vs. 0% with placebo. A greater proportion of FX-322-treated patients experienced hearing improvement at 90 days at 8 kHz. Patients were required to be stable across endpoints when measured for the 6 months before the study, and notably in placebo and non-injected ears, these endpoints were unchanged with no placebo-treated patient experiencing a response. Without committing to any specific theory, improved hearing in the ultra-high frequencies could be responsible for improvements in words in quiet and words in noise, a notion corroborated by Monson (2017) who found that listeners perform significantly better in noise when given increased access to frequencies above 8 kHz.

No difference in treatment effect was observed between the 2 dose volumes of FX-322. This is likely because as long as the round windows are covered diffusion into tissue sites should be concentration dependent as described by Fick's Law of Diffusion. Improvements in hearing with FX-322 occurred without any systemic effects and with only mild, transient, and short-term local effects at the time of injection.

In summary, this is the first evidence in human patients with hearing loss that drugs can induce a regenerative response in cochlear tissue and restore hearing function. In particular and surprisingly, the treatment improved word recognition in quiet and in noise. Since a major complaint of assistive-device users is the inability to hear in noisy environments (Kochkin 2000 and Lesica 2018), the improvements in word recognition were of notable interest.

### Example 3

### Efficacy and Duration of Hearing Loss Prior to Treatment

The performance of six individual responders in the Phase 1/2, double-blind, randomized, placebo-controlled study of FX-322 described in Example 2 is provided in Table 18, as an example of results. The results are provided in the context of the duration of the patient's hearing loss prior to the treatment, with patients having sudden or noise induced hearing loss showing improvement after treatment

An improvement in intelligibility (as assessed by WR score) was observed in patients who had experienced sensorineural hearing loss for several years. Surprisingly, an improvement in intelligibility was observed in a patient who had hearing loss for over 20 years. These results suggest that an improvement in hearing may provide a beneficial effect in patients with long-term sensorineural hearing loss.

TABLE 18
Examples of individual treatment effect in the context of disease duration

| | | | | Disease duration | | | |
|---|---|---|---|---|---|---|---|
| | Word recognition | | | | | | Duration |
| | | | Word | | | | of disease |
| | Baseline | Day 90 | count | % CFB | | Disease | in years |
| Patient | # (%) | # (%) | CFB | [1] | Consent date | onset [2] | [3] |
| 918 | 29 (58%) | 38 (76%) | 9 | 31.03 | 20 Jul. 2018 | 2015 | 3.06 |
| 932 | 8 (16%) | 12 (24%) | 4 | 50.00 | 29 Aug. 2018 | February 2017 | 1.54 |
| 919 | 7 (14%) | 16 (32%) | 9 | 128.57 | 24 Jul. 2018 | 2014 | 4.07 |
| 916 | 14 (28%) | 34 (68%) | 20 | 142.86 | 18 Jul. 2018 | 1997 | 21.05 |
| 936 | 20 (40%) | 39 (78%) | 19 | 95.00 | 24 Sep. 2018 | 2013 | 5.23 |
| 937 | 26 (52%) | 47 (94%) | 21 | 80.77 | 26 Sep. 2018 | January 2017 | 1.70 |

[1] Percent Change from baseline (CFB) is relative change and is calculated as 100 * [(Result − Baseline)/Baseline].
[2] Onset dates are imputed for missing values. If the month and day are missing, the middle of the year (July 1st) is imputed. If the day is missing, the middle of the month (15) is imputed.
[3] Years are calculated as (Onset − Consent + 1)/365.25.

### Example 4

A further responder definition was developed using the data in Example 2 which is based on improvements in speech perception (i.e., intelligibility) or improvements in audiometry (i.e., audibility).

### Improvement in Speech Perception

A responder was defined when showing either an improvement in Word Recognition in quiet or WIN from baseline to post-treatment.

For Word Recognition, a patient is considered a responder when the patient's post-treatment Word Recognition score falls outside the 95% confidence interval as defined by Thornton and Raffin (1978).

For WIN, a patient is considered a responder when the patient's WIN from baseline to post-treatment 50% SNR score improves by at least 3 dB (Wilson & McArdle, 2007).

### Improvement in Audiometry

A responder may also be defined according to improvements in Pure Tone Audiometry from baseline to post-treatment. A patient is considered a responder when pure tone thresholds improve (i.e., decrease) by 10 dB or more at 2 consecutive frequencies (e.g., 6 kHz and 8 kHz) or 15 dB or more at a single frequency.

### Example 5

### Study Design and Oversight

A Phase 2a, double-blind, randomized, placebo-controlled study is being carried out according to International Conference on Harmonisation guidelines, Good Clinical Practices, and the Declaration of Helsinki. The protocol and amendments are approved by the Institutional Review Board for participating investigators.

### Patient Population and Treatments

Adults aged 18-65 years are eligible for participation in the clinical trial if they have:
an established diagnosis of stable sensorineural hearing loss by standard audiometric measures for ≥6 months prior to the Screening visit (i.e., no changes in air conduction greater than 10 dB at a single frequency or greater than 5 dB at two contiguous frequencies from the prior audiogram to the Screening audiogram in the study ear,
a documented medical history consistent with hearing loss being caused by noise exposure or sudden sensorineural hearing loss
Pure Tone Audiometry (PTA) within 26-70 dB in the ear to be injected.

Further conditions for inclusion and exclusion are available at ClinicalTrials.gov, Identifier NCT04120116. Patients are randomized to one of four arms, as follows, each of which involves four intratympanic injections of a hydrogel formulation. The formulation of FX-322 in this study, and placebo are as follows:

### FX-322:

| Component | % w/w |
|---|---|
| CHIR99021 | 0.29% |
| Valproate Na | 8.03% |
| Poloxamer 407 | 14.97% |
| DMSO | 4.92% |
| Water | 71.78% |

### Placebo:

| Component | % w/w |
|---|---|
| Poloxamer 407 | 14.29% |
| Sodium Phosphate, Monobasic | 0.86% |
| Tromethamine | 1.30% |
| NaCl | 0.86% |
| DMSO | 5.30% |
| Water | 77.38% |

| Arm | Treatment |
|---|---|
| Experimetal arm 1: | FX-322-Dosing occurs once weekly for four |
| FX-322 Single Dose; | consecutive weeks. Patients receive one, two |
| Placebo Three Doses | or four doses of FX-322, followed by three, |
| | two or zero Placebo doses, respectively. |
| | Placebo-Dosing occurs once weekly for |
| | four consecutive weeks. Patients receive |
| | two, three or four doses of Placebo, preceded |
| | by two, one or zero Placebo doses, |
| | respectively. |
| Experimental arm 2: | FX-322-Dosing occurs once weekly for four |
| FX-322 Two Doses; | consecutive weeks. Patients receive one, two |
| Placebo Two Doses | or four doses of FX-322, followed by three, |
| | two or zero Placebo doses, respectively. |
| | Placebo-Dosing occurs once weekly for |
| | four consecutive weeks. Patients receive |
| | two, three or four doses of Placebo, preceded |
| | by two, one or zero Placebo doses, |
| | respectively. |
| Experimental arm 3: | FX-322-Dosing occurs once weekly for four |
| FX-322 Four Doses | consecutive weeks. Patients receive one, two |
| | or four doses of FX-322, followed by three, |
| | two or zero Placebo doses, respectively. |
| Placebo comparator: | Placebo-Dosing occurs once weekly for |
| Placebo Four Doses | four consecutive weeks, Patients receive |
| | two, three or four doses of Placebo, preceded |
| | by two, one or zero Placebo doses, |
| | respectively. |

### Study Assessments and Endpoints

### Primary Outcome Measures

Speech Intelligibility using the Word Recognition in Quiet test measured with Consonant-Nucleus-Consonant (CNC) word lists. Follow-up testing is performed up until 210 days post injection to quantify the ability of listeners to understand monosyllabic English words at a suprathreshold level. The WR test involves the administration of 50 monosyllabic words in a quiet background at a loud, but comfortable level. The words are played from a CD player at 30 dB above the pure tone average of 500, 1000, and 2000 Hz. The Maryland consonant-vowel nucleus-consonant (CNC) lists are used. One list is administered in each ear. Randomized lists are used across the study. The total number of words repeated correctly is recorded. An increase in words recognized following treatment compared to words recognized prior to treatment indicates an improvement when treated with the composition.
Speech intelligibility using the Words-in-Noise test measured with Consonant-Nucleus-Consonant (CNC) word lists. Follow-up testing is performed up until 210 days post injection to quantify the ability of listeners to understand monosyllabic words in background noise (Wilson, 2003). The WIN test involves the administration of 70 monosyllabic words in a multi-talker background. The level of the multi-talker babble is fixed at 80 dB SPL, with 3 females and 3 males talking about various topics, and the level of the CNC words is varied from 104 dB SPL (24 dB SNR) to 80 dB SPL (0 dB SNR) in a descending manner. Words are played from 24 dB signal-to-noise ratio (SNR) to 0 dB SNR in a descending manner. The results are analyzed for the SNR (dB) at which 50% correct word performance was achieved. A decrease in the SNR (dB) at which 50% correct word performance was achieved following treatment compared to the SNR (dB) at which 50% correct word performance was achieved prior to treatment indicates an improvement when treated with the composition.
Standard Pure Tone Audiometry is measured to determine a subject's threshold for hearing at standard frequencies (Hz). A decrease in a subject's thresholds following treatment compared to the threshold prior to treatment indicates an improvement.
Systemic and local safety are also assessed. For systemic safety, the number of patients with treatment-related adverse events is screened up until 210 days post injection and assessed by CTCAE v5.0. For local safety, the number of patients with abnormal changes from baseline in otoscopic examinations up until 210 days post injection is assessed using microscopic otoscopy to specifically record any abnormalities of the external ear canal, tympanic membrane and middle ear. Tympanometry is also used to assess local safety. Tympanometry tests the integrity of the tympanic membrane by varying air pressure in the ear canal. Middle ear compliance (mL), peak pressure (daPa), and ear canal volume (mL) are recorded.

### Secondary Outcome Measures

Extended high frequency pure-tone audiometry is performed to determine a subject's threshold for hearing at frequencies beyond those in standard pure tone audiometry (250-8000 Hz). Behavioral audiometric thresholds are determined at 9000, 10,000, 11,000, 12,000, 14,000, and 16,000 Hz and testing is performed using circumaural earphones at the screening visit. Day 15, Day 30, Day 60, Day 90, Day 120, Day 150, Day 180, and Day 210. A decrease in a subject's thresholds following treatment compared to the threshold prior to treatment indicates an improvement.
Tinnitus is measured using the Tinnitus Functional Index (TFI), with a scale ranging from 0 to 100 that defines severity categories based on 25 self-reported answers. The TFI assesses tinnitus experiences over the past week on 8 functional domains (intrusiveness, sense of control, cognitive interference, sleep disturbance, auditory difficulties, interference with relaxation, quality of life, and emotional distress). The TFI is administered at Baseline, Day 60, Day 120, and Day 210. A decrease in a subject's TFI score following treatment compared to the threshold prior to treatment indicates an improvement.
Hearing Handicap Inventory for Adults (HHIA) is assessed. The Hearing Handicap Inventory for Adults (HHIA) is a 25 item self-assessment scale composed of two subscales (emotional and social/situational). Pure-tone and speech audiometry measures in quiet are generally poor predictors of the impact that hearing loss has on communication abilities (Weinstein, 1984). Thus, the HHIA was developed to assess handicap experienced by adults with hearing loss (Newman et al., 1990). The HHIA shows responsiveness to rehabilitation efforts and serves as a guide in designing a patient-centered treatment program. The HHIA is administered at Baseline, Day 60, Day 120, and Day 210. The subject is asked to consider their hearing at present (i.e., over the last week) and identify problems that the hearing loss may be causing. The subject is also asked to not skip questions due to situation avoidance. The HHIA is completed by the participant with study staff present. The HHIA is immediately reviewed for clarity and completeness and any ambiguities resolved. The HHIA renders a score from 0 to 100. Scores from 0-16 represent no hearing handicap; scores from 18-42 represent mild-moderate handicap; and scores 44 and greater represent significant handicap. The standard error of measurement for the HHIA is 6 points. A decrease in a subject's HHIA score following treatment compared to the threshold prior to treatment indicates an improvement.
Hearing Screening Inventory (HSI). The Hearing Screening Inventory (HSI) is a 12-item self-report inventory to assess hearing impairment on a 5-point scale (Coren & Hakstian, 1992). The HIS is designed to correlate with audiometric measures such as pure tone audiometry and suprathreshold speech recognition rather than hearing handicap. The HSI is administered at Baseline and Day 210. The subject is asked to consider his/her haring experiences in the present (i.e., over the last week) and complete the 12 questions. The HSI has been designed for self-administration, but clarification about the meaning of the questions can be given if required. The HSI is immediately reviewed for completeness and clarity and any ambiguities resolved. Responses are coded from 1 to 5 with “never” or “good” as 1 and “always” or “very poor” as 5. The HSI renders a score from 12 to 60. Scores from 12-27 are classified as normal, and scores from 28-60 are classified as hearing impaired. A decrease in a subject's HSI score following treatment compared to the threshold prior to treatment indicates an improvement.

### REFERENCES

Ahmadzai N, Kilty S, Wolfe D. Bonaparte J. Schramm D, Fitzpatrick E, Lin V, Cheng W, Skidmore B, Moher D, Hutton B. A protocol for a network meta-analysis of interventions to treat patients with sudden sensorineural hearing loss. Syst Rev. 2018; 7(1):74.
Alexander T H, Harris J P. Incidence of sudden sensorineural hearing loss. Otol Neurotol Off Publ Am Otol Soc Am Neurotol Soc Eur Acad Otol Neurotol. 2013; 34:1586-9.
Badri R. Siegel J H, Wright B A. Auditory filter shapes and high-frequency hearing in adults who have impaired speech in noise performance despite clinically normal audiograms. J Acoust Soc Am. 129, 852-863 (2011).
Basner M, Brink M, Bristow A. de Kluizenaar Y, Finegold L, Hong J. Janssen S A, Klaeboe R, Leroux T, Liebl A, Matsui T, Schwela D, Sliwinska-Kowalska M, Sörqvist P. ICBEN review of research on the biological effects of noise 2011-2014. Noise Health. 2015; 17(75):57-82.
Bramhall N F, Shi F. Arnold K, Hochedlinger K, Edge A S. Lgr5-positive supporting cells generate new hair cells in the postnatal cochlea. Stem Cell Reports. 2014; 2(3):311-22.
Carney and Schlauch: Critical Difference Table for Word Recognition Testing Derived Using Computer Simulation; Journal of Speech, Language and Hearing Research; 2007; 50:1203-1209.
Carlsson P-I, Hjaldahl J, Magnuson A, et al. Severe to profound hearing impairment: quality of life, psychosocial consequences and audiological rehabilitation. Disabil Rehabil. 2015; 37(20):1849-56.
Cox B C, Chai R, Lenoir A, Liu Z, Zhang L, Nguyen D H, Chalasani K, Steigelman K A, Fang J. Rubel E W, Cheng A G, Zuo J. Spontaneous hair cell regeneration in the neonatal mouse cochlea in vivo. Development. 2014; 141(4):816-29.
Curhan S G, Willett W C, Grodstein F, Curhan G C. Longitudinal study of hearing loss and subjective cognitive function decline in men. Alzheimers Dement. 2019 Jan. 21. pii: S1552-5260(18)33606-9. doi: 10.1016/j.jalz.2018.11.004.
Edwards B. The distortion of auditory perception by sensorineural hearing impairment. (2003)
Fackrell K. Hall D A, Barry J G, Hoare D J, Performance of the Tinnitus Functional Index as a diagnostic instrument in a UK clinical population Hear Res. 2018; 358: 74-85;
Gygi B and Hall D A Background sounds and hearing-aid users: a scoping review. International Journal of Audiology, 2016; 55:1, 1-10
Health Quality Ontario. Bilateral cochlear implantation: a health technology assessment. Ont Health Technol Assess Ser. 2018; 18(6):1-139. Available from: http://www.hqontario.ca/evidenceto-improve-care/journal-ontario-health-technology-assessment-series
Henry J A, Stewart B J, Abrams H B, Newman C W, Griest S, Martin W H, Myers P J, Searchfield G. Tinnitus Functional Index—Development and Clinical Application. Audiology Today 2014:26(6):40-48.
Henry J A, Griest S, Thielman E, McMillan G, Kaelin C, Carlson K F. Tinnitus Functional Index—Development, validation, outcomes research, and clinical application. Hearing Research 2015; 334:58-64
Hirose K, Hartsock J J, Johnson S, Santi P, Salt A N. Systemic lipopolysaccharide compromises the blood-labyrinth barrier and increases entry of serum fluorescein into the perilymph. J Assoc Res Otolaryngol. 2014; 15:707-19.
Hoben R, Easow G, Pevzner S, Parker M A. Outer hair cell and auditory nerve function in speech recognition in quiet and in background noise. Front Neurosci. 11, 157 (2017). Jacquemin L, Mertens G, Van de Heyning P, Vanderveken O M, Topsakal V, De Hertogh, W, Michiels S, Van Rompaey V, Gilles A, Sensitivity to change and convergent validity of the Tinnitus Functional Index (TFI) and the Tinnitus Questionnaire (TQ): Clinical and research perspectives Hear Res. 2019; 382:107796
Kochkin S. MarkeTrak V: “Why my hearing aids are in the drawer”: The consumers' perspective. The Hearing Journal 53, 34-41 (2000).
Kujawa S G, Liberman M C. Adding insult to injury: cochlear nerve degeneration after “temporary” noise-induced hearing loss. J Neurosci. 29, 14077-14085 (2009).
Le T N, Straatman L V, Lea J, Westerberg B. Current insights in noise-induced hearing loss: a literature review of the underlying mechanism, pathophysiology, asymmetry, and management options. J Otolaryngol Head Neck Surg. 2017; 46(1):41.
Lesica N A Why do hearing aids fail to restore normal auditory perception? Trends Neurosci. 41, 174-185 (2018).
Lerner S. Limitations of conventional hearing aids: Examining common complaints and issues that can and cannot be remedied. Otolaryngol Clin North Am. 2019 Jan. 3. pii: S0030-6665(18)30242-1. doi: 10.1016/j.otc.2018.11.002.
Liberman M C, Dodds L W. Single-neuron labeling and chronic cochlear pathology. Hear Res. 16, 43-53 (1984).
Liljas A E M, Wannamethee S G, Papacosta O, et al. Social and lifestyle characteristics and burden of ill-health associated with self-reported hearing and vision impairments in older men in the British community: a cross-sectional study. Lancet. 2014; 384(2):S45.
McLean W J, Yin X, Lu L. Lenz D R, McLean D, Langer R, Karp J M, Edge A S B. Clonal expansion of Lgr5-positive cells from mammalian cochlea and high-purity generation of sensory hair cells. Cell Rep. 2017; 18(8):1917-29.
Meikle M B, Henry J A, Griest S E, Stewart B J, Abrams H B, McArdle R, Myers P J, Newman C W, Sandridge S, Turk D C, Folmer, R L, Frederick E J, House J W, Jacobson G P, Kinney S E, Martin W H, Nagler S M, Reich G E, Searchfield F, Sweetow R, Vernon J A. The Tinnitus Functional Index: Development of a New Clinical Measure for Chronic, Intrusive Tinnitus Ear & Hearing 2012 33(2): 153-176.
Monson B B, Hunter E J, Story B H. Horizontal directivity of low- and high-frequency energy in speech and singing. J Acoust Soc Am. 2012; 132(1):433-41.
Noble W. Self-assessment of hearing and related functions. Whurr. 1998
Plontke S K, Biegner T, Kammerer B, Delabar U, Salt A N. Dexamethasone concentration gradients along scala tympani after application to the round window membrane. Otol Neurotol. 2008; 29:401-6.
Pratt S R. Profound hearing loss: Addressing barriers to hearing healthcare. Semin Hear. 2018; 39(4):428-36.
Ryan A, Dallos P. Effect of absence of cochlear outer hair cells on behavioral auditory threshold. Nature 253, 44-46 (1975).
Salt A N, Hartsock J. Plontke S, LeBel C, Piu F. Distribution of dexamethasone and preservation of inner ear function following intratympanic delivery of a gel-based formulation. Audiol Neurootol. 2001; 16:323-35.
Salt A N, Gill R M, Hartsock J J. Perilymph Kinetics of FITC-Dextran Reveals Homeostasis Dominated by the Cochlear Aqueduct and Cerebrospinal Fluid. J Assoc Res Otolaryngol. 2015; 16:357-371.
Sawyer C S, Armitage C J, Munro K J, Singh G, Dawes P D. Correlates of hearing aid use in UK adults: self-reported hearing difficulties, social participation, living situation, health, and demographics. Ear Hear. 2019 Jan. 17. doi: 10.1097/AUD.0000000000000695.
Sha S H, Schacht J. Emerging therapeutic interventions against noise-induced hearing loss. Expert Opin Investig Drugs. 2017; 26(1):85-96.
Studebaker G A. A ‘rationalized arcsine transform. J Speech Lang Hear Res. 28, 455-462 (1985).
Thornton and Raffin. Speech-Discrimination scores modeled as a binomial variable. J. Speech Hear. Res. 1978 21:507-518
Wang Y, Hirose K, Liberman M C. Dynamics of noise-induced cellular injury and repair in the mouse cochlea. J Assoc Res Otolaryngol. 2002; 3(3):248-68.
Wang X, Dellamary L, Fernandez R, Harrop A, Keithley E M, Harris J P, Ye Q, Lichter J. LeBel C, Piu F. Dose-dependent sustained release of dexamethasone in inner ear cochlear fluids using a novel local delivery approach. Audiol Neurootol. 2009; 14; 393-401.
Willink A, Reed N S, Lin F R. Cost-benefit analysis of hearing care services: What is it worth to Medicare? J Am Geriatr Soc. 2019 Jan. 14. doi: 10.1111/jgs.15757.
Wilson B S, Tucci D L, Merson M H, O'Donoghue G M. Global hearing health care: New findings and perspectives. Lancet 2017; 390:2503-15.
Wilson and McArdle. Intra- and Inter-session Test, Retest Reliability of the Words-in-Noise (WIN) Test. J. Am. Acad. Audiol. 18:813-825 (2007)
Wu P Z, Liberman L D, Bennett K, de Gruttola V, O'Malley J T, Liberman M C. Primary neural degeneration in the human cochlea: evidence for hidden hearing loss in the aging ear. Neuroscience. 407, 8-20 (2019).

### NUMBERED EMBODIMENTS

1. A hair cell regeneration agent(s) for use in treating sensorineural hearing loss in a human patient, wherein optionally the sensorineural hearing loss is sudden sensorineural hearing loss.
2. A hair cell regeneration agent(s) for use in treating noise-induced sensorineural hearing loss in a human patient.
3. A hair cell regeneration agent(s) for use in treating sensorineural hearing loss in a human patient, wherein the average of the patient's hearing thresholds across 0.5 kHz, 1 kHz, 2 kHz and 4 kHz is at least 40 dB HL and no more than 70 dB HL when measured by pure tone audiometry prior to the treatment.
4. The hair cell regeneration agent(s) for use according to embodiment 3, wherein the average of the patient's hearing thresholds across 0.5 kHz, 1 kHz, 2 kHz and 4 kHz is at least 40 dB HL and no more than 55 dB HL when measured by pure tone audiometry prior to the treatment.
5. The hair cell regeneration agent(s) for use according to embodiment 3, wherein the average of the patient's hearing thresholds across 0.5 kHz, 1 kHz, 2 kHz and 4 kHz is at least 55 dB HL and no more than 70 dB HL when measured by pure tone audiometry prior to the treatment.
6. A hair cell regeneration agent(s) for use in treating sensorineural hearing loss in a human patient, wherein the patient has a hearing threshold of at least 40 dB HL at 4 kHz; and/or at least 40 dB HL at 6 kHz; and/or at least 40 dB HL at 8 kHz; when measured by pure tone audiometry prior to the treatment.
7. The hair cell regeneration agent(s) for use according to embodiment 6, wherein the patient has a hearing threshold of at least 40 dB HL at 4 kHz when measured by pure tone audiometry prior to the treatment.
8. The hair cell regeneration agent(s) for use according to embodiment 6 or embodiment 7, wherein the patient has a hearing threshold of at least 40 dB HL at 6 kHz, when measured by pure tone audiometry prior to the treatment.
9. The hair cell regeneration agent(s) for use according to any of embodiments 6-8, wherein the patient has a hearing threshold of at least 40 dB HL at 8 kHz, when measured by pure tone audiometry prior to the treatment.
10. The hair cell regeneration agent(s) for use according to any of embodiments 6-9, wherein the patient has an audiogram with hearing thresholds in the following ranges when measured by pure tone audiometry prior to the treatment:
   a. 8 kHz-40 dB HL to 95 dB HL; and/or
   b. 6 kHz-40 dB HL to 85 dB HL; and/or
   c. 4 kHz-40 dB HL to 80 dB HL; and/or
   d. 3 kHz-40 dB HL to 70 dB HL; and/or
   e. 2 kHz-40 dB HL to 70 dB HL; and/or
   f. 1 kHz-40 dB HL to 70 dB HL; and/or
   g. 0.5 kHz-40 dB HL to 70 dB HL; and/or
   h. 0.25 kHz-40 dB HL to 70 dB HL.
11. The hair cell regeneration agent(s) for use according to any of embodiments 6-10, wherein the patient has an audiogram with a hearing threshold in the range of 40 dB HL to 95 dB HL at 8 kHz when measured by pure tone audiometry prior to treatment.
12. The hair cell regeneration agent(s) for use according to any of embodiments 6-11, wherein the patient has an audiogram with a hearing threshold in the range of 40 dB HL to 85 dB HL at 6 kHz when measured by pure tone audiometry prior to treatment.
13. The hair cell regeneration agent(s) for use according to any of embodiments 6-12 wherein the patient has an audiogram with a hearing threshold in the range of 40 dB HL to 80 dB HL at 4 kHz when measured by pure tone audiometry prior to treatment.
14. The hair cell regeneration agent(s) for use according to any of embodiments 6-13, wherein the patient has an audiogram with a hearing threshold in the range of 40 dB HL to 70 dB HL at 3 kHz when measured by pure tone audiometry prior to treatment.
15. The hair cell regeneration agent(s) for use according to any of embodiments 6-14, wherein the patient has an audiogram with a hearing threshold in the range of 40 dB HL to 70 dB HL at 2 kHz when measured by pure tone audiometry prior to treatment.
16. The hair cell regeneration agent(s) for use according to any of embodiments 6-15, wherein the patient has an audiogram with a hearing threshold in the range of 40 dB HL to 70 dB HL at 1 kHz when measured by pure tone audiometry prior to treatment.
17. The hair cell regeneration agent(s) for use according to any of embodiments 6-16, wherein the patient has an audiogram with a hearing threshold in the range of 40 dB HL to 70 dB HL at 0.5 kHz when measured by pure tone audiometry prior to treatment.
18. The hair cell regeneration agent(s) for use according to any of embodiments 6-17, wherein the patient has an audiogram with a hearing threshold in the range of 40 dB HL to 70 dB HL at 0.25 kHz when measured by pure tone audiometry prior to treatment.
19. The hair cell regeneration agent(s) for use according to embodiment 10, wherein the patient has an audiogram with hearing thresholds in the following ranges when measured by pure tone audiometry prior to the treatment:
   a. 8 kHz-40 dB HL to 70 dB HL; and
   b. 6 kHz-40 dB HL to 70 dB HL; and
   c. 4 kHz-40 dB HL to 70 dB HL; and
   d. 3 kHz-40 dB HL to 70 dB HL; and
   e. 2 kHz-40 dB HL to 70 dB HL; and
   f. 1 kHz-40 dB HL to 70 dB HL; and
   g. 0.5 kHz-40 dB HL to 70 dB HL; and
   h. 0.25 kHz-40 dB HL to 70 dB HL.
20. A hair cell regeneration agent(s) for use in treating sensorineural hearing loss in a human patient, wherein the average of the patient's hearing thresholds across 0.5 kHz, 1 kHz, 2 kHz and 4 kHz is at least 25 dB HL and no more than 40 dB HL when measured by pure tone audiometry prior to the treatment.
21. A hair cell regeneration agent(s) for use in treating sensorineural hearing loss in a human patient, wherein the patient has a hearing threshold of at least 25 dB HL and no more than 40 dB HL at 4 kHz; and/or at least 25 dB HL and no more than 40 dB HL at 6 kHz; and/or at least 25 dB HL and no more than 40 dB HL at 8 kHz; when measured by pure tone audiometry prior to the treatment.
22. The hair cell regeneration agent(s) for use according to embodiment 21, wherein the patient has a hearing threshold of at least 25 dB HL and no more than 40 dB HL at 4 kHz when measured by pure tone audiometry prior to the treatment.
23. The hair cell regeneration agent(s) for use according to embodiment 21 or 22, wherein the patient has a hearing threshold of at least 25 dB HL and no more than 40 dB HL at 6 kHz when measured by pure tone audiometry prior to the treatment.
24. The hair cell regeneration agent(s) for use according to any of embodiments 21-23, wherein the patient has a hearing threshold of at least 25 dB HL and no more than 40 dB HL at 8 kHz when measured by pure tone audiometry prior to the treatment.
25. The hair cell regeneration agent(s) for use according to any of embodiments 21-24, wherein the patient has an audiogram with hearing thresholds in the following ranges when measured by pure tone audiometry prior to the treatment:
   a. 8 kHz-25 dB HL to 40 dB HL; and/or
   b. 6 kHz-25 dB HL to 40 dB HL; and/or
   c. 4 kHz-25 dB HL to 40 dB HL; and/or
   d. 3 kHz-25 dB HL to 40 dB HL; and/or
   e. 2 kHz-25 dB HL to 40 dB HL; and/or
   f. 1 kHz-25 dB HL to 40 dB HL; and/or
   g. 0.5 kHz-25 dB HL to 40 dB HL; and/or
   h. 0.25 kHz-25 dB HL to 40 dB HL.
26. The hair cell regeneration agent(s) for use according to any of embodiments 12-18 or 21-25, wherein the patient has an audiogram with hearing thresholds in the range of 25 dB HL to 40 dB HL at 8 kHz when measured by pure tone audiometry prior to the treatment.
27. The hair cell regeneration agent(s) for use according to any of embodiments 11, 13-18 or 21-26, wherein the patient has an audiogram with hearing thresholds in the range of 25 dB HL to 40 dB HL at 6 kHz when measured by pure tone audiometry prior to the treatment.
28. The hair cell regeneration agent(s) for use according to any of embodiments 11, 12, 14-18 or 21-27, wherein the patient has an audiogram with hearing thresholds in the range of 25 dB HL to 40 dB HL at 4 kHz when measured by pure tone audiometry prior to the treatment.
29. The hair cell regeneration agent(s) for use according to any of embodiments 11-13, 15-18, or 21-28, wherein the patient has an audiogram with hearing thresholds in the range of 25 dB HL to 40 dB HL at 3 kHz when measured by pure tone audiometry prior to the treatment.
30. The hair cell regeneration agent(s) for use according to any one of embodiments 11-14, 16-18, or 21-29, wherein the patient has an audiogram with hearing thresholds in the range of 25 dB HL to 40 dB HL at 2 kHz when measured by pure tone audiometry prior to the treatment.
31. The hair cell regeneration agent(s) for use according to any one of embodiments 11-15, 17, 18 or 21-30, wherein the patient has an audiogram with hearing thresholds in the range of 25 dB HL to 40 dB HL at 1 kHz when measured by pure tone audiometry prior to the treatment.
32. The hair cell regeneration agent(s) for use according to any one of embodiments 11-16, 18, or 21-31, wherein the patient has an audiogram with hearing thresholds in the range of 25 dB HL to 40 dB HL at 0.5 kHz when measured by pure tone audiometry prior to the treatment.
33. The hair cell regeneration agent(s) for use according to any one of embodiments 11-17 or 21-32, wherein the patient has an audiogram with hearing thresholds in the range of 25 dB HL to 40 dB HL at 0.25 kHz when measured by pure tone audiometry prior to the treatment.
34. The hair cell regeneration agent(s) for use according to embodiment 25, wherein the patient has an audiogram with hearing thresholds in the following ranges when measured by pure tone audiometry prior to the treatment:
   a. 8 kHz-25 dB HL to 40 dB HL; and
   b. 6 kHz-25 dB HL to 40 dB HL; and
   c. 4 kHz-25 dB HL to 40 dB HL; and
   d. 3 kHz-25 dB HL to 40 dB HL; and
   e. 2 kHz-25 dB HL to 40 dB HL; and
   f. 1 kHz-25 dB HL to 40 dB HL; and
   g. 0.5 kHz-25 dB HL to 40 dB HL; and
   h. 0.25 kHz-25 dB HL to 40 dB HL.
35. The hair cell regeneration agent(s) for use according to any preceding embodiment, wherein the patient has a standard word recognition score of 90% or less, 85% or less, or 80% or less, 70% or less, 60% or less, or 50% or less or 40% or less, or 30% or less prior to the treatment, for example, a standard word recognition score 40% or less, or 30% or less prior to the treatment.
36. The hair cell regeneration agent(s) for use according to any preceding embodiment, wherein the patient has a standard word recognition score of 90% or less prior to the treatment.
37. The hair cell regeneration agent(s) for use according to any preceding embodiment, wherein the patient has a standard word recognition score of 85% or less prior to the treatment.
38. The hair cell regeneration agent(s) for use according to any preceding embodiment, wherein the patient has a standard word recognition score of 80% or less prior to the treatment.
39. The hair cell regeneration agent(s) for use according to any preceding embodiment, wherein the patient has a standard word recognition score of 70% or less prior to the treatment.
40. The hair cell regeneration agent(s) for use according to any preceding embodiment, wherein the patient has a standard word recognition score of 60% or less prior to the treatment.
41. The hair cell regeneration agent(s) for use according to any preceding embodiment, wherein the patient has a standard word recognition score of 50% or less prior to the treatment.
42. The hair cell regeneration agent(s) for use according to any preceding embodiment, wherein the patient correctly identifies 48 or fewer words, 45 or fewer words, 42 or fewer words, or 40 or fewer words, 35 or fewer words, 30 or fewer words, 25 or fewer, 20 or fewer, or 15 or fewer words in a standard word recognition test for 50 words prior to the treatment for example, 20 or fewer or 15 or fewer words in a standard word recognition test for 50 words prior to the treatment.
43. The hair cell regeneration agent(s) for use according to any preceding embodiment, wherein the patient correctly identifies 45 or fewer words in a standard word recognition test for 50 words prior to the treatment.
44. The hair cell regeneration agent(s) for use according to any preceding embodiment, wherein the patient correctly identifies 42 or fewer words in a standard word recognition test for 50 words prior to the treatment.
45. The hair cell regeneration agent(s) for use according to any preceding embodiment, wherein the patient correctly identifies 40 or fewer words in a standard word recognition test for 50 words prior to the treatment.
46. The hair cell regeneration agent(s) for use according to any preceding embodiment, wherein the patient correctly identifies 35 or fewer words in a standard word recognition test for 50 words prior to the treatment.
47. The hair cell regeneration agent(s) for use according to any preceding embodiment, wherein the patient correctly identifies 30 or fewer words in a standard word recognition test for 50 words prior to the treatment.
48. The hair cell regeneration agent(s) for use according to any preceding embodiment, wherein the patient correctly identifies 25 or fewer words in a standard word recognition test for 50 words prior to the treatment.
49. A hair cell regeneration agent(s) for use in treating sensorineural hearing loss in a human patient, wherein the patient has a hearing threshold of between 40 dB HL to 70 dB HL at 16 kHz when measured by pure tone audiometry prior to the treatment.
50. The hair cell regeneration agent(s) for use according to embodiment 49, wherein the patient has an audiogram with hearing thresholds in the following ranges when measured by pure tone audiometry prior to the treatment:
   a. 14 kHz-40 dB HL to 85 dB HL; and/or
   b. 12 kHz-40 dB HL to 95 dB HL; and/or
   c. 10 kHz-40 dB HL to 95 dB HL.
51. The hair cell regeneration agent(s) for use according to embodiment 50, wherein the patient has an audiogram with hearing thresholds in the range of 40 dB HL to 85 dB HL at 14 kHz.
52. The hair cell regeneration agent(s) for use according to embodiment 50 or 51, wherein the patient has an audiogram with hearing thresholds in the range of 40 dB HL to 95 dB HL at 12 kHz.
53. The hair cell regeneration agent(s) for use according to any one of embodiments 50-52, wherein the patient has an audiogram with hearing thresholds in the range of 40 dB HL to 95 dB HL at 10 kHz.
54. The hair cell regeneration agent(s) for use according to any preceding embodiment, wherein the patient has a words-in-noise score of 90% or less, 80% or less, 70% or less, 60% or less, 50% or less, 40% or less, 30% or less, or 20% or less prior to the treatment, for example, a words-in-noise score of 20% or less prior to the treatment.
55. The hair cell regeneration agent(s) for use according to any preceding embodiment, wherein the patient has a words-in-noise score of 90% or less prior to treatment.
56. The hair cell regeneration agent(s) for use according to any preceding embodiment, wherein the patient has a words-in-noise score of 80% or less prior to the treatment.
57. The hair cell regeneration agent(s) for use according to any preceding embodiment, wherein the patient has a words-in-noise score of 70% or less prior to the treatment.
58. The hair cell regeneration agent(s) for use according to any preceding embodiment, wherein the patient has a words-in-noise score of 60% or less prior to the treatment.
59. The hair cell regeneration agent(s) for use according to any preceding embodiment, wherein the patient has a words-in-noise score of 50% or less prior to the treatment.
60. The hair cell regeneration agent(s) for use according to any preceding embodiment, wherein the patient has a words-in-noise score of 40% or less prior to the treatment.
61. The hair cell regeneration agent(s) for use according to any preceding embodiment, wherein the patient has a words-in-noise score of 30% or less prior to the treatment.
62. The hair cell regeneration agent(s) for use according to any preceding embodiment, wherein the patient correctly identifies 63 or fewer, 56 or fewer, 49 or fewer, 42 or fewer, 35 or fewer, 28 or fewer, or 21 or fewer words in a words-in-noise test for 70 words prior to the treatment.
63. The hair cell regeneration agent(s) for use according to any preceding embodiment, wherein the patient correctly identifies 63 or fewer words in a words-in-noise test for 70 words prior to treatment.
64. The hair cell regeneration agent(s) for use according to any preceding embodiment, wherein the patient correctly identifies 56 or fewer words in a words-in-noise test for 70 words prior to treatment.
65. The hair cell regeneration agent(s) for use according to any preceding embodiment, wherein the patient correctly identifies 49 or fewer words in a words-in-noise test for 70 words prior to treatment.
66. The hair cell regeneration agent(s) for use according to any preceding embodiment, wherein the patient correctly identifies 42 or fewer words in a words-in-noise test for 70 words prior to treatment.
67. The hair cell regeneration agent(s) for use according to any preceding embodiment, wherein the patient correctly identifies 35 or fewer words in a words-in-noise test for 70 words prior to treatment.
68. The hair cell regeneration agent(s) for use according to any preceding embodiment, wherein the patient correctly identifies 28 or fewer words in a words-in-noise test for 70 words prior to treatment.
69. The hair cell regeneration agent(s) for use according to any preceding embodiment, wherein the patient correctly identifies 21 or fewer words in a words-in-noise test for 70 words prior to treatment.
70. The hair cell regeneration agent(s) for use according to any preceding embodiment, wherein the patient correctly identifies 32 or fewer, 28 or fewer, 24 or fewer, 21 or fewer, 17 or fewer, 14 or fewer or 11 or fewer words in a words-in-noise test for 35 words prior to the treatment.
71. The hair cell regeneration agent(s) for use according to any preceding embodiment, wherein the patient correctly identifies 32 or fewer in a words-in-noise test for 35 words prior to the treatment.
72. The hair cell regeneration agent(s) for use according to any preceding embodiment, wherein the patient correctly identifies 28 or fewer in a words-in-noise test for 35 words prior to the treatment.
73. The hair cell regeneration agent(s) for use according to any preceding embodiment, wherein the patient correctly identifies 24 or fewer in a words-in-noise test for 35 words prior to the treatment.
74. The hair cell regeneration agent(s) for use according to any preceding embodiment, wherein the patient correctly identifies 21 or fewer in a words-in-noise test for 35 words prior to the treatment.
75. The hair cell regeneration agent(s) for use according to any preceding embodiment, wherein the patient correctly identifies 17 or fewer in a words-in-noise test for 35 words prior to the treatment.
76. The hair cell regeneration agent(s) for use according to any preceding embodiment, wherein the patient correctly identifies 14 or fewer in a words-in-noise test for 35 words prior to the treatment.
77. The hair cell regeneration agent(s) for use according to any preceding embodiment, wherein the patient correctly identifies 11 or fewer in a words-in-noise test for 35 words prior to the treatment.
78. The hair cell regeneration agent(s) for use according to any of embodiments 3-77, wherein the patient has sudden sensorineural hearing loss or noise-induced sensorineural hearing loss.
79. The hair cell regeneration agent(s) for use according to embodiment 78, wherein the patient has sudden sensorineural hearing loss.
80. The hair cell regeneration agent(s) for use according to embodiment 78, wherein the patient has noise-induced sensorineural hearing loss.
81. A hair cell regeneration agent(s) for use in treating hidden hearing loss in a human patient.
82. The hair cell regeneration agent(s) for use according to embodiment 81, wherein the patient has:
   (i) hearing thresholds of less than 25 dB HL at 0.25 kHz, 0.5 kHz, 1 kHz, 2 kHz, 3 kHz, 4 kHz, 6 kHz and 8 kHz when measured by pure tone audiometry prior to the treatment; and
   (ii) a words-in-noise score of 90% or less, 80% or less, 70% or less, 60% or less, 50% or less prior to the treatment;
   and wherein said treatment involves at least an improvement in words-in-noise score.
83. A hair cell regeneration agent(s) for use in treating a human patient, wherein the patient has:
   (i) hearing thresholds of less than 25 dB HL at 0.25 kHz, 0.5 kHz, 1 kHz, 2 kHz, 3 kHz, 4 kHz, 6 kHz and 8 kHz when measured by pure tone audiometry prior to the treatment; and
   (ii) a words-in-noise score of 90% or less, 80% or less, 70% or less, 60% or less, 50% or less prior to the treatment;
   and wherein said treatment involves at least an improvement in words-in-noise score.
84. The hair cell regeneration agent(s) for use according to embodiment 82 or embodiment 83, wherein the patient has a words-in-noise score of 90% or less prior to treatment.
85. The hair cell regeneration agent(s) for use according to embodiment 82 or embodiment 83, wherein the patient has a words-in-noise score of 80% or less prior to treatment.
86. The hair cell regeneration agent(s) for use according to embodiment 82 or embodiment 83, wherein the patient has a words-in-noise score of 70% or less prior to treatment.
87. The hair cell regeneration agent(s) for use according to embodiment 82 or embodiment 83, wherein the patient has a words-in-noise score of 60% or less prior to treatment.
88. The hair cell regeneration agent(s) for use according to embodiment 82 or embodiment 83, wherein the patient has a words-in-noise score of 50% or less prior to treatment.
89. The hair cell regeneration agent(s) for use according to any of embodiments 81-88, wherein the patient has a hearing threshold of between 40 dB HL to 70 dB HL at 16 kHz when measured by pure tone audiometry prior to the treatment.
90. The hair cell regeneration agent(s) for use according to embodiment 89, wherein the patient has an audiogram with hearing thresholds in the following ranges when measured by pure tone audiometry prior to the treatment:
   a. 14 kHz-40 dB HL to 85 dB HL; and/or
   b. 12 kHz-40 dB HL to 95 dB HL; and/or
   c. 10 kHz-40 dB HL to 95 dB HL.
91. The hair cell regeneration agent(s) for use according to embodiment 90, wherein the patient has an audiogram with hearing thresholds in the range of 40 dB HL to 85 dB HL at 14 kHz.
92. The hair cell regeneration agent(s) for use according to embodiment 90 or embodiment 91, wherein the patient has an audiogram with hearing thresholds in the range of 40 dB HL to 95 dB HL at 12 kHz.
93. The hair cell regeneration agent(s) for use according to any of embodiments 90-92, wherein the patient has an audiogram with hearing thresholds in the range of 40 dB HL to 95 dB HL at 10 kHz.
94. The hair cell regeneration agent(s) for use according to any preceding embodiment, wherein the patient's Signal-to-Noise ratio (SNR) for a predicted mean of 50% correct words in a words-in-noise test is about 25 dB, about 24 dB, about 23 dB, about 22 dB, about 21 dB, about 20 dB, about 19 dB, about 18 dB, about 17 dB, about 16 dB, about 15 dB, about 14 dB, about 13 dB, about 12 dB, about 11 dB, about 10 dB, about 9 dB, about 8 dB, about 7 dB, or about 6 dB, wherein the SNR is calculated using the Spearman-Kärber equation.
95. The hair cell regeneration agent(s) for use according to embodiment 94, wherein the patient's Signal-to-Noise ratio (SNR) for a predicted mean of 50% correct words in a words-in-noise test is about 25 dB, wherein the SNR is calculated using the Spearman-Kärber equation.
96. The hair cell regeneration agent(s) for use according to embodiment 94, wherein the patient's Signal-to-Noise ratio (SNR) for a predicted mean of 50% correct words in a words-in-noise test is about 24 dB, wherein the SNR is calculated using the Spearman-Kärber equation.
97. The hair cell regeneration agent(s) for use according to embodiment 94, wherein the patient's Signal-to-Noise ratio (SNR) for a predicted mean of 50% correct words in a words-in-noise test is about 23 dB, wherein the SNR is calculated using the Spearman-Kärber equation.
98. The hair cell regeneration agent(s) for use according to embodiment 94, wherein the patient's Signal-to-Noise ratio (SNR) for a predicted mean of 50% correct words in a words-in-noise test is about 22 dB, wherein the SNR is calculated using the Spearman-Kärber equation.
99. The hair cell regeneration agent(s) for use according to embodiment 94, wherein the patient's Signal-to-Noise ratio (SNR) for a predicted mean of 50% correct words in a words-in-noise test is about 21 dB, wherein the SNR is calculated using the Spearman-Kärber equation.
100. The hair cell regeneration agent(s) for use according to embodiment 94, wherein the patient's Signal-to-Noise ratio (SNR) for a predicted mean of 50% correct words in a words-in-noise test is about 20 dB, wherein the SNR is calculated using the Spearman-Kärber equation.
101. The hair cell regeneration agent(s) for use according to embodiment 94, wherein the patient's Signal-to-Noise ratio (SNR) for a predicted mean of 50% correct words in a words-in-noise test is about 19 dB, wherein the SNR is calculated using the Spearman-Kärber equation.
102. The hair cell regeneration agent(s) for use according to embodiment 94, wherein the patient's Signal-to-Noise ratio (SNR) for a predicted mean of 50% correct words in a words-in-noise test is about 18 dB, wherein the SNR is calculated using the Spearman-Kärber equation.
103. The hair cell regeneration agent(s) for use according to embodiment 94, wherein the patient's Signal-to-Noise ratio (SNR) for a predicted mean of 50% correct words in a words-in-noise test is about 17 dB, wherein the SNR is calculated using the Spearman-Kärber equation.
104. The hair cell regeneration agent(s) for use according to embodiment 94, wherein the patient's Signal-to-Noise ratio (SNR) for a predicted mean of 50% correct words in a words-in-noise test is about 16 dB, wherein the SNR is calculated using the Spearman-Kärber equation.
105. The hair cell regeneration agent(s) for use according to embodiment 94, wherein the patient's Signal-to-Noise ratio (SNR) for a predicted mean of 50% correct words in a words-in-noise test is about 15 dB, wherein the SNR is calculated using the Spearman-Kärber equation.
106. The hair cell regeneration agent(s) for use according to embodiment 94, wherein the patient's Signal-to-Noise ratio (SNR) for a predicted mean of 50% correct words in a words-in-noise test is about 14 dB, wherein the SNR is calculated using the Spearman-Kärber equation.
107. The hair cell regeneration agent(s) for use according to embodiment 94, wherein the patient's Signal-to-Noise ratio (SNR) for a predicted mean of 50% correct words in a words-in-noise test is about 13 dB, wherein the SNR is calculated using the Spearman-Kärber equation.
108. The hair cell regeneration agent(s) for use according to embodiment 94, wherein the patient's Signal-to-Noise ratio (SNR) for a predicted mean of 50% correct words in a words-in-noise test is about 12 dB, wherein the SNR is calculated using the Spearman-Kärber equation.
109. The hair cell regeneration agent(s) for use according to embodiment 94, wherein the patient's Signal-to-Noise ratio (SNR) for a predicted mean of 50% correct words in a words-in-noise test is about 11 dB, wherein the SNR is calculated using the Spearman-Kärber equation.
110. The hair cell regeneration agent(s) for use according to embodiment 94, wherein the patient's Signal-to-Noise ratio (SNR) for a predicted mean of 50% correct words in a words-in-noise test is about 10 dB, wherein the SNR is calculated using the Spearman-Kärber equation.
111. The hair cell regeneration agent(s) for use according to embodiment 94, wherein the patient's Signal-to-Noise ratio (SNR) for a predicted mean of 50% correct words in a words-in-noise test is about 9 dB, wherein the SNR is calculated using the Spearman-Kärber equation.
112. The hair cell regeneration agent(s) for use according to embodiment 94, wherein the patient's Signal-to-Noise ratio (SNR) for a predicted mean of 50% correct words in a words-in-noise test is about 8 dB, wherein the SNR is calculated using the Spearman-Kärber equation.
113. The hair cell regeneration agent(s) for use according to embodiment 94, wherein the patient's Signal-to-Noise ratio (SNR) for a predicted mean of 50% correct words in a words-in-noise test is about 7 dB, wherein the SNR is calculated using the Spearman-Kärber equation.
114. The hair cell regeneration agent(s) for use according to embodiment 94, wherein the patient's Signal-to-Noise ratio (SNR) for a predicted mean of 50% correct words in a words-in-noise test is about 6 dB, wherein the SNR is calculated using the Spearman-Kärber equation.
115. The hair cell regeneration agent(s) for use according to any preceding embodiment, wherein the hair cell regeneration agent(s) is a progenitor cell activation agent(s).
116. The hair cell regeneration agent(s) for use according to any preceding embodiment, wherein the hair cell regeneration agent(s) is a gamma secretase inhibitor.
117. The hair cell regeneration agent(s) for use according to embodiment 116, wherein the gamma secretase inhibitor is Semagacestat (LY 450139) or a pharmaceutically acceptable salt thereof.
118. The hair cell regeneration agent(s) for use according to embodiment 116, wherein the gamma secretase inhibitor is Begacestat (GSI-953) or a pharmaceutically acceptable salt thereof.
119. The hair cell regeneration agent(s) for use according to embodiment 116, wherein the gamma secretase inhibitor is Avagacestat (BMS-708163) or a pharmaceutically acceptable salt thereof.
120. The hair cell regeneration agent(s) for use according to embodiment 116, wherein the gamma secretase inhibitor is EVP-0962 or a pharmaceutically acceptable salt thereof.
121. The hair cell regeneration agent(s) for use according to embodiment 116, wherein the gamma secretase inhibitor is Crenigacestat LY 3039478 (JSMD194) or a pharmaceutically acceptable salt thereof.
122. The hair cell regeneration agent(s) for use according to embodiment 116, wherein the gamma secretase inhibitor is MK-0572 or a pharmaceutically acceptable salt thereof.
123. The hair cell regeneration agent(s) for use according to embodiment 116, wherein the gamma secretase inhibitor is NIC5-15 or a pharmaceutically acceptable salt thereof.
124. The hair cell regeneration agent(s) for use according to embodiment 116, wherein the gamma secretase inhibitor is NGP 555 or a pharmaceutically acceptable salt thereof.
125. The hair cell regeneration agent(s) for use according to embodiment 116, wherein the gamma secretase inhibitor is Nirogacestat (PF 030840140) or a pharmaceutically acceptable salt thereof.
126. The hair cell regeneration agent(s) for use according to embodiment 116, wherein the gamma secretase inhibitor is PF-06648671 or a pharmaceutically acceptable salt thereof.
127. The hair cell regeneration agent(s) for use according to embodiment 116, wherein the gamma secretase inhibitor is RO4929097 or a pharmaceutically acceptable salt thereof.
128. The hair cell regeneration agent(s) for use according to embodiment 116, wherein the gamma secretase inhibitor is BMS-905024 or a pharmaceutically acceptable salt thereof.
129. The hair cell regeneration agent(s) for use according to embodiment 116, wherein the gamma secretase inhibitor is BMS-932481 or a pharmaceutically acceptable salt thereof.
130. The hair cell regeneration agent(s) for use according to embodiment 116, wherein the gamma secretase inhibitor is BMS-986133 or a pharmaceutically acceptable salt thereof.
131. The hair cell regeneration agent(s) for use according to embodiment 116, wherein the gamma secretase inhibitor is BMS 299897 or a pharmaceutically acceptable salt thereof.
132. The hair cell regeneration agent(s) for use according to embodiment 116, wherein the gamma secretase inhibitor is BPN-15606 or a pharmaceutically acceptable salt thereof.
133. The hair cell regeneration agent(s) for use according to embodiment 116, wherein the gamma secretase inhibitor is Carprofen or a pharmaceutically acceptable salt thereof.
134. The hair cell regeneration agent(s) for use according to embodiment 116, wherein the gamma secretase inhibitor is CHF5022 or a pharmaceutically acceptable salt thereof.
135. The hair cell regeneration agent(s) for use according to embodiment 116, wherein the gamma secretase inhibitor is CHF5074 or a pharmaceutically acceptable salt thereof.
136. The hair cell regeneration agent(s) for use according to embodiment 116, wherein the gamma secretase inhibitor is Compound E (CAS No. 209986-17-4) or a pharmaceutically acceptable salt thereof.
137. The hair cell regeneration agent(s) for use according to embodiment 116, wherein the gamma secretase inhibitor is Compound W (CAS No. 173550-33-9) or a pharmaceutically acceptable salt thereof.
138. The hair cell regeneration agent(s) for use according to embodiment 116, wherein the gamma secretase inhibitor is DAPT or a pharmaceutically acceptable salt thereof.
139. The hair cell regeneration agent(s) for use according to embodiment 116, wherein the gamma secretase inhibitor is DBZ or a pharmaceutically acceptable salt thereof.
140. The hair cell regeneration agent(s) for use according to embodiment 116, wherein the gamma secretase inhibitor is E-2012 or a pharmaceutically acceptable salt thereof.
141. The hair cell regeneration agent(s) for use according to embodiment 116, wherein the gamma secretase inhibitor is EVP-A or a pharmaceutically acceptable salt thereof.
142. The hair cell regeneration agent(s) for use according to embodiment 116, wherein the gamma secretase inhibitor is EVP-B or a pharmaceutically acceptable salt thereof.
143. The hair cell regeneration agent(s) for use according to embodiment 116, wherein the gamma secretase inhibitor is EVP-0015962 or a pharmaceutically acceptable salt thereof.
144. The hair cell regeneration agent(s) for use according to embodiment 116, wherein the gamma secretase inhibitor is Flurizan™ or a pharmaceutically acceptable salt thereof.
145. The hair cell regeneration agent(s) for use according to embodiment 116, wherein the gamma secretase inhibitor is GSI-136 or a pharmaceutically acceptable salt thereof.
146. The hair cell regeneration agent(s) for use according to embodiment 116, wherein the gamma secretase inhibitor is indomethacin or a pharmaceutically acceptable salt thereof.
147. The hair cell regeneration agent(s) for use according to embodiment 116, wherein the gamma secretase inhibitor is JLK 6 or a pharmaceutically acceptable salt thereof.
148. The hair cell regeneration agent(s) for use according to embodiment 116, wherein the gamma secretase inhibitor is JNJ-40418677 or a pharmaceutically acceptable salt thereof.
149. The hair cell regeneration agent(s) for use according to embodiment 116, wherein the gamma secretase inhibitor is L-685,458 or a pharmaceutically acceptable salt thereof.
150. The hair cell regeneration agent(s) for use according to embodiment 116, wherein the gamma secretase inhibitor is Deshydroxy (LY-411575) or a pharmaceutically acceptable salt thereof.
151. The hair cell regeneration agent(s) for use according to embodiment 116, wherein the gamma secretase inhibitor is LY 411575 or a pharmaceutically acceptable salt thereof.
152. The hair cell regeneration agent(s) for use according to embodiment 116, wherein the gamma secretase inhibitor is MDL 28170 or a pharmaceutically acceptable salt thereof.
153. The hair cell regeneration agent(s) for use according to embodiment 116, wherein the gamma secretase inhibitor is MRK 560 or a pharmaceutically acceptable salt thereof.
154. The hair cell regeneration agent(s) for use according to embodiment 116, wherein the gamma secretase inhibitor is MW167 or a pharmaceutically acceptable salt thereof.
155. The hair cell regeneration agent(s) for use according to embodiment 116, wherein the gamma secretase inhibitor is NMK-T-057 or a pharmaceutically acceptable salt thereof.
156. The hair cell regeneration agent(s) for use according to embodiment 116, wherein the gamma secretase inhibitor is Suldinac sulfide or a pharmaceutically acceptable salt thereof.
157. A Wnt agonist and/or an epigenetic modulator for use in treating sensorineural hearing loss in a human patient, wherein said Wnt agonist and said epigenetic modulator are both administered to the patient, wherein optionally the sensorineural hearing loss is sudden sensorineural hearing loss.
158. A Wnt agonist and/or an epigenetic modulator for use in treating noise-induced sensorineural hearing loss in a human patient, wherein said Wnt agonist and said epigenetic modulator are both administered to the patient.
159. A Wnt agonist and/or an epigenetic modulator for use in treating sensorineural hearing loss in a human patient, wherein the average of the patient's hearing thresholds across 0.5 kHz, 1 kHz, 2 kHz and 4 kHz is at least 40 dB HL and no more than 70 dB HL when measured by pure tone audiometry prior to the treatment, wherein said Wnt agonist and said epigenetic modulator are both administered to the patient.
160. The Wnt agonist and/or the epigenetic modulator for use according to embodiment 159, wherein the average of the patient's hearing thresholds across 0.5 kHz, 1 kHz, 2 kHz and 4 kHz is at least 40 dB HL and no more than 55 dB HL when measured by pure tone audiometry prior to the treatment.
161. The Wnt agonist and/or the epigenetic modulator for use according to embodiment 159, wherein the average of the patient's hearing thresholds across 0.5 kHz, 1 kHz, 2 kHz and 4 kHz is at least 55 dB HL and no more than 70 dB HL when measured by pure tone audiometry prior to the treatment.
162. A Wnt agonist and/or an epigenetic modulator for use in treating sensorineural hearing loss in a human patient, wherein the patient has a hearing threshold of at least 40 dB HL at 4 kHz; and/or at least 40 dB HL at 6 kHz; and/or at least 40 dB HL at 8 kHz; when measured by pure tone audiometry prior to the treatment, wherein said Wnt agonist and said epigenetic modulator are both administered to the patient.
163. The Wnt agonist and/or the epigenetic modulator for use according to embodiment 162, wherein the patient has a hearing threshold of at least 40 dB HL at 4 kHz when measured by pure tone audiometry prior to the treatment.
164. The Wnt agonist and/or the epigenetic modulator for use according to embodiment 162 or embodiment 163, wherein the patient has a hearing threshold of at least 40 dB HL at 6 kHz when measured by pure tone audiometry prior to the treatment.
165. The Wnt agonist and/or the epigenetic modulator for use according to any of embodiments 162-164, wherein the patient has a hearing threshold of at least 40 dB HL at 8 kHz when measured by pure tone audiometry prior to the treatment.
166. The Wnt agonist and/or the epigenetic modulator for use according to any of embodiment 162-165, wherein the patient has an audiogram with hearing thresholds in the following ranges when measured by pure tone audiometry prior to the treatment:
   a. 8 kHz-40 dB HL to 95 dB HL; and/or
   b. 6 kHz-40 dB HL to 85 dB HL; and/or
   c. 4 kHz-40 dB HL to 80 dB HL; and/or
   d. 3 kHz-40 dB HL to 70 dB HL; and/or
   e. 2 kHz-40 dB HL to 70 dB HL; and/or
   f. 1 kHz-40 dB HL to 70 dB HL; and/or
   g. 0.5 kHz-40 dB HL to 70 dB HL; and/or
   h. 0.25 kHz-40 dB HL to 70 dB HL.
167. The Wnt agonist and/or the epigenetic modulator for use according to any of embodiments 162-166, wherein the patient has an audiogram with a hearing threshold in the range of 40 dB HL to 95 dB HL at 8 kHz when measured by pure tone audiometry prior to treatment.
168. The Wnt agonist and/or the epigenetic modulator for use according to any of embodiments 162-167, wherein the patient has an audiogram with a hearing threshold in the range of 40 dB HL to 85 dB HL at 6 kHz when measured by pure tone audiometry prior to treatment.
169. The Wnt agonist and/or the epigenetic modulator for use according to any of embodiments 162-168, wherein the patient has an audiogram with a hearing threshold in the range of 40 dB HL to 80 dB HL at 4 kHz when measured by pure tone audiometry prior to treatment.
170. The Wnt agonist and/or the epigenetic modulator for use according to any of embodiments 162-169, wherein the patient has an audiogram with a hearing threshold in the range of 40 dB HL to 70 dB HL at 3 kHz when measured by pure tone audiometry prior to treatment.
171. The Wnt agonist and/or the epigenetic modulator for use according to any of embodiments 162-170, wherein the patient has an audiogram with a hearing threshold in the range of 40 dB HL to 70 dB HL at 2 kHz when measured by pure tone audiometry prior to treatment.
172. The Wnt agonist and/or the epigenetic modulator for use according to any of embodiments 162-171, wherein the patient has an audiogram with a hearing threshold in the range of 40 dB HL to 70 dB HL at 1 kHz when measured by pure tone audiometry prior to treatment.
173. The Wnt agonist and/or the epigenetic modulator for use according to any of embodiments 162-172, wherein the patient has an audiogram with a hearing threshold in the range of 40 dB HL to 70 dB HL at 0.5 kHz when measured by pure tone audiometry prior to treatment.
174. The Wnt agonist and/or the epigenetic modulator for use according to any of embodiments 162-173, wherein the patient has an audiogram with a hearing threshold in the range of 40 dB HL to 70 dB HL at 0.25 kHz when measured by pure tone audiometry prior to treatment.
175. The Wnt agonist and/or the epigenetic modulator for use according to embodiment 166, wherein the patient has an audiogram with hearing thresholds in the following ranges when measured by pure tone audiometry prior to the treatment:
   a. 8 kHz-40 dB HL to 70 dB HL; and
   b. 6 kHz-40 dB HL to 70 dB HL; and
   c. 4 kHz-40 dB HL to 70 dB HL; and
   d. 3 kHz-40 dB HL to 70 dB HL; and
   e. 2 kHz-40 dB HL to 70 dB HL; and
   f. 1 kHz-40 dB HL to 70 dB HL; and
   g. 0.5 kHz-40 dB HL to 70 dB HL; and
   h. 0.25 kHz-40 dB HL to 70 dB HL.
176. A Wnt agonist and/or an epigenetic modulator for use in treating sensorineural hearing loss in a human patient, wherein the average of the patient's hearing thresholds across 0.5 kHz, 1 kHz, 2 kHz and 4 kHz is at least 25 dB HL and no more than 40 dB HL when measured by pure tone audiometry prior to the treatment, wherein said Wnt agonist and said epigenetic modifier are both administered to the patient.
177. A Wnt agonist and/or an epigenetic modulator for use in treating sensorineural hearing loss in a human patient, wherein the patient has a hearing threshold of at least 25 dB HL and no more than 40 dB HL at 4 kHz; and/or at least 25 dB HL and no more than 40 dB HL at 6 kHz; and/or at least 25 dB HL and no more than 40 dB HL at 8 kHz; when measured by pure tone audiometry prior to the treatment, wherein said Wnt agonist and said epigenetic modifier are both administered to the patient.
178. The Wnt agonist and/or the epigenetic modulator for use according to embodiment 177, wherein the patient has a hearing threshold of at least 25 dB HL and no more than 40 dB HL at 4 kHz when measured by pure tone audiometry prior to the treatment.
179. The Wnt agonist and/or the epigenetic modulator for use according to embodiment 177 or embodiment 178, wherein the patient has a hearing threshold of at least 25 dB HL and no more than 40 dB HL at 6 kHz when measured by pure tone audiometry prior to the treatment.
180. The Wnt agonist and/or the epigenetic modulator for use according to any of embodiments 177-179, wherein the patient has a hearing threshold of at least 25 dB HL and no more than 40 dB HL at 8 kHz when measured by pure tone audiometry prior to the treatment.
181. The Wnt agonist and/or the epigenetic modulator for use according to any of embodiments 177-180, wherein the patient has an audiogram with hearing thresholds in the following ranges when measured by pure tone audiometry prior to the treatment:
   a. 8 kHz-25 dB HL to 40 dB HL; and/or
   b. 6 kHz-25 dB HL to 40 dB HL; and/or
   c. 4 kHz-25 dB HL to 40 dB HL; and/or
   d. 3 kHz-25 dB HL to 40 dB HL; and/or
   e. 2 kHz-25 dB HL to 40 dB HL; and/or
   f. 1 kHz-25 dB HL to 40 dB HL; and/or
   g. 0.5 kHz-25 dB HL to 40 dB HL; and/or
   h. 0.25 kHz-25 dB HL to 40 dB HL.
182. The Wnt agonist and/or the epigenetic modulator for use according to any of embodiments 168-174 or 177-181, wherein the patient has an audiogram with a hearing threshold in the range of 25 dB HL to 40 dB HL at 8 kHz when measured by pure tone audiometry prior to treatment.
183. The Wnt agonist and/or the epigenetic modulator for use according to any of embodiments 167, 169-174 or 177-182, wherein the patient has an audiogram with a hearing threshold in the range of 25 dB HL to 40 dB HL at 6 kHz when measured by pure tone audiometry prior to treatment.
184. The Wnt agonist and/or the epigenetic modulator for use according to any of embodiments 167, 168, 170-174 or 177-183, wherein the patient has an audiogram with a hearing threshold in the range of 25 dB HL to 40 dB HL at 4 kHz when measured by pure tone audiometry prior to treatment.
185. The Wnt agonist and/or the epigenetic modulator for use according to any of embodiments 167-169, 171-174 or 177-184, wherein the patient has an audiogram with a hearing threshold in the range of 25 dB HL to 40 dB HL at 3 kHz when measured by pure tone audiometry prior to treatment.
186. The Wnt agonist and/or the epigenetic modulator for use according to any of embodiments 167-170, 172-174 or 177-185, wherein the patient has an audiogram with a hearing threshold in the range of 25 dB HL to 40 dB HL at 2 kHz when measured by pure tone audiometry prior to treatment.
187. The Wnt agonist and/or the epigenetic modulator for use according to any of embodiments 167-171, 173, 174 or 177-186, wherein the patient has an audiogram with a hearing threshold in the range of 25 dB HL to 40 dB HL at 1 kHz when measured by pure tone audiometry prior to treatment.
188. The Wnt agonist and/or the epigenetic modulator for use according to any of embodiments 167-172, 174 or 177-187, wherein the patient has an audiogram with a hearing threshold in the range of 25 dB HL to 40 dB HL at 0.5 kHz when measured by pure tone audiometry prior to treatment.
189. The Wnt agonist and/or the epigenetic modulator for use according to any of embodiments 167-173 or 177-188, wherein the patient has an audiogram with a hearing threshold in the range of 25 dB HL to 40 dB HL at 0.25 kHz when measured by pure tone audiometry prior to treatment.
190. The Wnt agonist and/or the epigenetic modulator for use according to embodiment 181, wherein the patient has an audiogram with hearing thresholds in the following ranges when measured by pure tone audiometry prior to the treatment:
   a. 8 kHz-25 dB HL to 40 dB HL; and
   b. 6 kHz-25 dB HL to 40 dB HL; and
   c. 4 kHz-25 dB HL to 40 dB HL; and
   d. 3 kHz-25 dB HL to 40 dB HL; and
   e. 2 kHz-25 dB HL to 40 dB HL; and
   f. 1 kHz-25 dB HL to 40 dB HL; and
   g. 0.5 kHz-25 dB HL to 40 dB HL; and
   h. 0.25 kHz-25 dB HL to 40 dB HL.
191. The Wnt agonist and/or the epigenetic modulator for use according to any of embodiments 157-190, wherein the patient has a standard word recognition score of 90% or less, 85% or less, or 80% or less, 70% or less, 60% or less, or 50% or less prior to the treatment.
192. The Wnt agonist and/or the epigenetic modulator for use according to any of embodiments 157-191, wherein the patient has a standard word recognition score of 90% or less prior to the treatment.
193. The Wnt agonist and/or the epigenetic modulator for use according to any of embodiments 157-192, wherein the patient has a standard word recognition score of 85% or less prior to the treatment.
194. The Wnt agonist and/or the epigenetic modulator for use according to any of embodiments 157-193, wherein the patient has a standard word recognition score of 80% or less prior to the treatment.
195. The Wnt agonist and/or the epigenetic modulator for use according to any of embodiments 157-194, wherein the patient has a standard word recognition score of 70% or less prior to the treatment.
196. The Wnt agonist and/or the epigenetic modulator for use according to any of embodiments 157-195, wherein the patient has a standard word recognition score of (6% or less prior to the treatment.
197. The Wnt agonist and/or the epigenetic modulator for use according to any of embodiments 157-196, wherein the patient has a standard word recognition score of 50% or less prior to the treatment.
198. The Wnt agonist and/or the epigenetic modulator for use according to any of embodiments 157-197 wherein the patient correctly identifies 45 or fewer, 42 or fewer, or 40 or fewer, 35 or fewer, 30 or fewer, or 25 or fewer words in a standard word recognition test for 50 words prior to the treatment.
199. The Wnt agonist and/or the epigenetic modulator for use according to any of embodiments 157-198, wherein the patient correctly identifies 45 or fewer in a standard word recognition test for 50 words prior to the treatment.
200. The Wnt agonist and/or the epigenetic modulator for use according to any of embodiments 157-199, wherein the patient correctly identifies 42 or fewer in a standard word recognition test for 50 words prior to the treatment.
201. The Wnt agonist and/or the epigenetic modulator for use according to any of embodiments 157-200, wherein the patient correctly identifies 40 or fewer in a standard word recognition test for 50 words prior to the treatment.
202. The Wnt agonist and/or the epigenetic modulator for use according to any of embodiments 157-201, wherein the patient correctly identifies 35 or fewer in a standard word recognition test for 50 words prior to the treatment.
203. The Wnt agonist and/or the epigenetic modulator for use according to any of embodiments 157-202, wherein the patient correctly identifies 30 or fewer in a standard word recognition test for 50 words prior to the treatment.
204. The Wnt agonist and/or the epigenetic modulator for use according to any of embodiments 157-203, wherein the patient correctly identifies 25 or fewer in a standard word recognition test for 50 words prior to the treatment.
205. A Wnt agonist and/or an epigenetic modulator for use in treating sensorineural hearing loss in a human patient, wherein the patient has a hearing threshold of between 40 dB HL to 70 dB HL at 16 kHz when measured by pure tone audiometry prior to the treatment, wherein said Wnt agonist and said epigenetic modifier are both administered to the patient.
206. The Wnt agonist and/or the epigenetic modulator for use according to embodiment 205, wherein the patient has an audiogram with hearing thresholds in the following ranges when measured by pure tone audiometry prior to the treatment:
   a. 14 kHz-40 dB HL to 85 dB HL; and/or
   b. 12 kHz-40 dB HL to 95 dB HL; and/or
   c. 10 kHz-40 dB HL to 95 dB HL.
207. The Wnt agonist and/or the epigenetic modulator for use according to embodiment 206, wherein the patient has an audiogram with a hearing threshold in the range of 40 dB HL to 85 dB HL at 14 kHz when measured by pure tone audiometry prior to the treatment.
208. The Wnt agonist and/or the epigenetic modulator for use according to embodiment 206 or embodiment 207, wherein the patient has an audiogram with a hearing threshold in the range of 40 dB HL to 95 dB HL at 12 kHz when measured by pure tone audiometry prior to the treatment.
209. The Wnt agonist and/or the epigenetic modulator for use according to any of embodiments 206-208, wherein the patient has an audiogram with a hearing threshold in the range of 40 dB HL to 95 dB HL at 10 kHz when measured by pure tone audiometry prior to the treatment.
210. The Wnt agonist and/or the epigenetic modulator for use according to any of embodiments 157-209, wherein the patient has a words-in-noise score of 90% or less, 80% or less, 70% or less, 60% or less, 50% or less, 40% or less, or 30% or less prior to the treatment.
211. The Wnt agonist and/or the epigenetic modulator for use according to any of embodiments 116-210, wherein the patient has a words-in-noise score of 90% or less prior to the treatment.
212. The Wnt agonist and/or the epigenetic modulator for use according to any of embodiments 157-211, wherein the patient has a words-in-noise score of 80% or less prior to the treatment.
213. The Wnt agonist and/or the epigenetic modulator for use according to any of embodiments 157-212, wherein the patient has a words-in-noise score of 70% or less prior to the treatment.
214. The Wnt agonist and/or the epigenetic modulator for use according to any of embodiments 157-213, wherein the patient has a words-in-noise score of 60% or less prior to the treatment.
215. The Wnt agonist and/or the epigenetic modulator for use according to any of embodiments 157-214, wherein the patient has a words-in-noise score of 50% or less prior to the treatment.
216. The Wnt agonist and/or the epigenetic modulator for use according to any of embodiments 157-215, wherein the patient has a words-in-noise score of 40% or less prior to the treatment.
217. The Wnt agonist and/or the epigenetic modulator for use according to any of embodiments 157-216, wherein the patient has a words-in-noise score of 30% or less prior to the treatment.
218. The Wnt agonist and/or the epigenetic modulator for use according to any of embodiments 157-217, wherein the patient correctly identifies 63 or fewer, 56 or fewer, 49 or fewer, 42 or fewer, 35 or fewer, 28 or fewer, or 21 or fewer words in a words-in-noise test for 70 words prior to the treatment.
219. The Wnt agonist and/or the epigenetic modulator for use according to any of embodiments 157-218, wherein the patient correctly identifies 63 or fewer words in a words-in-noise test for 70 words prior to the treatment.
220. The Wnt agonist and/or the epigenetic modulator for use according to any of embodiments 157-219, wherein the patient correctly identifies 56 or fewer words in a words-in-noise test for 70 words prior to the treatment.
221. The Wnt agonist and/or the epigenetic modulator for use according to any of embodiments 157-220, wherein the patient correctly identifies 49 or fewer words in a words-in-noise test for 70 words prior to the treatment.
222. The Wnt agonist and/or the epigenetic modulator for use according to any of embodiments 157-221, wherein the patient correctly identifies 42 or fewer words in a words-in-noise test for 70 words prior to the treatment.
223. The Wnt agonist and/or the epigenetic modulator for use according to any of embodiments 157-222, wherein the patient correctly identifies 35 or fewer words in a words-in-noise test for 70 words prior to the treatment.
224. The Wnt agonist and/or the epigenetic modulator for use according to any of embodiments 157-223, wherein the patient correctly identifies 28 or fewer words in a words-in-noise test for 70 words prior to the treatment.
225. The Wnt agonist and/or the epigenetic modulator for use according to any of embodiments 157-224, wherein the patient correctly identifies 21 or fewer words in a words-in-noise test for 70 words prior to the treatment.
226. The Wnt agonist and/or the epigenetic modulator for use according to any of embodiments 157-225, wherein the patient correctly identifies 32 or fewer, 28 or fewer, 24 or fewer, 21 or fewer, 17 or fewer, 14 or fewer or 11 or fewer words in a words-in-noise test for 35 words prior to the treatment.
227. The Wnt agonist and/or the epigenetic modulator for use according to any of embodiments 157-226, wherein the patient correctly identifies 32 or fewer words in a words-in-noise test for 35 words prior to the treatment.
228. The Wnt agonist and/or the epigenetic modulator for use according to any of embodiments 157-227, wherein the patient correctly identifies 28 or fewer words in a words-in-noise test for 35 words prior to the treatment.
229. The Wnt agonist and/or the epigenetic modulator for use according to any of embodiments 157-228, wherein the patient correctly identifies 24 or fewer words in a words-in-noise test for 35 words prior to the treatment.
230. The Wnt agonist and/or the epigenetic modulator for use according to any of embodiments 157-229, wherein the patient correctly identifies 21 or fewer words in a words-in-noise test for 35 words prior to the treatment.
231. The Wnt agonist and/or the epigenetic modulator for use according to any of embodiments 157-230, wherein the patient correctly identifies 17 or fewer words in a words-in-noise test for 35 words prior to the treatment.
232. The Wnt agonist and/or the epigenetic modulator for use according to any of embodiments 157-231, wherein the patient correctly identifies 14 or fewer words in a words-in-noise test for 35 words prior to the treatment.
233. The Wnt agonist and/or the epigenetic modulator for use according to any of embodiments 157-232, wherein the patient correctly identifies 11 or fewer words in a words-in-noise test for 35 words prior to the treatment.
234. The Wnt agonist and/or the epigenetic modulator for use according to any of embodiments 159-233, wherein the patient has sudden sensorineural hearing loss or noise-induced sensorineural hearing loss.
235. The Wnt agonist and/or the epigenetic modulator for use according to embodiment 234, wherein the patient has sudden sensorineural hearing loss.
236. The Wnt agonist and/or the epigenetic modulator for use according to embodiment 234, wherein the patient has noise-induced sensorineural hearing loss.
237. A Wnt agonist and/or an epigenetic modulator for use in treating hidden hearing loss in a human patient, wherein said Wnt agonist and said epigenetic modulator are both administered to the patient.
238. The Wnt agonist and/or the epigenetic modulator for use according to embodiment 237, wherein the patient has:
   (i) hearing thresholds of less than 25 dB HL at 0.25 kHz, 0.5 kHz, 1 kHz, 2 kHz, 3 kHz, 4 kHz, 6 kHz and 8 kHz when measured by pure tone audiometry prior to the treatment; and
   (ii) a words-in-noise score of 90% or less, 80% or less, 70% or less, 60% or less, 50% or less prior to the treatment;
   and wherein said treatment involves at least an improvement in words-in-noise score.
239. A Wnt agonist and/or an epigenetic modulator for use in treating a human patient, wherein the patient has:
   (i) hearing thresholds of less than 25 dB HL at 0.25 kHz, 0.5 kHz, 1 kHz, 2 kHz, 3 kHz, 4 kHz, 6 kHz and 8 kHz when measured by pure tone audiometry prior to the treatment; and
   (ii) a words-in-noise score of 90% or less, 80% or less, 70% or less, 60% or less, 50% or less prior to the treatment;
   and wherein said treatment involves at least an improvement in words-in-noise score, wherein said Wnt agonist and said epigenetic modulator are both administered to the patient.
240. The Wnt agonist and/or an epigenetic modulator for use according to embodiment 238 or embodiment 239 wherein the patient has a words-in-noise score of 90% or less prior to the treatment.
241. The Wnt agonist and/or an epigenetic modulator for use according to any of embodiments 238-240, wherein the patient has a words-in-noise score of 80% or less prior to the treatment.
242. The Wnt agonist and/or an epigenetic modulator for use according to any of embodiments 238-241, wherein the patient has a words-in-noise score of 70% or less prior to the treatment.
243. The Wnt agonist and/or an epigenetic modulator for use according to any of embodiments 238-242, wherein the patient has a words-in-noise score of 60% or less prior to the treatment.
244. The Wnt agonist and/or an epigenetic modulator for use according to any of embodiments 238-243, wherein the patient has a words-in-noise score of 50% or less prior to the treatment.
245. The Wnt agonist and/or the epigenetic modulator for use according to any of embodiments 237-244, wherein the patient has a hearing threshold of between 40 dB HL to 70 dB HL at 16 kHz when measured by pure tone audiometry prior to the treatment.
246. The Wnt agonist and/or the epigenetic modulator for use according to embodiment 245, wherein the patient has an audiogram with hearing thresholds in the following ranges when measured by pure tone audiometry prior to the treatment;
   a. 14 kHz-40 dB HL to 85 dB HL; and/or
   b. 12 kHz-40 dB HL to 95 dB HL; and/or
   c. 10 kHz-40 dB HL to 95 dB HL
247. The Wnt agonist and/or the epigenetic modulator for use according to embodiment 246, wherein the patient has an audiogram with hearing thresholds in the range 40 dB HL to 85 dB HL at 14 kHz when measured by pure tone audiometry prior to the treatment.
248. The Wnt agonist and/or the epigenetic modulator for use according to embodiment 246 or embodiment 247, wherein the patient has an audiogram with hearing thresholds in the range 40 dB HL to 95 dB HL at 12 kHz when measured by pure tone audiometry prior to the treatment.
249. The Wnt agonist and/or the epigenetic modulator for use according to any of embodiments 246-248, wherein the patient has an audiogram with hearing thresholds in the range 40 dB HL to 95 dB HL at 10 kHz when measured by pure tone audiometry prior to the treatment.
250. The Wnt agonist and/or the epigenetic modulator for use according to any of embodiments 157-249, wherein the patient's Signal-to-Noise ratio (SNR) for the predicted mean of 50% correct words in a words-in-noise test is about 25 dB, about 24 dB, about 23 dB, about 22 dB, about 21 dB, about 20 dB, about 19 dB, about 18 dB, about 17 dB, about 16 dB, about 15 dB about 14 dB, about 13 dB, about 12 dB, about 11 dB, about 10 dB, about 9 dB, about 8 dB, about 7 dB, or about 6 dB, wherein the SNR is calculated using the Spearman-Kärber equation.
251. The Wnt agonist and/or the epigenetic modulator for use according to embodiment 250, wherein the patient's Signal-to-Noise ratio (SNR) for the predicted mean of 50% correct words in a words-in-noise test is about 25 dB, wherein the SNR is calculated using the Spearman-Kärber equation.
252. The Wnt agonist and/or the epigenetic modulator for use according to embodiment 250, wherein the patient's Signal-to-Noise ratio (SNR) for the predicted mean of 50% correct words in a words-in-noise test is about 24 dB, wherein the SNR is calculated using the Spearman-Kärber equation.
253. The Wnt agonist and/or the epigenetic modulator for use according to embodiment 250, wherein the patient's Signal-to-Noise ratio (SNR) for the predicted mean of 50% correct words in a words-in-noise test is about 23 dB, wherein the SNR is calculated using the Spearman-Kärber equation.
254. The Wnt agonist and/or the epigenetic modulator for use according to embodiment 250, wherein the patient's Signal-to-Noise ratio (SNR) for the predicted mean of 50% correct words in a words-in-noise test is about 22 dB, wherein the SNR is calculated using the Spearman-Kärber equation.
255. The Wnt agonist and/or the epigenetic modulator for use according to embodiment 250, wherein the patient's Signal-to-Noise ratio (SNR) for the predicted mean of 50% correct words in a words-in-noise test is about 21 dB, wherein the SNR is calculated using the Spearman-Kärber equation.
256. The Wnt agonist and/or the epigenetic modulator for use according to embodiment 250, wherein the patient's Signal-to-Noise ratio (SNR) for the predicted mean of 50% correct words in a words-in-noise test is about 20 dB, wherein the SNR is calculated using the Spearman-Kärber equation.
257. The Wnt agonist and/or the epigenetic modulator for use according to embodiment 250, wherein the patient's Signal-to-Noise ratio (SNR) for the predicted mean of 50% correct words in a words-in-noise test is about 19 dB, wherein the SNR is calculated using the Spearman-Kärber equation.
258. The Wnt agonist and/or the epigenetic modulator for use according to embodiment 250, wherein the patient's Signal-to-Noise ratio (SNR) for the predicted mean of 50% correct words in a words-in-noise test is about 18 dB, wherein the SNR is calculated using the Spearman-Kärber equation.
259. The Wnt agonist and/or the epigenetic modulator for use according to embodiment 250, wherein the patient's Signal-to-Noise ratio (SNR) for the predicted mean of 50% correct words in a words-in-noise test is about 17 dB, wherein the SNR is calculated using the Spearman-Kärber equation.
260. The Wnt agonist and/or the epigenetic modulator for use according to embodiment 250, wherein the patient's Signal-to-Noise ratio (SNR) for the predicted mean of 50% correct words in a words-in-noise test is about 16 dB, wherein the SNR is calculated using the Spearman-Kärber equation.
261. The Wnt agonist and/or the epigenetic modulator for use according to embodiment 250, wherein the patient's Signal-to-Noise ratio (SNR) for the predicted mean of 50% correct words in a words-in-noise test is about 15 dB, wherein the SNR is calculated using the Spearman-Kärber equation.
262. The Wnt agonist and/or the epigenetic modulator for use according to embodiment 250, wherein the patient's Signal-to-Noise ratio (SNR) for the predicted mean of 50% correct words in a words-in-noise test is about 14 dB, wherein the SNR is calculated using the Spearman-Kärber equation.
263. The Wnt agonist and/or the epigenetic modulator for use according to embodiment 250, wherein the patient's Signal-to-Noise ratio (SNR) for the predicted mean of 50% correct words in a words-in-noise test is about 13 dB, wherein the SNR is calculated using the Spearman-Kärber equation.
264. The Wnt agonist and/or the epigenetic modulator for use according to embodiment, 209 wherein the patient's Signal-to-Noise ratio (SNR) for the predicted mean of 50% correct words in a words-in-noise test is about 12 dB, wherein the SNR is calculated using the Spearman-Kärber equation.
265. The Wnt agonist and/or the epigenetic modulator for use according to embodiment 250, wherein the patient's Signal-to-Noise ratio (SNR) for the predicted mean of 50% correct words in a words-in-noise test is about 11 dB, wherein the SNR is calculated using the Spearman-Kärber equation.
266. The Wnt agonist and/or the epigenetic modulator for use according to embodiment 250, wherein the patient's Signal-to-Noise ratio (SNR) for the predicted mean of 50% correct words in a words-in-noise test is about 10 dB, wherein the SNR is calculated using the Spearman-Kärber equation.
267. The Wnt agonist and/or the epigenetic modulator for use according to embodiment 250, wherein the patient's Signal-to-Noise ratio (SNR) for the predicted mean of 50% correct words in a words-in-noise test is about 9 dB, wherein the SNR is calculated using the Spearman-Kärber equation.
268. The Wnt agonist and/or the epigenetic modulator for use according to embodiment 250, wherein the patient's Signal-to-Noise ratio (SNR) for the predicted mean of 50% correct words in a words-in-noise test is about 8 dB, wherein the SNR is calculated using the Spearman-Kärber equation.
269. The Wnt agonist and/or the epigenetic modulator for use according to embodiment 250, wherein the patient's Signal-to-Noise ratio (SNR) for the predicted mean of 50% correct words in a words-in-noise test is about 7 dB, wherein the SNR is calculated using the Spearman-Kärber equation.
270. The Wnt agonist and/or the epigenetic modulator for use according to embodiment 250, wherein the patient's Signal-to-Noise ratio (SNR) for the predicted mean of 50% correct words in a words-in-noise test is about 6 dB, wherein the SNR is calculated using the Spearman-Kärber equation.
271. The Wnt agonist for use according to any of embodiments 157-270, wherein the Wnt agonist is separate from the epigenetic modulator.
272. The epigenetic modulator for use according to any of embodiments 157-270, wherein the epigenetic modulator is separate from the Wnt agonist.
273. The Wnt agonist and the epigenetic modulator for use according to any of embodiments 157-270, wherein the Wnt agonist and the epigenetic modulator are in combination.
274. The Wnt agonist and/or the epigenetic modulator for use according to any of embodiments 157-273, wherein the Wnt agonist is a GSK3 inhibitor or a pharmaceutically acceptable salt thereof.
275. The Wnt agonist and/or the epigenetic modulator for use according to any of embodiments 157-273, wherein the Wnt agonist is a GSK3-α inhibitor or a pharmaceutically acceptable salt thereof.
276. The Wnt agonist and/or the epigenetic modulator for use according to any of embodiments 157-273, wherein the Wnt agonist is a GSK3-β inhibitor or a pharmaceutically acceptable salt thereof.
277. The Wnt agonist and/or the epigenetic modulator for use according to any of embodiments 274, wherein the GSK3 inhibitor is AZD1080 or a pharmaceutically acceptable salt thereof.
278. The Wnt agonist and/or the epigenetic modulator for use according to any of embodiments 274, wherein the GSK3 inhibitor is LY2090314 or a pharmaceutically acceptable salt thereof.
279. The Wnt agonist and/or the epigenetic modulator for use according to any of embodiments 274, wherein the GSK3 inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione or a pharmaceutically acceptable salt thereof.
280. The Wnt agonist and/or the epigenetic modulator for use according to any of embodiments 274, wherein the GSK3 inhibitor is GSK3 inhibitor XXII or a pharmaceutically acceptable salt thereof
281. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(imidazo[1,2-a]pyridin-3-yl)-4-(2-(piperidine-1-carbonyl)-9-(trifluoromethyl)-1,2,3,4-tetrahydro-[1,4]diazepino[6,7,1-hi]indol-7-yl)-1H-pyrrole-2,5-dione or a pharmaceutically acceptable salt thereof.
282. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 7-(4-(imidazo[1,2-a]pyridin-3-yl)-2,5-dioxo-2,5-dihydro-1H-pyrrol-3-yl)-2-(piperidine-1-carbonyl)-1,2,3,4-tetrahydro-[1,4]diazepino[6,7,1-hi]indole-9-carbonitrile or a pharmaceutically acceptable salt thereof
283. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-ethynyl-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
284. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-amino-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
285. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 1-(9-fluoro-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-2-carbonyl)piperidine-4-carbaldehyde or a pharmaceutically acceptable salt thereof.
286. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(4-(hydroxymethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
287. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4,4-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
288. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(8-oxa-3-azabicyclo[3.2.1]octane-3-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
289. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(benzo[d]isoxazol-3-yl)-4-(9-fluoro-2-(piperidine-1-carbonyl)-1,2,3,4-tetrahydro-[1,4]diazepino[6,7,1-hi]indol-7-yl)-1H-pyrrole-2,5-dione or a pharmaceutically acceptable salt thereof.
290. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is N-(7-(4-(imidazo[1,2-a]pyridin-3-yl)-2,5-dioxo-2,5-dihydro-1H-pyrrol-3-yl)-2-(piperidine-1-carbonyl)-1,2,3,4-tetrahydro-[1,4]diazepino[6,7,1-hi]indol-9-yl)acetamide or a pharmaceutically acceptable salt thereof.
291. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-(difluoromethyl)-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
292. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(3,3-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
293. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-((1R,4R)-2,5-diazabicyclo[2.2.1]heptane-2-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
294. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 2-(8-oxa-3-azabicyclo[3.2.1]octane-3-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile or a pharmaceutically acceptable salt thereof.
295. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 2-(3,3-difluoropiperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile or a pharmaceutically acceptable salt thereof.
296. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 2-(4,4-difluoropiperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile or a pharmaceutically acceptable salt thereof.
297. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4,4-difluoropiperidine-1-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
298. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(8-oxa-3-azabicyclo[3.2.1]octane-3-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
299. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4-(aminomethyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
300. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4-(hydroxymethyl)piperidine-1-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
301. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 2-(4-(hydroxymethyl)piperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile or a pharmaceutically acceptable salt thereof.
302. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(3,3,4,4,5,5-hexafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
303. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(3,3,5,5-tetrafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
304. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(2,2,6,6-tetrafluoromorpholine-4-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
305. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4,4-difluoro-3-hydroxypiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
306. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4-(difluoro(hydroxy)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
307. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(6,6-difluoro-1,4-oxazepane-4-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
308. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-([1,2,4]triazolo[4,3-a]pyridin-3-yl)-4-(9-fluoro-2-(piperidine-1-carbonyl)-1,2,3,4-tetrahydro-[1,4]diazepino[6,7,1-hi]indol-7-yl)-1H-pyrrole-2,5-dione or a pharmaceutically acceptable salt thereof.
309. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(piperidine-1-carbonyl-d10)-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
310. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(piperidine-1-carbonyl)-1,2,3,4-tetrahydro-[1,4]diazepino[6,7,1-hi]indol-7-yl-3,3,4,4-d4)-4-(imidazo[1,2-a]pyridin-3-yl)-1H-pyrrole-2,5-dione or a pharmaceutically acceptable salt thereof
311. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(4-(2,2,2-trifluoro-1-hydroxyethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
312. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(4-((methylamino)methyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
313. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4-((dimethylamino)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
314. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4-aminopiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
315. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(4-(methylamino)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
316. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4-(dimethylamino)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
317. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 9-fluoro-7-(4-(imidazo[1,2-a]pyridin-3-yl)-2,5-dioxo-2,5-dihydro-1H-pyrrol-3-yl)-N-(piperidin-4-ylmethyl)-3,4-dihydro-[1,4]diazepino[6,7,1-hi]indole-2(1H)-carboxamide or a pharmaceutically acceptable salt thereof.
318. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 9-fluoro-7-(4-(imidazo[1,2-a]pyridin-3-yl)-2,5-dioxo-2,5-dihydro-1H-pyrrol-3-yl)-N-methyl-N-(piperidin-4-ylmethyl)-3,4-dihydro-[1,4]diazepino[6,7,1-hi]indole-2(1H)-carboxamide or a pharmaceutically acceptable salt thereof.
319. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 9-fluoro-7-(4-(imidazo[1,2-a]pyridin-3-yl)-2,5-dioxo-2,5-dihydro-1H-pyrrol-3-yl)-N-methyl-N-((1-methylpiperidin-4-yl)methyl)-3,4-dihydro-[1,4]diazepino[6,7,1-hi]indole-2(1H)-carboxamide or a pharmaceutically acceptable salt thereof.
320. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-((1R,4R)-5-methyl-2,5-diazabicyclo[2.2.1]heptane-2-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
321. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(2-methyl-2,8-diazaspiro[4.5]decane-8-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
322. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(8-methyl-2,8-diazaspiro[4.5]decane-2-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
323. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(imidazo[1,2-a]pyridin-3-yl)-4-(2-(2,2,6,6-tetrafluoromorpholine-4-carbonyl)-9-(trifluoromethyl)-1,2,3,4-tetrahydro-[1,4]diazepino[6,7,1-hi]indol-7-yl)-1H-pyrrole-2,5-dione or a pharmaceutically acceptable salt thereof.
324. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(6,6-difluoro-1,4-oxazepane-4-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione or a pharmaceutically acceptable salt thereof.
325. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 2-(4-(dimethylamino)piperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile or a pharmaceutically acceptable salt thereof.
326. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 9-cyano-7-(4-(imidazo[1,2-a]pyridin-3-yl)-2,5-dioxo-2,5-dihydro-1H-pyrrol-3-yl)-N-methyl-N-((1-methylpiperidin-4-yl)methyl)-3,4-dihydro-[1,4]diazepino[6,7,1-hi]indole-2(1H)-carboxamide or a pharmaceutically acceptable salt thereof.
327. The Wnt agonist and/or the epigenetic modulator for use according to embodiment 279, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 7-(4-(imidazo[1,2-a]pyridin-3-yl)-2,5-dioxo-2,5-dihydro-1H-pyrrol-3-yl)-2-(8-methyl-2,8-diazaspiro[4.5]decane-2-carbonyl)-1,2,3,4-tetrahydro-[1,4]diazepino[6,7,1-hi]indole-9-carbonitrile or a pharmaceutically acceptable salt thereof.
328. The Wnt agonist and/or the epigenetic modulator for use according to any of embodiments 157-273, wherein the Wnt agonist is an analogue of LY2090314 as known in the art.
329. The Wnt agonist and/or the epigenetic modulator for use according to any of embodiments 157-273, wherein the Wnt agonist is Wnt-1 or a pharmaceutically acceptable salt thereof.
330. The Wnt agonist and/or the epigenetic modulator for use according to any of embodiments 157-273, wherein the Wnt agonist is Wnt-Z/Irp or a pharmaceutically acceptable salt thereof.
331. The Wnt agonist and/or the epigenetic modulator for use according to any of embodiments 157-273, wherein the Wnt agonist is Wnt-2b/13 or a pharmaceutically acceptable salt thereof.
332. The Wnt agonist and/or the epigenetic modulator for use according to any of embodiments 157-273, wherein the Wnt agonist is Wnt-3/Int-4 or a pharmaceutically acceptable salt thereof.
333. The Wnt agonist and/or the epigenetic modulator for use according to any of embodiments 157-273, wherein the Wnt agonist is Wnt-3a or a pharmaceutically acceptable salt thereof.
334. The Wnt agonist and/or the epigenetic modulator for use according to any of embodiments 157-273, wherein the Wnt agonist is Wnt-4 or a pharmaceutically acceptable salt thereof.
335. The Wnt agonist and/or the epigenetic modulator for use according to any of embodiments 157-273, wherein the Wnt agonist is Wnt-5a or a pharmaceutically acceptable salt thereof.
336. The Wnt agonist and/or the epigenetic modulator for use according to any of embodiments 157-273, wherein the Wnt agonist is Wnt-5b or a pharmaceutically acceptable salt thereof.
337. The Wnt agonist and/or the epigenetic modulator for use according to any of embodiments 157-273, wherein the Wnt agonist is Wnt-6 or a pharmaceutically acceptable salt thereof.
338. The Wnt agonist and/or the epigenetic modulator for use according to any of embodiments 157-273, wherein the Wnt agonist is Wnt-7a or a pharmaceutically acceptable salt thereof.
339. The Wnt agonist and/or the epigenetic modulator for use according to any of embodiments 157-273, wherein the Wnt agonist is Wnt-7b or a pharmaceutically acceptable salt thereof.
340. The Wnt agonist and/or the epigenetic modulator for use according to any of embodiments 157-273, wherein the Wnt agonist is Wnt-8a/8d or a pharmaceutically acceptable salt thereof.
341. The Wnt agonist and/or the epigenetic modulator for use according to any of embodiments 157-273, wherein the Wnt agonist is Wnt-8b or a pharmaceutically acceptable salt thereof.
342. The Wnt agonist and/or the epigenetic modulator for use according to any of embodiments 157-273, wherein the Wnt agonist is Wnt-9a/14 or a pharmaceutically acceptable salt thereof
343. The Wnt agonist and/or the epigenetic modulator for use according to any of embodiments 157-273, wherein the Wnt agonist is Wnt-9b/14b/15 or a pharmaceutically acceptable salt thereof.
344. The Wnt agonist and/or the epigenetic modulator for use according to any of embodiments 157-273, wherein the Wnt agonist is Wnt-10a or a pharmaceutically acceptable salt thereof.
345. The Wnt agonist and/or the epigenetic modulator for use according to any of embodiments 157-273, wherein the Wnt agonist is Wnt-10b/12 or a pharmaceutically acceptable salt thereof.
346. The Wnt agonist and/or the epigenetic modulator for use according to any of embodiments 157-273, wherein the Wnt agonist is Wnt-11 or a pharmaceutically acceptable salt thereof.
347. The Wnt agonist and/or the epigenetic modulator for use according to any of embodiments 157-273, wherein the Wnt agonist is Wnt-16 or a pharmaceutically acceptable salt thereof.
348. The Wnt agonist and/or the epigenetic modulator for use according to any of embodiments 157-273, wherein the Wnt agonist is R-Spondin 1/2/3/4 or a pharmaceutically acceptable salt thereof
349. The Wnt agonist and/or the epigenetic modulator for use according to any of embodiments 157-273, wherein the Wnt agonist is Norrin or a pharmaceutically acceptable salt thereof.
350. The Wnt agonist and/or the epigenetic modulator for use according to any of embodiments 157-273, wherein the Wnt agonist is BML-284 or a pharmaceutically acceptable salt thereof.
351. The Wnt agonist and/or the epigenetic modulator for use according to any of embodiments 157-273, wherein the Wnt agonist is IQ 1 or a pharmaceutically acceptable salt thereof.
352. The Wnt agonist and/or the epigenetic modulator for use according to any of embodiments 157-273, wherein the Wnt agonist is DCA or a pharmaceutically acceptable salt thereof.
353. The Wnt agonist and/or the epigenetic modulator for use according to any of embodiments 157-273, wherein the Wnt agonist is QS 11 or a pharmaceutically acceptable salt thereof.
354. The Wnt agonist and/or the epigenetic modulator for use according to any of embodiments 157-273, wherein the Wnt agonist is WASP-1 or a pharmaceutically acceptable salt thereof.
355. The Wnt agonist and/or the epigenetic modulator for use according to any of embodiments 157-273, wherein the Wnt agonist is WAY 316606 or a pharmaceutically acceptable salt thereof.
356. The Wnt agonist and/or the epigenetic modulator for use according to any of embodiments 157-273, wherein the Wnt agonist is (Dimethylamino)propyl)-2-ethyl-5-(phenylsulfonyl)benzenesulfonamide or a pharmaceutically acceptable salt thereof.
357. The Wnt agonist and/or the epigenetic modulator for use according to any of embodiments 157-273, wherein the Wnt agonist is Cyclosporine A (CsA) or a pharmaceutically acceptable salt thereof.
358. The Wnt agonist and/or the epigenetic modulator for use according to any of embodiments 157-273, wherein the Wnt agonist is PSC833 (Valspodar) or a pharmaceutically acceptable salt thereof.
359. The Wnt agonist and/or the epigenetic modulator for use according to any of embodiments 157-273, wherein the Wnt agonist is a Cyclosporine analog.
360. The Wnt agonist and/or the epigenetic modulator for use according to any of embodiments 157-273, wherein the Wnt agonist is WAY-262611 or a pharmaceutically acceptable salt thereof.
361. The Wnt agonist and/or the epigenetic modulator for use according to any of embodiments 157-273, wherein the Wnt agonist is HLY78 or a pharmaceutically acceptable salt thereof.
362. The Wnt agonist and/or the epigenetic modulator for use according to any of embodiments 157-273, wherein the Wnt agonist is SKL2001 or a pharmaceutically acceptable salt thereof.
363. The Wnt agonist and/or the epigenetic modulator for use according to any of embodiments 157-273, wherein the Wnt agonist is Cpd1 or a pharmaceutically acceptable salt thereof.
364. The Wnt agonist and/or the epigenetic modulator for use according to any of embodiments 157-273, wherein the Wnt agonist is Cpd2 or a pharmaceutically acceptable salt thereof.
365. The Wnt agonist and/or the epigenetic modulator for use according to any of embodiments 157-273, wherein the Wnt agonist is ISX 9 or a pharmaceutically acceptable salt thereof.
366. The Wnt agonist and/or the epigenetic modulator for use according to any of embodiments 157-273, wherein the Wnt agonist is Selumetinib or a pharmaceutically acceptable salt thereof.
367. The Wnt agonist and/or the epigenetic modulator for use according to any of embodiments 157-273, wherein the Wnt agonist is Radicicol or a pharmaceutically acceptable salt thereof.
368. The Wnt agonist and/or the epigenetic modulator for use according to any of embodiments 157-273, wherein the Wnt agonist is AT 7519 or a pharmaceutically acceptable salt thereof.
369. The Wnt agonist and/or the epigenetic modulator for use according to any of embodiments 157-273, wherein the Wnt agonist is AZD1080 or a pharmaceutically acceptable salt thereof.
370. The Wnt agonist and/or the epigenetic modulator for use according to any of embodiments 157-273, wherein the Wnt agonist is Tivantinib or a pharmaceutically acceptable salt thereof.
371. The Wnt agonist and/or the epigenetic modulator for use according to any of embodiments 157-273, wherein the Wnt agonist is I5 or a pharmaceutically acceptable salt thereof.
372. The Wnt agonist and/or the epigenetic modulator for use according to any of embodiments 157-273, wherein the Wnt agonist is BRD4003 chiral or a pharmaceutically acceptable salt thereof.
373. The Wnt agonist and/or the epigenetic modulator for use according to any of embodiments 157-273, wherein the Wnt agonist is BRD1172 or a pharmaceutically acceptable salt thereof.
374. The Wnt agonist and/or the epigenetic modulator for use according to any of embodiments 157-273, wherein the Wnt agonist is BRD1652 or a pharmaceutically acceptable salt thereof.
375. The Wnt agonist and/or the epigenetic modulator for use according to any of embodiments 157-273, wherein the Wnt agonist is AR-A014418 or a pharmaceutically acceptable salt thereof.
376. The Wnt agonist and/or the epigenetic modulator for use according to any of embodiments 157-273, wherein the Wnt agonist is Bikinin or a pharmaceutically acceptable salt thereof.
377. The Wnt agonist and/or the epigenetic modulator for use according to any of embodiments 157-273, wherein the Wnt agonist is Hymenialdisine or a pharmaceutically acceptable salt thereof.
378. The Wnt agonist and/or the epigenetic modulator for use according to any of embodiments 157-273, wherein the Wnt agonist is Aloisine A or a pharmaceutically acceptable salt thereof.
379. The Wnt agonist and/or the epigenetic modulator for use according to any of embodiments 157-273, wherein the Wnt agonist is Aloisine B or a pharmaceutically acceptable salt thereof.
380. The Wnt agonist and/or the epigenetic modulator for use according to any of embodiments 157-273, wherein the Wnt agonist is TWS 119 or a pharmaceutically acceptable salt thereof.
381. The Wnt agonist and/or the epigenetic modulator for use according to any of embodiments 157-273, wherein the Wnt agonist is CT20026 or a pharmaceutically acceptable salt thereof.
382. The Wnt agonist and/or the epigenetic modulator for use according to any of embodiments 157-273, wherein the Wnt agonist is CHIR99021 or a pharmaceutically acceptable salt thereof.
383. The Wnt agonist and/or the epigenetic modulator for use according to any of embodiments 157-273, wherein the Wnt agonist is CHIR98014 or a pharmaceutically acceptable salt thereof.
384. The Wnt agonist and/or the epigenetic modulator for use according to any of embodiments 157-273, wherein the Wnt agonist is CHIR98023 or a pharmaceutically acceptable salt thereof.
385. The Wnt agonist and/or the epigenetic modulator for use according to any of embodiments 157-273, wherein the Wnt agonist is CHIR98024 or a pharmaceutically acceptable salt thereof
386. The Wnt agonist and/or the epigenetic modulator for use according to any of embodiments 157-273, wherein the Wnt agonist is CGP60474 or a pharmaceutically acceptable salt thereof.
387. The Wnt agonist and/or the epigenetic modulator for use according to any of embodiments 157-273, wherein the Wnt agonist is AZD2858 (AR28) or a pharmaceutically acceptable salt thereof.
388. The Wnt agonist and/or the epigenetic modulator for use according to any of embodiments 157-273, wherein the Wnt agonist is CID 755673 or a pharmaceutically acceptable salt thereof.
389. The Wnt agonist and/or the epigenetic modulator for use according to any of embodiments 157-273, wherein the Wnt agonist is TCS 2002 or a pharmaceutically acceptable salt thereof.
390. The Wnt agonist and/or the epigenetic modulator for use according to any of embodiments 157-273, wherein the Wnt agonist is Dibromocantharelline or a pharmaceutically acceptable salt thereof.
391. The Wnt agonist and/or the epigenetic modulator for use according to any of embodiments 157-273, wherein the Wnt agonist is ML320 or a pharmaceutically acceptable salt thereof.
392. The Wnt agonist and/or the epigenetic modulator for use according to any of embodiments 157-273, wherein the Wnt agonist is Flavopiridol or a pharmaceutically acceptable salt thereof.
393. The Wnt agonist and/or the epigenetic modulator for use according to any of embodiments 157-273, wherein the Wnt agonist is Hymenidin or a pharmaceutically acceptable salt thereof.
394. The Wnt agonist and/or the epigenetic modulator for use according to any of embodiments 157-273, wherein the Wnt agonist is 6-Bromoindirubin-3-acetoxime or a pharmaceutically acceptable salt thereof.
395. The Wnt agonist and/or the epigenetic modulator for use according to any of embodiments 157-273, wherein the Wnt agonist is Indirubin-3′-monoxime or a pharmaceutically acceptable salt thereof.
396. The Wnt agonist and/or the epigenetic modulator for use according to any of embodiments 157-273, wherein the Wnt agonist is 5-Iodo-indirubin-3′-monoxime or a pharmaceutically acceptable salt thereof.
397. The Wnt agonist and/or the epigenetic modulator for use according to any of embodiments 157-273, wherein the Wnt agonist is Indirubin-5-sulfonic acid sodium salt or a pharmaceutically acceptable salt thereof.
398. The Wnt agonist and/or the epigenetic modulator for use according to any of embodiments 157-273, wherein the Wnt agonist is Indirubin or a pharmaceutically acceptable salt thereof.
399. The Wnt agonist and/or the epigenetic modulator for use according to any of embodiments 157-273, wherein the Wnt agonist is Lithium Chloride or a pharmaceutically acceptable salt thereof.
400. The Wnt agonist and/or the epigenetic modulator for use according to any of embodiments 157-273, wherein the Wnt agonist is Beryllium or a pharmaceutically acceptable salt thereof.
401. The Wnt agonist and/or the epigenetic modulator for use according to any of embodiments 157-273, wherein the Wnt agonist is Zinc or a pharmaceutically acceptable salt thereof.
402. The Wnt agonist and/or the epigenetic modulator for use according to any of embodiments 157-273, wherein the Wnt agonist is Tungstate or a pharmaceutically acceptable salt thereof.
403. The Wnt agonist and/or the epigenetic modulator for use according to any of embodiments 157-273, wherein the Wnt agonist is GF109203x or a pharmaceutically acceptable salt thereof.
404. The Wnt agonist and/or the epigenetic modulator for use according to any of embodiments 157-273, wherein the Wnt agonist is Ro318220 or a pharmaceutically acceptable salt thereof.
405. The Wnt agonist and/or the epigenetic modulator for use according to any of embodiments 157-273, wherein the Wnt agonist is Bisindolylmaleimide X HCl or a pharmaceutically acceptable salt thereof.
406. The Wnt agonist and/or the epigenetic modulator for use according to any of embodiments 157-273, wherein the Wnt agonist is Enzastaurin or a pharmaceutically acceptable salt thereof
407. The Wnt agonist and/or the epigenetic modulator for use according to any of embodiments 157-273, wherein the Wnt agonist is SB-216763 or a pharmaceutically acceptable salt thereof.
408. The Wnt agonist and/or the epigenetic modulator for use according to any of embodiments 157-273, wherein the Wnt agonist is SB-415286 or a pharmaceutically acceptable salt thereof.
409. The Wnt agonist and/or the epigenetic modulator for use according to any of embodiments 157-273, wherein the Wnt agonist is 3F8 or a pharmaceutically acceptable salt thereof.
410. The Wnt agonist and/or the epigenetic modulator for use according to any of embodiments 157-273, wherein the Wnt agonist is TCS 21311 or a pharmaceutically acceptable salt thereof.
411. The Wnt agonist and/or the epigenetic modulator for use according to any of embodiments 157-273, wherein the Wnt agonist is LY2090314 or a pharmaceutically acceptable salt thereof.
412. The Wnt agonist and/or the epigenetic modulator for use according to any of embodiments 157-273, wherein the Wnt agonist is IM-12 or a pharmaceutically acceptable salt thereof.
413. The Wnt agonist and/or the epigenetic modulator for use according to any of embodiments 157-273, wherein the Wnt agonist is KT 5720 or a pharmaceutically acceptable salt thereof.
414. The Wnt agonist and/or the epigenetic modulator for use according to any of embodiments 157-273, wherein the Wnt agonist is Isogranulatimide or a pharmaceutically acceptable salt thereof.
415. The Wnt agonist and/or the epigenetic modulator for use according to any of embodiments 157-273, wherein the Wnt agonist is BIP-135 or a pharmaceutically acceptable salt thereof.
416. The Wnt agonist and/or the epigenetic modulator for use according to any of embodiments 157-273, wherein the Wnt agonist is CP21R7 or a pharmaceutically acceptable salt thereof.
417. The Wnt agonist and/or the epigenetic modulator for use according to any of embodiments 157-273, wherein the Wnt agonist is HB12 or a pharmaceutically acceptable salt thereof.
418. The Wnt agonist and/or the epigenetic modulator for use according to any of embodiments 157-273, wherein the Wnt agonist is DW12 or a pharmaceutically acceptable salt thereof.
419. The Wnt agonist and/or the epigenetic modulator for use according to any of embodiments 157-273, wherein the Wnt agonist is NP309 or a pharmaceutically acceptable salt thereof.
420. The Wnt agonist and/or the epigenetic modulator for use according to any of embodiments 157-273, wherein the Wnt agonist is (RRu)-HB1229 or a pharmaceutically acceptable salt thereof.
421. The Wnt agonist and/or the epigenetic modulator for use according to any of embodiments 157-273, wherein the Wnt agonist is (RRu)-NP549 or a pharmaceutically acceptable salt thereof.
422. The Wnt agonist and/or the epigenetic modulator for use according to any of embodiments 157-273, wherein the Wnt agonist is Staurosporine or a pharmaceutically acceptable salt thereof.
423. The Wnt agonist and/or the epigenetic modulator for use according to any of embodiments 157-273, wherein the Wnt agonist is Manzamine A or a pharmaceutically acceptable salt thereof.
424. The Wnt agonist and/or the epigenetic modulator for use according to any of embodiments 157-273, wherein the Wnt agonist is TC-G 24 or a pharmaceutically acceptable salt thereof
425. The Wnt agonist and/or the epigenetic modulator for use according to any of embodiments 157-273, wherein the Wnt agonist is SU9516 or a pharmaceutically acceptable salt thereof.
426. The Wnt agonist and/or the epigenetic modulator for use according to any of embodiments 157-273, wherein the Wnt agonist is AZD1080 or a pharmaceutically acceptable salt thereof
427. The Wnt agonist and/or the epigenetic modulator for use according to any of embodiments 157-273, wherein the Wnt agonist is Kenpaullone or a pharmaceutically acceptable salt thereof
428. The Wnt agonist and/or the epigenetic modulator for use according to any of embodiments 157-273, wherein the Wnt agonist is Cmpd 17b or a pharmaceutically acceptable salt thereof.
429. The Wnt agonist and/or the epigenetic modulator for use according to any of embodiments 157-273, wherein the Wnt agonist is Azakenpaullone or a pharmaceutically acceptable salt thereof.
430. The Wnt agonist and/or the epigenetic modulator for use according to any of embodiments 157-273, wherein the Wnt agonist is Alsterpaullone or a pharmaceutically acceptable salt thereof.
431. The Wnt agonist and/or the epigenetic modulator for use according to any of embodiments 157-273, wherein the Wnt agonist is Alsterpaullone CN Ethyl or a pharmaceutically acceptable salt thereof
432. The Wnt agonist and/or the epigenetic modulator for use according to any of embodiments 157-273, wherein the Wnt agonist is Cazpaullone or a pharmaceutically acceptable salt thereof.
433. The Wnt agonist and/or the epigenetic modulator for use according to any of embodiments 157-273, wherein the Wnt agonist is FRATtide or a pharmaceutically acceptable salt thereof.
434. The Wnt agonist and/or the epigenetic modulator for use according to any of embodiments 157-273, wherein the Wnt agonist is L803 or a pharmaceutically acceptable salt thereof.
435. The Wnt agonist and/or the epigenetic modulator for use according to any of embodiments 157-273, wherein the Wnt agonist is L803-mts or a pharmaceutically acceptable salt thereof.
436. The Wnt agonist and/or the epigenetic modulator for use according to any of embodiments 157-273, wherein the Wnt agonist is AT 7519 or a pharmaceutically acceptable salt thereof.
437. The Wnt agonist and/or the epigenetic modulator for use according to any of embodiments 157-273, wherein the Wnt agonist is NSC 693868 or a pharmaceutically acceptable salt thereof.
438. The Wnt agonist and/or the epigenetic modulator for use according to any of embodiments 157-273, wherein the Wnt agonist is VP0 or a pharmaceutically acceptable salt thereof. 7 or a pharmaceutically acceptable salt thereof.
439. The Wnt agonist and/or the epigenetic modulator for use according to any of embodiments 157-273, wherein the Wnt agonist is Palinurin or a pharmaceutically acceptable salt thereof.
440. The Wnt agonist and/or the epigenetic modulator for use according to any of embodiments 157-273, wherein the Wnt agonist is Tricantin or a pharmaceutically acceptable salt thereof.
441. The Wnt agonist and/or the epigenetic modulator for use according to any of embodiments 157-273, wherein the Wnt agonist is NP031115 or a pharmaceutically acceptable salt thereof.
442. The Wnt agonist and/or the epigenetic modulator for use according to any of embodiments 157-273, wherein the Wnt agonist is NP031112 (Tideglusib) or a pharmaceutically acceptable salt thereof.
443. The Wnt agonist and/or the epigenetic modulator for use according to any of embodiments 157-273, wherein the Wnt agonist is AR-A014418 or a pharmaceutically acceptable salt thereof.
444. The Wnt agonist and/or the epigenetic modulator for use according to any of embodiments 157-273, wherein the Wnt agonist is A-1070722 or a pharmaceutically acceptable salt thereof.
445. The Wnt agonist and/or the epigenetic modulator for use according to any of embodiments 157-273, wherein the Wnt agonist is Compound (R)-DW12 or a pharmaceutically acceptable salt thereof.
446. The Wnt agonist and/or the epigenetic modulator for use according to any of embodiments 157-273, wherein the Wnt agonist is Compound lambda-OS1 or a pharmaceutically acceptable salt thereof.
447. The Wnt agonist and/or the epigenetic modulator for use according to any of embodiments 157-273, wherein the Wnt agonist is GSK-3b XXII or a pharmaceutically acceptable salt thereof.
448. The Wnt agonist and/or the epigenetic modulator for use according to any of embodiments 157-273, wherein the Wnt agonist is GSK-3β Inhibitor XVIII or a pharmaceutically acceptable salt thereof.
449. The Wnt agonist and/or the epigenetic modulator for use according to any of embodiments 157-273, wherein the Wnt agonist is GSK-3β Inhibitor XVI or a pharmaceutically acceptable salt thereof.
450. The Wnt agonist and/or the epigenetic modulator for use according to any of embodiments 157-273, wherein the Wnt agonist is GSK-3β Inhibitor XVII or a pharmaceutically acceptable salt thereof.
451. The Wnt agonist and/or the epigenetic modulator for use according to any of embodiments 157-273, wherein the Wnt agonist is GSK-3 Inhibitor IX or a pharmaceutically acceptable salt thereof.
452. The Wnt agonist and/or the epigenetic modulator for use according to any of embodiments 157-273, wherein the Wnt agonist is GSK-3 Inhibitor X or a pharmaceutically acceptable salt thereof.
453. The Wnt agonist and/or the epigenetic modulator for use according to any of embodiments 157-273, wherein the Wnt agonist is GSK-3 Inhibitor 1 or a pharmaceutically acceptable salt thereof.
454. The Wnt agonist and/or the epigenetic modulator for use according to any of embodiments 157-273, wherein the Wnt agonist is GSK-3β Inhibitor XI or a pharmaceutically acceptable salt thereof.
455. The Wnt agonist and/or the epigenetic modulator for use according to any of embodiments 157-273, wherein the Wnt agonist is (RRu)-HB1229 or a pharmaceutically acceptable salt thereof
456. The Wnt agonist and/or the epigenetic modulator for use according to any of embodiments 157-273, wherein the Wnt agonist is (RRu)-NP549 or a pharmaceutically acceptable salt thereof.
457. The Wnt agonist and/or the epigenetic modulator for use according to any of embodiments 157-273, wherein the Wnt agonist is NP309 or a pharmaceutically acceptable salt thereof
458. The Wnt agonist and/or the epigenetic modulator for use according to any of embodiments 157-273, wherein the Wnt agonist is GSK-3 Inhibitor II or a pharmaceutically acceptable salt thereof.
459. The Wnt agonist and/or the epigenetic modulator for use according to any of embodiments 157-273, wherein the Wnt agonist is GSK-3 Inhibitor XIII or a pharmaceutically acceptable salt thereof.
460. The Wnt agonist and/or the epigenetic modulator for use according to any of embodiments 157-273, wherein the Wnt agonist is GSK-36 Inhibitor I or a pharmaceutically acceptable salt thereof.
461. The Wnt agonist and/or the epigenetic modulator for use according to any of embodiments 157-460, wherein the agent having activity as an epigenetic modulator is an LSD1 inhibitor or a pharmaceutically acceptable salt thereof.
462. The Wnt agonist and/or the epigenetic modulator for use according to any of embodiments 157-460, wherein the agent having activity as an epigenetic modulator is an HDAC inhibitor or a pharmaceutically acceptable salt thereof.
463. The Wnt agonist and/or the epigenetic modulator for use according to any of embodiments 157460, wherein the agent having activity as an epigenetic modulator is an EZH2 inhibitor or a pharmaceutically acceptable salt thereof.
464. The Wnt agonist and/or the epigenetic modulator for use according to any of embodiments 157460, wherein the agent having activity as an epigenetic modulator is a DOT1L inhibitor or a pharmaceutically acceptable salt thereof.
465. The Wnt agonist and/or the epigenetic modulator for use according to any of embodiments 157460, wherein the agent having activity as an epigenetic modulator is a KDM inhibitor or a pharmaceutically acceptable salt thereof.
466. A Wnt agonist and/or a TAZ activator or a pharmaceutically acceptable salt thereof for use in treating sensorineural hearing loss in a human patient as defined in any of embodiments 157-270, wherein said Wnt agonist and said TAZ activator are both administered to the patient.
467. The Wnt agonist and/or the epigenetic modulator for use according to any of embodiments 157-460, wherein the agent having activity as an epigenetic modulator is Valproic acid (VPA) or a pharmaceutically acceptable salt thereof, for example, sodium valproate.
468. The Wnt agonist and/or the epigenetic modulator or TAZ activator for use according to any of embodiments 157-467, wherein a second epigenetic modulator is also administered.
469. The Wnt agonist and/or the epigenetic modulator for use according to embodiment 461, wherein the LSD1 inhibitor is GSK2879552 or a pharmaceutically acceptable salt thereof
470. The Wnt agonist and/or the epigenetic modulator for use according to embodiment 461, wherein the LSD1 inhibitor is irreversible.
471. The Wnt agonist and/or the epigenetic modulator for use according to embodiment 461, wherein the LSD1 inhibitor is GSK-LSD1 or a pharmaceutically acceptable salt thereof.
472. The Wnt agonist and/or the epigenetic modulator for use according to embodiment 461, wherein the LSD1 inhibitor is Tranylcypromine (TCP) or a pharmaceutically acceptable salt thereof.
473. The Wnt agonist and/or the epigenetic modulator for use according to embodiment 461, wherein the LSD1 inhibitor is Phenelzine sulfate or a pharmaceutically acceptable salt thereof.
474. The Wnt agonist and/or the epigenetic modulator for use according to embodiment 461, wherein the agent having activity as an LSD1 inhibitor is ORY-2001 (Vafidemstat) or a pharmaceutically acceptable salt thereof.
475. The Wnt agonist and/or the epigenetic modulator for use according to embodiment 461, wherein the agent having activity as an LSD1 inhibitor is SP-2577 (Seclidemstat) or a pharmaceutically acceptable salt thereof.
476. The Wnt agonist and/or the epigenetic modulator for use according to embodiment 461, wherein the agent having activity as an LSD1 inhibitor is Osimertinib (AZD9291) or a pharmaceutically acceptable salt thereof.
477. The Wnt agonist and/or the epigenetic modulator for use according to embodiment 461, wherein the agent having activity as an LSD1 inhibitor is GCG-11047 (PG-11047) or a pharmaceutically acceptable salt thereof.
478. The Wnt agonist and/or the epigenetic modulator for use according to embodiment 461, wherein the agent having activity as an LSD1 inhibitor is ORY-1001 (RG6016, RO7051790, Iadademstat) or a pharmaceutically acceptable salt thereof.
479. The Wnt agonist and/or the epigenetic modulator for use according to embodiment 461, wherein the agent having activity as an LSD1 inhibitor is IMG-7289 or a pharmaceutically acceptable salt thereof.
480. The Wnt agonist and/or the epigenetic modulator for use according to embodiment 461, wherein the agent having activity as an LSD1 inhibitor is CC-90011 or a pharmaceutically acceptable salt thereof.
481. The Wnt agonist and/or the epigenetic modulator for use according to embodiment 461, wherein the agent having activity as an LSD1 inhibitor is INCB059872 or a pharmaceutically acceptable salt thereof.
482. The Wnt agonist and/or the epigenetic modulator for use according to embodiment 461, wherein the LSD-1 inhibitor is TCP trans chiral from J. American Chemical Society (2010), 132(19), 6827-6833 or a pharmaceutically acceptable salt thereof.
483. The Wnt agonist and/or the epigenetic modulator for use according to embodiment 461, wherein the LSD-1 inhibitor is TCP cis from Bioorganic Medicinal Chemistry 2008, 16(15), 7148-7166 or a pharmaceutically acceptable salt thereof.
484. The Wnt agonist and/or the epigenetic modulator for use according to embodiment 461, wherein the LSD-1 inhibitor is TCP cis chiral or a pharmaceutically acceptable salt thereof.
485. The Wnt agonist and/or the epigenetic modulator for use according to embodiment 461, wherein the LSD-1 inhibitor is RN-1 from Medicinal Research Reviews 2013, 33(4), 873-910, or a pharmaceutically acceptable salt thereof.
486. The Wnt agonist and/or the epigenetic modulator for use according to embodiment 461, wherein the LSD-1 inhibitor is compound 1 from PLoS One (2017), 12(2), e0170301, or a pharmaceutically acceptable salt thereof.
487. The Wnt agonist and/or the epigenetic modulator for use according to embodiment 461, wherein the LSD-1 inhibitor is compound 45 from Medicinal Research Reviews 2013, 33(4), 873-910, or a pharmaceutically acceptable salt thereof
488. The Wnt agonist and/or the epigenetic modulator for use according to embodiment 461, wherein the LSD-1 inhibitor is RN-7 from Epigenomics (2015), 7(8), 1379-1396, or a pharmaceutically acceptable salt thereof
489. The Wnt agonist and/or the epigenetic modulator for use according to embodiment 461, wherein the LSD-1 inhibitor is compound 5A from Future Med. Chem. (2017) 9(11), 1161-1174, or a pharmaceutically acceptable salt thereof
490. The Wnt agonist and/or the epigenetic modulator for use according to embodiment 461, wherein the LSD-1 inhibitor is compound 2 from Medicinal Research Reviews 2015, 35(3), 586-618, or a pharmaceutically acceptable salt thereof
491. The Wnt agonist and/or the epigenetic modulator for use according to embodiment 461, wherein the LSD-1 inhibitor is compound 12f from Med. Chem. Commun., 2015, 6, 665-670, or a pharmaceutically acceptable salt thereof.
492. The Wnt agonist and/or the epigenetic modulator for use according to embodiment 461, wherein the LSD-1 inhibitor is T-3775440 from Medicinal Research Reviews 2013, 33(4), 873-910, or a pharmaceutically acceptable salt thereof.
493. The Wnt agonist and/or the epigenetic modulator for use according to embodiment 461, wherein the LSD-1 inhibitor is OG-L002 or a pharmaceutically acceptable salt thereof.
494. The Wnt agonist and/or the epigenetic modulator for use according to embodiment 461, wherein the LSD-1 inhibitor is S2101 or a pharmaceutically acceptable salt thereof.
495. The Wnt agonist and/or the epigenetic modulator for use according to embodiment 461, wherein the LSD-1 inhibitor is NCL-1 or a pharmaceutically acceptable salt thereof.
496. The Wnt agonist and/or the epigenetic modulator for use according to embodiment 461, wherein the LSD-1 inhibitor is compound 9A from Bioorganic Medicinal Chemistry Letters 27 (2017) 2099-2101, or a pharmaceutically acceptable salt thereof.
497. The Wnt agonist and/or the epigenetic modulator for use according to embodiment 461, wherein the LSD-1 inhibitor is compound 191 from Chem. Pharm. Bull. 63, 882-889 (2015), or a pharmaceutically acceptable salt thereof.
498. The Wnt agonist and/or the epigenetic modulator for use according to embodiment 461, wherein the LSD-1 inhibitor is NCD-25 or a pharmaceutically acceptable salt thereof.
499. The Wnt agonist and/or the epigenetic modulator for use according to embodiment 461, wherein the LSD-1 inhibitor is NCD-38 or a pharmaceutically acceptable salt thereof.
500. The Wnt agonist and/or the epigenetic modulator for use according to embodiment 461, wherein the LSD-1 inhibitor is compound 14A from RSC Advances (2018), 8(3), 1666-1676, or a pharmaceutically acceptable salt thereof.
501. The Wnt agonist and/or the epigenetic modulator for use according to embodiment 461, wherein the LSD-1 inhibitor is compound 15A from RSC Advances (2018), 8(3), 1666-1676, or a pharmaceutically acceptable salt thereof.
502. The Wnt agonist and/or the epigenetic modulator for use according to embodiment 461, wherein the LSD-1 inhibitor is compound 15B from RSC Advances (2018), 8(3), 1666-1676, or a pharmaceutically acceptable salt thereof.
503. The Wnt agonist and/or the epigenetic modulator for use according to embodiment 461, wherein the LSD-1 inhibitor is compound 4 from Bioorganic Medicinal Chemistry Letters 28 (2018) 1001-1004, or a pharmaceutically acceptable salt thereof.
504. The Wnt agonist and/or the epigenetic modulator for use according to embodiment 461, wherein the LSD-1 inhibitor is pargyline or a pharmaceutically acceptable salt thereof.
505. The Wnt agonist and/or the epigenetic modulator for use according to embodiment 461, wherein the LSD-1 inhibitor is the peptide disclosed in Nature Structural & Molecular Biology, 2007, 14(6), 535, or a pharmaceutically acceptable salt thereof.
506. The Wnt agonist and/or the epigenetic modulator for use according to embodiment 461, wherein the LSD-1 inhibitor is bizine or a pharmaceutically acceptable salt thereof.
507. The Wnt agonist and/or the epigenetic modulator for use according to embodiment 461, wherein the LSD-1 inhibitor is compound 5a from Bioorganic Medicinal Chemistry Letters 26 (2016) 4552-4557, or a pharmaceutically acceptable salt thereof.
508. The Wnt agonist and/or the epigenetic modulator for use according to embodiment 461, wherein the LSD-1 inhibitor is compound 5n from Bioorganic Medicinal Chemistry Letters 26 (2016) 4552-4557, or a pharmaceutically acceptable salt thereof.
509. The Wnt agonist and/or the epigenetic modulator for use according to embodiment 461, wherein the LSD-1 inhibitor is SP-2509 (HCl-2509), or a pharmaceutically acceptable salt thereof.
510. The Wnt agonist and/or the epigenetic modulator for use according to embodiment 461, wherein the LSD-1 inhibitor is LSD1-IN-32 from J. Med. Chem. 2017, 60, 7984-7999, or a pharmaceutically acceptable salt thereof.
511. The Wnt agonist and/or the epigenetic modulator for use according to embodiment 461, wherein the LSD-1 inhibitor is LSD1-IN-11p from Bioorganic Medicinal Chemistry Letters 27 (2017) 3190-3195, or a pharmaceutically acceptable salt thereof.
512. The Wnt agonist and/or the epigenetic modulator for use according to embodiment 461, wherein the LSD-1 inhibitor is resveratrol or a pharmaceutically acceptable salt thereof.
513. The Wnt agonist and/or the epigenetic modulator for use according to embodiment 461, wherein the LSD-1 inhibitor is hydroxylamine or a pharmaceutically acceptable salt thereof.
514. The Wnt agonist and/or the epigenetic modulator for use according to embodiment 461, wherein the LSD-1 inhibitor is compound 8c from Bioorganic Medicinal Chemistry 2018, 26, 6000, or a pharmaceutically acceptable salt thereof
515. The Wnt agonist and/or the epigenetic modulator for use according to embodiment 461, wherein the LSD-1 inhibitor is CBB-1007 or a pharmaceutically acceptable salt thereof
516. The Wnt agonist and/or the epigenetic modulator for use according to embodiment 461, wherein the LSD-1 inhibitor is namoline from Int. J. Cancer 2012, 131, 2704-2709, or a pharmaceutically acceptable salt thereof.
517. The Wnt agonist and/or the epigenetic modulator for use according to embodiment 461, wherein the LSD-1 inhibitor is GSK-354 from Future Medicinal Chemistry (2017), 9(11), 1227-1242, or a pharmaceutically acceptable salt thereof
518. The Wnt agonist and/or the epigenetic modulator for use according to embodiment 461, wherein the LSD-1 inhibitor is GSK-690 from Future Medicinal Chemistry (2017), 9(11), 1227-1242, or a pharmaceutically acceptable salt thereof
519. The Wnt agonist and/or the epigenetic modulator for use according to embodiment 461, wherein the LSD-1 inhibitor is El1 or a pharmaceutically acceptable salt thereof.
520. The Wnt agonist and/or the epigenetic modulator for use according to embodiment 461, wherein the LSD-1 inhibitor is MC2694 or a pharmaceutically acceptable salt thereof.
521. The Wnt agonist and/or the epigenetic modulator for use according to embodiment 461, wherein the LSD-1 inhibitor is alpha-mangostin or a pharmaceutically acceptable salt thereof.
522. The Wnt agonist and/or the epigenetic modulator for use according to embodiment 461, wherein the LSD-1 inhibitor is compound 4 from European Journal of Medicinal Chemistry (2019), 162, 555-567, or a pharmaceutically acceptable salt thereof.
523. The Wnt agonist and/or the epigenetic modulator for use according to embodiment 461, wherein the LSD-1 inhibitor is compound 10d from Bioorganic Chemistry 2018, 78, 7-16, or a pharmaceutically acceptable salt thereof.
524. The Wnt agonist and/or the epigenetic modulator for use according to embodiment 461, wherein the LSD-1 inhibitor is compound 90 from J. Med. Chem. 2017, 60, 1673-1692, or a pharmaceutically acceptable salt thereof.
525. The Wnt agonist and/or the epigenetic modulator for use according to embodiment 461, wherein the LSD-1 inhibitor is compound 46 J. Med. Chem. 2017, 60, 1693-1715, or a pharmaceutically acceptable salt thereof.
526. The Wnt agonist and/or the epigenetic modulator for use according to embodiment 461, wherein the LSD-1 inhibitor is compound 49 J. Med. Chem. 2017, 60, 1693-1715, or a pharmaceutically acceptable salt thereof.
527. The Wnt agonist and/or the epigenetic modulator for use according to embodiment 461, wherein the LSD-1 inhibitor is compound 50 J. Med. Chem. 2017, 60, 1693-1715, or a pharmaceutically acceptable salt thereof
528. The Wnt agonist and/or the epigenetic modulator for use according to embodiment 461, wherein the LSD-1 inhibitor is polymyxin B from Future Medicinal Chemistry (2017), 9(11), 1227-1242, or a pharmaceutically acceptable salt thereof
529. The Wnt agonist and/or the epigenetic modulator for use according to embodiment 461, wherein the LSD-1 inhibitor is polymyxin E from Future Medicinal Chemistry (2017), 9(11), 1227-1242, or a pharmaceutically acceptable salt thereof
530. The Wnt agonist and/or the epigenetic modulator for use according to embodiment 461, wherein the LSD-1 inhibitor is baicalin or a pharmaceutically acceptable salt thereof.
531. The Wnt agonist and/or the epigenetic modulator for use according to embodiment 461, wherein the LSD-1 inhibitor is compound 16Q from Med. Chem. Commun., 2013, 4, 1513, or a pharmaceutically acceptable salt thereof.
532. The Wnt agonist and/or the epigenetic modulator for use according to embodiment 461, wherein the LSD-1 inhibitor is LSD1 inhibitor 24 or a pharmaceutically acceptable salt thereof.
533. The Wnt agonist and/or the epigenetic modulator for use according to embodiment 461, wherein the LSD-1 inhibitor is geranylgeranoic acid from Biochemical and Biophysical Research Communications 444 (2014) 24-29, or a pharmaceutically acceptable salt thereof.
534. The Wnt agonist and/or the epigenetic modulator for use according to embodiment 461, wherein the LSD-1 inhibitor is geranylgeraniol or a pharmaceutically acceptable salt thereof.
535. The Wnt agonist and/or the epigenetic modulator for use according to embodiment 461, wherein the LSD-1 inhibitor is thiocarbamate or a pharmaceutically acceptable salt thereof.
536. The Wnt agonist and/or the epigenetic modulator for use according to embodiment 461, wherein the LSD-1 inhibitor is thiourea or a pharmaceutically acceptable salt thereof.
537. The Wnt agonist and/or the epigenetic modulator for use according to embodiment 461, wherein the LSD-1 inhibitor is thienopyrrole or a pharmaceutically acceptable salt thereof.
538. The Wnt agonist and/or the epigenetic modulator for use according to embodiment 461, wherein the LSD-1 inhibitor is 4SC-202 or a pharmaceutically acceptable salt thereof.
539. The Wnt agonist and/or the epigenetic modulator for use according to embodiment 461, wherein the LSD-1 inhibitor is ORY-3001 or a pharmaceutically acceptable salt thereof
540. The Wnt agonist and/or the epigenetic modulator for use according to embodiment 461, wherein the LSD-1 inhibitor is JL1037 from Oncotarget 2017, 8(19), 31901-31914, or a pharmaceutically acceptable salt thereof.
541. The Wnt agonist and/or the epigenetic modulator for use according to embodiment 461, wherein the LSD-1 inhibitor is FLI-06 or a pharmaceutically acceptable salt thereof.
542. The Wnt agonist and/or the epigenetic modulator for use according to embodiment 461, wherein the LSD-1 inhibitor is rhodium complex 1 from J. Med. Chem. 2017, 60, 2597-2603, or a pharmaceutically acceptable salt thereof.
543. The Wnt agonist and/or the epigenetic modulator for use according to embodiment 462, wherein the HDAC inhibitor is Phenyl butyrate, or a pharmaceutically acceptable salt thereof.
544. The Wnt agonist and/or the epigenetic modulator for use according to embodiment 462, wherein the HDAC inhibitor is 2-hexyl-4-pentynoic acid, or a pharmaceutically acceptable salt thereof.
545. The Wnt agonist and/or the epigenetic modulator for use according to embodiment 462, wherein the HDAC inhibitor is Butyrate or a pharmaceutically acceptable salt thereof
546. The Wnt agonist and/or the epigenetic modulator for use according to embodiment 462, wherein the HDAC inhibitor is AN-9 or a pharmaceutically acceptable salt thereof
547. The Wnt agonist and/or the epigenetic modulator for use according to embodiment 462, wherein the HDAC inhibitor is Entinostat or a pharmaceutically acceptable salt thereof
548. The Wnt agonist and/or the epigenetic modulator for use according to embodiment 462, wherein the HDAC inhibitor is Mocetinostat or a pharmaceutically acceptable salt thereof.
549. The Wnt agonist and/or the epigenetic modulator for use according to embodiment 462, wherein the HDAC inhibitor is Tacedinaline or a pharmaceutically acceptable salt thereof.
550. The Wnt agonist and/or the epigenetic modulator for use according to embodiment 462, wherein the HDAC inhibitor is BML-210 or a pharmaceutically acceptable salt thereof.
551. The Wnt agonist and/or the epigenetic modulator for use according to embodiment 462, wherein the HDAC inhibitor is NKL 22 or a pharmaceutically acceptable salt thereof.
552. The Wnt agonist and/or the epigenetic modulator for use according to embodiment 462, wherein the HDAC inhibitor is RGFP109 or a pharmaceutically acceptable salt thereof.
553. The Wnt agonist and/or the epigenetic modulator for use according to embodiment 462, wherein the HDAC inhibitor is RGFP136 or a pharmaceutically acceptable salt thereof.
554. The Wnt agonist and/or the epigenetic modulator for use according to embodiment 462, wherein the HDAC inhibitor is RGFP966 or a pharmaceutically acceptable salt thereof.
555. The Wnt agonist and/or the epigenetic modulator for use according to embodiment 462, wherein the HDAC inhibitor is 4SC-202 or a pharmaceutically acceptable salt thereof.
556. The Wnt agonist and/or the epigenetic modulator for use according to embodiment 462, wherein the HDAC inhibitor is HDAC inhibitor IV or a pharmaceutically acceptable salt thereof.
557. The Wnt agonist and/or the epigenetic modulator for use according to embodiment 462, wherein the HDAC inhibitor is Chidamide or a pharmaceutically acceptable salt thereof.
558. The Wnt agonist and/or the epigenetic modulator for use according to embodiment 462, wherein the HDAC inhibitor is TC-H 106 or a pharmaceutically acceptable salt thereof.
559. The Wnt agonist and/or the epigenetic modulator for use according to embodiment 462, wherein the HDAC inhibitor is Romidepsin or a pharmaceutically acceptable salt thereof.
560. The Wnt agonist and/or the epigenetic modulator for use according to embodiment 462, wherein the HDAC inhibitor is Trapoxin A or a pharmaceutically acceptable salt thereof.
561. The Wnt agonist and/or the epigenetic modulator for use according to embodiment 462, wherein the HDAC inhibitor is HC Toxin or a pharmaceutically acceptable salt thereof.
562. The Wnt agonist and/or the epigenetic modulator for use according to embodiment 462, wherein the HDAC inhibitor is Apicidin or a pharmaceutically acceptable salt thereof.
563. The Wnt agonist and/or the epigenetic modulator for use according to embodiment 462, wherein the HDAC inhibitor is Thailandepsin A or a pharmaceutically acceptable salt thereof.
564. The Wnt agonist and/or the epigenetic modulator for use according to embodiment 462, wherein the HDAC inhibitor is Dihydrochlamydocin or a pharmaceutically acceptable salt thereof.
565. The Wnt agonist and/or the epigenetic modulator for use according to embodiment 462, wherein the HDAC inhibitor is (−)-Depudecin or a pharmaceutically acceptable salt thereof.
566. The Wnt agonist and/or the epigenetic modulator for use according to embodiment 462, wherein the HDAC inhibitor is Parthenolide or a pharmaceutically acceptable salt thereof.
567. The Wnt agonist and/or the epigenetic modulator for use according to embodiment 462, wherein the HDAC inhibitor is Trichostatin A (TSA) or a pharmaceutically acceptable salt thereof.
568. The Wnt agonist and/or the epigenetic modulator for use according to embodiment 462, wherein the HDAC inhibitor is SAHA (Zolinza, vorinostat) or a pharmaceutically acceptable salt thereof.
569. The Wnt agonist and/or the epigenetic modulator for use according to embodiment 462, wherein the HDAC inhibitor is 4-iodo-SAHA or a pharmaceutically acceptable salt thereof.
570. The Wnt agonist and/or the epigenetic modulator for use according to embodiment 462, wherein the HDAC inhibitor is SBHA or a pharmaceutically acceptable salt thereof.
571. The Wnt agonist and/or the epigenetic modulator for use according to embodiment 462, wherein the HDAC inhibitor is CBHA or a pharmaceutically acceptable salt thereof.
572. The Wnt agonist and/or the epigenetic modulator for use according to embodiment 462, wherein the HDAC inhibitor is LAQ-824 or a pharmaceutically acceptable salt thereof.
573. The Wnt agonist and/or the epigenetic modulator for use according to embodiment 462, wherein the HDAC inhibitor is PDX-101 (belinostat) or a pharmaceutically acceptable salt thereof.
574. The Wnt agonist and/or the epigenetic modulator for use according to embodiment 462, wherein the HDAC inhibitor is LBH-589 (panobinostat) or a pharmaceutically acceptable salt thereof.
575. The Wnt agonist and/or the epigenetic modulator for use according to embodiment 462, wherein the HDAC inhibitor is ITF2357 (Givinostat) or a pharmaceutically acceptable salt thereof.
576. The Wnt agonist and/or the epigenetic modulator for use according to embodiment 462, wherein the HDAC inhibitor is PCI-34051 or a pharmaceutically acceptable salt thereof
577. The Wnt agonist and/or the epigenetic modulator for use according to embodiment 462, wherein the HDAC inhibitor is PCI-24781 (Abexinostat) or a pharmaceutically acceptable salt thereof.
578. The Wnt agonist and/or the epigenetic modulator for use according to embodiment 462, wherein the HDAC inhibitor is Tubastatin A or a pharmaceutically acceptable salt thereof.
579. The Wnt agonist and/or the epigenetic modulator for use according to embodiment 462, wherein the HDAC inhibitor is CUDC-101 or a pharmaceutically acceptable salt thereof.
580. The Wnt agonist and/or the epigenetic modulator for use according to embodiment 462, wherein the HDAC inhibitor is Oxamflatin or a pharmaceutically acceptable salt thereof.
581. The Wnt agonist and/or the epigenetic modulator for use according to embodiment 462, wherein the HDAC inhibitor is ITF2357 or a pharmaceutically acceptable salt thereof.
582. The Wnt agonist and/or the epigenetic modulator for use according to embodiment 462, wherein the HDAC inhibitor is Bufexamac or a pharmaceutically acceptable salt thereof.
583. The Wnt agonist and/or the epigenetic modulator for use according to embodiment 462, wherein the HDAC inhibitor is APHA Compound 8 or a pharmaceutically acceptable salt thereof.
584. The Wnt agonist and/or the epigenetic modulator for use according to embodiment 462, wherein the HDAC inhibitor is Tubacin or a pharmaceutically acceptable salt thereof.
585. The Wnt agonist and/or the epigenetic modulator for use according to embodiment 462, wherein the HDAC inhibitor is Butyrylhydroxamic acid or a pharmaceutically acceptable salt thereof.
586. The Wnt agonist and/or the epigenetic modulator for use according to embodiment 462, wherein the HDAC inhibitor is MC 1568 or a pharmaceutically acceptable salt thereof.
587. The Wnt agonist and/or the epigenetic modulator for use according to embodiment 462, wherein the HDAC inhibitor is SB939 (Pracinostat) or a pharmaceutically acceptable salt thereof.
588. The Wnt agonist and/or the epigenetic modulator for use according to embodiment 462, wherein the HDAC inhibitor is 4SC-201 (Reminostat) or a pharmaceutically acceptable salt thereof.
589. The Wnt agonist and/or the epigenetic modulator for use according to embodiment 462, wherein the HDAC inhibitor is Tefinostat (CHR-2845) or a pharmaceutically acceptable salt thereof.
590. The Wnt agonist and/or the epigenetic modulator for use according to embodiment 462, wherein the HDAC inhibitor is CHR-3996 or a pharmaceutically acceptable salt thereof.
591. The Wnt agonist and/or the epigenetic modulator for use according to embodiment 462, wherein the HDAC inhibitor is NSC 57457 or a pharmaceutically acceptable salt thereof.
592. The Wnt agonist and/or the epigenetic modulator for use according to embodiment 462, wherein the HDAC inhibitor is CG200745 or a pharmaceutically acceptable salt thereof.
593. The Wnt agonist and/or the epigenetic modulator for use according to embodiment 462, wherein the HDAC inhibitor is ACY1215 or a pharmaceutically acceptable salt thereof.
594. The Wnt agonist and/or the epigenetic modulator for use according to embodiment 462, wherein the HDAC inhibitor is Nexturastat A or a pharmaceutically acceptable salt thereof.
595. The Wnt agonist and/or the epigenetic modulator for use according to embodiment 462, wherein the HDAC inhibitor is Droxinostat or a pharmaceutically acceptable salt thereof.
596. The Wnt agonist and/or the epigenetic modulator for use according to embodiment 462, wherein the HDAC inhibitor is Scriptaid or a pharmaceutically acceptable salt thereof.
597. The Wnt agonist and/or the epigenetic modulator for use according to embodiment 462, wherein the HDAC inhibitor is BRD9757 or a pharmaceutically acceptable salt thereof
598. The Wnt agonist and/or the epigenetic modulator for use according to embodiment 462, wherein the HDAC inhibitor is HPOB or a pharmaceutically acceptable salt thereof.
599. The Wnt agonist and/or the epigenetic modulator for use according to embodiment 462, wherein the HDAC inhibitor is CAY10603 or a pharmaceutically acceptable salt thereof.
600. The Wnt agonist and/or the epigenetic modulator for use according to embodiment 462, wherein the HDAC inhibitor is HDAC6 Inhibitor III or a pharmaceutically acceptable salt thereof.
601. The Wnt agonist and/or the epigenetic modulator for use according to embodiment 462, wherein the HDAC inhibitor is M 344 or a pharmaceutically acceptable salt thereof.
602. The Wnt agonist and/or the epigenetic modulator for use according to embodiment 462, wherein the HDAC inhibitor is 4-(dimethylamino)-N-[6-(hydroxyamino)-6-oxohexyl]-benzamide or a pharmaceutically acceptable salt thereof.
603. The Wnt agonist and/or the epigenetic modulator for use according to embodiment 462, wherein the HDAC inhibitor is (S)-HDAC-42 or a pharmaceutically acceptable salt thereof.
604. The Wnt agonist and/or the epigenetic modulator for use according to embodiment 462, wherein the HDAC inhibitor is HNHA or a pharmaceutically acceptable salt thereof.
605. The Wnt agonist and/or the epigenetic modulator for use according to embodiment 462, wherein the HDAC inhibitor is Pyroxamide or a pharmaceutically acceptable salt thereof.
606. The Wnt agonist and/or the epigenetic modulator for use according to embodiment 462, wherein the HDAC inhibitor is LMK235 or a pharmaceutically acceptable salt thereof.
607. The Wnt agonist and/or the epigenetic modulator for use according to embodiment 462, wherein the HDAC inhibitor is HDAC-IN-1 or a pharmaceutically acceptable salt thereof.
608. The Wnt agonist and/or the epigenetic modulator for use according to embodiment 462, wherein the HDAC inhibitor is VAHA or a pharmaceutically acceptable salt thereof.
609. The Wnt agonist and/or the epigenetic modulator for use according to embodiment 462, wherein the HDAC inhibitor is Ratjadone A or a pharmaceutically acceptable salt thereof.
610. The Wnt agonist and/or the epigenetic modulator for use according to embodiment 462, wherein the HDAC inhibitor is TCS HDAC6 20b or a pharmaceutically acceptable salt thereof.
611. The Wnt agonist and/or the epigenetic modulator for use according to embodiment 462, wherein the HDAC inhibitor is PTACH or a pharmaceutically acceptable salt thereof.
612. The Wnt agonist and/or the epigenetic modulator for use according to embodiment 462, wherein the HDAC inhibitor is KD 5170 or a pharmaceutically acceptable salt thereof.
613. The Wnt agonist and/or the epigenetic modulator for use according to embodiment 462, wherein the HDAC inhibitor is HDAC Inhibitor XXII or a pharmaceutically acceptable salt.
614. The Wnt agonist and/or the epigenetic modulator for use according to embodiment 462, wherein the HDAC inhibitor is SIRT1/2 Inhibitor VII or a pharmaceutically acceptable salt thereof.
615. The Wnt agonist and/or the epigenetic modulator for use according to embodiment 462, wherein the HDAC inhibitor is TMP269 or a pharmaceutically acceptable salt thereof.
616. The Wnt agonist and/or the epigenetic modulator for use according to embodiment 462, wherein the HDAC inhibitor is Tasquinimod or a pharmaceutically acceptable salt thereof.
617. The Wnt agonist and/or the epigenetic modulator for use according to embodiment 462, wherein the HDAC inhibitor is 2-(prop-2-yn-1-yl)octanoic acid or a pharmaceutically acceptable salt thereof.
618. The Wnt agonist and/or the epigenetic modulator for use according to embodiment 462, wherein the HDAC inhibitor is (S)-2-(prop-2-yn-1-yl)octanoic acid or a pharmaceutically acceptable salt thereof.
619. The Wnt agonist and/or the epigenetic modulator for use according to embodiment 462, wherein the HDAC inhibitor is (R)-2-(prop-2-yn-1-yl)octanoic acid or a pharmaceutically acceptable salt thereof.
620. The Wnt agonist and/or the epigenetic modulator for use according to embodiment 462, wherein the HDAC inhibitor is 2-(prop-2-yn-1-yl)heptanoic acid or a pharmaceutically acceptable salt thereof
621. The Wnt agonist and/or the epigenetic modulator for use according to embodiment 462, wherein the HDAC inhibitor is (S)-2-(prop-2-yn-1-yl)heptanoic acid or a pharmaceutically acceptable salt thereof.
622. The Wnt agonist and/or the epigenetic modulator for use according to embodiment 462, wherein the HDAC inhibitor is (R)-2-(prop-2-yn-1-yl)heptanoic acid or a pharmaceutically acceptable salt thereof.
623. The Wnt agonist and/or the epigenetic modulator for use according to embodiment 462, wherein the HDAC inhibitor is 2-fluoro-2-propyl Pentanoic acid or a pharmaceutically acceptable salt thereof
624. The Wnt agonist and/or the epigenetic modulator for use according to embodiment 462, wherein the HDAC inhibitor is HDAC Inhibitor II or a pharmaceutically acceptable salt thereof.
625. The Wnt agonist and/or the epigenetic modulator for use according to embodiment 462, wherein the HDAC inhibitor is HDAC Inhibitor VI or a pharmaceutically acceptable salt thereof.
626. The Wnt agonist and/or the epigenetic modulator for use according to embodiment 462, wherein the HDAC inhibitor is HDAC Inhibitor XXIV or a pharmaceutically acceptable salt thereof.
627. The Wnt agonist and/or the epigenetic modulator for use according to embodiment 462, wherein the HDAC inhibitor is HDAC6 Inhibitor III or a pharmaceutically acceptable salt thereof.
628. The Wnt agonist and/or the epigenetic modulator for use according to embodiment 463, wherein the EZH-2 inhibitor is GSK126 from GSK Phase I (GSK2816126) or a pharmaceutically acceptable salt thereof.
629. The Wnt agonist and/or the epigenetic modulator for use according to embodiment 463, wherein the EZH-2 inhibitor is SHR2554 or a pharmaceutically acceptable salt thereof.
630. The Wnt agonist and/or the epigenetic modulator for use according to embodiment 463, wherein the EZH-2 inhibitor is MAK683 or a pharmaceutically acceptable salt thereof.
631. The Wnt agonist and/or the epigenetic modulator for use according to embodiment 463, wherein the EZH-2 inhibitor is CPI-0169 or a pharmaceutically acceptable salt thereof.
632. The Wnt agonist and/or the epigenetic modulator for use according to embodiment 463, wherein the EZH-2 inhibitor is EPZ-011989 or a pharmaceutically acceptable salt thereof.
633. The Wnt agonist and/or the epigenetic modulator for use according to embodiment 463, wherein the EZH-2 inhibitor is EPZ-005687 or a pharmaceutically acceptable salt thereof.
634. The Wnt agonist and/or the epigenetic modulator for use according to embodiment 463, wherein the EZH-2 inhibitor is CPI-360 or a pharmaceutically acceptable salt thereof.
635. The Wnt agonist and/or the epigenetic modulator for use according to embodiment 463, wherein the EZH-2 inhibitor is CPI-169 or a pharmaceutically acceptable salt thereof.
636. The Wnt agonist and/or the epigenetic modulator for use according to embodiment 463, wherein the EZH-2 inhibitor is SKLB1049 or a pharmaceutically acceptable salt thereof.
637. The Wnt agonist and/or the epigenetic modulator for use according to embodiment 463, wherein the EZH-2 inhibitor is ZLD1039 or a pharmaceutically acceptable salt thereof.
638. The Wnt agonist and/or the epigenetic modulator for use according to embodiment 463, wherein the EZH-2 inhibitor is ZLD1122 or a pharmaceutically acceptable salt thereof.
639. The Wnt agonist and/or the epigenetic modulator for use according to embodiment 463, wherein the EZH-2 inhibitor is GSK503 from Arch Otolaryngol Head Neck Surg. 2001,127(4), 447-452, or a pharmaceutically acceptable salt thereof.
640. The Wnt agonist and/or the epigenetic modulator for use according to embodiment 463, wherein the EZH-2 inhibitor is GSK926 from ACS Med. Chem. Lett. 2012, 3, 1091-1096, or a pharmaceutically acceptable salt thereof.
641. The Wnt agonist and/or the epigenetic modulator for use according to embodiment 463, wherein the EZH-2 inhibitor is GSK343 from ACS Med. Chem. Lett. 2012, 3, 1091-1096, or a pharmaceutically acceptable salt thereof.
642. The Wnt agonist and/or the epigenetic modulator for use according to embodiment 463, wherein the EZH-2 inhibitor is EBI-2511, or a pharmaceutically acceptable salt thereof.
643. The Wnt agonist and/or the epigenetic modulator for use according to embodiment 463, wherein the EZH-2 inhibitor is (R)—OR—S1, or a pharmaceutically acceptable salt thereof
644. The Wnt agonist and/or the epigenetic modulator for use according to embodiment 463, wherein the EZH-2 inhibitor is A-395, or a pharmaceutically acceptable salt thereof.
645. The Wnt agonist and/or the epigenetic modulator for use according to embodiment 463, wherein the EZH-2 inhibitor is astemizole or a pharmaceutically acceptable salt thereof
646. The Wnt agonist and/or the epigenetic modulator for use according to embodiment 463, wherein the EZH-2 inhibitor is EED162 or a pharmaceutically acceptable salt thereof.
647. The Wnt agonist and/or the epigenetic modulator for use according to embodiment 463, wherein the EZH-2 inhibitor is CPI-0209 or a pharmaceutically acceptable salt thereof
648. The Wnt agonist and/or the epigenetic modulator for use according to embodiment 463, wherein the EZH-2 inhibitor is EED226 or a pharmaceutically acceptable salt thereof.
649. The Wnt agonist and/or the epigenetic modulator for use according to embodiment 463, wherein the EZH-2 inhibitor is DZNep or a pharmaceutically acceptable salt thereof
650. The Wnt agonist and/or the epigenetic modulator for use according to embodiment 463, wherein the EZH-2 inhibitor is UNC1999 or a pharmaceutically acceptable salt thereof.
651. The Wnt agonist and/or the epigenetic modulator for use according to embodiment 463, wherein the EZH-2 inhibitor is sinefungin or a pharmaceutically acceptable salt thereof
652. The Wnt agonist and/or the epigenetic modulator for use according to embodiment 463, wherein the EZH-2 inhibitor is tanshindiol B or a pharmaceutically acceptable salt thereof.
653. The Wnt agonist and/or the epigenetic modulator for use according to embodiment 463, wherein the EZH-2 inhibitor is tanshindiol C or a pharmaceutically acceptable salt thereof.
654. The Wnt agonist and/or the epigenetic modulator for use according to embodiment 463, wherein the EZH-2 inhibitor is Cmpd 44 from ACS Med. Chem. Lett. 2014, 5, 378-383, or a pharmaceutically acceptable salt thereof.
655. The Wnt agonist and/or the epigenetic modulator for use according to embodiment 463, wherein the EZH-2 inhibitor is MC1945 from Arch Otolaryngol Head Neck Surg. 2001,127(4), 447-452, or a pharmaceutically acceptable salt thereof.
656. The Wnt agonist and/or the epigenetic modulator for use according to embodiment 463, wherein the EZH-2 inhibitor is MC1947 from Arch Otolaryngol Head Neck Surg. 2001,127(4), 447-452, or a pharmaceutically acceptable salt thereof.
657. The Wnt agonist and/or the epigenetic modulator for use according to embodiment 463, wherein the EZH-2 inhibitor is MC1948 from Arch Otolaryngol Head Neck Surg. 2001,127(4), 447-452, or a pharmaceutically acceptable salt thereof.
658. The Wnt agonist and/or the epigenetic modulator for use according to embodiment 463, wherein the EZH-2 inhibitor is curcumin as described in European Journal of Pharmacology 2010, 637, 16-21, or a pharmaceutically acceptable salt thereof.
659. The Wnt agonist and/or the epigenetic modulator for use according to embodiment 463, wherein the EZH-2 inhibitor is (−)-Epigallocatechin-3-gallate (EGCG) from Carcinogenesis. 2011; 32: 1525-32, or a pharmaceutically acceptable salt thereof.
660. The Wnt agonist and/or the epigenetic modulator for use according to embodiment 463, wherein the EZH-2 inhibitor is sulforaphane from Mol Pharmacol. 2011, 80, 870-8, or a pharmaceutically acceptable salt thereof.
661. The Wnt agonist and/or the epigenetic modulator for use according to embodiment 463, wherein the EZH-2 inhibitor is SAH-EZH2 from Current Medicinal Chemistry (2016), 23(27), 3025-3043, or a pharmaceutically acceptable salt thereof.
662. The Wnt agonist and/or the epigenetic modulator for use according to embodiment 463, wherein the EZH2 inhibitor is an enzymatic inhibitor.
663. The Wnt agonist and/or the epigenetic modulator for use according to embodiment 463, wherein the EZH2 inhibitor is CPI-1205 or a pharmaceutically acceptable salt thereof.
664. The Wnt agonist and/or the epigenetic modulator for use according to embodiment 463, wherein the EZH2 inhibitor is El1 or a pharmaceutically acceptable salt thereof.
665. The Wnt agonist and/or the epigenetic modulator for use according to embodiment 463, wherein the EZH2 inhibitor is PF-06821497 or a pharmaceutically acceptable salt thereof
666. The Wnt agonist and/or the epigenetic modulator for use according to embodiment 463, wherein the EZH2 inhibitor is Tazemetostat or a pharmaceutically acceptable salt thereof
667. The Wnt agonist and/or the epigenetic modulator for use according to embodiment 463, wherein the EZH2 inhibitor is Valemetostat (DS-3201b, (R)—OR—S2) or a pharmaceutically acceptable salt thereof
668. The Wnt agonist and/or the epigenetic modulator for use according to embodiment 463, wherein the EZH2 inhibitor is EBI-2511 or a pharmaceutically acceptable salt thereof.
669. The Wnt agonist and/or the epigenetic modulator for use according to embodiment 464, wherein the DOT1L inhibitor is SYC-687 or a pharmaceutically acceptable salt thereof.
670. The Wnt agonist and/or the epigenetic modulator for use according to embodiment 464, wherein the DOT1L inhibitor is SYC-522 or a pharmaceutically acceptable salt thereof.
671. The Wnt agonist and/or the epigenetic modulator for use according to embodiment 464, wherein the DOT1L inhibitor is EPZ002696 or a pharmaceutically acceptable salt thereof.
672. The Wnt agonist and/or the epigenetic modulator for use according to embodiment 464, wherein the DOT1L inhibitor is EPZ004450 or a pharmaceutically acceptable salt thereof.
673. The Wnt agonist and/or the epigenetic modulator for use according to embodiment 464, wherein the DOT1L inhibitor is CN SAH or a pharmaceutically acceptable salt thereof.
674. The Wnt agonist and/or the epigenetic modulator for use according to embodiment 464, wherein the DOT1L inhibitor is SAH or a pharmaceutically acceptable salt thereof.
675. The Wnt agonist and/or the epigenetic modulator for use according to embodiment 464, wherein the DOT1L inhibitor is bromo-deaza-SAH or a pharmaceutically acceptable salt thereof.
676. The Wnt agonist and/or the epigenetic modulator for use according to embodiment 464, wherein the DOT1L inhibitor is compound 21 from ACS Medicinal Chemistry Letters (2018), 9(9), 895-900—Peptides, or a pharmaceutically acceptable salt thereof.
677. The Wnt agonist and/or the epigenetic modulator for use according to embodiment 464, wherein the DOT1L inhibitor is compound 28 from ACS Medicinal Chemistry Letters (2018), 9(9), 895-900—Peptides, or a pharmaceutically acceptable salt thereof.
678. The Wnt agonist and/or the epigenetic modulator for use according to embodiment 464, wherein the DOT1L inhibitor is compound 8H from Bioorganic Chemistry (2018), 80, 649-654, or a pharmaceutically acceptable salt thereof.
679. The Wnt agonist and/or the epigenetic modulator for use according to embodiment 464, wherein the DOT1L inhibitor is an S-adenosyl methionine (SAM) competitive inhibitor or a pharmaceutically acceptable salt thereof.
680. The Wnt agonist and/or the epigenetic modulator for use according to embodiment 464, wherein the DOT1L inhibitor is EPZ004777 or a pharmaceutically acceptable salt thereof.
681. The Wnt agonist and/or the epigenetic modulator for use according to embodiment 464, wherein the DOT1L inhibitor is Pinometostat (EPZ5676) or a pharmaceutically acceptable salt thereof.
682. The Wnt agonist and/or the epigenetic modulator for use according to embodiment 464, wherein the DOT1L inhibitor is SGC0946 or a pharmaceutically acceptable salt thereof.
683. The Wnt agonist and/or the epigenetic modulator for use according to embodiment 464, wherein the DOT1L inhibitor is compound 8H from Bioorganic Chemistry (2018), 80, 649-654, or a pharmaceutically acceptable salt thereof.
684. The Wnt agonist and/or the TAZ activator for use according to embodiment 466, wherein the TAZ activator is IBS008738 or a pharmaceutically acceptable salt thereof
685. The Wnt agonist and/or the TAZ activator for use according to embodiment 466, wherein the TAZ activator is TM-25659 or a pharmaceutically acceptable salt thereof.
686. The Wnt agonist and/or the TAZ activator for use according to embodiment 466, wherein the TAZ activator is TT10 or a pharmaceutically acceptable salt thereof.
687. The Wnt agonist and/or the TAZ activator for use according to embodiment 466, wherein the TAZ activator is IBS003031 or a pharmaceutically acceptable salt thereof.
688. The Wnt agonist and/or the TAZ activator for use according to embodiment 466, wherein the TAZ activator is TAZ12 or a pharmaceutically acceptable salt thereof.
689. The Wnt agonist and/or the TAZ activator for use according to embodiment 466, wherein the TAZ activator is TM-53 or a pharmaceutically acceptable salt thereof.
690. The Wnt agonist and/or the TAZ activator for use according to embodiment 466, wherein the TAZ activator is TM-54 or a pharmaceutically acceptable salt thereof.
691. The Wnt agonist and/or the TAZ activator for use according to embodiment 466, wherein the TAZ activator is (−)-epicatechin gallate or a pharmaceutically acceptable salt thereof.
692. The Wnt agonist and/or the TAZ activator for use according to embodiment 466, wherein the TAZ activator is Ethacridine or a pharmaceutically acceptable salt thereof.
693. The Wnt agonist and/or the TAZ activator for use according to embodiment 466, wherein the TAZ activator is kaempferol or a pharmaceutically acceptable salt thereof
694. The Wnt agonist and/or the TAZ activator for use according to embodiment 466, wherein the TAZ activator is KR62980 or a pharmaceutically acceptable salt thereof
695. The Wnt agonist and/or the TAZ activator for use according to embodiment 466, wherein the TAZ activator is phorbaketal A or a pharmaceutically acceptable salt thereof.
696. The Wnt agonist and/or the epigenetic modulator for use according to embodiment 465, wherein the KDM inhibitor is AS 8351 or a pharmaceutically acceptable salt thereof.
697. The Wnt agonist and/or the epigenetic modulator for use according to embodiment 465, wherein the KDM inhibitor is EPT-103182 or a pharmaceutically acceptable salt thereof.
698. The Wnt agonist and/or the epigenetic modulator for use according to embodiment 465, wherein the KDM inhibitor is TC-E 5002 or a pharmaceutically acceptable salt thereof.
699. The Wnt agonist and/or the epigenetic modulator for use according to embodiment 465, wherein the KDM inhibitor is CPI-455 or a pharmaceutically acceptable salt thereof.
700. The Wnt agonist and/or the epigenetic modulator for use according to embodiment 465, wherein the KDM inhibitor is CPI-4203 or a pharmaceutically acceptable salt thereof.
701. The Wnt agonist and/or the epigenetic modulator for use according to embodiment 465, wherein the KDM inhibitor is E67-2 or a pharmaceutically acceptable salt thereof.
702. The Wnt agonist and/or the epigenetic modulator for use according to embodiment 465, wherein the KDM inhibitor is GSK-467 or a pharmaceutically acceptable salt thereof.
703. The Wnt agonist and/or the epigenetic modulator for use according to embodiment 465, wherein the KDM inhibitor is GSK-J1 or a pharmaceutically acceptable salt thereof.
704. The Wnt agonist and/or the epigenetic modulator for use according to embodiment 465, wherein the KDM inhibitor is GSK-J4 or a pharmaceutically acceptable salt thereof.
705. The Wnt agonist and/or the epigenetic modulator for use according to embodiment 465, wherein the KDM inhibitor is KDM5-C49 or a pharmaceutically acceptable salt thereof.
706. The Wnt agonist and/or the epigenetic modulator for use according to embodiment 465, wherein the KDM inhibitor is KDM5-C50 or a pharmaceutically acceptable salt thereof.
707. The Wnt agonist and/or the epigenetic modulator for use according to embodiment 465, wherein the KDM inhibitor is KDOAM 25 or a pharmaceutically acceptable salt thereof.
708. The Wnt agonist and/or the epigenetic modulator for use according to embodiment 465, wherein the KDM inhibitor is N11 or a pharmaceutically acceptable salt thereof.
709. The Wnt agonist and/or the epigenetic modulator for use according to embodiment 465, wherein the KDM inhibitor is Daminozide or a pharmaceutically acceptable salt thereof.
710. The Wnt agonist and/or the epigenetic modulator for use according to embodiment 465, wherein the KDM inhibitor is JIB-04 or a pharmaceutically acceptable salt thereof.
711. The Wnt agonist and/or the epigenetic modulator for use according to embodiment 465, wherein the KDM inhibitor is Methylstat or a pharmaceutically acceptable salt thereof.
712. The Wnt agonist and/or the epigenetic modulator for use according to embodiment 465, wherein the KDM inhibitor is N71 or a pharmaceutically acceptable salt thereof.
713. The Wnt agonist and/or the epigenetic modulator for use according to embodiment 465, wherein the KDM inhibitor is NSC6369819 or a pharmaceutically acceptable salt thereof.
714. The hair cell regeneration agent(s) and/or the Wnt agonist and/or the epigenetic modulator for use according to any preceding embodiment, wherein the hair cell regeneration agent(s) and/or Wnt agonist and/or epigenetic modulator is at a concentration of about 0.001 μM to about 1,000 mM.
715. The hair cell regeneration agent(s) and/or the Wnt agonist and/or the epigenetic modulator for use according to any preceding embodiment, wherein the hair cell regeneration agent(s) and/or Wnt agonist and/or epigenetic modulator is at a concentration of about 0.01 μM to about 100,000 μM.
716. The hair cell regeneration agent(s) and/or the Wnt agonist and/or the epigenetic modulator for use according to any preceding embodiment, wherein the hair cell regeneration agent(s) and/or Wnt agonist and/or epigenetic modulator is at a concentration of about 0.1 μM to about 10,000 μM.
717. The hair cell regeneration agent(s) and/or the Wnt agonist and/or the epigenetic modulator for use according to any preceding embodiment, wherein the hair cell regeneration agent(s) and/or Wnt agonist and/or epigenetic modulator is at a concentration of about 1 μM to about 1,000 μM.
718. The hair cell regeneration agent(s) and/or the Wnt agonist and/or the epigenetic modulator for use according to any preceding embodiment, wherein the hair cell regeneration agent(s) and/or Wnt agonist and/or epigenetic modulator is at a concentration of about 1 μM to about 10 μM.
719. The hair cell regeneration agent(s) and/or the Wnt agonist and/or the epigenetic modulator for use according to any preceding embodiment, wherein the hair cell regeneration agent(s) and/or Wnt agonist and/or epigenetic modulator is at a concentration of about 10 μM to about 100 μM.
720. The hair cell regeneration agent(s) and/or the Wnt agonist and/or the epigenetic modulator for use according to any preceding embodiment, wherein the hair cell regeneration agent(s) and/or Wnt agonist and/or epigenetic modulator is at a concentration of about 100 μM to about 1 mM.
721. The hair cell regeneration agent(s) and/or the Wnt agonist and/or the epigenetic modulator for use according to any preceding embodiment, wherein the hair cell regeneration agent(s) and/or Wnt agonist and/or epigenetic modulator is at a concentration of about 1 mM to about 10 mM, for example, about 6.1 mM, about 6.2 mM, about 6.3 mM, about 6.4 mM, about 6.5 mM, about 6.6 mM, about 6.7 mM, about 6.8 mM, about 6.9 mM, about 7.0 mM, about 7.1 mM, about 7.2 mM, about 7.3 mM, about 7.4 mM, such as about 6.75 mM.
722. The hair cell regeneration agent(s) and/or the Wnt agonist and/or the epigenetic modulator for use according to any preceding embodiment, wherein the hair cell regeneration agent(s) and/or Wnt agonist and/or epigenetic modulator is at a concentration of about 10 mM to about 100 mM.
723. The hair cell regeneration agent(s) and/or the Wnt agonist and/or the epigenetic modulator for use according to any preceding embodiment, wherein the hair cell regeneration agent(s) and/or Wnt agonist and/or epigenetic modulator is at a concentration of about 10 mM to about 1,000 mM, for example, about 550 mM, about 560 mM, about 570 mM, about 580 mM, about 590 mM, about 600 mM, about 610 mM, about 620 mM, about 630 mM or about 640 mM, about 650 mM, about 660 mM, about 670 mM, about 680 mM, such as about 615 mM.
724. The hair cell regeneration agent(s) and/or the Wnt agonist and/or the epigenetic modulator for use according to any preceding embodiment, wherein the hair cell regeneration agent(s) and/or Wnt agonist and/or epigenetic modulator is at a concentration of about 100 mM to about 10,000 mM.
725. The hair cell regeneration agent(s) and/or the Wnt agonist and/or the epigenetic modulator for use according to any preceding embodiment, wherein the hair cell regeneration agent(s) and/or Wnt agonist and/or epigenetic modulator is at a concentration of about 1,000 mM to about 100,000 mM.
726. The hair cell regeneration agent(s) and/or the Wnt agonist and/or the epigenetic modulator for use according to any preceding embodiment, wherein the hair cell regeneration agent(s) and/or Wnt agonist and/or epigenetic modulator is at a concentration of about 10,000 mM to about 1,000,000 mM.
727. The hair cell regeneration agent(s) and/or the Wnt agonist and/or the epigenetic modulator for use according to any preceding embodiment, wherein said treatment provides an improved standard word recognition score for the patient, wherein said improvement, if tested, would be at least 10%, at least 20%, at least 30%, at least 50%, at least 70%, at least 100%, at least 200%, at least 300%, at least 400%, at least 500%, at least 600%, at least 700%, at least 800%, at least 900% wherein said percentage improvement is calculated using the following formula:

$$100 \times ( \frac{patient ’ s word recognition score after treatment}{patient ’ s word recognition score prior to treatment} - 1 )$$

728. The hair cell regeneration agent(s) and/or the Wnt agonist and/or the epigenetic modulator for use according to embodiment 727, wherein said treatment provides an improved standard word recognition score for the patient, wherein said improvement, if tested, would be at least 10%.
729. The hair cell regeneration agent(s) and/or the Wnt agonist and/or the epigenetic modulator for use according to embodiment 727 or embodiment 728, wherein said treatment provides an improved standard word recognition score for the patient, wherein said improvement, if tested, would be at least 20%.
730. The hair cell regeneration agent(s) and/or the Wnt agonist and/or the epigenetic modulator for use according to any of embodiments 727-729, wherein said treatment provides an improved standard word recognition score for the patient, wherein said improvement, if tested, would be at least 30%.
731. The hair cell regeneration agent(s) and/or the Wnt agonist and/or the epigenetic modulator for use according to any of embodiments 727-730, wherein said treatment provides an improved standard word recognition score for the patient, wherein said improvement, if tested, would be at least 50%.
732. The hair cell regeneration agent(s) and/or the Wnt agonist and/or the epigenetic modulator for use according to any of embodiments 727-731, wherein said treatment provides an improved standard word recognition score for the patient, wherein said improvement, if tested, would be at least 70%.
733. The hair cell regeneration agent(s) and/or the Wnt agonist and/or the epigenetic modulator for use according to any of embodiments 727-732, wherein said treatment provides an improved standard word recognition score for the patient, wherein said improvement, if tested, would be at least 100%.
734. The hair cell regeneration agent(s) and/or the Wnt agonist and/or the epigenetic modulator for use according to any preceding embodiment, wherein said treatment provides an improved standard word recognition for the patient, wherein said improvement, if tested, would be at least 5, at least 10, at least 15 words relative to the number of words recognized by the patient in a standard word recognition test of 50 words prior to the treatment.
735. The hair cell regeneration agent(s) and/or the Wnt agonist and/or the epigenetic modulator for use according to embodiment 734, wherein said treatment provides an improved standard word recognition for the patient, wherein said improvement, if tested, would be at least 5 words relative to the number of words recognized by the patient in a standard word recognition test of 50 words prior to the treatment.
736. The hair cell regeneration agent(s) and/or the Wnt agonist and/or the epigenetic modulator for use according to embodiment 734 or embodiment 735, wherein said treatment provides an improved standard word recognition for the patient, wherein said improvement, if tested, would be at least 10 words relative to the number of words recognized by the patient in a standard word recognition test of 50 words prior to the treatment.
737. The hair cell regeneration agent(s) and/or the Wnt agonist and/or the epigenetic modulator for use according to any of embodiments 734-736, wherein said treatment provides an improved standard word recognition for the patient, wherein said improvement, if tested, would be at least 15 words relative to the number of words recognized by the patient in a standard word recognition test of 50 words prior to the treatment.
738. The hair cell regeneration agent(s) and/or the Wnt agonist and/or the epigenetic modulator for use according to any preceding embodiment, wherein said treatment provides an improved words-in-noise score for the patient, wherein said improvement, if tested, would be at least 10%, at least 20%, at least 30%, at least 50%, at least 70%, at least 100%, at least 200%, at least 300%, at least 400%, at least 500%, at least 600%, at least 700%, at least 800%, at least 900% wherein said percentage improvement is calculated using the following formula:

$$100 \times ( \frac{patient ’ s words in noise score after treatment}{patient ’ s words in noise score prior to treatment} - 1 )$$

739. The hair cell regeneration agent(s) and/or the Wnt agonist and/or the epigenetic modulator for use according to embodiment 738, wherein said treatment provides an improved words-in-noise score for the patient, wherein said improvement, if tested, would be at least 10%.
740. The hair cell regeneration agent(s) and/or the Wnt agonist and/or the epigenetic modulator for use according to embodiment 738 or embodiment 739, wherein said treatment provides an improved words-in-noise score for the patient, wherein said improvement, if tested, would be at least 20%.
741. The hair cell regeneration agent(s) and/or the Wnt agonist and/or the epigenetic modulator for use according to any of embodiments 738-740, wherein said treatment provides an improved words-in-noise score for the patient, wherein said improvement, if tested, would be at least 30.
742. The hair cell regeneration agent(s) and/or the Wnt agonist and/or the epigenetic modulator for use according to any of embodiments 738-741, wherein said treatment provides an improved words-in-noise score for the patient, wherein said improvement, if tested, would be at least 50%.
743. The hair cell regeneration agent(s) and/or the Wnt agonist and/or the epigenetic modulator for use according to any of embodiments 738-742, wherein said treatment provides an improved words-in-noise score for the patient, wherein said improvement, if tested, would be at least 70%.
744. The hair cell regeneration agent(s) and/or the Wnt agonist and/or the epigenetic modulator for use according to any of embodiments 738-743, wherein said treatment provides an improved words-in-noise score for the patient, wherein said improvement, if tested, would be at least 100%.
745. The hair cell regeneration agent(s) and/or the Wnt agonist and/or the epigenetic modulator for use according to any preceding embodiment, wherein said treatment provides improved words-in-noise recognition for the patient, wherein said improvement, if tested, would be at least 5, at least 7, at least 10 words relative to the number of words recognized by the patient in a words-in-noise test of 70 words prior to the treatment.
746. The hair cell regeneration agent(s) and/or the Wnt agonist and/or the epigenetic modulator for use according to embodiment 745, wherein said treatment provides improved words-in-noise recognition for the patient, wherein said improvement, if tested, would be at least 5 words relative to the number of words recognized by the patient in a words-in-noise test of 70 words prior to the treatment.
747. The hair cell regeneration agent(s) and/or the Wnt agonist and/or the epigenetic modulator for use according to embodiment 745 or embodiment 746, wherein said treatment provides improved words-in-noise recognition for the patient, wherein said improvement, if tested, would be at least 7 words relative to the number of words recognized by the patient in a words-in-noise test of 70 words prior to the treatment.
748. The hair cell regeneration agent(s) and/or the Wnt agonist and/or the epigenetic modulator for use according to any of embodiments 745-747, wherein said treatment provides improved words-in-noise recognition for the patient, wherein said improvement, if tested, would be at least 10 words relative to the number of words recognized by the patient in a words-in-noise test of 70 words prior to the treatment.
749. The hair cell regeneration agent(s) and/or the Wnt agonist and/or the epigenetic modulator for use according to any preceding embodiment, wherein said treatment provides improved words-in-noise recognition for the patient, wherein said improvement, if tested, would be at least 2, at least 3, at least 5 words relative to the number of words recognized by the patient in a words-in-noise test of 35 words prior to the treatment.
750. The hair cell regeneration agent(s) and/or the Wnt agonist and/or the epigenetic modulator for use according to embodiment 749, wherein said treatment provides improved words-in-noise recognition for the patient, wherein said improvement, if tested, would be at least 2 words relative to the number of words recognized by the patient in a words-in-noise test of 35 words prior to the treatment.
751. The hair cell regeneration agent(s) and/or the Wnt agonist and/or the epigenetic modulator for use according to embodiment 749 or embodiment 750, wherein said treatment provides improved words-in-noise recognition for the patient, wherein said improvement, if tested, would be at least 3 words relative to the number of words recognized by the patient in a words-in-noise test of 35 words prior to the treatment.
752. The hair cell regeneration agent(s) and/or the Wnt agonist and/or the epigenetic modulator for use according to any of embodiments 749-751, wherein said treatment provides improved words-in-noise recognition for the patient, wherein said improvement, if tested, would be at least 5 words relative to the number of words recognized by the patient in a words-in-noise test of 35 words prior to the treatment.
753. The hair cell regeneration agent(s) and/or the Wnt agonist and/or the epigenetic modulator for use according to any preceding embodiment, wherein said treatment provides an improved Signal-to-Noise ratio (SNR) for the predicted mean of 50% correct words in a words-in-noise test for the patient, wherein said improvement, if tested, would be at least 0.5 dB, at least 1.0 dB, at least 1.5 dB, at least 2 dB, at least 2.5 dB, at least 3.0 dB, at least 3.5 dB, at least 4.0 dB, at least 4.5 dB, at least 5.0 dB, relative to the patient's SNR for the predicted mean of 50% correct words in a words-in-noise test prior to the treatment, wherein the SNR is calculated using the Spearman-Kärber equation.
754. The hair cell regeneration agent(s) and/or the Wnt agonist and/or the epigenetic modulator for use according to embodiment 753, wherein said treatment provides an improved Signal-to-Noise ratio (SNR) for the predicted mean of 500% correct words in a words-in-noise test for the patient, wherein said improvement, if tested, would be at least 0.5 dB, relative to the patient's SNR for the predicted mean of 50% correct words in a words-in-noise test prior to the treatment, wherein the SNR is calculated using the Spearman-Kärber equation.
755. The hair cell regeneration agent(s) and/or the Wnt agonist and/or the epigenetic modulator for use according to embodiment 753 or embodiment 754, wherein said treatment provides an improved Signal-to-Noise ratio (SNR) for the predicted mean of 50% correct words in a words-in-noise test for the patient, wherein said improvement, if tested, would be at least 1 dB, relative to the patient's SNR for the predicted mean of 50% correct words in a words-in-noise test prior to the treatment, wherein the SNR is calculated using the Spearman-Kärber equation.
756. The hair cell regeneration agent(s) and/or the Wnt agonist and/or the epigenetic modulator for use according to any of embodiments 753-755, wherein said treatment provides an improved Signal-to-Noise ratio (SNR) for the predicted mean of 50% correct words in a words-in-noise test for the patient, wherein said improvement, if tested, would be at least 1.5 dB, relative to the patient's SNR for the predicted mean of 50% correct words in a words-in-noise test prior to the treatment, wherein the SNR is calculated using the Spearman-Kärber equation.
757. The hair cell regeneration agent(s) and/or the Wnt agonist and/or the epigenetic modulator for use according to any of embodiments 753-756, wherein said treatment provides an improved Signal-to-Noise ratio (SNR) for the predicted mean of 50% correct words in a words-in-noise test for the patient, wherein said improvement, if tested, would be at least 2 dB, relative to the patient's SNR for the predicted mean of 50% correct words in a words-in-noise test prior to the treatment, wherein the SNR is calculated using the Spearman-Kärber equation.
758. The hair cell regeneration agent(s) and/or the Wnt agonist and/or the epigenetic modulator for use according to any of embodiments 753-757, wherein said treatment provides an improved Signal-to-Noise ratio (SNR) for the predicted mean of 50% correct words in a words-in-noise test for the patient, wherein said improvement, if tested, would be at least 2.5 dB, relative to the patient's SNR for the predicted mean of 50% correct words in a words-in-noise test prior to the treatment, wherein the SNR is calculated using the Spearman-Kärber equation.
759. The hair cell regeneration agent(s) and/or the Wnt agonist and/or the epigenetic modulator for use according to any of embodiments 753-758, wherein said treatment provides an improved Signal-to-Noise ratio (SNR) for the predicted mean of 50% correct words in a words-in-noise test for the patient, wherein said improvement, if tested, would be at least 3 dB, relative to the patient's SNR for the predicted mean of 50% correct words in a words-in-noise test prior to the treatment, wherein the SNR is calculated using the Spearman-Kärber equation.
760. The hair cell regeneration agent(s) and/or the Wnt agonist and/or the epigenetic modulator for use according to any of embodiments 753-759, wherein said treatment provides an improved Signal-to-Noise ratio (SNR) for the predicted mean of 50% correct words in a words-in-noise test for the patient, wherein said improvement, if tested, would be at least 3.5 dB, relative to the patient's SNR for the predicted mean of 50% correct words in a words-in-noise test prior to the treatment, wherein the SNR is calculated using the Spearman-Kärber equation.
761. The hair cell regeneration agent(s) and/or the Wnt agonist and/or the epigenetic modulator for use according to any of embodiments 753-760, wherein said treatment provides an improved Signal-to-Noise ratio (SNR) for the predicted mean of 50% correct words in a words-in-noise test for the patient, wherein said improvement, if tested, would be at least 4 dB, relative to the patient's SNR for the predicted mean of 50% correct words in a words-in-noise test prior to the treatment, wherein the SNR is calculated using the Spearman-Kärber equation.
762. The hair cell regeneration agent(s) and/or the Wnt agonist and/or the epigenetic modulator for use according to any of embodiments 753-761, wherein said treatment provides an improved Signal-to-Noise ratio (SNR) for the predicted mean of 50% correct words in a words-in-noise test for the patient, wherein said improvement, if tested, would be at least 4.5 dB, relative to the patient's SNR for the predicted mean of 50% correct words in a words-in-noise test prior to the treatment, wherein the SNR is calculated using the Spearman-Kärber equation.
763. The hair cell regeneration agent(s) and/or the Wnt agonist and/or the epigenetic modulator for use according to any of embodiments 753-762, wherein said treatment provides an improved Signal-to-Noise ratio (SNR) for the predicted mean of 50% correct words in a words-in-noise test for the patient, wherein said improvement, if tested, would be at least 5 dB, relative to the patient's SNR for the predicted mean of 50% correct words in a words-in-noise test prior to the treatment, wherein the SNR is calculated using the Spearman-Kärber equation.
764. The hair cell regeneration agent(s) and/or the Wnt agonist and/or the epigenetic modulator for use according to any of embodiments 738-763 wherein the average of the patient's hearing thresholds across 0.5 kHz, 1 kHz, 2 kHz and 4 kHz after the treatment, if tested, would be no more than 10 dB increased relative to the average of the patient's hearing thresholds across 0.5 kHz, 1 kHz, 2 kHz and 4 kHz prior to the treatment, wherein said hearing thresholds are measured by pure tone audiometry.
765. The hair cell regeneration agent(s) and/or the Wnt agonist and/or the epigenetic modulator for use according to any of embodiments 738-763, wherein the average of the patient's hearing thresholds across 0.5 kHz, 1 kHz, 2 kHz and 4 kHz after the treatment, if tested, would be no more than 5 dB increased or decreased to the average of the patient's hearing thresholds across 0.5 kHz, 1 kHz, 2 kHz and 4 kHz prior to the treatment, wherein said hearing thresholds are measured by pure tone audiometry.
766. The hair cell regeneration agent(s) and/or the Wnt agonist and/or the epigenetic modulator for use according to any preceding embodiment, wherein said treatment provides an improved hearing threshold at 4 kHz, 6 kHz and/or 8 kHz, wherein said improvement, if tested, would be at least 5 dB, at least 10 dB, at least 20 dB, at least 30 dB relative to the patient's hearing threshold at 4 kHz, 6 kHz and/or 8 kHz prior to the treatment, wherein said hearing threshold is measured by pure tone audiometry.
767. The hair cell regeneration agent(s) and/or the Wnt agonist and/or the epigenetic modulator for use according to embodiment 766, wherein said treatment provides an improved hearing threshold at 4 kHz, wherein said improvement, if tested, would be at least 5 dB relative to the patient's hearing threshold at 4 kHz prior to the treatment, wherein said hearing threshold is measured by pure tone audiometry.
768. The hair cell regeneration agent(s) and/or the Wnt agonist and/or the epigenetic modulator for use according to embodiment 766 or embodiment 767, wherein said treatment provides an improved hearing threshold at 4 kHz, wherein said improvement, if tested, would be at least 10 dB relative to the patient's hearing threshold at 4 kHz prior to the treatment, wherein said hearing threshold is measured by pure tone audiometry.
769. The hair cell regeneration agent(s) and/or the Wnt agonist and/or the epigenetic modulator for use according to any of embodiments 766-768, wherein said treatment provides an improved hearing threshold at 4 kHz, wherein said improvement, if tested, would be at least 20 dB relative to the patient's hearing threshold at 4 kHz prior to the treatment, wherein said hearing threshold is measured by pure tone audiometry.
770. The hair cell regeneration agent(s) and/or the Wnt agonist and/or the epigenetic modulator for use according to any of embodiments 766-769, wherein said treatment provides an improved hearing threshold at 4 kHz, wherein said improvement, if tested, would be at least 30 dB relative to the patient's hearing threshold at 4 kHz prior to the treatment, wherein said hearing threshold is measured by pure tone audiometry.
771. The hair cell regeneration agent(s) and/or the Wnt agonist and/or the epigenetic modulator for use according to any of embodiments 766-770, wherein said treatment provides an improved hearing threshold at 6 kHz, wherein said improvement, if tested, would be at least 5 dB relative to the patient's hearing threshold at 6 kHz prior to the treatment, wherein said hearing threshold is measured by pure tone audiometry.
772. The hair cell regeneration agent(s) and/or the Wnt agonist and/or the epigenetic modulator for use according to any of embodiments 766-771, wherein said treatment provides an improved hearing threshold at 6 kHz, wherein said improvement, if tested, would be at least 10 dB relative to the patient's hearing threshold at 6 kHz prior to the treatment, wherein said hearing threshold is measured by pure tone audiometry.
773. The hair cell regeneration agent(s) and/or the Wnt agonist and/or the epigenetic modulator for use according to any of embodiments 766-772, wherein said treatment provides an improved hearing threshold at 6 kHz, wherein said improvement, if tested, would be at least 20 dB relative to the patient's hearing threshold at 6 kHz prior to the treatment, wherein said hearing threshold is measured by pure tone audiometry.
774. The hair cell regeneration agent(s) and/or the Wnt agonist and/or the epigenetic modulator for use according to any of embodiments 766-773, wherein said treatment provides an improved hearing threshold at 6 kHz, wherein said improvement, if tested, would be at least 30 dB relative to the patient's hearing threshold at 6 kHz prior to the treatment, wherein said hearing threshold is measured by pure tone audiometry.
775. The hair cell regeneration agent(s) and/or the Wnt agonist and/or the epigenetic modulator for use according to any of embodiments 766-774, wherein said treatment provides an improved hearing threshold at 8 kHz, wherein said improvement, if tested, would be at least 5 dB relative to the patient's hearing threshold at 8 kHz prior to the treatment, wherein said hearing threshold is measured by pure tone audiometry.
776. The hair cell regeneration agent(s) and/or the Wnt agonist and/or the epigenetic modulator for use according to any of embodiments 766-775, wherein said treatment provides an improved hearing threshold at 8 kHz, wherein said improvement, if tested, would be at least 10 dB relative to the patient's hearing threshold at 8 kHz prior to the treatment, wherein said hearing threshold is measured by pure tone audiometry.
777. The hair cell regeneration agent(s) and/or the Wnt agonist and/or the epigenetic modulator for use according to any of embodiments 766-776, wherein said treatment provides an improved hearing threshold at 8 kHz, wherein said improvement, if tested, would be at least 20 dB relative to the patient's hearing threshold at 8 kHz prior to the treatment, wherein said hearing threshold is measured by pure tone audiometry.
778. The hair cell regeneration agent(s) and/or the Wnt agonist and/or the epigenetic modulator for use according to any of embodiments 766-777, wherein said treatment provides an improved hearing threshold at 8 kHz, wherein said improvement, if tested, would be at least 30 dB relative to the patient's hearing threshold at 8 kHz prior to the treatment, wherein said hearing threshold is measured by pure tone audiometry.
779. The hair cell regeneration agent(s) and/or the Wnt agonist and/or the epigenetic modulator for use according to any preceding embodiment, wherein said treatment provides an improvement to the average of the patient hearing thresholds across 4 kHz, 6 kHz and 8 kHz when measured by pure tone audiometry, wherein said improvement, if tested, would be at least 1, 2, 3, 4, 5, 6, 7, 8, 9, 10, 12, 15, 20, 25, 30 dB relative to the average of the patient hearing thresholds across 4 kHz, 6 kHz and 8 kHz when measured by pure tone audiometry prior to the treatment.
780. The hair cell regeneration agent(s) and/or the Wnt agonist and/or the epigenetic modulator for use according to embodiment 779, wherein said treatment provides an improved hearing threshold, wherein said improvement, if tested, would be at least 1 dB relative to the average of the patient hearing thresholds across 4 kHz, 6 kHz and 8 kHz when measured by pure tone audiometry prior to the treatment.
781. The hair cell regeneration agent(s) and/or the Wnt agonist and/or the epigenetic modulator for use according to embodiment 779 or embodiment 780, wherein said treatment provides an improved hearing threshold, wherein said improvement, if tested, would be at least 2 dB relative to the average of the patient hearing thresholds across 4 kHz, 6 kHz and 8 kHz when measured by pure tone audiometry prior to the treatment.
782. The hair cell regeneration agent(s) and/or the Wnt agonist and/or the epigenetic modulator for use according to any of embodiments 779-781, wherein said treatment provides an improved hearing threshold, wherein said improvement, if tested, would be at least 3 dB relative to the average of the patient hearing thresholds across 4 kHz, 6 kHz and 8 kHz when measured by pure tone audiometry prior to the treatment.
783. The hair cell regeneration agent(s) and/or the Wnt agonist and/or the epigenetic modulator for use according to any of embodiments 779-782, wherein said treatment provides an improved hearing threshold, wherein said improvement, if tested, would be at least 4 dB relative to the average of the patient hearing thresholds across 4 kHz, 6 kHz and 8 kHz when measured by pure tone audiometry prior to the treatment.
784. The hair cell regeneration agent(s) and/or the Wnt agonist and/or the epigenetic modulator for use according to any of embodiments 779-783, wherein said treatment provides an improved hearing threshold, wherein said improvement, if tested, would be at least 5 dB relative to the average of the patient hearing thresholds across 4 kHz, 6 kHz and 8 kHz when measured by pure tone audiometry prior to the treatment.
785. The hair cell regeneration agent(s) and/or the Wnt agonist and/or the epigenetic modulator for use according to any of embodiments 779-784, wherein said treatment provides an improved hearing threshold, wherein said improvement, if tested, would be at least 6 dB relative to the average of the patient hearing thresholds across 4 kHz, 6 kHz and 8 kHz when measured by pure tone audiometry prior to the treatment.
786. The hair cell regeneration agent(s) and/or the Wnt agonist and/or the epigenetic modulator for use according to any of embodiments 779-785, wherein said treatment provides an improved hearing threshold, wherein said improvement, if tested, would be at least 7 dB relative to the average of the patient hearing thresholds across 4 kHz, 6 kHz and 8 kHz when measured by pure tone audiometry prior to the treatment.
787. The hair cell regeneration agent(s) and/or the Wnt agonist and/or the epigenetic modulator for use according to any of embodiments 779-786, wherein said treatment provides an improved hearing threshold, wherein said improvement, if tested, would be at least 8 dB relative to the average of the patient hearing thresholds across 4 kHz, 6 kHz and 8 kHz when measured by pure tone audiometry prior to the treatment.
788. The hair cell regeneration agent(s) and/or the Wnt agonist and/or the epigenetic modulator for use according to any of embodiments 779-787, wherein said treatment provides an improved hearing threshold, wherein said improvement, if tested, would be at least 9 dB relative to the average of the patient hearing thresholds across 4 kHz, 6 kHz and 8 kHz when measured by pure tone audiometry prior to the treatment.
789. The hair cell regeneration agent(s) and/or the Wnt agonist and/or the epigenetic modulator for use according to any of embodiments 779-788, wherein said treatment provides an improved hearing threshold, wherein said improvement, if tested, would be at least 10 dB relative to the average of the patient hearing thresholds across 4 kHz, 6 kHz and 8 kHz when measured by pure tone audiometry prior to the treatment.
790. The hair cell regeneration agent(s) and/or the Wnt agonist and/or the epigenetic modulator for use according to any of embodiments 779-789, wherein said treatment provides an improved hearing threshold, wherein said improvement, if tested, would be at least 12 dB relative to the average of the patient hearing thresholds across 4 kHz, 6 kHz and 8 kHz when measured by pure tone audiometry prior to the treatment.
791. The hair cell regeneration agent(s) and/or the Wnt agonist and/or the epigenetic modulator for use according to any of embodiments 779-790, wherein said treatment provides an improved hearing threshold, wherein said improvement, if tested, would be at least 15 dB relative to the average of the patient hearing thresholds across 4 kHz, 6 kHz and 8 kHz when measured by pure tone audiometry prior to the treatment.
792. The hair cell regeneration agent(s) and/or the Wnt agonist and/or the epigenetic modulator for use according to any of embodiments 779-791, wherein said treatment provides an improved hearing threshold, wherein said improvement, if tested, would be at least 20 dB relative to the average of the patient hearing thresholds across 4 kHz, 6 kHz and 8 kHz when measured by pure tone audiometry prior to the treatment.
793. The hair cell regeneration agent(s) and/or the Wnt agonist and/or the epigenetic modulator for use according to any of embodiments 779-792, wherein said treatment provides an improved hearing threshold, wherein said improvement, if tested, would be at least 25 dB relative to the average of the patient hearing thresholds across 4 kHz, 6 kHz and 8 kHz when measured by pure tone audiometry prior to the treatment.
794. The hair cell regeneration agent(s) and/or the Wnt agonist and/or the epigenetic modulator for use according to any of embodiments 779-793, wherein said treatment provides an improved hearing threshold, wherein said improvement, if tested, would be at least 30 dB relative to the average of the patient hearing thresholds across 4 kHz, 6 kHz and 8 kHz when measured by pure tone audiometry prior to the treatment.
795. The hair cell regeneration agent(s) and/or the Wnt agonist and/or the epigenetic modulator for use according to any preceding embodiment, wherein said treatment provides the patient with hearing thresholds that provide an average value of less than 40 dB HL across 4 kHz, 6 kHz and 8 kHz when measured by pure tone audiometry after the treatment.
796. The hair cell regeneration agent(s) and/or the Wnt agonist and/or the epigenetic modulator for use according to embodiment 795, wherein said treatment provides the patient with hearing thresholds that provide an average value of less than 25 dB HL across 4 kHz, 6 kHz and 8 kHz when measured by pure tone audiometry after the treatment.
797. The hair cell regeneration agent(s) and/or the Wnt agonist and/or the epigenetic modulator for use according to any preceding embodiment, wherein said treatment also provides treatment for tinnitus, when assessed using one or more measures selected from the group consisting of: tinnitus functional index (TFI), tinnitus handicap index (THI), tinnitus reaction questionnaire (TRQ), tinnitus severity index (TSI), and tinnitus handicap questionnaire (THQ).
798. The hair cell regeneration agent(s) and/or the Wnt agonist and/or the epigenetic modulator for use according to any of embodiments 727-797 wherein said treatment, or said improvement, is observed in an individual patient.
799. The hair cell regeneration agent(s) and/or the Wnt agonist and/or the epigenetic modulator for use according to any of embodiments 727-797 wherein said treatment, or said improvement, is observed as an average across a patient population.
800. The hair cell regeneration agent(s) and/or the Wnt agonist and/or the epigenetic modulator for use according to any preceding embodiment, wherein an improvement in hearing is provided within 15, 30, 60 or 90 days.
801. The hair cell regeneration agent(s) and/or the Wnt agonist and/or the epigenetic modulator for use according to embodiment 800, wherein an improvement in hearing is provided within 15 days, wherein optionally said improvement is maintained at least until 90 days.
802. The hair cell regeneration agent(s) and/or the Wnt agonist and/or the epigenetic modulator for use according to embodiment 800, wherein an improvement in hearing is provided within 30 days, wherein optionally said improvement is maintained at least until 90 days.
803. The hair cell regeneration agent(s) and/or the Wnt agonist and/or the epigenetic modulator for use according to embodiment 800, wherein an improvement in hearing is provided within 60 days, wherein optionally said improvement is maintained at least until 90 days.
804. The hair cell regeneration agent(s) and/or the Wnt agonist and/or the epigenetic modulator for use according to embodiment 800, wherein an improvement in hearing is provided within 90 days.
805. The hair cell regeneration agent(s) and/or the Wnt agonist and/or the epigenetic modulator for use according to any of embodiments 800-804, wherein said improvement in hearing is as defined in any one of embodiments 727-799.
806. The hair cell regeneration agent(s) and/or the Wnt agonist and/or the epigenetic modulator for use according to any preceding embodiment, wherein the treatment comprises one or more administrations of the hair cell regeneration agent(s) and/or the Wnt agonist and/or the epigenetic modulator.
807. The hair cell regeneration agent(s) and/or the Wnt agonist and/or the epigenetic modulator for use according to embodiment 806, wherein the treatment comprises no more than a single administration.
808. The hair cell regeneration agent(s) and/or the Wnt agonist and/or the epigenetic modulator for use according to embodiment 806, wherein an improvement in hearing is provided by a single administration.
809. The hair cell regeneration agent(s) and/or the Wnt agonist and/or the epigenetic modulator for use according to any preceding embodiment, wherein the administration of the hair cell regeneration agent(s) and/or the Wnt agonist and/or the epigenetic modulator is to the middle ear.
810. The hair cell regeneration agent(s) and/or the Wnt agonist and/or the epigenetic modulator for use according to embodiment 809, wherein the administration to the middle ear is by intratympanic injection.
811. The hair cell regeneration agent(s) or Wnt agonist and/or epigenetic modulator for use according to any preceding embodiment, wherein the hair cell regeneration agent(s) and/or the Wnt agonist and/or the epigenetic modulator is present in the patient for no more than 24, or 12 or 6 hours post-administration.
812. The hair cell regeneration agent(s) or Wnt agonist and/or epigenetic modulator for use according to embodiment 811, wherein the hair cell regeneration agent(s) and/or the Wnt agonist and/or the epigenetic modulator is present in the patient for no more than 24 hours post-administration.
813. The hair cell regeneration agent(s) or Wnt agonist and/or epigenetic modulator for use according to embodiment 811, wherein the hair cell regeneration agent(s) and/or the Wnt agonist and/or the epigenetic modulator is present in the patient for no more than 12 hours post-administration.
814. The hair cell regeneration agent(s) or Wnt agonist and/or epigenetic modulator for use according to embodiment 811, wherein the hair cell regeneration agent(s) and/or the Wnt agonist and/or the epigenetic modulator is present in the patient for no more than 6 hours post-administration.
815. CHIR99021 or a pharmaceutically acceptable salt thereof and valproic acid (VPA) or a pharmaceutically acceptable salt thereof for use in treating sensorineural hearing loss in a human patient, wherein optionally the average of the patient's hearing thresholds across 0.5 kHz, 1 kHz, 2 kHz and 4 kHz is at least 40 dB HL and no more than 70 dB HL when measured by pure tone audiometry prior to the treatment.
816. CHIR99021 or a pharmaceutically acceptable salt thereof and valproic acid (VPA) or a pharmaceutically acceptable salt thereof for use according embodiment 815, wherein the patient has a standard word recognition score of 60% or less prior to the treatment.
817. CHIR99021 or a pharmaceutically acceptable salt thereof and valproic acid (VPA) or a pharmaceutically acceptable salt thereof for use according to embodiment 815 or embodiment 816, wherein the patient has a words-in-noise score of 50% or less prior to the treatment.
818. CHIR99021 or a pharmaceutically acceptable salt thereof and valproic acid (VPA) or a pharmaceutically acceptable salt thereof for use according to any of embodiments 815-817, wherein said treatment provides an improved standard word recognition score for the patient, wherein said improvement, if tested, would be at least 10%, wherein said percentage improvement is calculated using the following formula:

$$100 \times ( \frac{patient ’ s word recognition score after treatment}{patient ’ s word recognition score prior to treatment} - 1 )$$

819. CHIR99021 or a pharmaceutically acceptable salt thereof and valproic acid (VPA) or a pharmaceutically acceptable salt thereof for use according to any of embodiments 815-818, wherein said treatment provides an improved words-in-noise score for the patient, wherein said improvement, if tested, would be at least 10%, wherein said percentage improvement is calculated using the following formula:

$$100 \times ( \frac{patient ’ s words in noise score after treatment}{patient ’ s words in noise score prior to treatment} - 1 )$$

820. CHIR99021 or a pharmaceutically acceptable salt thereof and valproic acid (VPA) or a pharmaceutically acceptable salt thereof for use according to any of embodiments 815-819, wherein the average of the patient's hearing thresholds across 0.5 kHz, 1 kHz, 2 kHz and 4 kHz after the treatment, if tested, would be no more than 5 dB increased or decreased to the average of the patient's hearing thresholds across 0.5 kHz, 1 kHz, 2 kHz and 4 kHz prior to the treatment, wherein said hearing thresholds are measured by pure tone audiometry.
821. CHIR99021 or a pharmaceutically acceptable salt thereof and valproic acid (VPA) or a pharmaceutically acceptable salt thereof for use according to any of embodiments 815-820, wherein said treatment provides an improved hearing threshold at 8 kHz, wherein said improvement, if tested, would be at least 5 dB relative to the patient's hearing threshold at 8 kHz prior to the treatment, wherein said hearing threshold is measured by pure tone audiometry.
822. CHIR99021 or a pharmaceutically acceptable salt thereof and valproic acid (VPA) or a pharmaceutically acceptable salt thereof for use according to any of embodiments 815-821, wherein an improvement in hearing is provided within 90 days.
823. CHIR99021 or a pharmaceutically acceptable salt thereof and valproic acid (VPA) or a pharmaceutically acceptable salt thereof for use according to any of embodiments 815-822, wherein an improvement in hearing is provided by a single administration.
824. CHIR99021 or a pharmaceutically acceptable salt thereof and valproic acid (VPA) or a pharmaceutically acceptable salt thereof for use according to any of embodiments 815-823, wherein said improvement in hearing is as defined in any one of embodiments 818-821.
825. CHIR99021 or a pharmaceutically acceptable salt thereof and valproic acid (VPA) or a pharmaceutically acceptable salt thereof for use according to any of embodiments 815-824, wherein said compounds are administered to the middle ear.
826. CHIR99021 or a pharmaceutically acceptable salt thereof and valproic acid (VPA) or a pharmaceutically acceptable salt thereof for use according to embodiment 825, wherein administration to the middle ear is by intratympanic injection.
827. CHIR99021 or a pharmaceutically acceptable salt thereof and valproic acid (VPA) or a pharmaceutically acceptable salt thereof for use according to any of embodiments 815-826, wherein the pharmaceutically acceptable salt of valproic acid is sodium valproate.
828. CHIR99021 or a pharmaceutically acceptable salt thereof and valproic acid (VPA) or a pharmaceutically acceptable salt thereof for use according to any of embodiments 815-827, wherein the CHIR99021 is at a concentration of about 6.1 mM, about 6.2 mM, about 6.3 mM, about 6.4 mM, about 6.5 mM, about 6.6 mM, about 6.7 mM, about 6.8 mM, about 6.9 mM, about 7.0 mM, about 7.1 mM, about 7.2 mM, about 7.3 mM, about 7.4 mM, for example, about 6.75 mM.
829. CHIR99021 or a pharmaceutically acceptable salt thereof and valproic acid (VPA) or a pharmaceutically acceptable salt thereof for use according to any of embodiments 815-828, wherein the VPA is at a concentration of about 465 mM, about 475 mM, about 485 mM, about 495 mM, about 505 mM, about 515 mM, about 525 mM, about 535 mM, about 545 mM or about 555 mM, about 565 mM, about 575 mM, about 585 mM, about 595 mM, about 605 mM, for example, about 533 mM.
830. CHIR99021 or a pharmaceutically acceptable salt thereof and valproic acid (VPA) or a pharmaceutically acceptable salt thereof for use according to any of embodiments 815-829, wherein the CHIR99021 and the VPA are formulated as a single composition.
831. CHIR99021 or a pharmaceutically acceptable salt thereof and valproic acid (VPA) or a pharmaceutically acceptable salt thereof for use according to embodiment 830, wherein the composition comprises a poloxamer, for example, Poloxamer 407.
832. The hair cell regeneration agent(s) and/or the Wnt agonist and/or the epigenetic modulator for use according to any of embodiments 800-814, wherein the improvement is maintained until at least 90, 120, 180 or 365 days.
833. The hair cell regeneration agent(s) and/or the Wnt agonist and/or the epigenetic modulator for use according to embodiment 832, wherein the improvement is maintained until at least 90 days.
834. The hair cell regeneration agent(s) and/or the Wnt agonist and/or the epigenetic modulator for use according to embodiment 832, wherein the improvement is maintained until at least 120 days.
835. The hair cell regeneration agent(s) and/or the Wnt agonist and/or the epigenetic modulator for use according to embodiment 832, wherein the improvement is maintained until at least 180 days.
836. The hair cell regeneration agent(s) and/or the Wnt agonist and/or the epigenetic modulator for use according to embodiment 832, wherein the improvement is maintained until at least 365 days.
837. The hair cell regeneration agent(s) for use according to any preceding embodiment, wherein the hair cell regeneration agent(s) is a Notch inhibitor.
838. The hair cell regeneration agent(s) for use according to embodiment 837, wherein the Notch inhibitor is 3H4 MB or a pharmaceutically acceptable salt thereof.
839. The hair cell regeneration agent(s) for use according to embodiment 837, wherein the Notch inhibitor is BMS-871 or a pharmaceutically acceptable salt thereof.
840. The hair cell regeneration agent(s) for use according to embodiment 837, wherein the Notch inhibitor is EDD3 or a pharmaceutically acceptable salt thereof
841. The hair cell regeneration agent(s) for use according to embodiment 837, wherein the Notch inhibitor is ELN-46719 or a pharmaceutically acceptable salt thereof.
842. The hair cell regeneration agent(s) for use according to embodiment 837, wherein the Notch inhibitor is FLI-06 or a pharmaceutically acceptable salt thereof.
843. The hair cell regeneration agent(s) for use according to embodiment 837, wherein the Notch inhibitor is IMR-1 or a pharmaceutically acceptable salt thereof
844. The hair cell regeneration agent(s) for use according to embodiment 837, wherein the Notch inhibitor is JLK6 or a pharmaceutically acceptable salt thereof.
845. The hair cell regeneration agent(s) for use according to embodiment 837, wherein the Notch inhibitor is TAPI-1 or a pharmaceutically acceptable salt thereof.
846. The hair cell regeneration agent(s) for use according to any preceding embodiment, wherein the hair cell regeneration agent(s) is a Atoh1 activator.
847. The hair cell regeneration agent(s) and/or Wnt agonist and/or epigenetic modulator for use according to any preceding embodiment wherein the hair cell regeneration agent(s) and/or Wnt agonist and/or epigenetic modulator is administered at a volume of about 50 μL, about 100 μL, about 150 μL or about 200 μL.
848. The hair cell regeneration agent(s) and/or the Wnt agonist and/or the epigenetic modulator for use according to any preceding embodiment, wherein said treatment provides an improved standard word recognition score for the patient, wherein said improvement, if tested, is a score falling outside an 85%, 90%, 95%, 97.5% or 99% confidence interval for the patient's word recognition score prior to the treatment as defined by Thornton and Raffin (1978).
849. The hair cell regeneration agent(s) and/or the Wnt agonist and/or the epigenetic modulator for use according to embodiment 848, wherein said treatment provides an improved standard word recognition score for the patient, wherein said improvement, if tested, is a score falling outside an 85% confidence interval for the patient's word recognition score prior to the treatment as defined by Thornton and Raffin (1978).
850. The hair cell regeneration agent(s) and/or the Wnt agonist and/or the epigenetic modulator for use according to embodiment 848, wherein said treatment provides an improved standard word recognition score for the patient, wherein said improvement, if tested, is a score falling outside an 90% confidence interval for the patient's word recognition score prior to the treatment as defined by Thornton and Raffin (1978).
851. The hair cell regeneration agent(s) and/or the Wnt agonist and/or the epigenetic modulator for use according to embodiment 848, wherein said treatment provides an improved standard word recognition score for the patient, wherein said improvement, if tested, is a score falling outside an 95% confidence interval for the patient's word recognition score prior to the treatment as defined by Thornton and Raffin (1978).
852. The hair cell regeneration agent(s) and/or the Wnt agonist and/or the epigenetic modulator for use according to embodiment 848, wherein said treatment provides an improved standard word recognition score for the patient, wherein said improvement, if tested, is a score falling outside an 97.5% confidence interval for the patient's word recognition score prior to the treatment as defined by Thornton and Raffin (1978).
853. The hair cell regeneration agent(s) and/or the Wnt agonist and/or the epigenetic modulator for use according to embodiment 848, wherein said treatment provides an improved standard word recognition score for the patient, wherein said improvement, if tested, is a score falling outside an 99% confidence interval for the patient's word recognition score prior to the treatment as defined by Thornton and Raffin (1978).
854. The hair cell regeneration agent(s) and/or the Wnt agonist and/or the epigenetic modulator for use according to any preceding embodiment, wherein the treatment provides an improved standard word recognition score for the patient, wherein said improvement, if tested, would be at least 6%, at least 10%, at least 20%, at least 25%, at least 30%, at least 35%, at least 40%, at least 50%, wherein said percentage improvement is calculated using the following formula:

$$100 \times ( \frac{WR score post treatment - WR score prior to treatment}{number of words in standard word recognition test} )$$

855. The hair cell regeneration agent(s) and/or the Wnt agonist and/or the epigenetic modulator for use according to embodiment 854, treatment provides an improved standard word recognition score for the patient, wherein said improvement, if tested, would be at least 6%.
856. The hair cell regeneration agent(s) and/or the Wnt agonist and/or the epigenetic modulator for use according to embodiment 854 or embodiment 855, treatment provides an improved standard word recognition score for the patient, wherein said improvement, if tested, would be at least 10%.
857. The hair cell regeneration agent(s) and/or the Wnt agonist and/or the epigenetic modulator for use according to any of embodiments 854-856, treatment provides an improved standard word recognition score for the patient, wherein said improvement, if tested, would be at least 20%.
858. The hair cell regeneration agent(s) and/or the Wnt agonist and/or the epigenetic modulator for use according to any of embodiments 854-857, treatment provides an improved standard word recognition score for the patient, wherein said improvement, if tested, would be at least 30%.
859. The hair cell regeneration agent(s) and/or the Wnt agonist and/or the epigenetic modulator for use according to any of embodiments 854-858, treatment provides an improved standard word recognition score for the patient, wherein said improvement, if tested, would be at least 40%.
860. The hair cell regeneration agent(s) and/or the Wnt agonist and/or the epigenetic modulator for use according to any of embodiments 854-859, treatment provides an improved standard word recognition score for the patient, wherein said improvement, if tested, would be at least 50%.
861. The hair cell regeneration agent(s) and/or the Wnt agonist and/or the epigenetic modulator for use according to any preceding embodiment, wherein the treatment provides an improved words-in-noise score for the patient, wherein said improvement, if tested, would be at least 6%, at least 10%, at least 20%, at least 25%, at least 30%, at least 35%, at least 40%, at least 50%, wherein said percentage improvement is calculated using the following formula:

$$100 \times ( \frac{WIN score post treatment - WIN score prior to treatment}{number of words in words in noise test} )$$

862. The hair cell regeneration agent(s) and/or the Wnt agonist and/or the epigenetic modulator for use according to embodiment 861, treatment provides an improved words-in-noise score for the patient, wherein said improvement, if tested, would be at least 6%.
863. The hair cell regeneration agent(s) and/or the Wnt agonist and/or the epigenetic modulator for use according to embodiment 861 or embodiment 862, treatment provides an improved words-in-noise score for the patient, wherein said improvement, if tested, would be at least 10%.
864. The hair cell regeneration agent(s) and/or the Wnt agonist and/or the epigenetic modulator for use according to any of embodiments 861-863, treatment provides an improved words-in-noise score for the patient, wherein said improvement, if tested, would be at least 20%.
865. The hair cell regeneration agent(s) and/or the Wnt agonist and/or the epigenetic modulator for use according to any of embodiments 861-864, treatment provides an improved words-in-noise score for the patient, wherein said improvement, if tested, would be at least 30%.
866. The hair cell regeneration agent(s) and/or the Wnt agonist and/or the epigenetic modulator for use according to any of embodiments 861-865, treatment provides an improved words-in-noise score for the patient, wherein said improvement, if tested, would be at least 40%.
867. The hair cell regeneration agent(s) and/or the Wnt agonist and/or the epigenetic modulator for use according to any of embodiments 861-866, treatment provides an improved words-in-noise score for the patient, wherein said improvement, if tested, would be at least 50%.
868. The hair cell regeneration agent(s) and/or the Wnt agonist and/or the epigenetic modulator for use according to any preceding embodiment, wherein said improvement is observed in at least 20%, at least 30%, at least 40% or at least 50% of a patient population.
869. The hair cell regeneration agent(s) and/or the Wnt agonist and/or the epigenetic modulator for use according to any of embodiments 848-868, wherein the improvement is maintained until at least 90, 120, 180 or 365 days.
870. The hair cell regeneration agent(s) and/or the Wnt agonist and/or the epigenetic modulator for use according to embodiment 869, wherein the improvement is maintained until at least 90 days.
871. The hair cell regeneration agent(s) and/or the Wnt agonist and/or the epigenetic modulator for use according to embodiment 869, wherein the improvement is maintained until at least 120 days.
872. The hair cell regeneration agent(s) and/or the Wnt agonist and/or the epigenetic modulator for use according to embodiment 869, wherein the improvement is maintained until at least 180 days.
873. The hair cell regeneration agent(s) and/or the Wnt agonist and/or the epigenetic modulator for use according to embodiment 869, wherein the improvement is maintained until at least 365 days.

### ADDITIONAL NUMBERED EMBODIMENTS

1. CHIR99021 or a pharmaceutically acceptable salt thereof and valproic acid (VPA) or a pharmaceutically acceptable salt thereof for use in treating sensorineural hearing loss in a human patient, wherein the sensorineural hearing loss is moderate or moderately severe sensorineural hearing loss.
2. CHIR99021 or a pharmaceutically acceptable salt thereof and valproic acid (VPA) or a pharmaceutically acceptable salt thereof for use in treating sensorineural hearing loss in a human patient, wherein the average of the patient's hearing thresholds across 0.5 kHz, 1 kHz, 2 kHz and 4 kHz is greater than 40 dB HL and no more than 70 dB HL when measured by pure tone audiometry prior to the treatment.
3. CHIR99021 or a pharmaceutically acceptable salt thereof and valproic acid (VPA) or a pharmaceutically acceptable salt thereof for use in treating sensorineural hearing loss in a human patient, wherein the patient has a standard word recognition score of 60% or less prior to the treatment.
4. CHIR99021 or a pharmaceutically acceptable salt thereof and valproic acid (VPA) or a pharmaceutically acceptable salt thereof for use in treating sensorineural hearing loss in a human patient, wherein the patient has a words-in-noise score of 50% or less prior to the treatment.
5. CHIR99021 or a pharmaceutically acceptable salt thereof and valproic acid (VPA) or a pharmaceutically acceptable salt thereof for use in treating sensorineural hearing loss in a human patient, wherein
   the sensorineural hearing loss is moderate or moderately severe sensorineural hearing loss; the average of the patient's hearing thresholds across 0.5 kHz, 1 kHz, 2 kHz and 4 kHz is greater than 40 dB HL and no more than 70 dB HL when measured by pure tone audiometry prior to the treatment; and
   the patient has a standard word recognition score of 60% or less prior to the treatment or a words-in-noise score of 50% or less prior to the treatment.
6. CHIR99021 or a pharmaceutically acceptable salt thereof and valproic acid (VPA) or a pharmaceutically acceptable salt thereof for use according to any of embodiments 1-5, wherein the average of the patient's hearing thresholds across 0.5 kHz, 1 kHz, 2 kHz and 4 kHz is greater than 40 dB HL and no more than 55 dB HL when measured by pure tone audiometry prior to the treatment.
7. CHIR99021 or a pharmaceutically acceptable salt thereof and valproic acid (VPA) or a pharmaceutically acceptable salt thereof for use according to any of embodiments 1-5, wherein the average of the patient's hearing thresholds across 0.5 kHz, 1 kHz, 2 kHz and 4 kHz is greater than 55 dB HL and no more than 70 dB HL when measured by pure tone audiometry prior to the treatment.
8. CHIR99021 or a pharmaceutically acceptable salt thereof and valproic acid (VPA) or a pharmaceutically acceptable salt thereof for use according to any of embodiments 1-7, wherein an improvement in hearing is provided within 90 days.
9. CHIR99021 or a pharmaceutically acceptable salt thereof and valproic acid (VPA) or a pharmaceutically acceptable salt thereof for use according to any of embodiments 1-8, wherein an improvement in hearing is provided by a single administration.
10. CHIR99021 or a pharmaceutically acceptable salt thereof and valproic acid (VPA) or a pharmaceutically acceptable salt thereof for use according to any of embodiments 1-9, wherein said compounds are administered to the middle ear.
11. CHIR99021 or a pharmaceutically acceptable salt thereof and valproic acid (VPA) or a pharmaceutically acceptable salt thereof for use according to embodiment 10, wherein administration to the middle ear is by intratympanic injection.
12. CHIR99021 or a pharmaceutically acceptable salt thereof and valproic acid (VPA) or a pharmaceutically acceptable salt thereof for use according to any of embodiments 1-11, wherein the pharmaceutically acceptable salt of valproic acid is sodium valproate.
13. CHIR99021 or a pharmaceutically acceptable salt thereof and valproic acid (VPA) or a pharmaceutically acceptable salt thereof for use according to any of embodiments 1-12, wherein the CHIR99021 and the VPA are formulated as a single composition.
14. CHIR99021 or a pharmaceutically acceptable salt thereof and valproic acid (VPA) or a pharmaceutically acceptable salt thereof for use according to embodiment 13, wherein the composition comprises a Poloxamer 407.
15. CHIR99021 or a pharmaceutically acceptable salt thereof and valproic acid (VPA) or a pharmaceutically acceptable salt thereof for use according to any of embodiments 1-14, wherein the sensorineural hearing loss is sudden sensorineural hearing loss or noise-induced sensorineural hearing loss.
16. CHIR99021 or a pharmaceutically acceptable salt thereof and valproic acid (VPA) or a pharmaceutically acceptable salt thereof for use according to embodiment 15, wherein the sensorineural hearing loss is sudden sensorineural hearing loss.
17. CHIR99021 or a pharmaceutically acceptable salt thereof and valproic acid (VPA) or a pharmaceutically acceptable salt thereof for use according to embodiment 15, wherein the sensorineural hearing loss is noise-induced sensorineural hearing loss.
18. CHIR99021 or a pharmaceutically acceptable salt thereof and valproic acid (VPA) or a pharmaceutically acceptable salt thereof for use according to any of embodiments 1-17, wherein said treatment also provides treatment for tinnitus, when assessed using one or more measures selected from the group consisting of: tinnitus functional index (TFI), tinnitus handicap index (THI), tinnitus reaction questionnaire (TRQ), tinnitus severity index (TSI), and tinnitus handicap questionnaire (THQ).
19. CHIR99021 or a pharmaceutically acceptable salt thereof and valproic acid (VPA) or a pharmaceutically acceptable salt thereof for use according to any of embodiments 1-18, wherein said treatment also provides treatment for tinnitus, when assessed using tinnitus functional index (TFI).
20. CHIR99021 or a pharmaceutically acceptable salt thereof and valproic acid (VPA) or a pharmaceutically acceptable salt thereof for use in treating tinnitus in a human patient with sensorineural hearing loss, wherein the tinnitus is diagnosed using one or more of the methods selected from the group consisting of: tinnitus functional index (TFI), tinnitus handicap index (THI), tinnitus reaction questionnaire (TRQ), tinnitus severity index (TSI), and tinnitus handicap questionnaire (THQ).
21. CHIR99021 or a pharmaceutically acceptable salt thereof and valproic acid (VPA) or a pharmaceutically acceptable salt thereof for use according to embodiment 20, wherein the tinnitus is diagnosed using tinnitus functional index (TFI).
22. CHIR99021 or a pharmaceutically acceptable salt thereof and valproic acid (VPA) or a pharmaceutically acceptable salt thereof for use according to embodiment 20 or embodiment 21, wherein the sensorineural hearing loss is mild, moderate or moderately severe sensorineural hearing loss.
23. CHIR99021 or a pharmaceutically acceptable salt thereof and valproic acid (VPA) or a pharmaceutically acceptable salt thereof for use according to embodiments 20-22, wherein the average of the patient's hearing thresholds across 0.5 kHz, 1 kHz, 2 kHz and 4 kHz is greater than 25 dB HL and no more than 70 dB HL when measured by pure tone audiometry prior to the treatment.
24. CHIR99021 or a pharmaceutically acceptable salt thereof and valproic acid (VPA) or a pharmaceutically acceptable salt thereof for use according to any of embodiments 20-22, wherein the average of the patient's hearing thresholds across 0.5 kHz, 1 kHz, 2 kHz and 4 kHz is greater than 25 dB HL and no more than 40 dB HL when measured by pure tone audiometry prior to the treatment.
25. CHIR99021 or a pharmaceutically acceptable salt thereof and valproic acid (VPA) or a pharmaceutically acceptable salt thereof for use according to any of embodiments 20-22, wherein the average of the patient's hearing thresholds across 0.5 kHz, 1 kHz, 2 kHz and 4 kHz is greater than 40 dB HL and no more than 55 dB HL when measured by pure tone audiometry prior to the treatment.
26. CHIR99021 or a pharmaceutically acceptable salt thereof and valproic acid (VPA) or a pharmaceutically acceptable salt thereof for use according to any of embodiments 20-22, wherein the average of the patient's hearing thresholds across 0.5 kHz, 1 kHz, 2 kHz and 4 kHz is greater than 55 dB HL and no more than 70 dB HL when measured by pure tone audiometry prior to the treatment.
27. CHIR99021 or a pharmaceutically acceptable salt thereof and valproic acid (VPA) or a pharmaceutically acceptable salt thereof for use according to any of embodiments 20-26, wherein the sensorineural hearing loss is sudden sensorineural hearing loss or noise-induced sensorineural hearing loss.
28. CHIR99021 or a pharmaceutically acceptable salt thereof and valproic acid (VPA) or a pharmaceutically acceptable salt thereof for use according to embodiment 27, wherein the sensorineural hearing loss is sudden sensorineural hearing loss.
29. CHIR99021 or a pharmaceutically acceptable salt thereof and valproic acid (VPA) or a pharmaceutically acceptable salt thereof for use according to embodiment 27, wherein the sensorineural hearing loss is noise-induced sensorineural hearing loss.
30. A pharmaceutical composition comprising CHIR99021 or a pharmaceutically acceptable salt thereof and VPA or a pharmaceutically acceptable salt thereof for treating sensorineural hearing loss in a human patient, wherein the sensorineural hearing loss is moderate or moderately severe sensorineural hearing loss.
31. A pharmaceutical composition comprising CHIR99021 or a pharmaceutically acceptable salt thereof and VPA or a pharmaceutically acceptable salt thereof for treating sensorineural hearing loss in a human patient, wherein the average of the patient's hearing thresholds across 0.5 kHz, 1 kHz, 2 kHz and 4 kHz is greater than 40 dB HL and no more than 70 dB HL when measured by pure tone audiometry prior to the treatment.
32. A pharmaceutical composition comprising CHIR99021 or a pharmaceutically acceptable salt thereof and VPA or a pharmaceutically acceptable salt thereof for treating sensorineural hearing loss in a human patient, wherein the patient has a standard word recognition score of 60% or less prior to the treatment.
33. A pharmaceutical composition comprising CHIR99021 or a pharmaceutically acceptable salt thereof and VPA or a pharmaceutically acceptable salt thereof for treating sensorineural hearing loss in a human patient, wherein the patient has a words-in-noise score of 50% or less prior to the treatment.
34. A pharmaceutical composition comprising CHIR99021 or a pharmaceutically acceptable salt thereof and VPA or a pharmaceutically acceptable salt thereof for treating sensorineural hearing loss in a human patient, wherein
   the sensorineural hearing loss is moderate or moderately severe sensorineural hearing loss; the average of the patient's hearing thresholds across 0.5 kHz, 1 kHz, 2 kHz and 4 kHz is greater than 40 dB HL and no more than 70 dB HL when measured by pure tone audiometry prior to the treatment; and
   the patient has a standard word recognition score of 60% or less prior to the treatment or a words-in-noise score of 50% or less prior to the treatment.
35. The pharmaceutical composition for use according to any of embodiments 30-34, wherein the average of the patient's hearing thresholds across 0.5 kHz, 1 kHz, 2 kHz and 4 kHz is greater than 40 dB HL and no more than 55 dB HL when measured by pure tone audiometry prior to the treatment.
36. The pharmaceutical composition for use according to any of embodiments 30-34, wherein the average of the patient's hearing thresholds across 0.5 kHz, 1 kHz, 2 kHz and 4 kHz is greater than 55 dB HL and no more than 70 dB HL when measured by pure tone audiometry prior to the treatment.
37. The pharmaceutical composition for use according to any of embodiments 30-36, wherein an improvement in hearing is provided within 90 days.
38. The pharmaceutical composition for use according to any of embodiments 30-37, wherein an improvement in hearing is provided by a single administration.
39. The pharmaceutical composition for use according to any of embodiments 30-38, wherein said compounds are administered to the middle ear.
40. The pharmaceutical composition for use according to embodiment 39 wherein administration to the middle ear is by intratympanic injection.
41. The pharmaceutical composition for use according to any of embodiments 30-40, wherein the pharmaceutically acceptable salt of valproic acid is sodium valproate.
42. The pharmaceutical composition for use according to any of embodiments 3041, wherein the composition comprises a Poloxamer 407.
43. The pharmaceutical composition for use according to any of embodiments 30-42, wherein the sensorineural hearing loss is sudden sensorineural hearing loss or noise-induced sensorineural hearing loss.
44. The pharmaceutical composition for use according to embodiment 43, wherein the sensorineural hearing loss is sudden sensorineural hearing loss.
45. The pharmaceutical composition for use according to embodiment 43, wherein the sensorineural hearing loss is noise-induced sensorineural hearing loss.
46. The pharmaceutical composition for use according to any of embodiments 3045, wherein said treatment also provides treatment for tinnitus, when assessed using one or more measures selected from the group consisting of: tinnitus functional index (TFI), tinnitus handicap index (THI), tinnitus reaction questionnaire (TRQ), tinnitus severity index (TSI), and tinnitus handicap questionnaire (THQ).
47. The pharmaceutical composition for use according to any of embodiments 30-46, wherein said treatment also provides treatment for tinnitus, when assessed using tinnitus functional index (TFI).
48. A pharmaceutical composition comprising CHIR99021 or a pharmaceutically acceptable salt thereof and VPA or a pharmaceutically acceptable salt thereof for treating tinnitus in a human patient with sensorineural hearing loss, wherein the tinnitus is diagnosed using one or more of the methods selected from the group consisting of: tinnitus functional index (TFI), tinnitus handicap index (THI), tinnitus reaction questionnaire (TRQ), tinnitus severity index (TSI), and tinnitus handicap questionnaire (THQ).
49. The pharmaceutical composition for use according to embodiment 48, wherein the tinnitus is diagnosed using tinnitus functional index (TFI).
50. The pharmaceutical composition for use according to embodiment 48 or embodiment 49, wherein the sensorineural hearing loss is mild, moderate or moderately severe sensorineural hearing loss.
51. The pharmaceutical composition for use according to any of embodiments 48-50, wherein the average of the patient's hearing thresholds across 0.5 kHz, 1 kHz, 2 kHz and 4 kHz is greater than 25 dB HL and no more than 70 dB HL when measured by pure tone audiometry prior to the treatment.
52. The pharmaceutical composition for use according to any of embodiments 48-50, wherein the average of the patient's hearing thresholds across 0.5 kHz, 1 kHz, 2 kHz and 4 kHz is greater than 25 dB HL and no more than 40 dB HL when measured by pure tone audiometry prior to the treatment.
53. The pharmaceutical composition for use according to any of embodiments 48-50, wherein the average of the patient's hearing thresholds across 0.5 kHz, 1 kHz, 2 kHz and 4 kHz is greater than 40 dB HL and no more than 55 dB HL when measured by pure tone audiometry prior to the treatment.
54. The pharmaceutical composition for use according to any of embodiments 48-50, wherein the average of the patient's hearing thresholds across 0.5 kHz, 1 kHz, 2 kHz and 4 kHz is greater than 55 dB HL and no more than 70 dB HL when measured by pure tone audiometry prior to the treatment.
55. The pharmaceutical composition for use according to any of embodiments 48-54, wherein the sensorineural hearing loss is sudden sensorineural hearing loss or noise-induced sensorineural hearing loss.
56. The pharmaceutical composition for use according to embodiment 55, wherein the sensorineural hearing loss is sudden sensorineural hearing loss.
57. The pharmaceutical composition for use according to embodiment 55, wherein the sensorineural hearing loss is noise-induced sensorineural hearing loss.
58. A method of treating sensorineural hearing loss in a human patient by administering CHIR99021 or a pharmaceutically acceptable salt thereof and valproic acid (VPA) or a pharmaceutically acceptable salt thereof to said patient, wherein the sensorineural hearing loss is moderate or moderately severe sensorineural hearing loss.
59. A method of treating sensorineural hearing loss in a human patient by administering CHIR99021 or a pharmaceutically acceptable salt thereof and valproic acid (VPA) or a pharmaceutically acceptable salt thereof to said patient, wherein the average of the patient's hearing thresholds across 0.5 kHz, 1 kHz, 2 kHz and 4 kHz is greater than 40 dB HL and no more than 70 dB HL when measured by pure tone audiometry prior to the treatment.
60. A method of treating sensorineural hearing loss in a human patient by administering CHIR99021 or a pharmaceutically acceptable salt thereof and valproic acid (VPA) or a pharmaceutically acceptable salt thereof to said patient, wherein the patient has a standard word recognition score of 60% or less prior to the treatment.
61. A method of treating sensorineural hearing loss in a human patient by administering CHIR99021 or a pharmaceutically acceptable salt thereof and valproic acid (VPA) or a pharmaceutically acceptable salt thereof to said patient, wherein the patient has a words-in-noise score of 50% or less prior to the treatment.
62. A method of treating sensorineural hearing loss in a human patient by administering CHIR99021 or a pharmaceutically acceptable salt thereof and valproic acid (VPA) or a pharmaceutically acceptable salt thereof to said patient, wherein
   the sensorineural hearing loss is moderate or moderately severe sensorineural hearing loss; the average of the patient's hearing thresholds across 0.5 kHz, 1 kHz, 2 kHz and 4 kHz is greater than 40 dB HL and no more than 70 dB HL when measured by pure tone audiometry prior to the treatment; and
   the patient has a standard word recognition score of 60% or less prior to the treatment or a words-in-noise score of 50% or less prior to the treatment.
63. The method of any of embodiments 58-62, wherein the average of the patient's hearing thresholds across 0.5 kHz, 1 kHz, 2 kHz and 4 kHz is greater than 40 dB HL and no more than 55 dB HL when measured by pure tone audiometry prior to the treatment.
64. The method of any of embodiments 58-62, wherein the average of the patient's hearing thresholds across 0.5 kHz, 1 kHz, 2 kHz and 4 kHz is greater than 55 dB HL and no more than 70 dB HL when measured by pure tone audiometry prior to the treatment.
65. The method of any of embodiments 58-64, wherein an improvement in hearing is provided within 90 days.
66. The method of any of embodiments 58-65, wherein an improvement in hearing is provided by a single administration.
67. The method of any of embodiments 58-66, wherein said compounds are administered to the middle ear.
68. The method of embodiment 67, wherein administration to the middle ear is by intratympanic injection.
69. The method of any of embodiments 58-68, wherein the pharmaceutically acceptable salt of valproic acid is sodium valproate.
70. The method of any of embodiments 58-69, wherein the CHIR99021 and the VPA are formulated as a single composition.
71. The method of embodiment 70, wherein the composition comprises a Poloxamer 407.
72. The method of any of embodiments 58-71, wherein the sensorineural hearing loss is sudden sensorineural hearing loss or noise-induced sensorineural hearing loss.
73. The method of embodiment 72, wherein the sensorineural hearing loss is sudden sensorineural hearing loss.
74. The method of embodiment 72, wherein the sensorineural hearing loss is noise-induced sensorineural hearing loss.
75. The method of any of embodiments 58-74, wherein said treatment also provides treatment for tinnitus, when assessed using one or more measures selected from the group consisting of: tinnitus functional index (TFI), tinnitus handicap index (THI), tinnitus reaction questionnaire (TRQ), tinnitus severity index (TSI), and tinnitus handicap questionnaire (THQ).
76. A method according to any of embodiments 58-76, wherein said treatment also provides treatment for tinnitus, when assessed using tinnitus functional index (TFI).
77. A method of treating tinnitus in a human patient with sensorineural hearing loss by administering CHIR99021 or a pharmaceutically acceptable salt thereof and valproic acid (VPA) or a pharmaceutically acceptable salt thereof to said patient, wherein the tinnitus is diagnosed using one or more of the methods selected from the group consisting of: tinnitus functional index (TFI), tinnitus handicap index (THI), tinnitus reaction questionnaire (TRQ), tinnitus severity index (TSI), and tinnitus handicap questionnaire (THQ).
78. The method of embodiment 77, wherein the tinnitus is diagnosed using tinnitus functional index (TFI).
79. The method of embodiment 77 or embodiment 78, wherein the sensorineural hearing loss is mild, moderate or moderately severe sensorineural hearing loss.
80. The method of any of embodiments 77-79, wherein the average of the patient's hearing thresholds across 0.5 kHz, 1 kHz, 2 kHz and 4 kHz is greater than 25 dB HL and no more than 70 dB HL when measured by pure tone audiometry prior to the treatment.
81. The method of any of embodiments 77-79, wherein the average of the patient's hearing thresholds across 0.5 kHz, 1 kHz, 2 kHz and 4 kHz is greater than 25 dB HL and no more than 40 dB HL when measured by pure tone audiometry prior to the treatment.
82. The method of any of embodiments 77-79, wherein the average of the patient's hearing thresholds across 0.5 kHz, 1 kHz, 2 kHz and 4 kHz is greater than 40 dB HL and no more than 55 dB HL when measured by pure tone audiometry prior to the treatment.
83. The method of any of embodiments 77-79, wherein the average of the patient's hearing thresholds across 0.5 kHz, 1 kHz, 2 kHz and 4 kHz is greater than 55 dB HL and no more than 70 dB HL when measured by pure tone audiometry prior to the treatment.
84. The method of any of embodiments 77-83, wherein the sensorineural hearing loss is sudden sensorineural hearing loss or noise-induced sensorineural hearing loss.
85. The method of embodiment 84, wherein the sensorineural hearing loss is sudden sensorineural hearing loss.
86. The method of embodiment 84, wherein the sensorineural hearing loss is noise-induced sensorineural hearing loss.
87. Use of CHIR99021 or a pharmaceutically acceptable salt thereof and VPA or a pharmaceutically acceptable salt thereof for the manufacture of a pharmaceutical composition for treating sensorineural hearing loss in a human patient, wherein the sensorineural hearing loss is moderate or moderately severe sensorineural hearing loss.
88. Use of CHIR99021 or a pharmaceutically acceptable salt thereof and VPA or a pharmaceutically acceptable salt thereof for the manufacture of a pharmaceutical composition for treating sensorineural hearing loss in a human patient, wherein the average of the patient's hearing thresholds across 0.5 kHz, 1 kHz, 2 kHz and 4 kHz is greater than 40 dB HL and no more than 70 dB HL when measured by pure tone audiometry prior to the treatment.
89. Use of CHIR99021 or a pharmaceutically acceptable salt thereof and VPA or a pharmaceutically acceptable salt thereof for the manufacture of a pharmaceutical composition for treating sensorineural hearing loss in a human patient, wherein the patient has a standard word recognition score of 60% or less prior to the treatment.
90. Use of CHIR99021 or a pharmaceutically acceptable salt thereof and VPA or a pharmaceutically acceptable salt thereof for the manufacture of a pharmaceutical composition for treating sensorineural hearing loss in a human patient, wherein the patient has a words-in-noise score of 50% or less prior to the treatment.
91. Use of CHIR99021 or a pharmaceutically acceptable salt thereof and VPA or a pharmaceutically acceptable salt thereof for the manufacture of a pharmaceutical composition for treating sensorineural hearing loss in a human patient, wherein
   the sensorineural hearing loss is moderate or moderately severe sensorineural hearing loss; the average of the patient's hearing thresholds across 0.5 kHz, 1 kHz, 2 kHz and 4 kHz is greater than 40 dB HL and no more than 70 dB HL when measured by pure tone audiometry prior to the treatment; and
   the patient has a standard word recognition score of 60% or less prior to the treatment or a words-in-noise score of 50% or less prior to the treatment.
92. Use of CHIR99021 or a pharmaceutically acceptable salt thereof and VPA or a pharmaceutically acceptable salt thereof for treating sensorineural hearing loss in a human patient, wherein the sensorineural hearing loss is moderate or moderately severe sensorineural hearing loss.
93. Use of CHIR99021 or a pharmaceutically acceptable salt thereof and VPA or a pharmaceutically acceptable salt thereof for treating sensorineural hearing loss in a human patient, wherein the average of the patient's hearing thresholds across 0.5 kHz, 1 kHz, 2 kHz and 4 kHz is greater than 40 dB HL and no more than 70 dB HL when measured by pure tone audiometry prior to the treatment.
94. Use of CHIR99021 or a pharmaceutically acceptable salt thereof and VPA or a pharmaceutically acceptable salt thereof for treating sensorineural hearing loss in a human patient, wherein the patient has a standard word recognition score of 60% or less prior to the treatment.
95. Use of CHIR99021 or a pharmaceutically acceptable salt thereof and VPA or a pharmaceutically acceptable salt thereof for treating sensorineural hearing loss in a human patient, wherein the patient has a words-in-noise score of 50% or less prior to the treatment.
96. Use of CHIR99021 or a pharmaceutically acceptable salt thereof and VPA or a pharmaceutically acceptable salt thereof for treating sensorineural hearing loss in a human patient, wherein
   the sensorineural hearing loss is moderate or moderately severe sensorineural hearing loss; the average of the patient's hearing thresholds across 0.5 kHz, 1 kHz, 2 kHz and 4 kHz is greater than 40 dB HL and no more than 70 dB HL when measured by pure tone audiometry prior to the treatment; and
   the patient has a standard word recognition score of 60% or less prior to the treatment or a words-in-noise score of 50% or less prior to the treatment.
97. The use of any of embodiments 87-96, wherein the average of the patient's hearing thresholds across 0.5 kHz, 1 kHz, 2 kHz and 4 kHz is greater than 40 dB HL and no more than 55 dB HL when measured by pure tone audiometry prior to the treatment.
98. The use of any of embodiments 87-96, wherein the average of the patient's hearing thresholds across 0.5 kHz, 1 kHz, 2 kHz and 4 kHz is greater than 55 dB HL and no more than 70 dB HL when measured by pure tone audiometry prior to the treatment.
99. The use of any of embodiments 87-98, wherein an improvement in hearing is provided within 90 days.
100. The use of any of embodiments 87-99, wherein an improvement in hearing is provided by a single administration.
101. The use of any of embodiments 87-100, wherein said compounds are administered to the middle ear.
102. The use of embodiment 101, wherein administration to the middle ear is by intratympanic injection.
103. The use of any of embodiments 87-102, wherein the pharmaceutically acceptable salt of valproic acid is sodium valproate.
104. The use of any of embodiments 87-103, wherein the CHIR99021 and the VPA are formulated as a single composition.
105. The use of embodiment 104, wherein the composition comprises a Poloxamer 407.
106. The use of any of embodiments 87-105, wherein the sensorineural hearing loss is sudden sensorineural hearing loss or noise-induced sensorineural hearing loss.
107. The use of embodiment 106, wherein the sensorineural hearing loss is sudden sensorineural hearing loss.
108. The use of embodiment 106, wherein the sensorineural hearing loss is noise-induced sensorineural hearing loss.
109. The use of any of embodiments 87-108, wherein said treatment also provides treatment for tinnitus, when assessed using one or more measures selected from the group consisting of: tinnitus functional index (TFI), tinnitus handicap index (THI), tinnitus reaction questionnaire (TRQ), tinnitus severity index (TSI), and tinnitus handicap questionnaire (THQ).
110. The use of any of embodiments 87-109, wherein said treatment also provides treatment for tinnitus, when assessed using tinnitus functional index (TFI).
111. Use of CHIR99021 or a pharmaceutically acceptable salt thereof and VPA or a pharmaceutically acceptable salt thereof for the manufacture of a pharmaceutical composition for treating tinnitus in a human patient with sensorineural hearing loss, wherein the tinnitus is diagnosed using one or more of the methods selected from the group consisting of: tinnitus functional index (TFI), tinnitus handicap index (THI), tinnitus reaction questionnaire (TRQ), tinnitus severity index (TSI), and tinnitus handicap questionnaire (THQ).
112. Use of CHIR99021 or a pharmaceutically acceptable salt thereof and VPA or a pharmaceutically acceptable salt thereof for treating tinnitus in a human patient with sensorineural hearing loss, wherein the tinnitus is diagnosed using one or more of the methods selected from the group consisting of: tinnitus functional index (TFI), tinnitus handicap index (THI), tinnitus reaction questionnaire (TRQ), tinnitus severity index (TSI), and tinnitus handicap questionnaire (THQ).
113. The use of embodiment 111 or embodiment 112, wherein the tinnitus is diagnosed using tinnitus functional index (TFI).
114. The use of any of embodiments 111-113, wherein the sensorineural hearing loss is mild, moderate or moderately severe sensorineural hearing loss.
115. The use of any of embodiments 111-114, wherein the average of the patient's hearing thresholds across 0.5 kHz, 1 kHz, 2 kHz and 4 kHz is greater than 25 dB HL and no more than 70 dB HL when measured by pure tone audiometry prior to the treatment.
116. The use of any of embodiments 111-114, wherein the average of the patient's hearing thresholds across 0.5 kHz, 1 kHz, 2 kHz and 4 kHz is greater than 25 dB HL and no more than 40 dB HL when measured by pure tone audiometry prior to the treatment.
117. The use of any of embodiments 111-114, wherein the average of the patient's hearing thresholds across 0.5 kHz, 1 kHz, 2 kHz and 4 kHz is greater than 40 dB HL and no more than 55 dB HL when measured by pure tone audiometry prior to the treatment.
118. The use of any of embodiments 111-114, wherein the average of the patient's hearing thresholds across 0.5 kHz, 1 kHz, 2 kHz and 4 kHz is greater than 55 dB HL and no more than 70 dB HL when measured by pure tone audiometry prior to the treatment.
119. The use of any of embodiments 111-118, wherein the sensorineural hearing loss is sudden sensorineural hearing loss or noise-induced sensorineural hearing loss.
120. The use of embodiment 119, wherein the sensorineural hearing loss is sudden sensorineural hearing loss.
121. The use of embodiment 119, wherein the sensorineural hearing loss is noise-induced sensorineural hearing loss.
122. A hair cell regeneration agent(s) for use in treating sensorineural hearing loss in a human patient, wherein the sensorineural hearing loss is moderate or moderately severe sensorineural hearing loss
123. A hair cell regeneration agent(s) for use in treating sensorineural hearing loss in a human patient, wherein the average of the patients hearing thresholds across 0.5 kHz, 1 kHz, 2 kHz and 4 kHz is greater than 40 dB HL and no more than 70 dB HL when measured by pure tone audiometry prior to the treatment.
124. A hair cell regeneration agent(s) for use in treating sensorineural hearing loss in a human patient, wherein the patient has a standard word recognition score of 60% or less prior to the treatment.
125. A hair cell regeneration agent(s) for use in treating sensorineural hearing loss in a human patient, wherein the patient has a words-in-noise score of 50% or less prior to the treatment.
126. The hair cell regeneration agent(s) for use according to any of embodiments 122-125, wherein said treatment provides an improved standard word recognition score for the patient, wherein said improvement, if tested, would be at least 10%, wherein said percentage improvement is calculated using the following formula:

$$100 \times ( \frac{patient ’ s word recognition score after treatment}{patient ’ s word recognition score prior to treatment} - 1 )$$

127. The hair cell regeneration agent(s) for use according to any of embodiments 122-126, wherein said treatment provides an improved words-in-noise score for the patient, wherein said improvement, if tested, would be at least 10%, wherein said percentage improvement is calculated using the following formula:

$$100 \times ( \frac{patient ’ s words in noise score after treatment}{patient ’ s words in noise score prior to treatment} - 1 )$$

wherein said treatment optionally also provides the improvement specified in embodiment 126.
128. The hair cell regeneration agent(s) for use according to any of embodiments 122-127, wherein said treatment provides an improved hearing threshold at 8 kHz, wherein said improvement, if tested, would be at least 5 dB relative to the patient's hearing threshold at 8 kHz prior to the treatment, wherein said hearing threshold is measured by pure tone audiometry, wherein said treatment optionally also provides the improvement specified in embodiment 126 or embodiment 127.
129. A hair cell regeneration agent(s) for use in treating sensorineural hearing loss in a human patient, wherein said treatment provides an improved standard word recognition score for the patient, wherein said improvement, if tested, would be at least 10%, wherein said percentage improvement is calculated using the following formula:

$$100 \times ( \frac{patient ’ s word recognition score after treatment}{patient ’ s word recognition score prior to treatment} - 1 )$$

130. A hair cell regeneration agent(s) for use in treating sensorineural hearing loss in a human patient, wherein said treatment provides an improved words-in-noise score for the patient, wherein said improvement, if tested, would be at least 10%, wherein said percentage improvement is calculated using the following formula:

$$100 \times ( \frac{patient ’ s words in noise score after treatment}{patient ’ s words in noise score prior to treatment} - 1 )$$

wherein said treatment optionally also provides the improvement specified in embodiment 129.
131. A hair cell regeneration agent(s) for use in treating sensorineural hearing loss in a human patient, wherein said treatment provides an improved hearing threshold at 8 kHz, wherein said improvement, if tested, would be at least 5 dB relative to the patient's hearing threshold at 8 kHz prior to the treatment, wherein said hearing threshold is measured by pure tone audiometry, wherein said treatment optionally also provides the improvement specified in embodiment 129 or embodiment 130.
132. The hair cell regeneration agent(s) for use according to any of embodiments 122-131, wherein an improvement in hearing is provided within 90 days.
133. The hair cell regeneration agent(s) for use according to any of embodiments 122-132, wherein an improvement in hearing is provided by a single administration.
134. The hair cell regeneration agent(s) for use according to any of embodiments 122-133, wherein said compounds are administered to the middle ear.
135. The hair cell regeneration agent(s) for use according to embodiment 134 wherein administration to the middle ear is by intratympanic injection.
136. The hair cell regeneration agent(s) for use according to any of embodiments 122-135, wherein the hair cell regeneration agent(s) is a combination of agents formulated as a single composition.
137. The hair cell regeneration agent(s) for use according to any of embodiments 122-136, wherein said treatment also provides treatment for tinnitus, when assessed using one or more measures selected from the group consisting of: tinnitus functional index (TFI), tinnitus handicap index (THI), tinnitus reaction questionnaire (TRQ), tinnitus severity index (TSI), and tinnitus handicap questionnaire (THQ).
138. A hair cell regeneration agent(s) for use in treating tinnitus in a human patient with sensorineural hearing loss, wherein the tinnitus is diagnosed using one or more of the methods selected from the group consisting of: tinnitus functional index (TFI), tinnitus handicap index (THI), tinnitus reaction questionnaire (TRQ), tinnitus severity index (TSI), and tinnitus handicap questionnaire (THQ).
139. The hair cell regeneration agent(s) for use according to embodiment 138, wherein the sensorineural hearing loss is mild, moderate or moderately severe sensorineural hearing loss.

1. CHIR99021 or a pharmaceutically acceptable salt thereof and valproic acid (VPA) or a pharmaceutically acceptable salt thereof for use in treating sensorineural hearing loss in a human patient, wherein the sensorineural hearing loss is moderate or moderately severe sensorineural hearing loss.

2. CHIR99021 or a pharmaceutically acceptable salt thereof and valproic acid (VPA) or a pharmaceutically acceptable salt thereof for use in treating sensorineural hearing loss in a human patient, wherein the average of the patient's hearing thresholds across 0.5 kHz, 1 kHz, 2 kHz and 4 kHz is greater than 40 dB HL and no more than 70 dB HL when measured by pure tone audiometry prior to the treatment.

3. CHIR99021 or a pharmaceutically acceptable salt thereof and valproic acid (VPA) or a pharmaceutically acceptable salt thereof for use in treating sensorineural hearing loss in a human patient, wherein the patient has a standard word recognition score of 60% or less prior to the treatment.

4. CHIR99021 or a pharmaceutically acceptable salt thereof and valproic acid (VPA) or a pharmaceutically acceptable salt thereof for use in treating sensorineural hearing loss in a human patient, wherein the patient has a words-in-noise score of 50% or less prior to the treatment.

5. CHIR99021 or a pharmaceutically acceptable salt thereof and valproic acid (VPA) or a pharmaceutically acceptable salt thereof for use in treating sensorineural hearing loss in a human patient, wherein
the sensorineural hearing loss is moderate or moderately severe sensorineural hearing loss;
the average of the patient's hearing thresholds across 0.5 kHz, 1 kHz, 2 kHz and 4 kHz is greater than 40 dB HL and no more than 70 dB HL when measured by pure tone audiometry prior to the treatment; and
the patient has a standard word recognition score of 60% or less prior to the treatment or a words-in-noise score of 50% or less prior to the treatment.

6. CHIR99021 or a pharmaceutically acceptable salt thereof and valproic acid (VPA) or a pharmaceutically acceptable salt thereof for use according to any of claims 1-5, wherein the average of the patient's hearing thresholds across 0.5 kHz, 1 kHz, 2 kHz and 4 kHz is greater than 40 dB HL and no more than 55 dB HL when measured by pure tone audiometry prior to the treatment.

7. CHIR99021 or a pharmaceutically acceptable salt thereof and valproic acid (VPA) or a pharmaceutically acceptable salt thereof for use according to any of claims 1-5, wherein the average of the patient's hearing thresholds across 0.5 kHz, 1 kHz, 2 kHz and 4 kHz is greater than 55 dB HL and no more than 70 dB HL when measured by pure tone audiometry prior to the treatment.

8. CHIR99021 or a pharmaceutically acceptable salt thereof and valproic acid (VPA) or a pharmaceutically acceptable salt thereof for use according to any of claims 1-7, wherein an improvement in hearing is provided within 90 days.

9. CHIR99021 or a pharmaceutically acceptable salt thereof and valproic acid (VPA) or a pharmaceutically acceptable salt thereof for use according to any of claims 1-8, wherein an improvement in hearing is provided by a single administration.

10. CHIR99021 or a pharmaceutically acceptable salt thereof and valproic acid (VPA) or a pharmaceutically acceptable salt thereof for use according to any of claims 1-9, wherein said compounds are administered to the middle ear.

11. CHIR99021 or a pharmaceutically acceptable salt thereof and valproic acid (VPA) or a pharmaceutically acceptable salt thereof for use according to claim 10, wherein administration to the middle ear is by intratympanic injection.

12. CHIR99021 or a pharmaceutically acceptable salt thereof and valproic acid (VPA) or a pharmaceutically acceptable salt thereof for use according to any of claims 1-11, wherein the pharmaceutically acceptable salt of valproic acid is sodium valproate.

13. CHIR99021 or a pharmaceutically acceptable salt thereof and valproic acid (VPA) or a pharmaceutically acceptable salt thereof for use according to any of claims 1-12, wherein the CHIR99021 and the VPA are formulated as a single composition.

14. CHIR99021 or a pharmaceutically acceptable salt thereof and valproic acid (VPA) or a pharmaceutically acceptable salt thereof for use according to claim 13, wherein the composition comprises a Poloxamer 407.

15. CHIR99021 or a pharmaceutically acceptable salt thereof and valproic acid (VPA) or a pharmaceutically acceptable salt thereof for use according to any of claims 1-14, wherein the sensorineural hearing loss is sudden sensorineural hearing loss or noise-induced sensorineural hearing loss.

16. CHIR99021 or a pharmaceutically acceptable salt thereof and valproic acid (VPA) or a pharmaceutically acceptable salt thereof for use according to claim 15, wherein the sensorineural hearing loss is sudden sensorineural hearing loss.

17. CHIR99021 or a pharmaceutically acceptable salt thereof and valproic acid (VPA) or a pharmaceutically acceptable salt thereof for use according to claim 15, wherein the sensorineural hearing loss is noise-induced sensorineural hearing loss.

18. CHIR99021 or a pharmaceutically acceptable salt thereof and valproic acid (VPA) or a pharmaceutically acceptable salt thereof for use according to any of claims 1-17, wherein said treatment also provides treatment for tinnitus, when assessed using one or more measures selected from the group consisting of: tinnitus functional index (TFI), tinnitus handicap index (THI), tinnitus reaction questionnaire (TRQ), tinnitus severity index (TSI), and tinnitus handicap questionnaire (THQ).

19. CHIR99021 or a pharmaceutically acceptable salt thereof and valproic acid (VPA) or a pharmaceutically acceptable salt thereof for use according to any of claims 1-18, wherein said treatment also provides treatment for tinnitus, when assessed using tinnitus functional index (TFI).

20. CHIR99021 or a pharmaceutically acceptable salt thereof and valproic acid (VPA) or a pharmaceutically acceptable salt thereof for use in treating tinnitus in a human patient with sensorineural hearing loss, wherein the tinnitus is diagnosed using one or more of the methods selected from the group consisting of: tinnitus functional index (TFI), tinnitus handicap index (THI), tinnitus reaction questionnaire (TRQ), tinnitus severity index (TSI), and tinnitus handicap questionnaire (THQ).

21. CHIR99021 or a pharmaceutically acceptable salt thereof and valproic acid (VPA) or a pharmaceutically acceptable salt thereof for use according to claim 20, wherein the tinnitus is diagnosed using tinnitus functional index (TFI).

22. CHIR99021 or a pharmaceutically acceptable salt thereof and valproic acid (VPA) or a pharmaceutically acceptable salt thereof for use according to claim 20 or claim 21, wherein the sensorineural hearing loss is mild, moderate or moderately severe sensorineural hearing loss.

23. CHIR99021 or a pharmaceutically acceptable salt thereof and valproic acid (VPA) or a pharmaceutically acceptable salt thereof for use according to claims 20-22, wherein the average of the patient's hearing thresholds across 0.5 kHz, 1 kHz, 2 kHz and 4 kHz is greater than 25 dB HL and no more than 70 dB HL when measured by pure tone audiometry prior to the treatment.

24. CHIR99021 or a pharmaceutically acceptable salt thereof and valproic acid (VPA) or a pharmaceutically acceptable salt thereof for use according to any of claims 20-22, wherein the average of the patient's hearing thresholds across 0.5 kHz, 1 kHz, 2 kHz and 4 kHz is greater than 25 dB HL and no more than 40 dB HL when measured by pure tone audiometry prior to the treatment.

25. CHIR99021 or a pharmaceutically acceptable salt thereof and valproic acid (VPA) or a pharmaceutically acceptable salt thereof for use according to any of claims 20-22, wherein the average of the patient's hearing thresholds across 0.5 kHz, 1 kHz, 2 kHz and 4 kHz is greater than 40 dB HL and no more than 55 dB HL when measured by pure tone audiometry prior to the treatment.

26. CHIR99021 or a pharmaceutically acceptable salt thereof and valproic acid (VPA) or a pharmaceutically acceptable salt thereof for use according to any of claims 20-22, wherein the average of the patient's hearing thresholds across 0.5 kHz, 1 kHz, 2 kHz and 4 kHz is greater than 55 dB HL and no more than 70 dB HL when measured by pure tone audiometry prior to the treatment.

27. CHIR99021 or a pharmaceutically acceptable salt thereof and valproic acid (VPA) or a pharmaceutically acceptable salt thereof for use according to any of claims 20-26, wherein the sensorineural hearing loss is sudden sensorineural hearing loss or noise-induced sensorineural hearing loss.

28. CHIR99021 or a pharmaceutically acceptable salt thereof and valproic acid (VPA) or a pharmaceutically acceptable salt thereof for use according to claim 27, wherein the sensorineural hearing loss is sudden sensorineural hearing loss.

29. CHIR99021 or a pharmaceutically acceptable salt thereof and valproic acid (VPA) or a pharmaceutically acceptable salt thereof for use according to claim 27, wherein the sensorineural hearing loss is noise-induced sensorineural hearing loss.

30. A hair cell regeneration agent(s) for use in treating sensorineural hearing loss in a human patient, wherein the sensorineural hearing loss is moderate or moderately severe sensorineural hearing loss

31. A hair cell regeneration agent(s) for use in treating sensorineural hearing loss in a human patient, wherein the average of the patient's hearing thresholds across 0.5 kHz, 1 kHz, 2 kHz and 4 kHz is greater than 40 dB HL and no more than 70 dB HL when measured by pure tone audiometry prior to the treatment.

32. A hair cell regeneration agent(s) for use in treating sensorineural hearing loss in a human patient, wherein the patient has a standard word recognition score of 60% or less prior to the treatment.

33. A hair cell regeneration agent(s) for use in treating sensorineural hearing loss in a human patient, wherein the patient has a words-in-noise score of 50% or less prior to the treatment.

34. The hair cell regeneration agent(s) for use according to any of claims 30-33, wherein said treatment provides an improved standard word recognition score for the patient, wherein said improvement, if tested, would be at least 10%, wherein said percentage improvement is calculated using the following formula:
$$100 \times ( \frac{patient ’ s word recognition score after treatment}{patient ’ s word recognition score prior to treatment} - 1 )$$

35. The hair cell regeneration agent(s) for use according to any of claims 30-34, wherein said treatment provides an improved words-in-noise score for the patient, wherein said improvement, if tested, would be at least 10%, wherein said percentage improvement is calculated using the following formula:
$$100 \times ( \frac{patient ’ s words in noise score after treatment}{patient ’ s words in noise score prior to treatment} - 1 )$$
wherein said treatment optionally also provides the improvement specified in claim 34.

36. The hair cell regeneration agent(s) for use according to any of claims 30-35, wherein said treatment provides an improved hearing threshold at 8 kHz, wherein said improvement, if tested, would be at least 5 dB relative to the patient's hearing threshold at 8 kHz prior to the treatment, wherein said hearing threshold is measured by pure tone audiometry, wherein said treatment optionally also provides the improvement specified in claim 34 or claim 35.

37. A hair cell regeneration agent(s) for use in treating sensorineural hearing loss in a human patient, wherein said treatment provides an improved standard word recognition score for the patient, wherein said improvement, if tested, would be at least 10%, wherein said percentage improvement is calculated using the following formula:
$$100 \times ( \frac{patient ’ s word recognition score after treatment}{patient ’ s word recognition score prior to treatment} - 1 )$$

38. A hair cell regeneration agent(s) for use in treating sensorineural hearing loss in a human patient, wherein said treatment provides an improved words-in-noise score for the patient, wherein said improvement, if tested, would be at least 10%, wherein said percentage improvement is calculated using the following formula:
$$100 \times ( \frac{patient ’ s words in noise score after treatment}{patient ’ s words in noise score prior to treatment} - 1 )$$
wherein said treatment optionally also provides the improvement specified in claim 37.

39. A hair cell regeneration agent(s) for use in treating sensorineural hearing loss in a human patient, wherein said treatment provides an improved hearing threshold at 8 kHz, wherein said improvement, if tested, would be at least 5 dB relative to the patient's hearing threshold at 8 kHz prior to the treatment, wherein said hearing threshold is measured by pure tone audiometry, wherein said treatment optionally also provides the improvement specified in claim 37 or claim 38.

40. The hair cell regeneration agent(s) for use according to any of claims 30-39, wherein an improvement in hearing is provided within 90 days.

41. The hair cell regeneration agent(s) for use according to any of claims 30-40, wherein an improvement in hearing is provided by a single administration.

42. The hair cell regeneration agent(s) for use according to any of claims 30-41, wherein said compounds are administered to the middle ear.

43. The hair cell regeneration agent(s) for use according to claim 42 wherein administration to the middle ear is by intratympanic injection.

44. The hair cell regeneration agent(s) for use according to any of claims 30-43, wherein the hair cell regeneration agent(s) is a combination of agents formulated as a single composition.

45. The hair cell regeneration agent(s) for use according to any of claims 30-44, wherein said treatment also provides treatment for tinnitus, when assessed using one or more measures selected from the group consisting of: tinnitus functional index (TFI), tinnitus handicap index (THI), tinnitus reaction questionnaire (TRQ), tinnitus severity index (TSI), and tinnitus handicap questionnaire (THQ).

46. A hair cell regeneration agent(s) for use in treating tinnitus in a human patient with sensorineural hearing loss, wherein the tinnitus is diagnosed using one or more of the methods selected from the group consisting of; tinnitus functional index (TFI), tinnitus handicap index (THI), tinnitus reaction questionnaire (TRQ), tinnitus severity index (TSI), and tinnitus handicap questionnaire (THQ).

47. The hair cell regeneration agent(s) for use according to claim 47, wherein the sensorineural hearing loss is mild, moderate or moderately severe sensorineural hearing loss.
